SEQUENCE LISTING (282 sequences)

SEQ ID NO: 1 - protein, 397 aa, synthetic construct
MNSNKDKIKVIKVFEAFAGIGSQFKALKNIARSKNWEIQHSGMVEWFVDAIVSYVAIHSKNFNPKIERLDRDILSISNDSKMPISEYGIKKINNTIKASYLNYAKKHFNNLFDIKKVNKDNFPKNIDIFTYSFPCQDLSVQGLQKGIDKELNTRSGLLWEIERILEEIKNSFSKEEMPKYLLMENVKNLLSHKNKKNYNTWLKQLEKFGYKSKTYLLNSKNFDNCQNRERVFCLSIRDDYLEKTGFKFKELEKVKNPPKKIKDILVDSSNYKYLNLNKYETTTFRETKSNIISRPLKNYTTFNSENYVYNINGIGPTLTASGANSRIKIETQQGVRYLTPLECFKYMQFDVNDFKKVQSTNLISENKMIYIAGKSIPVKILEAIFNTLEFVNNEELE

SEQ ID NO: 2 - protein, 397 aa, synthetic construct
MNSNKDKIKVIKVFEAFAGIGSQFKALKNIARSKNWEIQHSGMVEWFVDAIVSYVAIHSKNFNPKIERLDRDILSISNDSKMPISEYGIKKINNTIKASYLNYAKKHFNNLFDIKKVNKDNFPKNIDIFTYSFPCQDLSVQGLQKGIDKELNTRSGLLWEIERILEEIKNSFSKEEMPKYLLMENVKNLLSHKNKKNYNTWLKQLEKFGYKSKTYLLNSKNFDNCQNRERVFCLSIRDDYLEKTGFKFKELEKVKNPPKKIKDILVDSSNYKYLNLNKYETTTFRETKSNIISRPLKNYTTFNSENYVYNINGIGPTLTASGANSRIKIETQQGVRYLTPLECFKYMQFDVNDFKKVQSTNLISENKMIYIAGRSIPVKILEAIFNTLEFVNNEELE

SEQ ID NO: 3 - protein, 507 aa, synthetic construct
GGSQSQNGKCEGCNPDKDEAPYYTHLGAGPDVAAIRTLMEERYGEKGKAIRIEKVIYTGKEGKSSQGCPIAKWVYRRSSEEEKLLCLVRVRPNHTCETAVMVIAIMLWDGIPKLLASELYSELTDILGKCGICTNRRCSQNETRNCCCQGENPETCGASFSFGCSWSMYYNGCKFARSKKPRKFRLHGAEPKEEERLGSHLQNLATVIAPIYKKLAPDAYNNQVEFEHQAPDCCLGLKEGRPFSGVTACLDFSAHSHRDQQNMPNGSTVVVTLNREDNREVGAKPEDEQFHVLPMYIIAPEDEFGSTEGQEKKIRMGSIEVLQSFRRRRVIRIGELPKSCEVSGQDAAAVQEIEYWSDSEHNFQDPCIGGVAIAPTHGSILIECAKCEVHATTKVNDPDRNHPTRISLVLYRHKNLFLPKHCLALWEAKMAEKARKEEECGKNGSDHVSQKNHGKQEKREPTGPQEPSYLRFIQSLAENTGSVTTDSTVTTSPYAFTQVTGPYNTFV

SEQ ID NO: 4 - protein, 871 aa, synthetic construct
QSQNGKCEGCNPDKDEAPYYTHLGAGPDVAAIRTLMEERYGEKGKAIRIEKVIYTGKEGKSSQGCPIAKWVYRRSSEEEKLLCLVRVRPNHTCETAVMVIAIMLWDGIPKLLASELYSELTDILGKCGICTNRRCSQNETRNCCCQGENPETCGASFSFGCSWSMYYNGCKFARSKKPRKFRLHGAEPKEEERLGSHLQNLATVIAPIYKKLAPDAYNNQVEFEHQAPDCCLGLKEGRPFSGVTACLDFSAHSHRDQQNMPNGSTVVVTLNREDNREVGAKPEDEQFHVLPMYIIAPEDEFGSTEGQEKKIRMGSIEVLQSFRRRRVIRIGELPKSCKKKAEPKKAKTKKAARKRSSLENCSSRTEKGKSSSHTKLMENASHMKQMTAQPQLSGPVIRQPPTLQRHLQQGQRPQQPQPPQPQPQTTPQPQPQPQHIMPGNSQSVGSHCSGSTSVYTRQPTPHSPYPSSAHTSDIYGDTNHVNFYPTSSHASGSYLNPSNYMNPYLGLLNQNNQYAPFPYNGSVPVDNGSPFLGSYSPQAQSRDLHRYPNQDHLTNQNLPPIHTLHQQTFGDSPSKYLSYGNQNMQRDAFTTNSTLKPNVHHLATFSPYPTPKMDSHFMGAASRSPYSHPHTDYKTSEHHLPSHTIYSYTAAASGSSSSHAFHNKENDNIANGLSRVLPGFNHDRTASAQELLYSLTGSSQEKQPEVSGQDAAAVQEIEYWSDSEHNFQDPCIGGVAIAPTHGSILIECAKCEVHATTKVNDPDRNHPTRISLVLYRHKNLFLPKHCLALWEAKMAEKARKEEECGKNGSDHVSQKNHGKQEKREPTGPQEPSYLRFIQSLAENTGSVTTDSTVTTSPYAFTQVTGPYNTFV

SEQ ID NO: 5 - protein, 474 aa, Homo sapiens
MDYKDDDDKHMGGSDFPSCRCVEQIIEKDEGPFYTHLGAGPNVAAIREIMEERFGQKGKAIRIERVIYTGKEGKSSQGCPIAKWVVRRSSSEEKLLCLVRERAGHTCEAAVIVILILVWEGIPLSLADKLYSELTETLRKYGTLTNRRCALNEERTCACQGLDPETCGASFSFGCSWSMYYNGCKFARSKIPRKFKLLGDDPKEEEKLESHLQNLSTLMAPTYKKLAPDAYNNQIEYEHRAPECRLGLKEGRPFSGVTACLDFCAHAHRDLHNMQNGSTLVCTLTREDNREFGGKPEDEQLHVLPLYKVSDVDEFGSVEAQEEKKRSGAIQVLSSFRRKVRMLAEPVKTCRQRKLEAKKAAAEKLSGGGGSGGGGSGGGGSDEVWSDSEQSFLDPDIGGVAVAPTHGSILIECAKRELHATTPLKNPNRNHPTRISLVFYQHKSMNEPKHGLALWEAKMAEKAREKEEECEKYG

SEQ ID NO: 6 - protein, 474 aa, synthetic construct
MDYKDDDDKHMGGSDFPSCRCVEQIIEKDEGPFYTHLGAGPNVAAIREIMEERFGQKGKAIRIERVIYTGKEGKSSQGCPIAKWVVRRSSSEEKLLCLVRERAGHTCEAAVIVILILVWEGIPLSLADKLYSELTETLRKYGTLTNRRCALNEERTCACQGLDPETCGASFSFGCSWSMYYNGCKFARSKIPRKFKLLGDDPKEEEKLESHLQNLSTLMAPTYKKLAPDAYNNQIEYEHRAPECRLGLKEGRPFSGVTACLDFCAHAHRDLHNMQNGSTLVCTLTREDNREFGGKPEDEQLHVLPLYKVSDVDEFGSVEAQEEKKRSGAIQVLSSFRRKVRMLAEPVKTCRQRKLEAKKAAAEKLSGGGGSGGGGSGGGGSDEVWSDSEQSFLDPDIGGVAVAPTHGSILIECAKRELHATTPLKNPNRNHPTRISLAFYQHKSMNEPKHGLALWEAKMAEKAREKEEECEKYG

SEQ ID NO: 7 - protein, 474 aa, synthetic construct
MDYKDDDDKHMGGSDFPSCRCVEQIIEKDEGPFYTHLGAGPNVAAIREIMEERFGQKGKAIRIERVIYTGKEGKSSQGCPIAKWVVRRSSSEEKLLCLVRERAGHTCEAAVIVILILVWEGIPLSLADKLYSELTETLRKYGTLTNRRCALNEERTCACQGLDPETCGASFSFGCSWSMYYNGCKFARSKIPRKFKLLGDDPKEEEKLESHLQNLSTLMAPTYKKLAPDAYNNQIEYEHRAPECRLGLKEGRPFSGVTACLDFCAHAHRDLHNMQNGSTLVCTLTREDNREFGGKPEDEQLHVLPLYKVSDVDEFGSVEAQEEKKRSGAIQVLSSFRRKVRMLAEPVKTCRQRKLEAKKAAAEKLSGGGGSGGGGSGGGGSDEVWSDSEQSFLDPDIGGVAVAPTHGSILIECAKRELHATTPLKNPNRNHPTRISLCFYQHKSMNEPKHGLALWEAKMAEKAREKEEECEKYG

SEQ ID NO: 8 - protein, 474 aa, synthetic construct
MDYKDDDDKHMGGSDFPSCRCVEQIIEKDEGPFYTHLGAGPNVAAIREIMEERFGQKGKAIRIERVIYTGKEGKSSQGCPIAKWVVRRSSSEEKLLCLVRERAGHTCEAAVIVILILVWEGIPLSLADKLYSELTETLRKYGTLTNRRCALNEERTCACQGLDPETCGASFSFGCSWSMYYNGCKFARSKIPRKFKLLGDDPKEEEKLESHLQNLSTLMAPTYKKLAPDAYNNQIEYEHRAPECRLGLKEGRPFSGVTACLDFCAHAHRDLHNMQNGSTLVCTLTREDNREFGGKPEDEQLHVLPLYKVSDVDEFGSVEAQEEKKRSGAIQVLSSFRRKVRMLAEPVKTCRQRKLEAKKAAAEKLSGGGGSGGGGSGGGGSDEVWSDSEQSFLDPDIGGVAVAPTHGSILIECAKRELHATTPLKNPNRNHPTRISLGFYQHKSMNEPKHGLALWEAKMAEKAREKEEECEKYG

SEQ ID NO: 9 - protein, 474 aa, synthetic construct
MDYKDDDDKHMGGSDFPSCRCVEQIIEKDEGPFYTHLGAGPNVAAIREIMEERFGQKGKAIRIERVIYTGKEGKSSQGCPIAKWVVRRSSSEEKLLCLVRERAGHTCEAAVIVILILVWEGIPLSLADKLYSELTETLRKYGTLTNRRCALNEERTCACQGLDPETCGASFSFGCSWSMYYNGCKFARSKIPRKFKLLGDDPKEEEKLESHLQNLSTLMAPTYKKLAPDAYNNQIEYEHRAPECRLGLKEGRPFSGVTACLDFCAHAHRDLHNMQNGSTLVCTLTREDNREFGGKPEDEQLHVLPLYKVSDVDEFGSVEAQEEKKRSGAIQVLSSFRRKVRMLAEPVKTCRQRKLEAKKAAAEKLSGGGGSGGGGSGGGGSDEVWSDSEQSFLDPDIGGVAVAPTHGSILIECAKRELHATTPLKNPNRNHPTRISLIFYQHKSMNEPKHGLALWEAKMAEKAREKEEECEKYG

SEQ ID NO: 10 - protein, 474 aa, synthetic construct
MDYKDDDDKHMGGSDFPSCRCVEQIIEKDEGPFYTHLGAGPNVAAIREIMEERFGQKGKAIRIERVIYTGKEGKSSQGCPIAKWVVRRSSSEEKLLCLVRERAGHTCEAAVIVILILVWEGIPLSLADKLYSELTETLRKYGTLTNRRCALNEERTCACQGLDPETCGASFSFGCSWSMYYNGCKFARSKIPRKFKLLGDDPKEEEKLESHLQNLSTLMAPTYKKLAPDAYNNQIEYEHRAPECRLGLKEGRPFSGVTACLDFCAHAHRDLHNMQNGSTLVCTLTREDNREFGGKPEDEQLHVLPLYKVSDVDEFGSVEAQEEKKRSGAIQVLSSFRRKVRMLAEPVKTCRQRKLEAKKAAAEKLSGGGGSGGGGSGGGGSDEVWSDSEQSFLDPDIGGVAVAPTHGSILIECAKRELHATTPLKNPNRNHPTRISLPFYQHKSMNEPKHGLALWEAKMAEKAREKEEECEKYG

SEQ ID NO: 11 - protein, 474 aa, synthetic construct
MDYKDDDDKHMGGSDFPSCRCVEQIIEKDEGPFYTHLGAGPNVAAIREIMEERFGQKGKAIRIERVIYTGKEGKSSQGCPIAKWVVRRSSSEEKLLCLVRERAGHTCEAAVIVILILVWEGIPLSLADKLYSELTETLRKYGTLTNRRCALNEERTCACQGLDPETCGASFSFGCSWSMYYNGCKFARSKIPRKFKLLGDDPKEEEKLESHLQNLSTLMAPTYKKLAPDAYNNQIEYEHRAPECRLGLKEGRPFSGVSACLDFCAHAHRDLHNMQNGSTLVCTLTREDNREFGGKPEDEQLHVLPLYKVSDVDEFGSVEAQEEKKRSGAIQVLSSFRRKVRMLAEPVKTCRQRKLEAKKAAAEKLSGGGGSGGGGSGGGGSDEVWSDSEQSFLDPDIGGVAVAPTHGSILIECAKRELHATTPLKNPNRNHPTRISLVFYQHKSMNEPKHGLALWEAKMAEKAREKEEECEKYG

SEQ ID NO: 12 - protein, 878 aa, synthetic construct
MGGSDFPSCRCVEQIIEKDEGPFYTHLGAGPNVAAIREIMEERFGQKGKAIRIERVIYTGKEGKSSQGCPIAKWVVRRSSSEEKLLCLVRERAGHTCEAAVIVILILVWEGIPLSLADKLYSELTETLRKYGTLTNRRCALNEERTCACQGLDPETCGASFSFGCSWSMYYNGCKFARSKIPRKFKLLGDDPKEEEKLESHLQNLSTLMAPTYKKLAPDAYNNQIEYEHRAPECRLGLKEGRPFSGVSACLDFCAHAHRDLHNMQNGSTLVCTLTREDNREFGGKPEDEQLHVLPLYKVSDVDEFGSVEAQEEKKRSGAIQVLSSFRRKVRMLAEPVKTCRQRKLEAKKAAAEKLSSLENSSNKNEKEKSAPSRTKQTENASQAKQLAELLRLSGPVMQQSQQPQPLQKQPPQPQQQQRPQQQQPHHPQTESVNSYSASGSTNPYMRRPNPVSPYPNSSHTSDIYGSTSPMNFYSTSSQAAGSYLNSSNPMNPYPGLLNQNTQYPSYQCNGNLSVDNCSPYLGSYSPQSQPMDLYRYPSQDPLSKLSLPPIHTLYQPRFGNSQSFTSKYLGYGNQNMQGDGFSSCTIRPNVHHVGKLPPYPTHEMDGHFMGATSRLPPNLSNPNMDYKNGEHHSPSHIIHNYSAAPGMFNSSLHALHLQNKENDMLSHTANGLSKMLPALNHDRTACVQGGLHKLSDANGQEKQPLALVQGVASGAEDNDEVWSDSEQSFLDPDIGGVAVAPTHGSILIECAKRELHATTPLKNPNRNHPTRISLVFYQHKSMNEPKHGLALWEAKMAEKAREKEEECEKYGPDYVPQKSHGKKVKREPAEPHETSEPTYLRFIKSLAERTMSVTTDSTVTTSPYAFTRVTGPYNRYI

SEQ ID NO: 13 - protein, 9 aa, synthetic construct
FSGVTACLD

SEQ ID NO: 14 - protein, 9 aa, synthetic construct
FSGVSACLD

SEQ ID NO: 15 - DNA, 20 bp, synthetic construct
  source 1..20: mol_type = other DNA
actccgccgtgtatctctgt

SEQ ID NO: 16 - DNA, 20 bp, synthetic construct
  source 1..20: mol_type = other DNA
actctgccgtgtatctctgt

SEQ ID NO: 17 - DNA, 20 bp, synthetic construct
  source 1..20: mol_type = other DNA
actcggccgtgtatctctgt

SEQ ID NO: 18 - DNA, 20 bp, synthetic construct
  source 1..20: mol_type = other DNA
actcagccgtgtatctctgt

SEQ ID NO: 19 - DNA, 20 bp, synthetic construct
  source 1..20: mol_type = other DNA
actcagctgtgtatctctgt

SEQ ID NO: 20 - DNA, 20 bp, synthetic construct
  source 1..20: mol_type = other DNA
actcagccgtgtatctccgt

SEQ ID NO: 21 - DNA, 20 bp, synthetic construct
  source 1..20: mol_type = other DNA
actcagccgcgtatctccgt

SEQ ID NO: 22 - DNA, 20 bp, synthetic construct
  source 1..20: mol_type = other DNA
actcggccatgtatcgctgt

SEQ ID NO: 23 - DNA, 20 bp, synthetic construct
  source 1..20: mol_type = other DNA
actcagccatgtatcgctgt

SEQ ID NO: 24 - DNA, 20 bp, synthetic construct
  source 1..20: mol_type = other DNA
actcggccatgtatctctgt

SEQ ID NO: 25 - DNA, 20 bp, synthetic construct
  source 1..20: mol_type = other DNA
actcagccgtgtatctctac

SEQ ID NO: 26 - DNA, 20 bp, synthetic construct
  source 1..20: mol_type = other DNA
actcagccctgtatctctgc

SEQ ID NO: 27 - DNA, 20 bp, synthetic construct
  source 1..20: mol_type = other DNA
acttggctgtgtatctctgt

SEQ ID NO: 28 - DNA, 20 bp, synthetic construct
  source 1..20: mol_type = other DNA
actcggccctgtatctctgc

SEQ ID NO: 29 - DNA, 20 bp, synthetic construct
  source 1..20: mol_type = other DNA
actcggccctgtatctctgt

SEQ ID NO: 30 - DNA, 20 bp, synthetic construct
  source 1..20: mol_type = other DNA
actcggccctctatctctgt

SEQ ID NO: 31 - DNA, 20 bp, synthetic construct
  source 1..20: mol_type = other DNA
actcggccctttatctttgc

SEQ ID NO: 32 - DNA, 20 bp, synthetic construct
  source 1..20: mol_type = other DNA
acacggcactgtatctctgc

SEQ ID NO: 33 - DNA, 20 bp, synthetic construct
  source 1..20: mol_type = other DNA
actcagctgtgtacttctgt

SEQ ID NO: 34 - DNA, 20 bp, synthetic construct
  source 1..20: mol_type = other DNA
actcagctgtgtatttttgt

SEQ ID NO: 35 - DNA, 20 bp, synthetic construct
  source 1..20: mol_type = other DNA
actcagctttgtatttctgt

SEQ ID NO: 36 - DNA, 20 bp, synthetic construct
  source 1..20: mol_type = other DNA
actctgctgtgtatttctgt

SEQ ID NO: 37 - DNA, 20 bp, synthetic construct
  source 1..20: mol_type = other DNA
actcagccatgtacttctgt

SEQ ID NO: 38 - DNA, 20 bp, synthetic construct
  source 1..20: mol_type = other DNA
actcagccctgtacttctgt

SEQ ID NO: 39 - DNA, 20 bp, synthetic construct
  source 1..20: mol_type = other DNA
actctggcttctatctctgt

SEQ ID NO: 40 - DNA, 20 bp, synthetic construct
  source 1..20: mol_type = other DNA
acgcagccatgtacctgtgt

SEQ ID NO: 41 - DNA, 20 bp, synthetic construct
  source 1..20: mol_type = other DNA
acacagccatgtacctgtgt

SEQ ID NO: 42 - DNA, 20 bp, synthetic construct
  source 1..20: mol_type = other DNA
acgcagccatgtaccagtgt

SEQ ID NO: 43 - DNA, 20 bp, synthetic construct
  source 1..20: mol_type = other DNA
attcggcagcttatttctgt

SEQ ID NO: 44 - DNA, 20 bp, synthetic construct
  source 1..20: mol_type = other DNA
attctggagtttatttctgt

SEQ ID NO: 45 - DNA, 20 bp, synthetic construct
  source 1..20: mol_type = other DNA
attcagcagtgtatttttgt

SEQ ID NO: 46 - DNA, 20 bp, synthetic construct
  source 1..20: mol_type = other DNA
agacatctgtgtacttctgt

SEQ ID NO: 47 - DNA, 20 bp, synthetic construct
  source 1..20: mol_type = other DNA
agacatctgtgtacttgtgt

SEQ ID NO: 48 - DNA, 20 bp, synthetic construct
  source 1..20: mol_type = other DNA
aaacatctgtgtacttctgt

SEQ ID NO: 49 - DNA, 20 bp, synthetic construct
  source 1..20: mol_type = other DNA
agacatctgtatacttctgt

SEQ ID NO: 50 - DNA, 20 bp, synthetic construct
  source 1..20: mol_type = other DNA
agacttctgtttacttctgt

SEQ ID NO: 51 - DNA, 20 bp, synthetic construct
  source 1..20: mol_type = other DNA
agacctctctgtacttctgt

SEQ ID NO: 52 - DNA, 20 bp, synthetic construct
  source 1..20: mol_type = other DNA
agacatctatgtacctctgt

SEQ ID NO: 53 - DNA, 20 bp, synthetic construct
  source 1..20: mol_type = other DNA
agacagctctttacttctgt

SEQ ID NO: 54 - DNA, 20 bp, synthetic construct
  source 1..20: mol_type = other DNA
agacatctgtatatttctgc

SEQ ID NO: 55 - DNA, 20 bp, synthetic construct
  source 1..20: mol_type = other DNA
agacatctgtgtatttctgc

SEQ ID NO: 56 - DNA, 20 bp, synthetic construct
  source 1..20: mol_type = other DNA
atacctctcagtacctctgt

SEQ ID NO: 57 - DNA, 20 bp, synthetic construct
  source 1..20: mol_type = other DNA
cgacagctttctatctctgt

SEQ ID NO: 58 - DNA, 20 bp, synthetic construct
  source 1..20: mol_type = other DNA
gcttctacatctgcagtgct

SEQ ID NO: 59 - DNA, 20 bp, synthetic construct
  source 1..20: mol_type = other DNA
acagcagcttctacatctgc

SEQ ID NO: 60 - DNA, 20 bp, synthetic construct
  source 1..20: mol_type = other DNA
acagcagcatatatctctgc

SEQ ID NO: 61 - DNA, 20 bp, synthetic construct
  source 1..20: mol_type = other DNA
cccgctcccagacatctgtg

SEQ ID NO: 62 - DNA, 20 bp, synthetic construct
  source 1..20: mol_type = other DNA
actcggccgtgtatctctgt

SEQ ID NO: 63 - DNA, 20 bp, synthetic construct
  source 1..20: mol_type = other DNA
actcagctgtgtatctctgt

SEQ ID NO: 64 - DNA, 20 bp, synthetic construct
  source 1..20: mol_type = other DNA
actcagccgcgtatctccgt

SEQ ID NO: 65 - DNA, 20 bp, synthetic construct
  source 1..20: mol_type = other DNA
actcagccatgtatcgctgt

SEQ ID NO: 66 - DNA, 20 bp, synthetic construct
  source 1..20: mol_type = other DNA
actcagccgtgtatctctac

SEQ ID NO: 67 - DNA, 20 bp, synthetic construct
  source 1..20: mol_type = other DNA
actcagccctgtatctctgc

SEQ ID NO: 68 - DNA, 20 bp, synthetic construct
  source 1..20: mol_type = other DNA
acttggctgtgtatctctgt

SEQ ID NO: 69 - DNA, 20 bp, synthetic construct
  source 1..20: mol_type = other DNA
actcggccctctatctctgt

SEQ ID NO: 70 - DNA, 20 bp, synthetic construct
  source 1..20: mol_type = other DNA
actcggccctttatctttgc

SEQ ID NO: 71 - DNA, 20 bp, synthetic construct
  source 1..20: mol_type = other DNA
acacggcactgtatctctgc

SEQ ID NO: 72 - DNA, 20 bp, synthetic construct
  source 1..20: mol_type = other DNA
actcagctgtgtacttctgt

SEQ ID NO: 73 - DNA, 20 bp, synthetic construct
  source 1..20: mol_type = other DNA
actcagctgtgtatttttgt

SEQ ID NO: 74 - DNA, 20 bp, synthetic construct
  source 1..20: mol_type = other DNA
actcagctttgtatttctgt

SEQ ID NO: 75 - DNA, 20 bp, synthetic construct
  source 1..20: mol_type = other DNA
actctgctgtgtatttctgt

SEQ ID NO: 76 - DNA, 20 bp, synthetic construct
  source 1..20: mol_type = other DNA
actcagccctgtacttctgt

SEQ ID NO: 77 - DNA, 20 bp, synthetic construct
  source 1..20: mol_type = other DNA
actctggcttctatctctgt

SEQ ID NO: 78 - DNA, 20 bp, synthetic construct
  source 1..20: mol_type = other DNA
acgcagccatgtacctgtgt

SEQ ID NO: 79 - DNA, 20 bp, synthetic construct
  source 1..20: mol_type = other DNA
acgcagccatgtaccagtgt

SEQ ID NO: 80 - DNA, 20 bp, synthetic construct
  source 1..20: mol_type = other DNA
attcggcagcttatttctgt

SEQ ID NO: 81 - DNA, 20 bp, synthetic construct
  source 1..20: mol_type = other DNA
attctggagtttatttctgt

SEQ ID NO: 82 - DNA, 20 bp, synthetic construct
  source 1..20: mol_type = other DNA
attcagcagtgtatttttgt

SEQ ID NO: 83 - DNA, 20 bp, synthetic construct
  source 1..20: mol_type = other DNA
agacatctgtgtacttctgt

SEQ ID NO: 84 - DNA, 20 bp, synthetic construct
  source 1..20: mol_type = other DNA
agacatctgtgtacttgtgt

SEQ ID NO: 85 - DNA, 20 bp, synthetic construct
  source 1..20: mol_type = other DNA
agacttctgtttacttctgt

SEQ ID NO: 86 - DNA, 20 bp, synthetic construct
  source 1..20: mol_type = other DNA
agacctctctgtacttctgt

SEQ ID NO: 87 - DNA, 20 bp, synthetic construct
  source 1..20: mol_type = other DNA
agacatctatgtacctctgt

SEQ ID NO: 88 - DNA, 20 bp, synthetic construct
  source 1..20: mol_type = other DNA
agacagctctttacttctgt

SEQ ID NO: 89 - DNA, 20 bp, synthetic construct
  source 1..20: mol_type = other DNA
agacatctgtgtatttctgc

SEQ ID NO: 90 - DNA, 20 bp, synthetic construct
  source 1..20: mol_type = other DNA
atacctctcagtacctctgt

SEQ ID NO: 91 - DNA, 20 bp, synthetic construct
  source 1..20: mol_type = other DNA
cgacagctttctatctctgt

SEQ ID NO: 92 - DNA, 20 bp, synthetic construct
  source 1..20: mol_type = other DNA
gcttctacatctgcagtgct

SEQ ID NO: 93 - DNA, 20 bp, synthetic construct
  source 1..20: mol_type = other DNA
acagcagcttctacatctgc

SEQ ID NO: 94 - DNA, 20 bp, synthetic construct
  source 1..20: mol_type = other DNA
acagcagcatatatctctgc

SEQ ID NO: 95 - DNA, 20 bp, synthetic construct
  source 1..20: mol_type = other DNA
cccgctcccagacatctgtg

SEQ ID NO: 96 - DNA, 20 bp, synthetic construct
  source 1..20: mol_type = other DNA
  modified_base 5: mod_base = i
  modified_base 8..9: mod_base = i
actcngcnntgtatctctgt

SEQ ID NO: 97 - DNA, 20 bp, synthetic construct
  source 1..20: mol_type = other DNA
  modified_base 10: mod_base = i
actcagccgngtatctccgt

SEQ ID NO: 98 - DNA, 20 bp, synthetic construct
  source 1..20: mol_type = other DNA
actcggccatgtatcgctgt

SEQ ID NO: 99 - DNA, 20 bp, synthetic construct
  source 1..20: mol_type = other DNA
actcagccgtgtatctctac

SEQ ID NO: 100 - DNA, 20 bp, synthetic construct
  source 1..20: mol_type = other DNA
  modified_base 5: mod_base = i
  modified_base 11: mod_base = i
  modified_base 17: mod_base = i
actcngccctntatctntgc

SEQ ID NO: 101 - DNA, 20 bp, synthetic construct
  source 1..20: mol_type = other DNA
acacggcactgtatctctgc

SEQ ID NO: 102 - DNA, 20 bp, synthetic construct
  source 1..20: mol_type = other DNA
actcagctgtgtatttttgt

SEQ ID NO: 103 - DNA, 20 bp, synthetic construct
  source 1..20: mol_type = other DNA
actctggcttctatctctgt

SEQ ID NO: 104 - DNA, 20 bp, synthetic construct
  source 1..20: mol_type = other DNA
  modified_base 3: mod_base = i
  modified_base 16..17: mod_base = i
acncagccatgtaccnntgt

SEQ ID NO: 105 - DNA, 20 bp, synthetic construct
  source 1..20: mol_type = other DNA
  modified_base 10..11: mod_base = i
  modified_base 17: mod_base = i
attcggcagnntatttntgt

SEQ ID NO: 106 - DNA, 20 bp, synthetic construct
  source 1..20: mol_type = other DNA
attctggagtttatttctgt

SEQ ID NO: 107 - DNA, 20 bp, synthetic construct
  source 1..20: mol_type = other DNA
  modified_base 9: mod_base = i
  modified_base 14: mod_base = i
  modified_base 16: mod_base = i
agacatctntgtacntntgt

SEQ ID NO: 108 - DNA, 20 bp, synthetic construct
  source 1..20: mol_type = other DNA
  modified_base 5..6: mod_base = i
  modified_base 9: mod_base = i
agacnnctntttacttctgt

SEQ ID NO: 109 - DNA, 20 bp, synthetic construct
  source 1..20: mol_type = other DNA
  modified_base 8..9: mod_base = i
actcagcnntgtacttctgt

SEQ ID NO: 110 - DNA, 20 bp, synthetic construct
  source 1..20: mol_type = other DNA
  modified_base 11: mod_base = i
agacatctgtntatttctgc

SEQ ID NO: 111 - DNA, 20 bp, synthetic construct
  source 1..20: mol_type = other DNA
atacctctcagtacctctgt

SEQ ID NO: 112 - DNA, 20 bp, synthetic construct
  source 1..20: mol_type = other DNA
cgacagctttctatctctgt

SEQ ID NO: 113 - DNA, 20 bp, synthetic construct
  source 1..20: mol_type = other DNA
gcttctacatctgcagtgct

SEQ ID NO: 114 - DNA, 20 bp, synthetic construct
  source 1..20: mol_type = other DNA
  modified_base 11: mod_base = i
  modified_base 14..15: mod_base = i
acagcagcttntanntctgc

SEQ ID NO: 115 - DNA, 20 bp, synthetic construct
  source 1..20: mol_type = other DNA
cccgctcccagacatctgtg

SEQ ID NO: 116 - DNA, 20 bp, synthetic construct
  source 1..20: mol_type = other DNA
atgctgctgtgtactactgc

SEQ ID NO: 117 - DNA, 20 bp, synthetic construct
  source 1..20: mol_type = other DNA
actcagccacctacttctgt

SEQ ID NO: 118 - DNA, 20 bp, synthetic construct
  source 1..20: mol_type = other DNA
actcagctatctacttctgt

SEQ ID NO: 119 - DNA, 20 bp, synthetic construct
  source 1..20: mol_type = other DNA
atgctgctgagtacttctgt

SEQ ID NO: 120 - DNA, 20 bp, synthetic construct
  source 1..20: mol_type = other DNA
atgcgtctgagtacttctgt

SEQ ID NO: 121 - DNA, 20 bp, synthetic construct
  source 1..20: mol_type = other DNA
atacaggccactacctctgt

SEQ ID NO: 122 - DNA, 20 bp, synthetic construct
  source 1..20: mol_type = other DNA
actccgctgtgtacttctgt

SEQ ID NO: 123 - DNA, 20 bp, synthetic construct
  source 1..20: mol_type = other DNA
actcagccacctacctctgt

SEQ ID NO: 124 - DNA, 20 bp, synthetic construct
  source 1..20: mol_type = other DNA
atacaggcctctacctctgt

SEQ ID NO: 125 - DNA, 20 bp, synthetic construct
  source 1..20: mol_type = other DNA
actctgccgtgtactactgc

SEQ ID NO: 126 - DNA, 20 bp, synthetic construct
  source 1..20: mol_type = other DNA
actcagctgtctacttttgt

SEQ ID NO: 127 - DNA, 20 bp, synthetic construct
  source 1..20: mol_type = other DNA
actcagcaatgtatttctgt

SEQ ID NO: 128 - DNA, 20 bp, synthetic construct
  source 1..20: mol_type = other DNA
atgtaggaacctacttctgt

SEQ ID NO: 129 - DNA, 20 bp, synthetic construct
  source 1..20: mol_type = other DNA
attcagccacctatttctgt

SEQ ID NO: 130 - DNA, 20 bp, synthetic construct
  source 1..20: mol_type = other DNA
atgcaggcatctacctctgt

SEQ ID NO: 131 - DNA, 20 bp, synthetic construct
  source 1..20: mol_type = other DNA
acacagctgagtacttctgt

SEQ ID NO: 132 - DNA, 20 bp, synthetic construct
  source 1..20: mol_type = other DNA
attcagccacatacctctgt

SEQ ID NO: 133 - DNA, 20 bp, synthetic construct
  source 1..20: mol_type = other DNA
atgtaggcatctacttctgt

SEQ ID NO: 134 - DNA, 20 bp, synthetic construct
  source 1..20: mol_type = other DNA
acgcggctgagtacttctgt

SEQ ID NO: 135 - DNA, 20 bp, synthetic construct
  source 1..20: mol_type = other DNA
actcaggcgtttatttctgt

SEQ ID NO: 136 - DNA, 20 bp, synthetic construct
  source 1..20: mol_type = other DNA
actcggccgtctacctctgt

SEQ ID NO: 137 - DNA, 20 bp, synthetic construct
  source 1..20: mol_type = other DNA
acactgcgatgtatttctgt

SEQ ID NO: 138 - DNA, 20 bp, synthetic construct
  source 1..20: mol_type = other DNA
actcagcaatgtacttctgt

SEQ ID NO: 139 - DNA, 20 bp, synthetic construct
  source 1..20: mol_type = other DNA
actcagcagtatacttctgt

SEQ ID NO: 140 - DNA, 20 bp, synthetic construct
  source 1..20: mol_type = other DNA
actcagccgtgtactactgt

SEQ ID NO: 141 - DNA, 20 bp, synthetic construct
  source 1..20: mol_type = other DNA
acacggctgagtacttctgt

SEQ ID NO: 142 - DNA, 20 bp, synthetic construct
  source 1..20: mol_type = other DNA
actcagccacatacctctgt

SEQ ID NO: 143 - DNA, 20 bp, synthetic construct
  source 1..20: mol_type = other DNA
actcagctacctacctctgt

SEQ ID NO: 144 - DNA, 20 bp, synthetic construct
  source 1..20: mol_type = other DNA
actctgcagtgtacttctgt

SEQ ID NO: 145 - DNA, 20 bp, synthetic construct
  source 1..20: mol_type = other DNA
acactgctgtgtactattgc

SEQ ID NO: 146 - DNA, 20 bp, synthetic construct
  source 1..20: mol_type = other DNA
actcggccatctacctctgt

SEQ ID NO: 147 - DNA, 20 bp, synthetic construct
  source 1..20: mol_type = other DNA
actctgcctcttacttctgc

SEQ ID NO: 148 - DNA, 20 bp, synthetic construct
  source 1..20: mol_type = other DNA
tctctgccacctacttctgt

SEQ ID NO: 149 - DNA, 20 bp, synthetic construct
  source 1..20: mol_type = other DNA
actctgcctcttacctctgt

SEQ ID NO: 150 - DNA, 20 bp, synthetic construct
  source 1..20: mol_type = other DNA
acactgctgtgtactactgc

SEQ ID NO: 151 - DNA, 20 bp, synthetic construct
  source 1..20: mol_type = other DNA
actccgctttgtacttctgt

SEQ ID NO: 152 - DNA, 20 bp, synthetic construct
  source 1..20: mol_type = other DNA
actcagccacttatctctgt

SEQ ID NO: 153 - DNA, 20 bp, synthetic construct
  source 1..20: mol_type = other DNA
atgctgctgtttactactgt

SEQ ID NO: 154 - DNA, 20 bp, synthetic construct
  source 1..20: mol_type = other DNA
acccggctgagtacttctgt

SEQ ID NO: 155 - DNA, 20 bp, synthetic construct
  source 1..20: mol_type = other DNA
attcagccacctacctctgt

SEQ ID NO: 156 - DNA, 20 bp, synthetic construct
  source 1..20: mol_type = other DNA
actctgccgtctacatctgt

SEQ ID NO: 157 - DNA, 20 bp, synthetic construct
  source 1..20: mol_type = other DNA
actcaggaacctacttctgc

SEQ ID NO: 158 - DNA, 20 bp, synthetic construct
  source 1..20: mol_type = other DNA
atgccgcgatgtatttctgt

SEQ ID NO: 159 - DNA, 20 bp, synthetic construct
  source 1..20: mol_type = other DNA
attcagcctcctacatctgt

SEQ ID NO: 160 - DNA, 20 bp, synthetic construct
  source 1..20: mol_type = other DNA
actcggctgtctacttctgt

SEQ ID NO: 161 - DNA, 20 bp, synthetic construct
  source 1..20: mol_type = other DNA
acactgcttcttacttctgt

SEQ ID NO: 162 - DNA, 20 bp, synthetic construct
  source 1..20: mol_type = other DNA
actcagcggtgtacttctgt

SEQ ID NO: 163 - DNA, 20 bp, synthetic construct
  source 1..20: mol_type = other DNA
actcagccatgtattactgt

SEQ ID NO: 164 - DNA, 20 bp, synthetic construct
  source 1..20: mol_type = other DNA
actccgctgtgtacttctgt

SEQ ID NO: 165 - DNA, 20 bp, synthetic construct
  source 1..20: mol_type = other DNA
actcagcggtgtacttctgt

SEQ ID NO: 166 - DNA, 20 bp, synthetic construct
  source 1..20: mol_type = other DNA
actcggccgtctacctctgt

SEQ ID NO: 167 - DNA, 20 bp, synthetic construct
  source 1..20: mol_type = other DNA
actcagccacctacctctgt

SEQ ID NO: 168 - DNA, 20 bp, synthetic construct
  source 1..20: mol_type = other DNA
acgcggctgagtacttctgt

SEQ ID NO: 169 - DNA, 20 bp, synthetic construct
  source 1..20: mol_type = other DNA
actcagcagtatacttctgt

SEQ ID NO: 170 - DNA, 20 bp, synthetic construct
  source 1..20: mol_type = other DNA
actcagctatctacttctgt

SEQ ID NO: 171 - DNA, 20 bp, synthetic construct
  source 1..20: mol_type = other DNA
actctgccgtctacatctgt

SEQ ID NO: 172 - DNA, 20 bp, synthetic construct
  source 1..20: mol_type = other DNA
actcagcaatgtacttctgt

SEQ ID NO: 173 - DNA, 20 bp, synthetic construct
  source 1..20: mol_type = other DNA
actctgccgtgtactactgc

SEQ ID NO: 174 - DNA, 20 bp, synthetic construct
  source 1..20: mol_type = other DNA
acactgcttcttacttctgt

SEQ ID NO: 175 - DNA, 20 bp, synthetic construct
  source 1..20: mol_type = other DNA
attcagccacctatttctgt

SEQ ID NO: 176 - DNA, 20 bp, synthetic construct
  source 1..20: mol_type = other DNA
actctgcctcttacctctgt

SEQ ID NO: 177 - DNA, 20 bp, synthetic construct
  source 1..20: mol_type = other DNA
actcggctgtctacttctgt

SEQ ID NO: 178 - DNA, 20 bp, synthetic construct
  source 1..20: mol_type = other DNA
atacaggcctctacctctgt

SEQ ID NO: 179 - DNA, 20 bp, synthetic construct
  source 1..20: mol_type = other DNA
actcagccgtgtactactgt

SEQ ID NO: 180 - DNA, 20 bp, synthetic construct
  source 1..20: mol_type = other DNA
acactgcgatgtatttctgt

SEQ ID NO: 181 - DNA, 20 bp, synthetic construct
  source 1..20: mol_type = other DNA
atgctgctgtgtactactgc

SEQ ID NO: 182 - DNA, 20 bp, synthetic construct
  source 1..20: mol_type = other DNA
attcagcctcctacatctgt

SEQ ID NO: 183 - DNA, 20 bp, synthetic construct
  source 1..20: mol_type = other DNA
actctgcagtgtacttctgt

SEQ ID NO: 184 - DNA, 20 bp, synthetic construct
  source 1..20: mol_type = other DNA
atgccgcgatgtatttctgt

SEQ ID NO: 185 - DNA, 20 bp, synthetic construct
  source 1..20: mol_type = other DNA
atgctgctgtttactactgt

SEQ ID NO: 186 - DNA, 20 bp, synthetic construct
  source 1..20: mol_type = other DNA
actcagccacttatctctgt

SEQ ID NO: 187 - DNA, 20 bp, synthetic construct
  source 1..20: mol_type = other DNA
acactgctgtgtactactgc

SEQ ID NO: 188 - DNA, 20 bp, synthetic construct
  source 1..20: mol_type = other DNA
atgcaggcatctacctctgt

SEQ ID NO: 189 - DNA, 20 bp, synthetic construct
  source 1..20: mol_type = other DNA
actcagccatgtattactgt

SEQ ID NO: 190 - DNA, 20 bp, synthetic construct
  source 1..20: mol_type = other DNA
actctgcctcttacttctgc

SEQ ID NO: 191 - DNA, 20 bp, synthetic construct
  source 1..20: mol_type = other DNA
atgtaggcatctacttctgt

SEQ ID NO: 192 - DNA, 20 bp, synthetic construct
  source 1..20: mol_type = other DNA
acactgctgtgtactattgc

SEQ ID NO: 193 - DNA, 20 bp, synthetic construct
  source 1..20: mol_type = other DNA
actcaggcgtttatttctgt

SEQ ID NO: 194 - DNA, 20 bp, synthetic construct
  source 1..20: mol_type = other DNA
actcagctgtctacttttgt

SEQ ID NO: 195 - DNA, 20 bp, synthetic construct
  source 1..20: mol_type = other DNA
actcaggaacctacttctgc

SEQ ID NO: 196 - DNA, 20 bp, synthetic construct
  source 1..20: mol_type = other DNA
atgcgtctgagtacttctgt

SEQ ID NO: 197 - DNA, 20 bp, synthetic construct
  source 1..20: mol_type = other DNA
atgctgctgagtacttctgt

SEQ ID NO: 198 - DNA, 20 bp, synthetic construct
  source 1..20: mol_type = other DNA
atgtaggaacctacttctgt

SEQ ID NO: 199 - DNA, 20 bp, synthetic construct
  source 1..20: mol_type = other DNA
acacagctgagtacttctgt

SEQ ID NO: 200 - DNA, 20 bp, synthetic construct
  source 1..20: mol_type = other DNA
tctctgccacctacttctgt

SEQ ID NO: 201 - DNA, 20 bp, synthetic construct
  source 1..20: mol_type = other DNA
attcagccacatacctctgt

SEQ ID NO: 202 - DNA, 20 bp, synthetic construct
  source 1..20: mol_type = other DNA
  modified_base 5: mod_base = i
  modified_base 8: mod_base = i
  modified_base 11: mod_base = i
actcngcngtntacttctgt

SEQ ID NO: 203 - DNA, 20 bp, synthetic construct
  source 1..20: mol_type = other DNA
  modified_base 10..11: mod_base = i
  modified_base 15: mod_base = i
actcagccanntacntctgt

SEQ ID NO: 204 - DNA, 20 bp, synthetic construct
  source 1..20: mol_type = other DNA
  modified_base 3: mod_base = i
  modified_base 5: mod_base = i
acncngctgagtacttctgt

SEQ ID NO: 205 - DNA, 20 bp, synthetic construct
  source 1..20: mol_type = other DNA
  modified_base 9: mod_base = i
  modified_base 14: mod_base = i
  modified_base 16: mod_base = i
actcagccntgtantnctgt

SEQ ID NO: 206 - DNA, 20 bp, synthetic construct
  source 1..20: mol_type = other DNA
  modified_base 10..11: mod_base = i
  modified_base 16: mod_base = i
actctgccgnntactnctgc

SEQ ID NO: 207 - DNA, 20 bp, synthetic construct
  source 1..20: mol_type = other DNA
  modified_base 3: mod_base = i
  modified_base 9..10: mod_base = i
atncaggcnnctacctctgt

SEQ ID NO: 208 - DNA, 20 bp, synthetic construct
  source 1..20: mol_type = other DNA
  modified_base 2..3: mod_base = i
  modified_base 17: mod_base = i
annctgctgtgtactantgc

SEQ ID NO: 209 - DNA, 20 bp, synthetic construct
  source 1..20: mol_type = other DNA
  modified_base 9: mod_base = i
  modified_base 14..15: mod_base = i
attcagccncctanntctgt

SEQ ID NO: 210 - DNA, 20 bp, synthetic construct
  source 1..20: mol_type = other DNA
acactgcgatgtatttctgt

SEQ ID NO: 211 - DNA, 20 bp, synthetic construct
  source 1..20: mol_type = other DNA
  modified_base 8: mod_base = i
  modified_base 10: mod_base = i
atgtaggnanctacttctgt

SEQ ID NO: 212 - DNA, 20 bp, synthetic construct
  source 1..20: mol_type = other DNA
actcaggcgtttatttctgt

SEQ ID NO: 213 - DNA, 20 bp, synthetic construct
  source 1..20: mol_type = other DNA
actctgcctcttacctctgt

SEQ ID NO: 214 - DNA, 20 bp, synthetic construct
  source 1..20: mol_type = other DNA
actcagctgtctacttttgt

SEQ ID NO: 215 - DNA, 20 bp, synthetic construct
  source 1..20: mol_type = other DNA
atgccgcgatgtatttctgt

SEQ ID NO: 216 - DNA, 20 bp, synthetic construct
  source 1..20: mol_type = other DNA
actcaggaacctacttctgc

SEQ ID NO: 217 - DNA, 20 bp, synthetic construct
  source 1..20: mol_type = other DNA
atgcgtctgagtacttctgt

SEQ ID NO: 218 - DNA, 20 bp, synthetic construct
  source 1..20: mol_type = other DNA
acactgcttcttacttctgt

SEQ ID NO: 219 - DNA, 20 bp, synthetic construct
  source 1..20: mol_type = other DNA
tctctgccacctacttctgt

SEQ ID NO: 220 - DNA, 20 bp, synthetic construct
  source 1..20: mol_type = other DNA
atgctgctgtttactactgt

SEQ ID NO: 221 - DNA, 20 bp, synthetic construct
  source 1..20: mol_type = other DNA
atgaggtggtgtaccactgt

SEQ ID NO: 222 - DNA, 20 bp, synthetic construct
  source 1..20: mol_type = other DNA
attctgggttctattactgt

SEQ ID NO: 223 - DNA, 20 bp, synthetic construct
  source 1..20: mol_type = other DNA
actttggggtctattactgt

SEQ ID NO: 224 - DNA, 20 bp, synthetic construct
  source 1..20: mol_type = other DNA
actctggggtctattactgt

SEQ ID NO: 225 - DNA, 20 bp, synthetic construct
  source 1..20: mol_type = other DNA
acatagctacctactactgt

SEQ ID NO: 226 - DNA, 20 bp, synthetic construct
  source 1..20: mol_type = other DNA
acatggccgtttactactgt

SEQ ID NO: 227 - DNA, 20 bp, synthetic construct
  source 1..20: mol_type = other DNA
attctggggtctattactgt

SEQ ID NO: 228 - DNA, 20 bp, synthetic construct
  source 1..20: mol_type = other DNA
actctggagtctattactgt

SEQ ID NO: 229 - DNA, 20 bp, synthetic construct
  source 1..20: mol_type = other DNA
actctggggtctattactgt

SEQ ID NO: 230 - DNA, 20 bp, synthetic construct
  source 1..20: mol_type = other DNA
atgaggtggtgtaccactgt

SEQ ID NO: 231 - DNA, 20 bp, synthetic construct
  source 1..20: mol_type = other DNA
acatggccgtttactactgt

SEQ ID NO: 232 - DNA, 20 bp, synthetic construct
  source 1..20: mol_type = other DNA
acatagctacctactactgt

SEQ ID NO: 233 - DNA, 20 bp, synthetic construct
  source 1..20: mol_type = other DNA
attctgggttctattactgt

SEQ ID NO: 234 - DNA, 20 bp, synthetic construct
  source 1..20: mol_type = other DNA
  modified_base 2: mod_base = i
  modified_base 4: mod_base = i
  modified_base 8: mod_base = i
antntggngtctattactgt

SEQ ID NO: 235 - DNA, 20 bp, synthetic construct
  source 1..20: mol_type = other DNA
acatagctacctactactgt

SEQ ID NO: 236 - DNA, 20 bp, synthetic construct
  source 1..20: mol_type = other DNA
atgaggtggtgtaccactgt

SEQ ID NO: 237 - DNA, 20 bp, synthetic construct
  source 1..20: mol_type = other DNA
acatggccgtttactactgt

SEQ ID NO: 238 - DNA, 20 bp, synthetic construct
  source 1..20: mol_type = other DNA
attcagcaaagtacttttgt

SEQ ID NO: 239 - DNA, 20 bp, synthetic construct
  source 1..20: mol_type = other DNA
atgccgcgatgtatttctgt

SEQ ID NO: 240 - DNA, 20 bp, synthetic construct
  source 1..20: mol_type = other DNA
actctgcagtgtacttctgt

SEQ ID NO: 241 - DNA, 20 bp, synthetic construct
  source 1..20: mol_type = other DNA
actcggccatctacctctgt

SEQ ID NO: 242 - DNA, 20 bp, synthetic construct
  source 1..20: mol_type = other DNA
actcagccacctacttctgt

SEQ ID NO: 243 - DNA, 20 bp, synthetic construct
  source 1..20: mol_type = other DNA
actcagcaatgtatttctgt

SEQ ID NO: 244 - DNA, 20 bp, synthetic construct
  source 1..20: mol_type = other DNA
actcggccgtctacctctgt

SEQ ID NO: 245 - DNA, 20 bp, synthetic construct
  source 1..20: mol_type = other DNA
acagtgccacttactactgt

SEQ ID NO: 246 - DNA, 20 bp, synthetic construct
  source 1..20: mol_type = other DNA
atgaagggtcttactactgt

SEQ ID NO: 247 - DNA, 20 bp, synthetic construct
  source 1..20: mol_type = other DNA
actcagcaatgtacttctgt

SEQ ID NO: 248 - DNA, 20 bp, synthetic construct
  source 1..20: mol_type = other DNA
actcggccgtctacctctgt

SEQ ID NO: 249 - DNA, 20 bp, synthetic construct
  source 1..20: mol_type = other DNA
actcagcaatgtacttctgt

SEQ ID NO: 250 - DNA, 20 bp, synthetic construct
  source 1..20: mol_type = other DNA
actctgcagtgtacttctgt

SEQ ID NO: 251 - DNA, 20 bp, synthetic construct
  source 1..20: mol_type = other DNA
atgccgcgatgtatttctgt

SEQ ID NO: 252 - DNA, 20 bp, synthetic construct
  source 1..20: mol_type = other DNA
atgaagggtcttactactgt

SEQ ID NO: 253 - DNA, 20 bp, synthetic construct
  source 1..20: mol_type = other DNA
actcagccacctacttctgt

SEQ ID NO: 254 - DNA, 20 bp, synthetic construct
  source 1..20: mol_type = other DNA
acagtgccacttactactgt

SEQ ID NO: 255 - DNA, 20 bp, synthetic construct
  source 1..20: mol_type = other DNA
attcagcaaagtacttttgt

SEQ ID NO: 256 - DNA, 20 bp, synthetic construct
  source 1..20: mol_type = other DNA
  modified_base 9: mod_base = i
actcggccntctacctctgt

SEQ ID NO: 257 - DNA, 20 bp, synthetic construct
  source 1..20: mol_type = other DNA
  modified_base 10..11: mod_base = i
  modified_base 14: mod_base = i
actcagcaanntanttctgt

SEQ ID NO: 258 - DNA, 20 bp, synthetic construct
  source 1..20: mol_type = other DNA
atgccgcgatgtatttctgt

SEQ ID NO: 259 - DNA, 20 bp, synthetic construct
  source 1..20: mol_type = other DNA
actctgcagtgtacttctgt

SEQ ID NO: 260 - DNA, 20 bp, synthetic construct
  source 1..20: mol_type = other DNA
atgaagggtcttactactgt

SEQ ID NO: 261 - DNA, 20 bp, synthetic construct
  source 1..20: mol_type = other DNA
acagtgccacttactactgt

SEQ ID NO: 262 - DNA, 20 bp, synthetic construct
  source 1..20: mol_type = other DNA
attcagcaaagtacttttgt

SEQ ID NO: 263 - DNA, 20 bp, synthetic construct
  source 1..20: mol_type = other DNA
ccagcactgagagccgggtc

SEQ ID NO: 264 - DNA, 20 bp, synthetic construct
  source 1..20: mol_type = other DNA
ccagaaccaggagtcctccg

SEQ ID NO: 265 - DNA, 20 bp, synthetic construct
  source 1..20: mol_type = other DNA
cgagcaccaggagccgcgtg

SEQ ID NO: 266 - DNA, 20 bp, synthetic construct
  source 1..20: mol_type = other DNA
ccagtacggtcagcctagag

SEQ ID NO: 267 - DNA, 20 bp, synthetic construct
  source 1..20: mol_type = other DNA
cgagcactgtcagccgggtg

SEQ ID NO: 268 - DNA, 20 bp, synthetic construct
  source 1..20: mol_type = other DNA
ctgtgaccgtgagcctggtg

SEQ ID NO: 269 - DNA, 20 bp, synthetic construct
  source 1..20: mol_type = other DNA
ctacaacggttaacctggtc

SEQ ID NO: 270 - DNA, 20 bp, synthetic construct
  source 1..20: mol_type = other DNA
ctacaacagtgagccaactt

SEQ ID NO: 271 - DNA, 20 bp, synthetic construct
  source 1..20: mol_type = other DNA
ctgtcacagtgagcctggtc

SEQ ID NO: 272 - DNA, 20 bp, synthetic construct
  source 1..20: mol_type = other DNA
ctacaactgtgagtctggtg

SEQ ID NO: 273 - DNA, 20 bp, synthetic construct
  source 1..20: mol_type = other DNA
ctaggatggagagtcgagtc

SEQ ID NO: 274 - DNA, 20 bp, synthetic construct
  source 1..20: mol_type = other DNA
ccaagacagagagctgggtt

SEQ ID NO: 275 - DNA, 20 bp, synthetic construct
  source 1..20: mol_type = other DNA
ccagcacggtcagcctgctg

SEQ ID NO: 276 - DNA, 20 bp, synthetic construct
  source 1..20: mol_type = other DNA
ctagcacggtgagccgtgtc

SEQ ID NO: 277 - DNA, 30 bp, synthetic construct
  source 1..30: mol_type = other DNA
ctggagttcagacgtgtgctcttccgatct

SEQ ID NO: 278 - DNA, 17 bp, synthetic construct
  source 1..17: mol_type = other DNA
ggagctgtcgttcactc

SEQ ID NO: 279 - DNA, 66 bp, synthetic construct
  source 1..66: mol_type = other DNA
caagcagaagacggcatacgagatnnnnnnnngtgactggagttcagacgtgtgctcttccgatct

SEQ ID NO: 280 - DNA, 70 bp, synthetic construct
  source 1..70: mol_type = other DNA
aatgatacggcgaccaccgagatctacacnnnnnnnnacactctttccgatctggagctgtcgttcactc

SEQ ID NO: 281 - protein, 12 aa, synthetic construct
CASTGGYGATGE

SEQ ID NO: 282 - DNA, 60 bp, synthetic construct
  source 1..60: mol_type = other DNA
ctcccagacatctgtgtacttctgtgccagcaccggggggtacggcgcgaccggggcgct

(19) United States
(12) Patent Application Publication
(10) Pub. No.: US 2026/0234606 A1
(43) Pub. Date: Aug. 13, 2026

(54) METHODS FOR DETECTING NUCLEIC ACID VARIANTS

(71) Applicant: GUARDANT HEALTH, INC., Palo Alto, CA (US)

(72) Inventor: Andrew KENNEDY, San Diego, CA (US)

(21) Appl. No.: 19/655,248

(22) Filed: Apr. 22, 2026

Related U.S. Application Data
(63) Continuation of application No. PCT/US2024/053101, filed on Oct. 25, 2024.
(60) Provisional application No. 63/593,447, filed on Oct. 26, 2023.

(51) Int. Cl. C12N 15/10

(52) U.S. Cl. CPC C12N 15/1065

(57) ABSTRACT

Provided herein is a DNA analysis method for DNA in an amplified, adapted library, comprising partitioning the amplified, adapted library into at least a first subsample and a second subsample; capturing a plurality of target regions comprising sequence-variable target regions from the first subsample, thereby providing captured regions; performing multiplex amplification of segments comprising recombined CDR3 sequences from the second subsample using a plurality of first primers that bind V regions and a plurality of second primers that bind J regions, thereby providing CDR3-enriched DNA; and sequencing the captured regions and the CDR3-enriched DNA.

### CROSS-REFERENCE TO RELATED APPLICATIONS

The present application is a Continuation of International Patent Application No. PCT/US2024/053101, filed Oct. 25, 2024, which claims the benefit of priority of U.S. Provisional Patent Application No. 63/593,447, filed Oct. 26, 2023, which is incorporated by reference herein in its entirety for all purposes.

### FIELD OF THE INVENTION

The present disclosure provides methods related to analyzing DNA, such as cell-free DNA. In some embodiments, multiplex amplification of segments (e.g., comprising recombined CDR3 sequences) is performed. In some embodiments, multiplex amplification of a second plurality of target regions that may comprise a structural variation is performed. In some embodiments, a plurality of target regions comprising sequence-variable target regions is captured. In some embodiments, the DNA is from a subject having or suspected of having cancer, and/or the DNA includes DNA from cancer cells.

### SEQUENCE LISTING

The present application is filed with a Sequence Listing in electronic format. The Sequence Listing is provided as a file entitled “GH0182.xml” created on Oct. 15, 2024, which is 271,725 bytes in size. The information in the electronic format of the sequence listing is incorporated herein by reference in its entirety.

### INTRODUCTION AND SUMMARY

Cancer is responsible for millions of deaths per year worldwide. Early cancer detection may result in improved outcomes because early-stage cancer tends to be more susceptible to treatment.

Improperly controlled cell growth is a hallmark of cancer. Cancer is usually caused by the accumulation of mutations within an individual's normal cells, at least some resulting in improperly regulated cell division. Such mutations commonly include single nucleotide variations (SNVs), gene fusions, insertions and deletions (indels), transversions, translocations, and inversions. Cancers derived from certain cell types, such as lymphocytes, may also comprise recombined CDR3 sequences that can be useful, e.g., for detecting or identifying cancer cells. Cancers may also exhibit an accumulation of epigenetic changes, including modification of cytosine (e.g., 5-methylcytosine, 5-hydroxymethylcytosine, and other more oxidized forms) and association of DNA with chromatin proteins and transcription factors.

Thus, cancer can be indicated by non-sequence modifications, such as methylation. Examples of methylation changes in cancer include local gains of DNA methylation, e.g., in the CpG islands at the transcription start sites of genes involved in normal growth control, DNA repair, cell cycle regulation, and/or cell differentiation. Hypermethylation can be associated with an aberrant loss of transcriptional capacity of involved genes and occurs at least as frequently as point mutations and deletions as a cause of altered gene expression. Furthermore, without wishing to be bound by any particular theory, cells in or around a cancer or neoplasm may shed more DNA than cells of the same tissue type in a healthy subject. The DNA from such cells may differ epigenetically from shed DNA in a healthy subject. As such, the distribution of epigenetically modified (e.g., methylated) DNA in certain DNA samples, such as cell-free DNA (cfDNA), may change upon carcinogenesis. Thus, sufficiently sensitive epigenetic (e.g., DNA methylation) profiling can be used to detect aberrant methylation in DNA of a sample.

Biopsies represent a traditional approach for detecting or diagnosing cancer in which cells or tissue are extracted from a possible cancer site and analyzed for relevant phenotypic and/or genotypic features. Biopsies have the drawback of being invasive.

Cancer detection based on analysis of body fluids (“liquid biopsies”), such as blood, is an intriguing alternative based on the observation that DNA from cancer cells is released into body fluids. A liquid biopsy is noninvasive (sometimes requiring only a blood draw). However, it has been challenging to develop accurate and sensitive methods for analyzing liquid biopsy material because the amount of nucleic acids released into body fluids is low and variable, as is recovery of nucleic acids from such fluids in analyzable form. These sources of variation can obscure the predictive value of mutations (e.g., rearrangements, such as translocations and indels) among samples. Such mutations may include biomarkers that can be used to evaluate whether a subject diagnosed with, or suspected of having signs of, a cancer will benefit from a specific type of cancer therapy, such as Immuno-Oncology (I-O) therapy. Isolating and processing cell-free DNA useful for further analysis in liquid biopsy procedures can be a useful part of these methods. Accordingly, there is a need for improved methods and compositions for analyzing cell-free DNA, e.g., in liquid biopsies.

To identify recombined CDR3 sequences, simple capture of V, D, or J segments will not necessarily distinguish them from uninformative germline sequences, e.g., from cells of non-lymphocytic lineages. Accordingly, multiplex amplification using primers that specifically amplify recombined CDR3 sequences can be used in some embodiments disclosed herein.

With regard to identifying structural variations, DNA breakpoints can vary; therefore, effective methods of detecting rearrangements in DNA should capture as many breakpoints as possible. Existing methods may not be selective for rearrangements and may result in significant detection of wild type DNA and, thus poor signal-to-noise ratios. In some embodiments, the methods herein comprise multiplex amplification of DNA comprising a rearrangement and detection of the primer-extended products.

The methods herein can provide combined information about CDR3 sequences and/or DNA rearrangements and other modifications, including but not limited to sequence variations.

Existing methods may not provide for capture and analysis of modifications such as sequence variations in sequence-variable target regions and/or epigenetic variations in epigenetic target regions together with analysis of CDR3 sequences and/or DNA rearrangements from a single sample.

The present disclosure aims to meet the need for improved analysis of DNA comprising a rearrangement and/or recombined CDR3 sequences together with other modifications such as sequence variations in sequence-variable target regions and/or epigenetic variations in epigenetic target regions, such as cell-free DNA, provide other benefits, or at least provide the public with a useful choice. Accordingly, the following exemplary embodiments are provided.

Embodiment 1 is a method of analyzing DNA in an amplified, adapted library, the method comprising:
a) partitioning the amplified, adapted library into at least a first subsample and a second subsample;
b) capturing a plurality of target regions comprising sequence-variable target regions and/or epigenetic target regions from the first subsample, thereby providing captured regions;
c) performing multiplex amplification of segments comprising recombined CDR3 sequences from the second subsample using a plurality of first primers that bind V regions and a plurality of second primers that bind J regions, thereby providing CDR3-enriched DNA; and
d) sequencing the captured regions and the CDR3-enriched DNA.
Embodiment 1.1 is the method of embodiment 1, wherein the plurality of target regions comprises sequence-variable target regions.
Embodiment 2 is the method of embodiment 1 or 1.1, wherein the plurality of first primers comprises from 10 to 150, from 15 to 125, from 20 to 100, from 30 to 90, from 40 to 80, or from 50 to 70 primers that bind V regions.
Embodiment 3 is the method of any one of the preceding embodiments, wherein the plurality of second primers comprises from 5 to 30, from 10 to 25, or from 10 to 20 primers that bind J regions.
Embodiment 4 is the method of any one of the preceding embodiments, wherein the captured regions and the CDR3-enriched DNA are pooled and sequenced together.
Embodiment 5 is the method of any one of embodiments 1 to 4, wherein the captured regions and the CDR3-enriched DNA are sequenced separately.
Embodiment 6 is the method of any one of the preceding embodiments, comprising detecting a presence or absence of a DNA molecule that comprises a structural variation from the first subsample.
Embodiment 7 is a method of analyzing DNA in an amplified, adapted library, the method comprising:
a) partitioning the amplified, adapted library into at least a first subsample and a second subsample;
b) capturing a first plurality of target regions comprising sequence-variable target regions and/or epigenetic target regions from the first subsample, thereby providing captured regions;
c) performing multiplex amplification of a second plurality of target regions that may comprise a structural variation from the second subsample using a plurality of first primers and a plurality of second primers that anneal to the second plurality of target regions, thereby providing structural variation-enriched DNA; and
d) sequencing the captured regions and the structural variation-enriched DNA.
Embodiment 7.1 is the method of embodiment 7, wherein the first plurality of target regions comprises sequence-variable target regions.
Embodiment 8 is the method of embodiment 7 or 7.1, wherein the captured regions and the structural variation-enriched DNA are pooled and sequenced together.
Embodiment 9 is the method of embodiment 7 or 7.1, wherein the captured regions and the structural variation-enriched DNA are sequenced separately.
Embodiment 10 is the method of any one of embodiments 7 to 9, wherein at least a portion of the plurality of first primers and the plurality of second primers do not exponentially amplify a target region that does not comprise a structural variation.
Embodiment 11 is the method of any of embodiments 6 to 10, wherein the structural variation comprises a rearrangement, an insertion, or a deletion.
Embodiment 12 is the method of the immediately preceding embodiment, wherein the rearrangement comprises translocations, gene fusions, duplications, copy-number variants, or inversions.
Embodiment 13 is the method of any one of the preceding embodiments, wherein a third subsample of the amplified, adapted library is retained as a backup.
Embodiment 14 is the method of any one of the preceding embodiments, wherein the method comprises preparing the amplified, adapted library by ligating adaptors to DNA, thereby producing adapted DNA, followed by amplifying the adapted DNA.
Embodiment 15 is the method of embodiment 14, wherein the adapted DNA comprises molecular barcodes.
Embodiment 16 is the method of any one of the preceding embodiments, wherein the amplified, adapted library is prepared from cfDNA.
Embodiment 17 is the method of embodiment 16, wherein the method comprises preparing the amplified, adapted library by ligating adaptors to cfDNA, thereby producing adapted cfDNA, followed by amplifying the adapted cfDNA.
Embodiment 18 is the method of any one of the preceding embodiments, wherein the captured regions are amplified prior to sequencing.
Embodiment 19 is the method of any one of the preceding embodiments, wherein the amplified, adapted library is prepared from DNA from a subject having or suspected of having a cancer.
Embodiment 20 is the method of any one of the preceding embodiments, further comprising determining a likelihood that the subject has a cancer.
Embodiment 21 is the method of any one of embodiments 19 to 20, wherein the cancer is a lymphocytic cancer.
Embodiment 22 is the method of the immediately preceding embodiment, wherein the lymphocytic cancer is a leukemia, a lymphoma, or a myeloma.
Embodiment 23 is the method of any one of embodiments 19 to 20, wherein the cancer is a lymphoma.
Embodiment 24 is the method of embodiment 23, wherein the lymphoma is B-cell lymphoma, non-Hodgkin lymphoma, diffuse large B-cell lymphoma, Mantle cell lymphoma, T cell lymphoma, non-Hodgkin lymphoma, precursor T-lymphoblastic lymphoma/leukemia, or peripheral T cell lymphoma.
Embodiment 25 is the method of any one of the preceding embodiments, wherein the CDR3 is a part of a T cell receptor (TCR), TCR beta chain, B cell receptor, immunoglobulin, B cell receptor heavy chain, or immunoglobulin heavy chain.
Embodiment 26 is a method of analyzing DNA in an amplified, adapted library, the method comprising:
a) preparing the amplified, adapted library by ligating adaptors to cfDNA from a subject having or suspected of having a cancer, thereby producing adapted cfDNA, followed by amplifying the adapted cfDNA;
b) partitioning the amplified, adapted library into at least first and second subsamples;
c) capturing a plurality of target regions comprising sequence-variable target regions and/or epigenetic target regions from the first subsample, thereby providing captured regions;
d) performing multiplex amplification of segments comprising recombined CDR3 sequences from the second subsample using a plurality of first primers that bind V regions and a plurality of second primers that bind J regions, thereby providing CDR3-enriched DNA;
e) sequencing the captured regions and the CDR3-enriched DNA.
Embodiment 26.1 is the method of embodiment 26, wherein the plurality of target regions comprises sequence-variable target regions.
Embodiment 27 is the method of embodiment 26 or 26.1, wherein the captured regions and the CDR3-enriched DNA are pooled and sequenced together.
Embodiment 28 is the method of embodiment 26 or 26.1, wherein the captured regions and the CDR3-enriched DNA are sequenced separately.
Embodiment 29 is the method of any one of the preceding embodiments, further comprising capturing epigenetic target regions from the DNA of the adapted, amplified DNA library and amplifying and sequencing the epigenetic target regions.
Embodiment 30 is the method of the immediately preceding embodiment, wherein the epigenetic target regions comprise hypermethylation variable target regions, hypomethylation variable target regions, methylation control target regions, or fragmentation variable target regions.
Embodiment 31 is the method of any one of the preceding embodiments, further comprising quantifying a somatic mutation load using a plurality of captured regions comprising the sequence-variable target regions.
Embodiment 32 is the method of any one of the preceding embodiments, wherein each of the plurality of the first and second primers comprises at least 20 linked nucleosides.
Embodiment 33 is the method of any one of the preceding embodiments, wherein each of the plurality of the first and second primers consists of 20 to 60 linked nucleosides.
Embodiment 34 is the method of any of the preceding embodiments, wherein the plurality of the first and second primers are resistant to 5′ exonucleolysis.
Embodiment 35 is the method of any of the preceding embodiments, comprising differentially tagging and pooling the first subsample and second subsample.
Embodiment 36 is the method of embodiment 34, wherein the pool comprises less than or equal to about 45%, 40%, 35%, 30%, 25%, 20%, 15%, 10%, or 5% of the DNA of the second subsample.
Embodiment 37 is the method of the immediately preceding embodiment, wherein the pool comprises about 70-90%, about 75-85%, or about 80% of the DNA of the second subsample.
Embodiment 38 is the method of embodiment 34, wherein the pool comprises substantially all of the DNA of the first subsample.
Embodiment 39 is the method of any one of the preceding embodiments, wherein the detecting comprises generating a plurality of sequencing reads; and the method further comprises mapping the plurality of sequence reads to one or more reference sequences to generate mapped sequence reads, and processing the mapped sequence reads to determine the likelihood that the subject has cancer.
Embodiment 40 is the method of the immediately preceding embodiment, further comprising detecting a presence or absence of DNA originating or derived from a tumor cell using the mapped sequence reads.
Embodiment 41 is the method of the immediately preceding embodiment, further comprising determining a cancer recurrence score that is indicative of the presence or absence of the DNA originating or derived from the tumor cell for the test subject, optionally further comprising determining a cancer recurrence status based on the cancer recurrence score, wherein the cancer recurrence status of the test subject is determined to be at risk for cancer recurrence when a cancer recurrence score is determined to be at or above a predetermined threshold or the cancer recurrence status of the test subject is determined to be at lower risk for cancer recurrence when the cancer recurrence score is below the predetermined threshold.
Embodiment 42 is the method of the immediately preceding embodiment, further comprising comparing the cancer recurrence score of the test subject with a predetermined cancer recurrence threshold, wherein the test subject is classified as a candidate for a subsequent cancer treatment when the cancer recurrence score is above the cancer recurrence threshold or not a candidate for a subsequent cancer treatment when the cancer recurrence score is below the cancer recurrence threshold.

In some embodiments, the results of the methods disclosed herein are used as an input to generate a report. The report may be in a paper or electronic format. For example, true copy number variation, as obtained by the methods disclosed herein, or information derived therefrom, can be displayed directly in such a report. Alternatively or additionally, diagnostic information or therapeutic recommendations which are at least in part based on the methods disclosed herein can be included in the report.

The various steps of the methods disclosed herein may be carried out at the same or different times, in the same or different geographical locations, e.g. countries, and/or by the same or different people.

Additional advantages will be set forth in part in the description which follows or may be learned by practice. The advantages will be realized and attained by means of the elements and combinations particularly pointed out in the appended claims.

### BRIEF DESCRIPTION OF THE DRAWINGS

FIG. 1A illustrates an exemplary workflow according to certain embodiments of the disclosure, beginning with DNA isolated from a sample and ligated to adapters for sequencing library preparation, then amplified to provide an amplified DNA library. The DNA is then partitioned into at least a first subsample and a second subsample, and an optional third subsample, e.g., to be retained as a backup. Sequence-variable target regions are captured and amplified from the first subsample, and recombined CDR3 sequences are amplified through multiplex PCR from the second subsample. Then, the captured regions and the CDR3-enriched DNA are pooled and sequenced together.

FIG. 1B illustrates an exemplary workflow according to certain embodiments of the disclosure, beginning with DNA isolated from a sample and ligated to adapters for sequencing library preparation, then amplified to provide an amplified DNA library. The DNA is then partitioned into at least a first subsample and a second subsample, and an optional third subsample, e.g., to be retained as a backup. Sequence-variable target regions are captured and amplified from the first subsample, and recombined CDR3 sequences are amplified through multiplex PCR from the second subsample. Then, the captured regions and the CDR3-enriched DNA are sequenced separately.

FIG. 1C illustrates certain features of primer sets according to certain embodiments of the disclosure. Exemplary CDR3+ readout: CASTGGYGATGE (SEQ ID NO: 281); CTCCCAGACATCTGTGTACTTCTGTGCCAGCACCGGGGGGTACGGCGCGACCGGGG CGCT (SEQ ID NO: 282).

FIG. 2 is a schematic diagram of an example of a system suitable for use with some embodiments of the disclosure.

### Detailed Description of Certain Embodiments

Reference will now be made in detail to certain embodiments of the disclosure. While the disclosure will be described in conjunction with such embodiments, it will be understood that they are not intended to limit the disclosure to those embodiments. On the contrary, the disclosure is intended to cover all alternatives, modifications, and equivalents, which may be included within the disclosure as defined by the appended claims.

Before describing the present teachings in detail, it is to be understood that the disclosure is not limited to specific compositions or process steps, as such may vary. It should be noted that, as used in this specification and the appended claims, the singular form “a”, “an” and “the” include plural references unless the context clearly dictates otherwise. Thus, for example, reference to “a nucleic acid” includes a plurality of nucleic acids

Numeric ranges are inclusive of the numbers defining the range. Measured and measurable values are understood to be approximate, taking into account significant digits and the error associated with the measurement.

Unless specifically noted in the above specification, embodiments in the specification that recite “comprising” various components are also contemplated as “consisting of” or “consisting essentially of” the recited components.

The section headings used herein are for organizational purposes and are not to be construed as limiting the disclosed subject matter in any way.

All patents, patent applications, websites, other publications or documents and the like cited herein whether supra or infra, are expressly incorporated by reference in their entirety for all purposes to the same extent as if each individual item were specifically and individually indicated to be so incorporated by reference. If different versions of a publication, website or the like are published at different times, the version most recently published at the effective filing date of the application is meant, unless otherwise indicated.

### I. Definitions

“Cell-free DNA,” “cfDNA molecules,” or simply “cfDNA” include DNA molecules that naturally occur in a subject in extracellular form (e.g., in blood, serum, plasma, or other bodily fluids such as lymph, cerebrospinal fluid, urine, or sputum). While the cfDNA previously existed in a cell or cells in a large complex biological organism, e.g., a mammal, it has undergone release from the cell(s) into a fluid found in the organism, and may be obtained from a sample of the fluid without the need to perform an in vitro cell lysis step. cfDNA molecules may occur as DNA fragments.

As used herein, “primer-annealed DNA” means DNA to which at least one primer is annealed.

As used herein, a DNA polymerase that is “5′ to 3′ exonuclease negative” does not have significant 5′ to 3′ exonuclease activity. A DNA polymerase that is “strand displacement negative” does not have significant helicase activity to displace a strand, such as a primer, annealed to DNA. In some embodiments, a 5′ to 3′ exonuclease negative, strand displacement negative DNA polymerase is used for primer extension in order to prevent extension of primers annealed to wild type target regions.

As used herein, a “primer-extended product,” when referring to primers that anneal to at least one target region, means a nucleic acid strand formed by extension of a primer annealed to a DNA target region. In some embodiments, a primer-extended product is a significant primer-extended product or is formed by significant primer extension, meaning that the resulting nucleic acid strand has sufficient additional length (e.g., at least 10, 15, 20, 30, 40, 50, 60, 75, or 100 nucleotides in addition to the length of the original primer) to be detected and/or identified using methods described herein. In some embodiments, significant primer extension results in primer-extended products comprising a capture moiety present at a low percentage in the deoxynucleoside triphosphate mixture. In some embodiments, primer extension that results in no primer-extended products or only short primer-extended products that do not comprise the capture moiety occurs on target regions comprising a completely tiled primer-annealed target region.

As used herein, “adjacent” nucleosides or oligonucleotides are nucleosides or oligonucleotides that are next to each other, with no intervening nucleosides. For example, “adjacent” nucleosides may be covalently linked together within a nucleic acid or oligonucleotide, or they may be unlinked but are next to each other because they are annealed to or hybridized to adjacent linked nucleosides of a nucleic acid. “Adjacent” oligonucleotides may likewise be linked together or unlinked to each other but annealed to or hybridized to adjacent, linked portions of a nucleic acid.

As used herein, “partitioning” of nucleic acids, such as DNA molecules, means separating, fractionating, sorting, or enriching a sample or population of nucleic acids into a plurality of subsamples or subpopulations of nucleic acids. Partitioning may simply divide a sample into two or more equivalent subsamples or may separate the sample into subsamples with different features based on one or more modifications or features that is in different proportions in each of the plurality of subsamples or subpopulations. Partitioning may include physically partitioning nucleic acid molecules based on the presence or absence of one or more methylated nucleobases. A sample or population may be partitioned into one or more partitioned subsamples or subpopulations based on a characteristic that is indicative of a genetic or epigenetic change or a disease state.

As used herein, a modification or other feature is present in “a greater proportion” in a first sample or population of nucleic acid than in a second sample or population when the fraction of nucleotides with the modification or other feature is higher in the first sample or population than in the second population. For example, if in a first sample, one tenth of the nucleotides are mC, and in a second sample, one twentieth of the nucleotides are mC, then the first sample comprises the cytosine modification of 5-methylation in a greater proportion than the second sample.

As used herein, the form of the “originally isolated” sample refers to the composition or chemical structure of a sample at the time it was isolated and before undergoing any procedure that changes the chemical structure of the isolated sample. Similarly, a feature that is “originally present” in a molecule refers to a feature present in an “original molecule” or in molecules “originally comprising” the feature before the molecule undergoes any procedure that changes the chemical structure of the molecule.

As used herein, “without substantially altering base pairing specificity” of a given nucleobase means that a majority of molecules comprising that nucleobase that can be sequenced do not have alterations of the base pairing specificity of the given nucleobase relative to its base pairing specificity as it was in the originally isolated sample. In some embodiments, 75%, 90%, 95%, or 99% of molecules comprising that nucleobase that can be sequenced do not have alterations of the base pairing specificity relative to its base pairing specificity as it was in the originally isolated sample. As used herein, “altered base pairing specificity” of a given nucleobase means that a majority of molecules comprising that nucleobase that can be sequenced have a base pairing specificity at that nucleobase relative to its base pairing specificity in the originally isolated sample.

As used herein, “base pairing specificity” refers to the standard DNA base (A, C, G, or T) for which a given base most preferentially pairs. For example, unmodified cytosine and 5-methylcytosine have the same base pairing specificity (i.e., specificity for G) whereas uracil and cytosine have different base pairing specificity because uracil has base pairing specificity for A while cytosine has base pairing specificity for G. The ability of uracil to form a wobble pair with G is irrelevant because uracil nonetheless most preferentially pairs with A among the four standard DNA bases.

As used herein, a “combination” comprising a plurality of members refers to either of a single composition comprising the members or a set of compositions in proximity, e.g., in separate containers or compartments within a larger container, such as a multiwell plate, tube rack, refrigerator, freezer, incubator, water bath, ice bucket, machine, or other form of storage.

The “capture yield” of a collection of probes for a given target region set refers to the amount (e.g., amount relative to another target region set or an absolute amount) of nucleic acid corresponding to the target region set that the collection of probes captures under typical conditions. Exemplary typical capture conditions are an incubation of the sample nucleic acid and probes at 65° C. for 10-18 hours in a small reaction volume (about 20 μL) containing stringent hybridization buffer. The capture yield may be expressed in absolute terms or, for a plurality of collections of probes, relative terms. When capture yields for a plurality of sets of target regions are compared, they are normalized for the footprint size of the target region set (e.g., on a per-kilobase basis). Thus, for example, if the footprint sizes of first and second target regions are 50 kb and 500 kb, respectively (giving a normalization factor of 0.1), then the DNA corresponding to the first target region set is captured with a higher yield than DNA corresponding to the second target region set when the mass per volume concentration of the captured DNA corresponding to the first target region set is more than 0.1 times the mass per volume concentration of the captured DNA corresponding to the second target region set. As a further example, using the same footprint sizes, if the captured DNA corresponding to the first target region set has a mass per volume concentration of 0.2 times the mass per volume concentration of the captured DNA corresponding to the second target region set, then the DNA corresponding to the first target region set was captured with a two-fold greater capture yield than the DNA corresponding to the second target region set.

As used herein, a “label” is a capture moiety, fluorophore, oligonucleotide, or other moiety that facilitates detection, separation, or isolation of that to which it is attached.

“Capturing” one or more target nucleic acids refers to preferentially isolating or separating the one or more target nucleic acids from non-target nucleic acids.

A “captured set” of nucleic acids or “captured” nucleic acids refers to nucleic acids that have undergone capture.

As used herein, a “capture moiety” is a molecule that allows affinity separation of molecules, such as nucleic acids, linked to the capture moiety from molecules lacking the capture moiety. Exemplary capture moieties include biotin, which allows affinity separation by binding to streptavidin linked or linkable to a solid phase or an oligonucleotide, which allows affinity separation through binding to a complementary oligonucleotide linked or linkable to a solid phase.

As used herein, a “tag” is a molecule, such as a nucleic acid, label, fluorophore, or peptide, containing information that indicates a feature of the molecule to which the tag is associated. For example, molecules can bear a sample tag (which distinguishes molecules in one sample from those in a different sample), a molecular tag/molecular barcode/barcode (which distinguishes different molecules from one another (in both unique and non-unique tagging scenarios), a purification tag, and/or a detectable tag or label.

A “target region” refers to a genomic locus targeted for identification and/or capture, for example, by using primers and/or probes (e.g., through sequence complementarity). A “target region set” or “set of target regions” refers to a plurality of genomic loci targeted for identification and/or capture, for example, by using a set of primers and/or probes (e.g., through sequence complementarity).

As used herein, a DNA “structural variation” is a mutation comprising a DNA sequence not present in the wild-type genome other than a point mutation (e.g., in which at least 5, 10, 20, or 50 contiguous nucleotides are different relative to the wild type sequence at the corresponding locus). Examples of DNA structural variations include rearrangements, such as translocations, insertions, deletions, duplications, copy-number variants, and inversions. As used herein, a DNA “rearrangement” is a structural variation, wherein the DNA sequence comprises two adjacent sequence portions that are not adjacent to each other in the germline genomic DNA. In some embodiments, a rearrangement is a translocation, gene fusion, insertion, deletion, or inversion. Exemplary rearrangements include products of a translocation, gene fusion, and VDJ recombination. In some embodiments, the rearrangement is the product of a translocation comprising fusion of two intronic regions. In some embodiments, the rearrangement is a product of VDJ recombination comprising adjacent J exonic regions. A molecule comprising a structural variation may be referred to as a structural variant. In some embodiments, an insertion is an insertion of at least 5, 10, 20, 30, 40, 50, 60, 70, 80, 90, or 100 nucleotides. In some embodiments, a deletion affects sequence spanning the end of the target region, so as to result in a primer being unblocked and undergoing extension in a method described herein.

“Corresponding to a target region set” means that a nucleic acid, such as cfDNA, originated from a locus in the target region set or specifically binds one or more primers or probes for the target region set.

“Specifically binds” in the context of a primer, a probe, or other oligonucleotide and a target sequence means that under appropriate hybridization conditions, the primer, oligonucleotide, or probe hybridizes to its target sequence, or replicates thereof, to form a stable hybrid, while at the same time formation of stable non-target hybrids is minimized. Thus, a primer or probe hybridizes to a target sequence or replicate thereof to a sufficiently greater extent than to a non-target sequence, to ultimately enable capture or detection of the target sequence. Appropriate hybridization conditions are well-known in the art, may be predicted based on sequence composition, or can be determined by using routine testing methods (see, e.g., Sambrook et al., Molecular Cloning, A Laboratory Manual, 2nd ed. (Cold Spring Harbor Laboratory Press, Cold Spring Harbor, NY, 1989) at §§ 1.90-1.91, 7.37-7.57, 9.47-9.51 and 11.47-11.57, particularly §§ 9.50-9.51, 11.12-11.13, 11.45-11.47 and 11.55-11.57, incorporated by reference herein).

“Sequence-variable target regions” refer to target regions that may exhibit changes in sequence such as nucleotide substitutions (i.e., single nucleotide variations), insertions, deletions, or gene fusions or transpositions in neoplastic cells (e.g., tumor cells and cancer cells) relative to normal cells. A sequence-variable target region set is a set of sequence-variable target regions. In some embodiments, the sequence-variable target regions are target regions that may exhibit changes that affect less than or equal to 50 contiguous nucleotides, e.g., less than or equal to 40, 30, 20, 10, 5, 4, 3, or 2 nucleotides, or that affect 1 nucleotide.

“Epigenetic target regions” refers to target regions that may show sequence-independent changes across tissue types (e.g., a target region having a different extent of methylation in a solid tissue type than in hematopoietic cells) or differences in neoplastic cells, such as tumor cells or cancer cells, relative to normal cells. In some embodiments, epigenetic target regions show sequence-independent differences in cfDNA originating from tissue types that ordinarily do not substantially contribute to cfDNA, such as lung, colon, etc., relative to background cfDNA, such as cfDNA that originated from hematopoietic cells. In some embodiments, epigenetic target regions show sequence-independent differences in cfDNA from subjects having cancer relative to cfDNA from healthy subjects. Examples of sequence-independent changes include, but are not limited to, changes in methylation (increases or decreases), nucleosome distribution, cfDNA fragmentation patterns, CCCTC-binding factor (“CTCF”) binding, transcription start sites, and regulatory protein binding regions. An epigenetic target region set is a set of epigenetic target regions. Epigenetic target region sets thus include, but are not limited to, hypermethylation variable target region sets, hypomethylation variable target region sets, and fragmentation variable target region sets, such as CTCF binding sites and transcription start sites. For present purposes, loci susceptible to neoplasia-, tumor-, or cancer-associated focal amplifications and/or gene fusions may also be included in an epigenetic target region set because detection of a change in copy number by sequencing or a fused sequence that maps to more than one locus in a reference genome tends to be more similar to detection of exemplary epigenetic changes discussed above than detection of nucleotide substitutions, insertions, or deletions, e.g., in that the focal amplifications and/or gene fusions can be detected at a relatively shallow depth of sequencing because their detection does not depend on the accuracy of base calls at one or a few individual positions.

As used herein, an “epigenetic feature” refers to any feature of DNA or chromatin other than primary sequence (i.e., the sequence of A, C, G, and T bases). Epigenetic features include covalent modifications of bases, such as methylation, and modifications and positioning of histones and other stably DNA-associated proteins.

As used herein, a “differentially methylated region” (DMR) refers to a region of DNA having a detectably different degree of methylation in at least one cell or tissue type relative to the degree of methylation in the same region of DNA from at least one other cell or tissue type; or having a detectably different degree of methylation in at least one cell or tissue type obtained from a subject having a disease or disorder relative to the degree of methylation in the same region of DNA in the same cell or tissue type obtained from a healthy subject. In some embodiments, a DMR has a detectably higher degree of methylation (e.g., hypermethylated region) in at least one cell or tissue type relative to the degree of methylation in the same region of DNA from at least one other cell or tissue type or from the same cell or tissue type from a healthy subject. In some embodiments, a DMR has a detectably lower degree of methylation (e.g., hypomethylated region) in at least one cell or tissue type relative to the degree of methylation in the same region of DNA from at least one other cell or tissue type or from the same cell or tissue type from a healthy subject.

As used herein, “type-specific” in the context of an epigenetic variation means an epigenetic variation that is present at a detectably different degree in one cell or tissue type, or in a plurality of related cell or tissue types, relative to other cell or tissue types. Similarly, a “type-specific epigenetic target region” is an epigenetic target region that has a detectably different epigenetic characteristic in one cell or tissue type, or in a plurality of related cell or tissue types, relative to other cell or tissue types. Exemplary epigenetic characteristics are discussed in the definition of epigenetic target regions set forth above. For example, a “type-specific differentially methylated region” is a region of DNA that has a detectably different degree of methylation in one cell or tissue type, or in a plurality of related cell or tissue types, relative to other cell or tissue types. Examples of a type-specific differentially methylated region include tissue-specific differentially methylated regions, including those associated with copy-number gain in early cancer. In some embodiments, capturing, identification, and/or detection of type-specific differentially methylated regions facilitates identification of the cell or tissue type from which the DNA originated. The cell or tissue from which a type-specific differentially methylated region originated may be a wild type cell or tissue or a neoplastic cell or tissue. In another example, a “type-specific fragment” of DNA is a DNA fragment arising from a type-specific fragmentation pattern that is present at a detectably different degree in one cell or tissue type, or in a plurality of related cell or tissue types, relative to other cell or tissue types. In some embodiments, a type-specific fragment is only present in the specific cell or tissue type(s). In some embodiments, a type-specific fragment is present to a detectably greater extent in the specific cell or tissue type(s).

DNA is “derived from cancerous cells” if it originated from a tumor cell. Cell free DNA derived from cancerous cells includes ctDNA or circulating tumor DNA. Tumor cells are neoplastic cells that originated from a tumor, regardless of whether they remain in the tumor or become separated from the tumor (as in the cases, e.g., of metastatic cancer cells and circulating tumor cells).

The term “methylation” or “DNA methylation” refers to addition of a methyl group to a nucleotide base in a nucleic acid molecule. In some embodiments, methylation refers to addition of a methyl group to a cytosine at a CpG site (cytosine-phosphate-guanine site (i.e., a cytosine followed by a guanine in a 5′→3′ direction of the nucleic acid sequence)). In some embodiments, DNA methylation refers to addition of a methyl group to adenine, such as in N⁶-methyladenine (6 mA). In some embodiments, DNA methylation is 5-methylation (modification of the carbon in the 5^{th} position of the cytosine ring). In some embodiments, 5-methylation refers to addition of a methyl group to the 5C position of the cytosine to create 5-methylcytosine (5mC). In some embodiments, methylation comprises a derivative of 5mC. Derivatives of 5mC include, but are not limited to, 5-hydroxymethylcytosine (5-hmC), 5-formylcytosine (5-fC), and 5-caryboxylcytosine (5-caC). In some embodiments, DNA methylation is 3C methylation (modification of the carbon in the 3^{rd} position of the cytosine ring). In some embodiments, 3C methylation comprises addition of a methyl group to the 3C position of the cytosine to generate 3-methylcytosine (3mC). Methylation can also occur at non-CpG sites, for example, methylation can occur at a CpA, CpT, or CpC site. DNA methylation can change the activity of methylated DNA region. For example, when DNA in a promoter region is methylated, transcription of the gene may be repressed. DNA methylation is critical for normal development and abnormality in methylation may disrupt epigenetic regulation. The disruption, e.g., repression, in epigenetic regulation may cause diseases, such as cancer. Promoter methylation in DNA may be indicative of cancer.

The “modified nucleoside profile of DNA” means the position and identity of the nucleoside and the modification status of the nucleoside, such as methylations, within a DNA sequence. As described herein, different modification sensitive sequencing methods can be used to detect such modifications. This includes methods which involve conversion followed by sequencing detect one or more different types of modified or unmodified nucleoside. For example, the TAPS method detects, but does not distinguish between, 5-methylcytosine (5mC) and 5-hydroxymethyl-cytosine (5hmC). Hence, a method for analyzing the modified nucleoside profile of DNA in a sample typically means identifying particular modifications or groups of modification, such as 5mC and/or 5hmC. Modified nucleosides are identified according to the specific method/conversion procedure being used as described above. This generally involves comparing sequence data obtained from DNA that has been subjected to a conversion procedure to a reference sequence. Typically, the method involves (i) comparing the sequence data with (A) one or more pre-determined reference sequence; or (B) sequence data obtained by sequencing a sub-sample of the DNA that was not subjected to the conversion procedure, for example a subsample that was separated before subjecting a separate subsample to the conversion procedure, for example as described herein; and (ii) identifying point differences between the converted DNA sequences and the reference sequence(s) (A) or non-converted DNA sequences (B) as nucleosides (in the initial sample) having a modification status that permits a change in base pairing specificity on exposure to the conversion procedure.

As used herein, “modified cytosine” refers to a cytosine in which at least one position of the cytosine has been substituted with a chemical moiety, such as a methyl or hydroxymethyl, that is different from the substituent at that position in unmodified cytosine. For the avoidance of doubt, “modified cytosine” does not include unmodified cytosine.

The term “hypermethylation” refers to an increased level or degree of methylation of nucleic acid molecule(s) relative to the other nucleic acid molecules within a population (e.g., sample) of nucleic acid molecules. In some embodiments, hypermethylated DNA can include DNA molecules comprising at least 1 methylated residue, at least 2 methylated residues, at least 3 methylated residues, at least 5 methylated residues, or at least 10 methylated residues.

As used herein, “type-specific hypermethylation” means an increased level or degree of methylation of nucleic acid molecules in at one cell or tissue type, or in a plurality of related cell or tissue types, relative to other cell or tissue types. In some embodiments, capturing, identification, and/or detection of type-specific hypermethylated regions facilitates identification of the cell or tissue type from which the nucleic acid molecules originated. The cell or tissue from which a type-specific hypermethylated region originated may be a wild type cell or tissue or a neoplastic cell or tissue.

The term “hypomethylation” refers to a decreased level or degree of methylation of nucleic acid molecule(s) relative to the other nucleic acid molecules within a population (e.g., sample) of nucleic acid molecules. In some embodiments, hypomethylated DNA includes unmethylated DNA molecules. In some embodiments, hypomethylated DNA can include DNA molecules comprising 0 methylated residues, at most 1 methylated residue, at most 2 methylated residues, at most 3 methylated residues, at most 4 methylated residues, or at most 5 methylated residues.

As used herein, “type-specific hypomethylation” means a decreased level or degree of methylation of nucleic acid molecules in at one cell or tissue type, or in a plurality of related cell or tissue types, relative to other cell or tissue types. In some embodiments, capturing, identification, and/or detection of type-specific hypomethylated regions facilitates identification of the cell or tissue type from which the nucleic acid molecules originated. The cell or tissue from which a type-specific hypomethylated region originated may be a wild type cell or tissue or a neoplastic cell or tissue.

As used herein, “methylation status” can refer to the presence or absence of methyl group on a DNA base (e.g. cytosine) at a particular genomic position in a nucleic acid molecule. It can also refer to the degree of methylation in a nucleic acid sequence (e.g., highly methylated, low methylated, intermediately methylated or unmethylated nucleic acid molecules). The methylation status can also refer to the number of nucleotides methylated in a particular nucleic acid molecule.

As used herein, “mutation” refers to a variation from a known reference sequence and includes mutations such as, for example, single nucleotide variants (SNVs), and insertions or deletions (indels). A mutation can be a germline or somatic mutation. In some embodiments, a reference sequence for purposes of comparison is a wildtype genomic sequence of the species of the subject providing a test sample, typically the human genome.

As used herein, the terms “neoplasm” and “tumor” are used interchangeably. They refer to abnormal growth of cells in a subject. A neoplasm or tumor can be benign, potentially malignant, or malignant. A malignant tumor is referred to as a cancer or a cancerous tumor.

As used herein, “next-generation sequencing” or “NGS” refers to sequencing technologies having increased throughput as compared to traditional Sanger- and capillary electrophoresis-based approaches, for example, with the ability to generate hundreds of thousands of relatively small sequence reads at a time. Some examples of next-generation sequencing techniques include, but are not limited to, sequencing by synthesis, sequencing by ligation, and sequencing by hybridization. In some embodiments, next-generation sequencing includes the use of instruments capable of sequencing single molecules. Examples of commercially available instruments for performing next-generation sequencing include, but are not limited to, NextSeq, HiSeq, NovaSeq, MiSeq, Ion PGM and Ion GeneStudio S5.

As used herein, “nucleic acid tag” refers to a short nucleic acid (e.g., less than about 500 nucleotides, about 100 nucleotides, about 50 nucleotides, or about 10 nucleotides in length), used to distinguish nucleic acids from different samples (e.g., representing a sample index), distinguish nucleic acids from different partitions (e.g., representing a partition tag) or different nucleic acid molecules in the same sample (e.g., representing a molecular barcode), of different types, or which have undergone different processing. The nucleic acid tag comprises a predetermined, fixed, non-random, random or semi-random oligonucleotide sequence. Such nucleic acid tags may be used to label different nucleic acid molecules or different nucleic acid samples or sub-samples. Nucleic acid tags can be single-stranded, double-stranded, or at least partially double-stranded. Nucleic acid tags optionally have the same length or varied lengths. Nucleic acid tags can also include double-stranded molecules having one or more blunt-ends, include 5′ or 3′ single-stranded regions (e.g., an overhang), and/or include one or more other single-stranded regions at other locations within a given molecule. Nucleic acid tags can be attached to one end or to both ends of the other nucleic acids (e.g., sample nucleic acids to be amplified and/or sequenced). Nucleic acid tags can be decoded to reveal information such as the sample of origin, form, or processing of a given nucleic acid. For example, nucleic acid tags can also be used to enable pooling and/or parallel processing of multiple samples comprising nucleic acids bearing different molecular barcodes and/or sample indexes in which the nucleic acids are subsequently being deconvolved by detecting (e.g., reading) the nucleic acid tags. Nucleic acid tags can also be referred to as identifiers (e.g. molecular identifier, sample identifier). Additionally, or alternatively, nucleic acid tags can be used as molecular identifiers (e.g., to distinguish between different molecules or amplicons of different parent molecules in the same sample or sub-sample). This includes, for example, uniquely tagging different nucleic acid molecules in a given sample, or non-uniquely tagging such molecules. In the case of non-unique tagging applications, a limited number of tags (i.e., molecular barcodes) may be used to tag each nucleic acid molecule such that different molecules can be distinguished based on their endogenous sequence information (for example, start and/or stop positions where they map to a selected reference genome, a sub-sequence of one or both ends of a sequence, and/or length of a sequence) in combination with at least one molecular barcode. Typically, a sufficient number of different molecular barcodes are used such that there is a low probability (e.g., less than about a 10%, less than about a 5%, less than about a 1%, or less than about a 0.1% chance) that any two molecules may have the same endogenous sequence information (e.g., start and/or stop positions, subsequences of one or both ends of a sequence, and/or lengths) and also have the same molecular barcode. Terms such as “library adapters having distinct molecular barcodes” encompass library adapters for uniquely or non-uniquely tagging molecules, in that regardless of whether the adapters are for unique or non-unique tagging, distinct barcodes will be present in the population of adapters.

As used herein, DNA that is “not immobilized” or that is “free in solution” refers to DNA that is not bound covalently or non-covalently to a solid support, such as a bead. Such DNA may be free in solution during any step (such as all steps) of the disclosed methods.

The terms “agent that recognizes a modified nucleobase in DNA,” such as an “agent that recognizes a modified cytosine in DNA” refers to a molecule or reagent that binds to or detects one or more modified nucleobases in DNA, such as methyl cytosine.

A “modified nucleobase” is a nucleobase that comprises a difference in chemical structure from an unmodified nucleobase. In the case of DNA, an unmodified nucleobase is adenine, cytosine, guanine, or thymine. In some embodiments, a modified nucleobase is a modified cytosine. In some embodiments, a modified nucleobase is a methylated nucleobase. In some embodiments, a modified cytosine is a methyl cytosine, e.g., a 5-methyl cytosine. In such embodiments, the cytosine modification is a methyl. Agents that recognize a methyl cytosine in DNA include but are not limited to “methyl binding reagents,” which refer herein to reagents that bind to a methyl cytosine. Methyl binding reagents include but are not limited to methyl binding domains (MBDs) and methyl binding proteins (MBPs). In some such embodiments, the DNA may be single-stranded or double-stranded. Suitable agents include agents that recognize modified nucleotides in double-stranded DNA, single-stranded DNA, and both double-stranded and single-stranded DNA.

As used herein, “polynucleotide”, “nucleic acid”, “nucleic acid molecule”, or “oligonucleotide” refers to a linear polymer of nucleosides (including deoxyribonucleosides, ribonucleosides, or analogs thereof) joined by inter-nucleosidic linkages. Typically, a polynucleotide comprises at least three nucleosides. Oligonucleotides often range in size from a few monomeric units, e.g., 3-4, to hundreds of monomeric units. Whenever a polynucleotide is represented by a sequence of letters, such as “ATGCCTG”, the nucleotides are in 5′→3′ order from left to right, and in the case of DNA, “A” denotes deoxyadenosine, “C” denotes deoxycytidine, “G” denotes deoxyguanosine, and “T” denotes deoxythymidine, unless otherwise noted. The letters A, C, G, and T may be used to refer to the bases themselves, to nucleosides, or to nucleotides comprising the bases.

As used herein, “processing” refers to a set of steps used to generate a library of nucleic acids that is suitable for sequencing. The set of steps can include, but are not limited to, partitioning, end repairing, addition of sequencing adapters, tagging, and/or PCR amplification of nucleic acids.

As used herein, “quantitative measure” refers to an absolute or relative measure. A quantitative measure can be, without limitation, a number, a statistical measurement (e.g., frequency, mean, median, standard deviation, or quantile), or a degree or a relative quantity (e.g., high, medium, and low). A quantitative measure can be a ratio of two quantitative measures. A quantitative measure can be a linear combination of quantitative measures. A quantitative measure may be a normalized measure.

As used herein, “reference sequence” refers to a known sequence used for purposes of comparison with experimentally determined sequences. For example, a known sequence can be an entire genome, a chromosome, or any segment thereof. A reference sequence can align with a single contiguous sequence of a genome or chromosome or chromosome arm or can include non-contiguous segments that align with different regions of a genome or chromosome. Examples of reference sequences include, for example, human genomes, such as, hg19 and hg38.

As used herein, “sample” means anything capable of being analyzed by the methods and/or systems disclosed herein.

As used herein, an “original sample” is a sample (e.g., of blood, plasma, or serum) as originally obtained from a source, such as a subject, tissue, or cell.

As used herein, “sequencing” refers to any of a number of technologies used to determine the sequence (e.g., the identity and order of monomer units) of a biomolecule, e.g., a nucleic acid such as DNA or RNA. Examples of sequencing methods include, but are not limited to, targeted sequencing, single molecule real-time sequencing, exon or exome sequencing, intron sequencing, electron microscopy-based sequencing, panel sequencing, transistor-mediated sequencing, direct sequencing, random shotgun sequencing, Sanger dideoxy termination sequencing, whole-genome sequencing, sequencing by hybridization, pyrosequencing, duplex sequencing, cycle sequencing, single-base extension sequencing, solid-phase sequencing, high-throughput sequencing, massively parallel signature sequencing, emulsion PCR, co-amplification at lower denaturation temperature-PCR (COLD-PCR), multiplex PCR, sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, 454 sequencing, Solexa Genome Analyzer sequencing, SOLiD™ sequencing, MS-PET sequencing, and a combination thereof. In some embodiments, sequencing can be performed by a gene analyzer such as, for example, gene analyzers commercially available from Illumina, Inc., Pacific Biosciences, Inc., or Applied Biosystems/Thermo Fisher Scientific, among many others.

As used herein, “sequence information” in the context of a nucleic acid polymer means the order and identity of monomer units (e.g., nucleotides, etc.) in that polymer.

As used herein, the terms “somatic mutation” or “somatic variation” are used interchangeably. They refer to a mutation in the genome that occurs after conception. Somatic mutations can occur in any cell of the body except germ cells and accordingly, are not passed on to progeny.

As used herein, “subject” refers to an animal, such as a mammalian species (e.g., human) or avian (e.g., bird) species, or other organism, such as a plant. More specifically, a subject can be a vertebrate, e.g., a mammal such as a mouse, a primate, a simian or a human. Animals include farm animals (e.g., production cattle, dairy cattle, poultry, horses, pigs, and the like), sport animals, and companion animals (e.g., pets or support animals). A subject can be a healthy individual, an individual that has or is suspected of having a disease or a predisposition to the disease, or an individual in need of therapy or suspected of needing therapy. The terms “individual” or “patient” are intended to be interchangeable with “subject”. For example, a subject can be an individual who has been diagnosed with having a cancer, is going to receive a cancer therapy, and/or has received at least one cancer therapy. The subject can be in remission of a cancer. As another example, the subject can be an individual who is diagnosed of having an autoimmune disease. As another example, the subject can be a female individual who is pregnant or who is planning on getting pregnant, who may have been diagnosed of or suspected of having a disease, e.g., a cancer, an auto-immune disease.

As used herein, “tumor fraction” refers to the proportion of cfDNA molecules that originated from tumor cells for a given sample, or sample-region pair.

As used herein, an “asymmetric adapter” is a double stranded adapter in which the two strands are not completely complementary or are otherwise distinguishable such that synthesis of a complementary sequence of one strand of the adapter results in a sequence that is distinguishable from the sequence of the other strand of the adapter. Examples of asymmetric adapters are Y-shaped adapters and bubble adapters.

As used herein, a “Y-shaped adapter” refers to an adapter comprising two DNA strands comprising complementary and non-complementary parts, wherein the non-complementary parts form single-stranded arms. The adapter can be attached to a sample or insert DNA molecule, e.g., by ligation, such that the complementary (double-stranded) part of the adapter is proximal to the sample or insert DNA molecule. Prior to attachment, the double stranded portion of the Y-shaped adapter may have a blunt end or an overhang, e.g., of one to three nucleotides. The single stranded arms may or may not be of identical length.

As used herein, a “bubble adapter” refers to an adapter comprising two DNA strands comprising a non-complementary part flanked by complementary parts, such that the adapter has a single stranded region located between double-stranded regions. The adapter can be attached to a sample or insert DNA molecule, e.g., by ligation, such that one of the complementary (double-stranded) parts of the adapter is proximal to the sample or insert DNA molecule. Prior to attachment, the double stranded portion of the Y-shaped adapter that would be attached to the insert or sample molecule may have a blunt end or an overhang, e.g., of one to three nucleotides. The single stranded portions of the two strands may or may not be of identical length.

The terms “or a combination thereof” and “or combinations thereof” as used herein refers to any and all permutations and combinations of the listed terms preceding the term. For example, “A, B, C, or combinations thereof” is intended to include at least one of: A, B, C, AB, AC, BC, or ABC, and if order is important in a particular context, also BA, CA, CB, ACB, CBA, BCA, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

“Buffy coat” refers to the portion of a blood (such as whole blood) or bone marrow sample that contains all or most of the white blood cells and platelets of the sample. The buffy coat fraction of a sample can be prepared from the sample using centrifugation, which separates sample components by density. For example, following centrifugation of a whole blood sample, the buffy coat fraction is situated between the plasma and erythrocyte (red blood cell) layers. The buffy coat can contain both mononuclear (e.g., T cells, B cells, NK cells, dendritic cells, and monocytes) and polymorphonuclear (e.g., granulocytes such as neutrophils and eosinophils) white blood cells.

As used herein, “leukapheresis” refers to a procedure in which white blood cells (leukocytes) are isolated from a sample of blood collected from a subject. Leukapheresis may be performed, e.g., obtain cells for research, diagnostic, prognostic, or monitoring purposes, such as those described herein. Thus, as used herein, a “leukapheresis sample” refers to a sample comprising leukocytes collected from a subject using leukapheresis.

As used herein, “peripheral blood mononuclear cells” or “PBMCs” refers to immune cells having a single, round nucleus that originate in bone marrow and are found in the peripheral circulation. Such cells include, e.g., lymphocytes (T cells, B cells, and NK cells) as well as monocytes, and are isolated from blood samples (such as from a whole blood sample collected from a subject) using density gradient centrifugation.

As used herein, “amplify,” “amplifying,” or “amplification” refers to a process by which extra or multiple copies of a particular polynucleotide are formed. Amplification methods can include any suitable methods known in the art. As used herein, a nucleic acid molecule amplified using “methylation-preserving amplification” substantially maintains its methylation status post-amplification.

A “X₁nnnX₂ mutation” in a specified polypeptide as used herein, where X₁ and X₂ are amino acids and nnn is a position in an amino acid sequence, refers to a substitution in the polypeptide of amino acid X₁ present at position nnn of the full-length wild-type polypeptide with amino acid X₂. The polypeptide is the human polypeptide unless indicated otherwise. The polypeptide comprising the X₁nnnX₂ mutation may, but does not necessarily, comprise additional differences from the wild-type sequence, including but not limited to truncations and deletions as well as other substitutions. For example, a “T1372S mutation” in TET2 refers to a substitution in a TET2 enzyme of the threonine present at position 1372 of the full-length wild-type human TET2 enzyme with a serine. Position 1372 of wild-type human TET2 aligns to position 258 and 248, respectively, of the truncated TET2 sequences disclosed as SEQ ID NOs: 23 and 24 of U.S. Pat. No. 10,961,525. The immediate wild-type sequence context of position 1372 of human TET2 is FSGVTACLD (SEQ ID NO: 13) where the T is at position 1372. Thus, a TET2 enzyme comprising a T1372S mutation may comprise the sequence FSGVSACLD (SEQ ID NO: 14) or optionally a variant of SEQ ID NO: 14 in which at least 5, 6, 7, or 8 positions match SEQ ID NO: 14 including position 5. Similarly, a “V1900X₂ mutation” where X₂ is A, C, G, I, or P in TET2 refers to a substitution in a TET2 enzyme of the valine present at position 1900 of the full-length wild-type human TET2 enzyme with an alanine, cysteine, glycine, isoleucine, or proline.

The term “methylation-dependent nuclease” refers to a nuclease that preferentially cuts methylated DNA relative to unmethylated DNA. For example, a methylation-dependent nuclease may cut at or near a recognition sequence such as a restriction site in a manner dependent on methylation of at least one of the nucleobases in the recognition sequence, such as a cytosine. In some embodiments, the nucleolytic activity of the methylation-dependent nuclease is at least 10, 20, 50, or 100-fold higher on a methylated recognition site relative to an unmethylated control in a standard nucleolysis assay. Methylation-dependent nucleases include methylation-dependent restriction enzymes.

As used herein, “methylation-dependent restriction enzyme” or “MDRE” refers to a restriction enzyme that is dependent on methylation of the DNA (e.g. cytosine methylation) i.e., the presence or absence of methyl group in a nucleotide base alters the rate at which the enzyme cleaves the target DNA. In some embodiments, the methylation dependent restriction enzymes do not cleave the DNA if a particular nucleotide base is unmethylated at the recognition sequence. For example, MspJI is a methylation dependent restriction enzyme with a recognition sequence “mCNNR(N9)” and it does not cleave DNA if the absence of the methylated cytosine (mC) in the recognition sequence.

The term “methylation-sensitive nuclease” refers to a nuclease that preferentially cuts unmethylated DNA relative to methylated DNA. For example, a methylation-sensitive nuclease may cut at or near a recognition sequence such as a restriction site in a manner dependent on lack of methylation of at least one of the nucleobases in the recognition sequence, such as a cytosine. In some embodiments, the nucleolytic activity of the methylation-sensitive nuclease is at least 10, 20, 50, or 100-fold higher on an unmethylated recognition site relative to a methylated control in a standard nucleolysis assay. Methylation-sensitive nucleases include methylation-sensitive restriction enzymes.

“Solid tissue” or “solid tissue cells” as used herein means tissue or cells, respectively, in or derived from a solid tissue. Solid tissue cells exclude circulating cell types, such as cells normally present in blood or lymph. Examples of solid tissue types include but are not limited to colon, lung, breast, skin, prostate, stomach, pancreas, bladder, kidney, and liver.

A “reaction cleanup” refers to the removal of contaminants such as salts, enzymes, unincorporated dNTPs, primers, ethidium bromide, and other impurities that can interfere with downstream analysis. For example, when a reaction cleanup is performed between end repair and an A-tailing reaction, it removes unincorporated dNTPs such that the A-tailing reaction can be performed solely in the presence of dATP (i.e. not dCTP, dGTP and dCTP, as used in the end tailing reaction). Reaction cleanups can be performed using commercially available kits such as MinElute Reaction Cleanup Kit (Qiagen).

“Regions of the end-repaired DNA that were synthesized during the end repair reaction”, also referred to as “repaired regions” or “synthesized regions,” refer to regions of the DNA that were not present in the DNA prior to the end repair and A-tailing reactions. They are regions which have been synthesized by the polymerases used in the end repair and/or A tailing reactions, if present. In instances where the A-tailing is performed in the same tube as the end repair reaction, all four types of dNTPs will be present, and thus the polymerases used for A-tailing may generate synthesized regions, e.g. through nick translation. In instances where the A-tailing is performed separately to the end repair reaction, and these steps are separated by a reaction cleanup, only dATP will be present in the A-tailing reaction, and thus the polymerases used for A-tailing will not typically generate synthesized regions because the dNTP components are not all present in the A-tailing reaction mix.

A “type of dNTP” refers to a dNTP comprising a specific base, including A, T, G or C. Accordingly, wherein an end repair reaction is performed with dNTPs, wherein at least one type of dNTP comprises a modified base, the end repair reaction may be performed using dCTP comprising 5mC, and dATP, dTTP and dGTP all comprising non-modified bases.

“Capable of identifying the base modification in the at least one type of dNTP” refers to the ability of a modification-sensitive sequencing method to detect the presence or absence of the base modification in the at least one type of dNTP comprising a modified base used in the end repair. This detection of the base modification may be direct, such as in nanopore sequencing or single molecule real time sequencing, wherein the sequencing data itself indicates the presence or absence of a base modification. Alternatively, the detection of the base modification may be indirect, for example wherein the method involves a conversion procedure which alters the base pairing specificity dependent on the base modification status. It is these changes in base pairing specificity which can be detected by the sequencing method, e.g. through the comparison of the sequencing data to a reference sequence. Moreover, a modification-sensitive sequencing method is capable of identifying the base modification in the at least one type of dNTP regardless of whether it can distinguish one base modification from all other base modifications. For example, one form of modification-sensitive sequencing is sequencing after bisulfite conversion. This method is capable of distinguishing 5hmC and 5mC from unmethylated cytosine, but cannot distinguish 5hmC from 5mC.

Bases of the “same identity” refer to the same base, regardless of modification status of that base. For example, cytosine is considered to be the “same identity” as 5-methylcytosine (5mC) and/or 5-hydroxymethyl-cytosine (5hmC), despite them having different modification statuses.

“Or” is used in the inclusive sense, i.e., equivalent to “and/or,” unless the context requires otherwise.

### II. Exemplary Methods

### A. Overview; Detection Steps

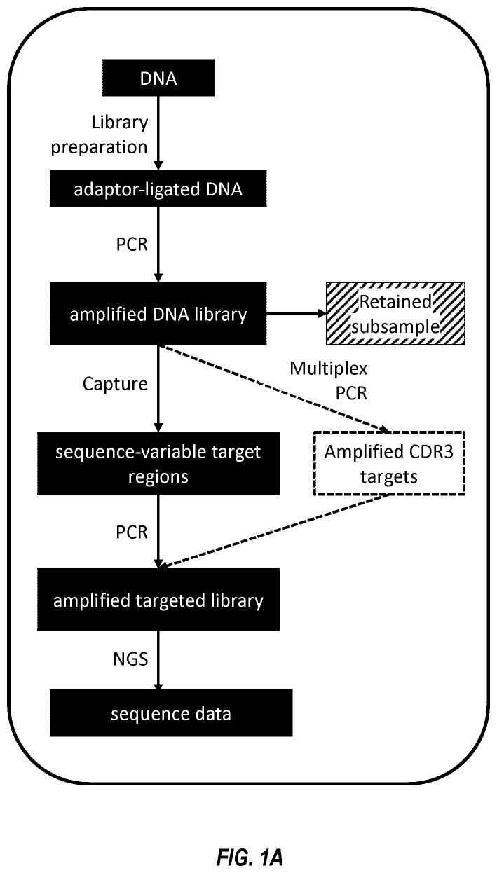

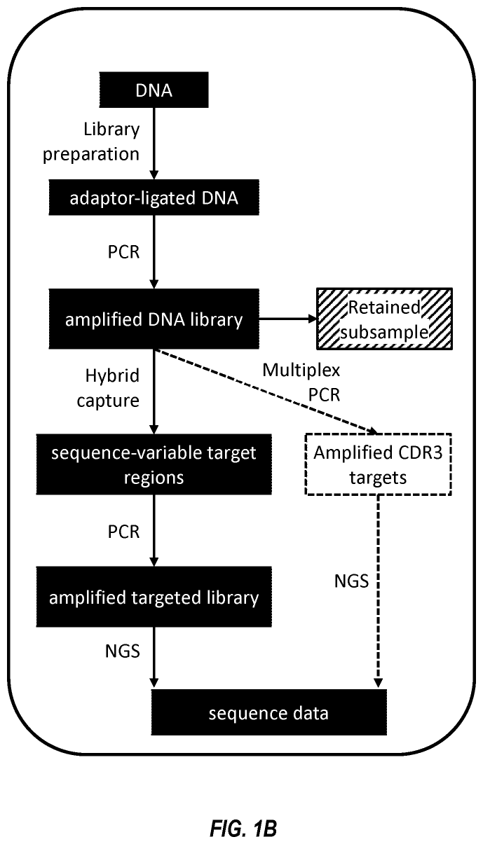

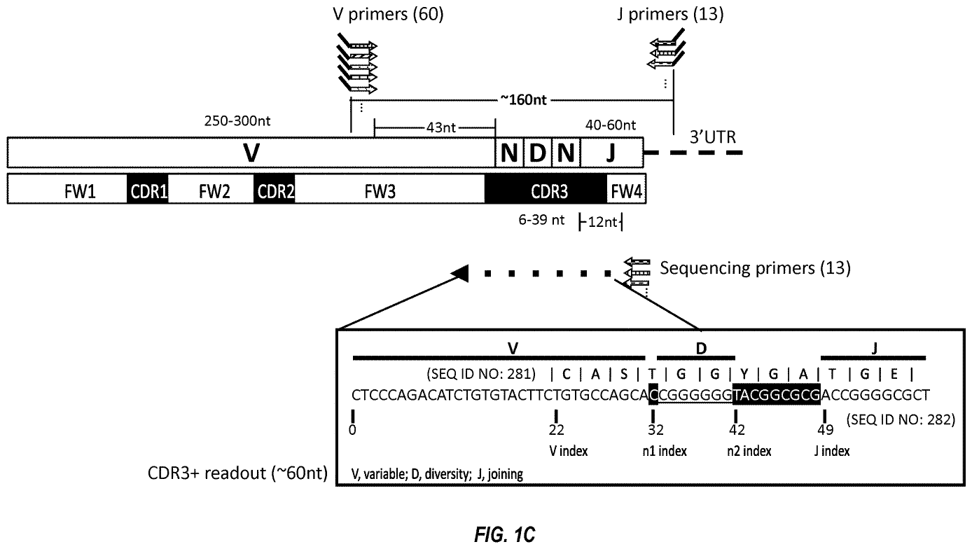

In some embodiments, methods disclosed herein include steps of partitioning an amplified, adapted DNA library into at least a first subsample and a second subsample. For example, as illustrated in FIG. 1A, DNA isolated from a sample can be ligated to adapters for sequencing library preparation, and then the adapted DNA can be amplified (e.g., LP-PCR) to provide an amplified, adapted library. The amplified, adapted library may be partitioned into at least a first subsample and a second subsample, and an optional third subsample that can be retained as a backup. A plurality of target regions comprising sequence-variable target regions from the first subsample can be captured from the first subsample, thereby providing captured regions. Segments comprising recombined CDR3 sequences can be amplified from the second subsample by multiplex amplification using a plurality of first primers that bind V regions and a plurality of second primers that bind J regions, thereby providing CDR3-enriched DNA. The multiplex amplification can be exponential. The plurality of first primers that bind V regions can be oppositely oriented relative to the plurality of second primers that bind J regions (e.g., the plurality of first primers that bind V regions can be forward or sense primers and the plurality of second primers that bind J regions can be reverse or antisense primers). The captured regions and the CDR3-enriched DNA can be sequenced. In such embodiments, as illustrated in FIG. 1A, the captured regions and the CDR3-enriched DNA can be pooled and sequenced together.

In some other embodiments, as illustrated in FIG. 1B, DNA isolated from a sample can be ligated to adapters for sequencing library preparation, and then the adapted DNA can be amplified (e.g., LP-PCR) to provide an amplified, adapted library. The amplified, adapted library may be partitioned into at least a first subsample and a second subsample, and an optional third subsample that can be retained, e.g., as a backup. A plurality of target regions comprising sequence-variable target regions from the first subsample can be captured from the first subsample, thereby providing captured regions. Segments comprising recombined CDR3 sequences can be amplified from the second subsample by multiplex amplification using a plurality of first primers that bind V regions and a plurality of second primers that bind J regions, thereby providing CDR3-enriched DNA. The multiplex amplification can be exponential. The plurality of first primers that bind V regions can be oppositely oriented relative to the plurality of second primers that bind J regions (e.g., the plurality of first primers that bind V regions can be forward or sense primers and the plurality of second primers that bind J regions can be reverse or antisense primers). The captured regions and the CDR3-enriched DNA can be sequenced separately.

As illustrated in FIG. 1C, the first primers that bind V regions (V primers) and the second primers that bind J regions (J primers) can be designed using the method, for example, described in Montagne et al., EBioMedicine 59 (2020) 102972. Exemplary first primers that bind V regions (V primers) and second primers that bind J regions (J primers) can include those described in Montagne et al., EBioMedicine 59 (2020) 102972 and listed in Tables 1-3. In some embodiments, the plurality of first primers can include from 10 to 150, from 15 to 125, from 20 to 100, from 30 to 90, from 40 to 80, or from 50 to 70 primers that bind V regions. In some embodiments, the plurality of first primers can include about 10, about 20, about 30, about 40, about 45, about 50, about 52, about 54, about 55, about 56, about 57, about 58, about 59, about 60, about 61, about 62, about 63, about 65, about 70, about 75, about 80, about 90, about 100, about 110, about 120, about 130, about 140, or about 150 primers that bind V regions. In some embodiments, the plurality of second primers can include from 5 to 30, from 10 to 25, or from 10 to 20 primers that bind J regions. In some embodiments, the plurality of second primers can include about 5, about 10, about 11, about 12, about 13, about 14, about 15, about 20, or about 30 primers that bind J regions.

TABLE 1
Human TCRB V primers and adapter sequences
human TCRB primers
Adapter sequence for TCRB constant region PCRI primer (add to 5′ end)

| Sequence | Adapter name |
|---|---|
| ctggagttcagacgtgtgctcttccgatct | ad_min |
| (SEQ ID NO: 277) | |

Adapter sequence for V primers (add to 5′ end)

| Sequence | Adapter name |
|---|---|
| ggagctgtcgttcactc | |
| (SEQ ID NO: 278) | AD1.2 |

Full: TCRB V primers

| | IMGT Accession\| | Unique | Gel |
|---|---|---|---|
| Primer Sequence | Associated V allele | FR3 order | order |
| actccgccgtgtatctctgt | X61441\|TRBV7-8*02 | 1 | 25 |
| (SEQ ID NO: 15) | M11953\|TRBV7-8*01 | | |
| | M27384\|TRBV7-8*03 | | |
| actctgccgtgtatctctgt | X74843\|TRBV7-3*04 | 2 | 20 |
| (SEQ ID NO: 16) | | | |
| actcggccgtgtatctctgt | X58797\|TRBV11-3*02 | 3 | 47 |
| (SEQ ID NO: 17) | X61442\|TRBV7-2*01 | | |
| | L36190\|TRBV7-2*02 | | |
| | X58796\|TRBV11-2*03 | | |
| | M27387\|TRBV7-2*04 | | |
| | U03115\|TRBV11-3*01 | | |
| | L36092\|TRBV11-2*01 | | |
| | U07975\|TRBV7-2*03 | | |
| | M33235\|TRBV11-2*02 | | |
| | MGT000021\|TRBV11-2*04 | | |
| | AB305924\|TRBV11-3*04 | | |
| actcagccgtgtatctctgt | M13550\|TRBV7-3*05 | 4 | 4 |
| (SEQ ID NO: 18) | X61440\|TRBV7-3*01 | | |
| actcagctgtgtatctctgt | L36092\|TRBV7-4*01 | 5 | 36 |
| (SEQ ID NO: 19) | | | |
| actcagccgtgtatctccgt | M97943\|TRBV7-3*02 | 6 | 45 |
| (SEQ ID NO: 20) | | | |
| actcagccgcgtatctccgt | AF009660\|TRBV7-3*03 | 7 | 15 |
| (SEQ ID NO: 21) | | | |
| actcggccatgtatcgctgt | X58806\|TRBV7-6*02 | 8 | 23 |
| (SEQ ID NO: 22) | L36092\|TRBV7-6*01 | | |
| actcagccatgtatcgctgt | L36092\|TRBV7-7*01 | 9 | 37 |
| (SEQ ID NO: 23) | X57607\|TRBV7-7*02 | | |
| actcggccatgtatctctgt | M33233\|TRBV11-1*01 | 10 | 26 |
| (SEQ ID NO: 24) | L14854\|TRBV7-9*07 | | |
| | M27385\|TRBV7-9*05 | | |
| | M14261\|TRBV7-9*04 | | |
| | AF009663\|TRBV7-9*03 | | |
| | M62377\|TRBV11-3*03 | | |
| | L36092\|TRBV7-9*01 | | |
| | M15564\|TRBV7-9*02 | | |
| | X74844\|TRBV7-9*06 | | |
| actcagccgtgtatctctac | U03115\|TRBV17*01 | 11 | 34 |
| (SEQ ID NO: 25) | IMGT000021\|TRBV17*02 | | |
| actcagccctgtatctctgc | U07977\|TRBV4-1*01 | 12 | 5 |
| (SEQ ID NO: 26) | M13855\|TRBV4-1*02 | | |
| acttggctgtgtatctctgt | X61444\|TRBV7-1*01 | 13 | 33 |
| (SEQ ID NO: 27) | | | |
| actcggccctgtatctctgc | X58812\|TRBV4-3*02 | 14 | 27 |
| (SEQ ID NO: 28) | L06888\|TRBV4-3*03 | | |
| | X57616\|TRBV4-3*04 | | |
| | U07978\|TRBV4-3*01 | | |
| actcggccctgtatctctgt | X58811\|TRBV4-2*02 | 15 | 1 |
| (SEQ ID NO: 29) | U07975\|TRBV4-2*01 | | |
| | X57611\|TRBV5-5*02 | | |
| | X57615\|TRBV5-4*02 | | |
| | X58803\|TRBV5-8*02 | | |
| | L36092\|TRBV5-8*01 | | |
| | X58804\|TRBV5-4*04 | | |
| | L36092\|TRBV5-5*01 | | |
| | L36092\|TRBV5-4*01 | | |
| | AF009660\|TRBV5-3*02 | | |
| | X58801\|TRBV5-5*03 | | |
| | S50547\|TRBV5-4*03 | | |
| | X61439\|TRBV5-3*01 | | |
| actcggccctctatctctgt | L36092\|TRBV5-6*01 | 16 | 18 |
| (SEQ ID NO: 30) | L36092\|TRBV5-7*01 | | |
| actcggccctttatctttgc | L36092\|TRBV5-1*01 | 17 | 40 |
| (SEQ ID NO: 31) | M14271\|TRBV5-1*02 | | |
| acacggcactgtatctctgc | L36092\|TRBV23-1*01 | 18 | 39 |
| (SEQ ID NO: 32) | | | |
| actcagctgtgtacttctgt | K02546\|TRBV12-4*01 | 19 | 31 |
| (SEQ ID NO: 33) | X07192\|TRBV12-3*01 | | |
| | M14264\|TRBV12-4*02 | | |
| actcagctgtgtatttttgt | X07223\|TRBV12-5*01 | 20 | 14 |
| (SEQ ID NO: 34) | | | |
| actcagctttgtatttctgt | L36092\|TRBV9*01 | 21 | 19 |
| (SEQ ID NO: 35) | M27380\|TRBV9*03 | | |
| | AF009660\|TRBV9*02 | | |
| actctgctgtgtatttctgt | L06889\|TRBV3-1*02 | 22 | 28 |
| (SEQ ID NO: 36) | U07977\|TRBV3-1*01 | | |
| actcagccatgtacttctgt | M64351\|TRBV2*03 | 23 | 44 |
| (SEQ ID NO: 37) | L36092\|TRBV2*01 | | |
| | M62379\|TRBV2*02 | | |
| actcagccctgtacttctgt | U03115\|TRBV13*01 | 24 | 9 |
| (SEQ ID NO: 38) | M62378\|TRBV13*02 | | |
| actctggcttctatctctgt | L36092\|TRBV30*01 | 25 | 22 |
| (SEQ ID NO: 39) | M13554\|TRBV30*04 | | |
| | L06893\|TRBV30*05 | | |
| | Z13967\|TRBV30*02 | | |
| acgcagccatgtacctgtgt | X58800\|TRBV15*02 | 26 | 43 |
| (SEQ ID NO: 40) | IMGT000021\|TRBV15*04 | | |
| acacagccatgtacctgtgt | U03115\|TRBV15*01 | 27 | 3 |
| (SEQ ID NO: 41) | | | |
| acgcagccatgtaccagtgt | M62376\|TRBV15*03 | 28 | 42 |
| (SEQ ID NO: 42) | | | |
| attcggcagcttatttctgt | L36092\|TRBV18*01 | 29 | 16 |
| (SEQ ID NO: 43) | | | |
| attctggagtttatttctgt | X57722\|TRBV14*02 | 30 | 46 |
| (SEQ ID NO: 44) | X06154\|TRBV14*01 | | |
| attcagcagtgtatttttgt | L26054\|TRBV16*03 | 31 | 7 |
| (SEQ ID NO: 45) | L26231\|TRBV16*01 | | |
| agacatctgtgtacttctgt | AF009662\|TRBV6-6*02 | 32 | 29 |
| (SEQ ID NO: 46) | X58815\|TRBV6-6*03 | | |
| | L33102\|TRBV10-3*04 | | |
| | L36092\|TRBV6-6*01 | | |
| | U17047\|TRBV10-3*02 | | |
| | U03115\|TRBV10-3*01 | | |
| | AF009660\|TRBV6-4*02 | | |
| | X61446\|TRBV6-1*01 | | |
| | L06892\|TRBV6-6*05 | | |
| | X74848\|TRBV6-6*04 | | |
| | L33101\|TRBV10-3*03 | | |
| | X61653\|TRBV6-4*01 | | |
| | L36092\|TRBV6-5*01 | | |
| agacatctgtgtacttgtgt | L36092\|TRBV6-8*01 | 33 | 21 |
| (SEQ ID NO: 47) | | | |
| aaacatctgtgtacttctgt | U07978\|TRBV6-3*01 | 34 | 24 |
| (SEQ ID NO: 48) | X61445\|TRBV6-2*01 | | |
| agacatctgtatacttctgt | X61447\|TRBV6-9*01 | 35 | 30 |
| (SEQ ID NO: 49) | | | |
| agacttctgtttacttctgt | L36092\|TRBV6-7*01 | 36 | 10 |
| (SEQ ID NO: 50) | | | |
| agacctctctgtacttctgt | L36092\|TRBV27*01 | 37 | 6 |
| (SEQ ID NO: 51) | | | |
| agacatctatgtacctctgt | U08314\|TRBV28*01 | 38 | 38 |
| (SEQ ID NO: 52) | | | |
| agacagctctttacttctgt | M11951\|TRBV24-1*01 | 39 | 8 |
| (SEQ ID NO: 53) | IMGT000021\|TRBV24-1*02 | | |
| agacatctgtatatttctgc | L36092\|TRBV10-1*01 | 40 | 35 |
| (SEQ ID NO: 54) | AF009660\|TRBV10-1*02 | | |
| agacatctgtgtatttctgc | L36092\|TRBV10-2*01 | 41 | 32 |
| (SEQ ID NO: 55) | IMGT000021\|TRBV10-2*03 | | |
| atacctctcagtacctctgt | L36092\|TRBV25-1*01 | 42 | 41 |
| (SEQ ID NO: 56) | | | |
| cgacagctttctatctctgt | U48259\|TRBV19*02 | 43 | 2 |
| (SEQ ID NO: 57) | M97725\|TRBV19*03 | | |
| | L36092\|TRBV19*01 | | |
| gcttctacatctgcagtgct | X72719\|TRBV20-1*02 | 44 | 17 |
| (SEQ ID NO: 58) | | | |
| acagcagcttctacatctgc | D13088\|TRBV20-1*06 | 45 | 12 |
| (SEQ ID NO: 59) | X57604\|TRBV20-1*05 | | |
| | M14263\|TRBV20-1*04 | | |
| | M11955\|TRBV20-1*01 | | |
| | X74852\|TRBV20-1*07 | | |
| | M11954\|TRBV20-1*03 | | |
| acagcagcatatatctctgc | L36092\|TRBV29-1*01 | 46 | 13 |
| (SEQ ID NO: 60) | X04926\|TRBV29-1*03 | | |
| | M13847\|TRBV29-1*02 | | |
| cccgctcccagacatctgtg | U17048\|TRBV10-2*02 | 47 | 11 |
| (SEQ ID NO: 61) | | | |

1MM: TCRB V primers

| | Heatmap | Gel |
|---|---|---|
| Primer Sequence | Order | Order |
| actcggccgtgtatctctgt (SEQ ID NO: 62) | 1 | 25 |
| actcagctgtgtatctctgt (SEQ ID NO: 63) | 2 | 19 |
| actcagccgcgtatctccgt (SEQ ID NO: 64) | 3 | 9 |
| actcagccatgtatcgctgt (SEQ ID NO: 65) | 4 | 20 |
| actcagccgtgtatctctac (SEQ ID NO: 66) | 5 | 26 |
| actcagccctgtatctctgc (SEQ ID NO: 67) | 6 | 1 |
| acttggctgtgtatctctgt (SEQ ID NO: 68) | 7 | 18 |
| actcggccctctatctctgt (SEQ ID NO: 69) | 8 | 11 |
| actcggccctttatctttgc (SEQ ID NO: 70) | 9 | 22 |
| acacggcactgtatctctgc (SEQ ID NO: 71) | 10 | 30 |
| actcagctgtgtacttctgt (SEQ ID NO: 72) | 11 | 16 |
| actcagctgtgtatttttgt (SEQ ID NO: 73) | 12 | 28 |
| actcagctttgtatttctgt (SEQ ID NO: 74) | 13 | 12 |
| actctgctgtgtatttctgt (SEQ ID NO: 75) | 14 | 14 |
| actcagccctgtacttctgt (SEQ ID NO: 76) | 15 | 5 |
| actctggcttctatctctgt (SEQ ID NO: 77) | 16 | 27 |
| acgcagccatgtacctgtgt (SEQ ID NO: 78) | 17 | 24 |
| acgcagccatgtaccagtgt (SEQ ID NO: 79) | 18 | 23 |
| attcggcagcttatttctgt (SEQ ID NO: 80) | 19 | 10 |
| attctggagtttatttctgt (SEQ ID NO: 81) | 20 | 29 |
| attcagcagtgtatttttgt (SEQ ID NO: 82) | 21 | 3 |
| agacatctgtgtacttctgt (SEQ ID NO: 83) | 22 | 15 |
| agacatctgtgtacttgtgt (SEQ ID NO: 84) | 23 | 13 |
| agacttctgtttacttctgt (SEQ ID NO: 85) | 24 | 6 |
| agacctctctgtacttctgt (SEQ ID NO: 86) | 25 | 2 |
| agacatctatgtacctctgt (SEQ ID NO: 87) | 26 | 21 |
| agacagctctttacttctgt (SEQ ID NO: 88) | 27 | 4 |
| agacatctgtgtatttctgc (SEQ ID NO: 89) | 28 | 17 |
| atacctctcagtacctctgt (SEQ ID NO: 90) | 29 | 34 |
| cgacagctttctatctctgt (SEQ ID NO: 91) | 30 | 33 |
| gcttctacatctgcagtgct (SEQ ID NO: 92) | 31 | 31 |
| acagcagcttctacatctgc (SEQ ID NO: 93) | 32 | 7 |
| acagcagcatatatctctgc (SEQ ID NO: 94) | 33 | 8 |
| cccgctcccagacatctgtg (SEQ ID NO: 95) | 34 | 32 |

Compact: TCRB V primers

| | Heatmap | Gel |
|---|---|---|
| Primer Sequence | Order | Order |
| actcigciitgtatctctgt (SEQ ID NO: 96) | 1 | 1 |
| actcagccgigtatctccgt (SEQ ID NO: 97) | 2 | 7 |
| actcggccatgtatcgctgt (SEQ ID NO: 98) | 3 | 11 |
| actcagccgtgtatctctac (SEQ ID NO: 99) | 4 | 10 |
| actcigccctitatctitgc (SEQ ID NO: 100) | 5 | 5 |
| acacggcactgtatctctgc (SEQ ID NO: 101) | 6 | 16 |
| actcagctgtgtatttttgt (SEQ ID NO: 102) | 7 | 14 |
| actctggcttctatctctgt (SEQ ID NO: 103) | 8 | 12 |
| acicagccatgtacciitgt (SEQ ID NO: 104) | 9 | 4 |
| attcggcagiitatttitgt (SEQ ID NO: 105) | 10 | 8 |
| attctggagtttatttctgt (SEQ ID NO: 106) | 11 | 15 |
| agacatctitgtacititgt (SEQ ID NO: 107) | 12 | 3 |
| agaciictitttacttctgt (SEQ ID NO: 108) | 13 | 13 |
| actcagciitgtacttctgt (SEQ ID NO: 109) | 14 | 2 |
| agacatctgtitatttctgc (SEQ ID NO: 110) | 15 | 6 |
| atacctctcagtacctctgt (SEQ ID NO: 111) | 16 | 20 |
| cgacagctttctatctctgt (SEQ ID NO: 112) | 17 | 19 |
| gcttctacatctgcagtgct (SEQ ID NO: 113) | 18 | 17 |
| acagcagcttitaiitctgc (SEQ ID NO: 114) | 19 | 9 |
| cccgctcccagacatctgtg (SEQ ID NO: 115) | 20 | 18 |

PCR2 primers

| Primer Sequence: X refers to barcode nucleotides | Adapters |
|---|---|
| caagcagaagacggcatacgagatXXXXXXXXgtgactggagttcagacgtg | P7_ad_min |
| tgctcttccgatct (SEQ ID NO: 279) | |
| aatgatacggcgaccaccgagatctacacXXXXXXXXacactctttccgatc | P5_AD1.2 |
| tggagctgtcgttcactc (SEQ ID NO: 280) | |

TABLE 2
Human TCRA, TCRG, and TCRD primers. Adapter sequences can be found in Table 1.
Human TCRA, TCRG, and TCRD primers
Full: human TCRA V primers

| | IMGT Accession\| |
|---|---|
| Primer Sequence | Associated V allele |
| atgctgctgtgtactactgc | AE000660\|TRAV26-2*01 |
| (SEQ ID NO: 116) | |
| actcagccacctacttctgt | M17660\|TRAV23/DV6*02 |
| (SEQ ID NO: 117) | AE000660\|TRAV23/DV6*01 |
| | M97704\|TRAV23/DV6*03 |
| actcagctatctacttctgt | AE000659\|TRAV5*01 |
| (SEQ ID NO: 118) | |
| atgctgctgagtacttctgt | AE000659\|TRAV8-3*01 |
| (SEQ ID NO: 119) | AE000660\|TRAV8-7*01 |
| | M35617\|TRAV8-3*02 |
| atgcgtctgagtacttctgt | L06885\|TRAV8-3*03 |
| (SEQ ID NO: 120) | |
| atacaggccactacctctgt | X04957\|TRAV27*02 |
| (SEQ ID NO: 121) | |
| actccgctgtgtacttctgt | AE000659\|TRAV9-1*01 |
| (SEQ ID NO: 122) | |
| actcagccacctacctctgt | X58736\|TRAV21*02 |
| (SEQ ID NO: 123) | AE000660\|TRAV21*01 |
| atacaggcctctacctctgt | AE000660\|TRAV27*01 |
| (SEQ ID NO: 124) | D13075\|TRAV27*03 |
| actctgccgtgtactactgc | AE000660\|TRAV18*01 |
| (SEQ ID NO: 125) | |
| actcagctgtctacttttgt | M17658\|TRAV13-2*02 |
| (SEQ ID NO: 126) | AE000659\|TRAV13-2*01 |
| actcagcaatgtatttctgt | AE000659\|TRAV14/DV4*02 |
| (SEQ ID NO: 127) | M21624\|TRAV14/DV4*03 |
| atgtaggaacctacttctgt | AE000660\|TRAV25*01 |
| (SEQ ID NO: 128) | |
| attcagccacctatttctgt | AE000659\|TRAV7*01 |
| (SEQ ID NO: 129) | |
| atgcaggcatctacctctgt | AE000660\|TRAV34*01 |
| (SEQ ID NO: 130) | |
| acacagctgagtacttctgt | AE000659\|TRAV8-1*01 |
| (SEQ ID NO: 131) | |
| attcagccacatacctctgt | M17661\|TRAV24*02 |
| (SEQ ID NO: 132) | |
| atgtaggcatctacttctgt | AE000660\|TRAV35*01 |
| (SEQ ID NO: 133) | X58738\|TRAV35*02 |
| acgcggctgagtacttctgt | M12959\|TRAV8-4*04 |
| (SEQ ID NO: 134) | X63455\|TRAV8-4*05 |
| | M17650\|TRAV8-2*02 |
| | M12423\|TRAV8-4*02 |
| | D13077\|TRAV8-4*03 |
| | AE000659\|TRAV8-2*01 |
| | AE000659\|TRAV8-4*01 |
| | K02777\|TRAV8-4*06 |
| actcaggcgtttatttctgt | AE000660\|TRAV22*01 |
| (SEQ ID NO: 135) | |
| actcggccgtctacctctgt | Z46643\|TRAV36/DV7*04 |
| | X58767\|TRAV36/DV7*03 |
| (SEQ ID NO: 136) | X61070\|TRAV36/DV7*02 |
| acactgcgatgtatttctgt | AE000661\|TRAV38-1*01 |
| (SEQ ID NO: 137) | L06880\|TRAV38-1*04 |
| | M95394\|TRAV38-1*03 |
| | M64355\|TRAV38-1*02 |
| actcagcaatgtacttctgt | M21626\|TRAV14/DV4*01 |
| (SEQ ID NO: 138) | |
| actcagcagtatacttctgt | AE000660\|TRAV19*01 |
| (SEQ ID NO: 139) | |
| actcagccgtgtactactgt | X73521\|TRAV40*01 |
| (SEQ ID NO: 140) | |
| acacggctgagtacttctgt | X02850\|TRAV8-6*01 |
| (SEQ ID NO: 141) | AE000659\|TRAV8-6*02 |
| actcagccacatacctctgt | AE000660\|TRAV24*01 |
| (SEQ ID NO: 142) | |
| actcagctacctacctctgt | AE000659\|TRAV6*01 |
| (SEQ ID NO: 143) | X58747\|TRAV6*02 |
| | Z49060\|TRAV6*03 |
| | Y10410\|TRAV6*05 |
| | Y10409\|TRAV6*04 |
| actctgcagtgtacttctgt | S81645\|TRAV29/DV5*02 |
| (SEQ ID NO: 144) | AE000660\|TRAV29/DV5*01 |
| acactgctgtgtactattgc | AE000660\|TRAV26-1*01 |
| (SEQ ID NO: 145) | M27371\|TRAV26-1*02 |
| | L06886\|TRAV26-1*03 |
| actcggccatctacctctgt | AE000660\|TRAV36/DV7*01 |
| (SEQ ID NO: 146) | |
| actctgcctcttacttctgc | X04939\|TRAV1-1*02 |
| (SEQ ID NO: 147) | AE000658\|TRAV1-1*01 |
| tctctgccacctacttctgt | AE000661\|TRAV39*01 |
| (SEQ ID NO: 148) | |
| actctgcctcttacctctgt | AE000658\|TRAV1-2*01 |
| (SEQ ID NO: 149) | |
| acactgctgtgtactactgc | AE000658\|TRAV4*01 |
| (SEQ ID NO: 150) | |
| actccgctttgtacttctgt | AE000658\|TRAV3*01 |
| (SEQ ID NO: 151) | |
| actcagccacttatctctgt | X70305\|TRAV20*04 |
| (SEQ ID NO: 152) | S60789\|TRAV20*03 |
| | AE000660\|TRAV20*01 |
| | X68696\|TRAV20*02 |
| atgctgctgtttactactgt | AE000658\|TRAV2*01 |
| (SEQ ID NO: 153) | M17659\|TRAV2*02 |
| acccggctgagtacttctgt | M17665\|TRAV8-4*07 |
| (SEQ ID NO: 154) | |
| attcagccacctacctctgt | AE000659\|TRAV12-2*01 |
| (SEQ ID NO: 155) | AE000659\|TRAV12-1*01 |
| | M17657\|TRAV12-1*02 |
| | M17656\|TRAV12-3*02 |
| | M81774\|TRAV12-2*02 |
| | X04946\|TRAV12-2*03 |
| | AE000659\|TRAV12-3*01 |
| actctgccgtctacatctgt | AE000661\|TRAV41*01 |
| (SEQ ID NO: 156) | |
| actcaggaacctacttctgc | L06883\|TRAV30*03 |
| (SEQ ID NO: 157) | AE000660\|TRAV30*01 |
| | X58768\|TRAV30*02 |
| atgccgcgatgtatttctgt | AE000661\|TRAV38-2/DV8*01 |
| (SEQ ID NO: 158) | |
| attcagcctcctacatctgt | AE000659\|TRAV10*01 |
| (SEQ ID NO: 159) | |
| actcggctgtctacttctgt | AE000659\|TRAV13-1*01 |
| (SEQ ID NO: 160) | X04954\|TRAV13-1*02 |
| acactgcttcttacttctgt | AE000660\|TRAV17*01 |
| (SEQ ID NO: 161) | |
| actcagcggtgtacttctgt | L06882\|TRAV9-2*04 |
| | X58745\|TRAV9-2*02 |
| (SEQ ID NO: 162) | L06881\|TRAV9-2*03 |
| | AE000659\|TRAV9-2*01 |
| actcagccatgtattactgt | AE000659\|TRAV16*01 |
| (SEQ ID NO: 163) | |

1MM: human TCRA V primers
actccgctgtgtacttctgt (SEQ ID NO: 164)
actcagcggtgtacttctgt (SEQ ID NO: 165)
actcggccgtctacctctgt (SEQ ID NO: 166)
actcagccacctacctctgt (SEQ ID NO: 167)
acgcggctgagtacttctgt (SEQ ID NO: 168)
actcagcagtatacttctgt (SEQ ID NO: 169)
actcagctatctacttctgt (SEQ ID NO: 170)
actctgccgtctacatctgt (SEQ ID NO: 171)
actcagcaatgtacttctgt (SEQ ID NO: 172)
actctgccgtgtactactgc (SEQ ID NO: 173)
acactgcttcttacttctgt (SEQ ID NO: 174)
attcagccacctatttctgt (SEQ ID NO: 175)
actctgcctcttacctctgt (SEQ ID NO: 176)
actcggctgtctacttctgt (SEQ ID NO: 177)
atacaggcctctacctctgt (SEQ ID NO: 178)
actcagccgtgtactactgt (SEQ ID NO: 179)
acactgcgatgtatttctgt (SEQ ID NO: 180)
atgctgctgtgtactactgc (SEQ ID NO: 181)
attcagcctcctacatctgt (SEQ ID NO: 182)
actctgcagtgtacttctgt (SEQ ID NO: 183)
atgccgcgatgtatttctgt (SEQ ID NO: 184)
atgctgctgtttactactgt (SEQ ID NO: 185)
actcagccacttatctctgt (SEQ ID NO: 186)
acactgctgtgtactactgc (SEQ ID NO: 187)
atgcaggcatctacctctgt (SEQ ID NO: 188)
actcagccatgtattactgt (SEQ ID NO: 189)
actctgcctcttacttctgc (SEQ ID NO: 190)
atgtaggcatctacttctgt (SEQ ID NO: 191)
acactgctgtgtactattgc (SEQ ID NO: 192)
actcaggcgtttatttctgt (SEQ ID NO: 193)
actcagctgtctacttttgt (SEQ ID NO: 194)
actcaggaacctacttctgc (SEQ ID NO: 195)
atgcgtctgagtacttctgt (SEQ ID NO: 196)
atgctgctgagtacttctgt (SEQ ID NO: 197)
atgtaggaacctacttctgt (SEQ ID NO: 198)
acacagctgagtacttctgt (SEQ ID NO: 199)
tctctgccacctacttctgt (SEQ ID NO: 200)
attcagccacatacctctgt (SEQ ID NO: 201)
Compact: human TCRA V primers
actcigcigtitacttctgt (SEQ ID NO: 202)
actcagccaiitacitctgt (SEQ ID NO: 203)
acicigctgagtacttctgt (SEQ ID NO: 204)
actcagccitgtaitictgt (SEQ ID NO: 205)
actctgccgiitactictgc (SEQ ID NO: 206)
aticaggciictacctctgt (SEQ ID NO: 207)
aiictgctgtgtactaitgc (SEQ ID NO: 208)
attcagccicctaiitctgt (SEQ ID NO: 209)
acactgcgatgtatttctgt (SEQ ID NO: 210)
atgtaggiaictacttctgt (SEQ ID NO: 211)
actcaggcgtttatttctgt (SEQ ID NO: 212)
actctgcctcttacctctgt (SEQ ID NO: 213)
actcagctgtctacttttgt (SEQ ID NO: 214)
atgccgcgatgtatttctgt (SEQ ID NO: 215)
actcaggaacctacttctgc (SEQ ID NO: 216)
atgcgtctgagtacttctgt (SEQ ID NO: 217)
acactgcttcttacttctgt (SEQ ID NO: 218)
tctctgccacctacttctgt (SEQ ID NO: 219)
atgctgctgtttactactgt (SEQ ID NO: 220)
Full: human TCRG V primers

| | IMGT Accession\| |
|---|---|
| Primer Sequence | Associated V allele |
| atgaggtggtgtaccactgt (SEQ ID NO: | Y11227\|TRGV11*01 |
| 221) | AF159056\|TRGV11*02 |
| attctgggttctattactgt (SEQ ID NO: | M12949\|TRGV1*01 |
| 222) | |
| actttggggtctattactgt (SEQ ID NO: | IMGT000011\|TRGV2*03 |
| 223) | |
| actctggggtctattactgt (SEQ ID NO: | M27337\|TRGV2*02 |
| 224) | M13429\|TRGV2*01 |
| | M13434\|TRGV8*01 |
| acatagctacctactactgt (SEQ ID NO: | X15274\|TRGV9*02 |
| 225) | X07205\|TRGV9*01 |
| acatggccgtttactactgt (SEQ ID NO: | X74798\|TRGV10*02 |
| 226) | X07206\|TRGV10*01 |
| attctggggtctattactgt (SEQ ID NO: | X13355\|TRGV5*01 |
| 227) | X04038\|TRGV3*02 |
| | M13430\|TRGV3*01 |
| actctggagtctattactgt (SEQ ID NO: | X13354\|TRGV4*02 |
| 228) | X15272\|TRGV4*01 |

1MM: human TCRG V primers
actctggggtctattactgt (SEQ ID NO: 229)
atgaggtggtgtaccactgt (SEQ ID NO: 230)
acatggccgtttactactgt (SEQ ID NO: 231)
acatagctacctactactgt (SEQ ID NO: 232)
attctgggttctattactgt (SEQ ID NO: 233)
Compact: human TCRG V primers
aititggigtctattactgt (SEQ ID NO: 234)
acatagctacctactactgt (SEQ ID NO: 235)
atgaggtggtgtaccactgt (SEQ ID NO: 236)
acatggccgtttactactgt (SEQ ID NO: 237)
Full: human TCRD V primers

| | IMGT Accession\| |
|---|---|
| Primer Sequence | Associated V allele |
| attcagcaaagtacttttgt (SEQ ID NO: | M22198\|TRDV1*01 |
| 238) | |
| atgccgcgatgtatttctgt (SEQ ID NO: | AE000661\|TRAV38-2/DV8*01 |
| 239) | |
| actctgcagtgtacttctgt (SEQ ID NO: | S81645\|TRAV29/DV5*02 |
| 240) | AE000660\|TRAV29/DV5*01 |
| actcggccatctacctctgt (SEQ ID NO: | AE000660\|TRAV36/DV7*01 |
| 241) | |
| actcagccacctacttctgt (SEQ ID NO: | M97704\|TRAV23/DV6*03 |
| 242) | Y10411\|TRAV23/DV6*04 |
| | AE000660\|TRAV23/DV6*01 |
| | M17660\|TRAV23/DV6*02 |
| actcagcaatgtatttctgt (SEQ ID NO: | AE000659\|TRAV14/DV4*02 |
| 243) | M21624\|TRAV14/DV4*03 |
| actcggccgtctacctctgt (SEQ ID NO: | Z46643\|TRAV36/DV7*04 |
| 244) | X58767\|TRAV36/DV7*03 |
| | X61070\|TRAV36/DV7*02 |
| acagtgccacttactactgt (SEQ ID NO: | M23326\|TRDV3*01 |
| 245) | X15261\|TRDV3*02 |
| atgaagggtcttactactgt (SEQ ID NO: | X15207\|TRDV2*01 |
| 246) | Y13426\|TRDV2*02 |
| | AE000661\|TRDV2-03 |
| actcagcaatgtacttctgt (SEQ ID NO: | M21626\|TRAV14/DV4*01 |
| 247) | |

1MM: human TCRD V primers
actcggccgtctacctctgt (SEQ ID NO: 248)
actcagcaatgtacttctgt (SEQ ID NO: 249)
actctgcagtgtacttctgt (SEQ ID NO: 250)
atgccgcgatgtatttctgt (SEQ ID NO: 251)
atgaagggtcttactactgt (SEQ ID NO: 252)
actcagccacctacttctgt (SEQ ID NO: 253)
acagtgccacttactactgt (SEQ ID NO: 254)
attcagcaaagtacttttgt (SEQ ID NO: 255)
Compact: human TCRD V primers
actcggccitctacctctgt (SEQ ID NO: 256)
actcagcaaiitaittctgt (SEQ ID NO: 257)
atgccgcgatgtatttctgt (SEQ ID NO: 258)
actctgcagtgtacttctgt (SEQ ID NO: 259)
atgaagggtcttactactgt (SEQ ID NO: 260)
acagtgccacttactactgt (SEQ ID NO: 261)
attcagcaaagtacttttgt (SEQ ID NO: 262)

TABLE 3
Human TCRB J primers. Adapter sequences can be found in Table 1
(e.g., adapter sequences for the constant region primers).
human TCRB J primers

| | IMGT Accession\| |
|---|---|
| Primer Sequence | Associated J allele |
| ccagcactgagagccgggtc | X02987\|TRBJ2-4*01 |
| (SEQ ID NO: 263) | |
| ccagaaccaggagtcctccg | X02987\|TRBJ2-2P*01 |
| (SEQ ID NO: 264) | |
| cgagcaccaggagccgcgtg | X02987\|TRBJ2-5*01 |
| (SEQ ID NO: 265) | |
| ccagtacggtcagcctagag | X02987\|TRBJ2-2*01 |
| (SEQ ID NO: 266) | |
| cgagcactgtcagccgggtg | X02987\|TRBJ2-3*01 |
| (SEQ ID NO: 267) | |
| ctgtgaccgtgagcctggtg | X02987\|TRBJ2-7*02 |
| (SEQ ID NO: 268) | M14159\|TRBJ2-7*01 |
| ctacaacggttaacctggtc | K02545\|TRBJ1-2*01 |
| (SEQ ID NO: 269) | |
| ctacaacagtgagccaactt | M14158\|TRBJ1-3*01 |
| (SEQ ID NO: 270) | |
| ctgtcacagtgagcctggtc | M14158\|TRBJ1-6*01 |
| (SEQ ID NO: 271) | L36092\|TRBJ1-6*02 |
| ctacaactgtgagtctggtg | K02545\|TRBJ1-1*01 |
| (SEQ ID NO: 272) | |
| ctaggatggagagtcgagtc | M14158\|TRBJ1-5*01 |
| (SEQ ID NO: 273) | |
| ccaagacagagagctgggtt | M14158\|TRBJ1-4*01 |
| (SEQ ID NO: 274) | |
| ccagcacggtcagcctgctg | X02987\|TRBJ2-6*01 |
| (SEQ ID NO: 275) | |
| ctagcacggtgagccgtgtc | X02987\|TRBJ2-1*01 |
| (SEQ ID NO: 276) | |

In some embodiments, a method of analyzing DNA in an amplified, adapted library is provided. Methods disclosed herein include steps of partitioning an amplified, adapted DNA library into at least a first subsample and a second subsample. DNA isolated from a sample can be ligated to adapters for sequencing library preparation. Then, the adapted DNA can be amplified (e.g., LP-PCR) to provide an amplified, adapted library. The amplified, adapted library may be partitioned into at least a first subsample comprising sequence-variable target regions, a second subsample that may comprise a structural variation and an optional third subsample that can be retained as a test backup. A plurality of target regions comprising sequence-variable target regions from the first subsample can be captured, thereby providing captured regions. A second plurality of target regions that may comprise a structural variation from the second subsample can be amplified by multiplex amplification using a plurality of first primers and a plurality of second primers that anneal to the second plurality of target regions, thereby providing structural variation-enriched DNA. The multiplex amplification can be exponential. The plurality of first primers can be oppositely oriented relative to the plurality of second primers when annealed to target regions that comprise a structural variation (e.g., the plurality of first primers can be forward or sense primers and the plurality of second primers can be reverse or antisense primers, or vice versa). The captured regions and the structural variation-enriched DNA can be sequenced. In some embodiments, the captured regions and the structural variation-enriched DNA can be pooled and sequenced together. Alternatively, in some other embodiments, the captured regions and the structural variation-enriched DNA can be sequenced separately. In some embodiments, at least a portion of the plurality of first primers and second primers do not exponentially amplify a target region that does not comprise a structural variation. For example, when annealed to a wild type target region, extension is blocked by a downstream primer. When annealed to a target region comprising a structural variation, the primer closest to the breakpoint of the structural variation can be extended because there is no annealed downstream primer in a position to block it. This can result in incorporation of a labeled (e.g., biotinylated) nucleotide, and complexes of such extended primers and the molecules to which they are annealed can then be captured or enriched, e.g., for further analysis such as sequencing. In some embodiments, a majority (e.g., at least 60%, 70%, 80%, 90%, or 95%) of the plurality of first primers and the plurality of second primers annealed to a wild type target region are blocked by a downstream primer. This means the distinct species of primer in the plurality; the specified portion can anneal to wild-type sequence upstream of another primer to be blocked from extension (a small amount of extension may be permitted depending on the amount of separation between primers, but the wild type target region cannot be exponentially amplified). The presence of short primer-extended products and unextended primers can be minimized by performing an amplification that is dependent on the presence of adapter sequences at both ends of the DNA for exponential amplification, e.g., such that unextended primers or short primer-extended products (which resulted from extension that was blocked before reaching an adapter sequence in the template molecule) are not exponentially amplified or are amplified to a lesser extent than primer-extended products that comprise adapter sequences at both ends.

Extension being blocked by a downstream primer when the plurality of first primers or the plurality of second primers are annealed to a wild-type sequence means that the 5′ to 3′ exonuclease negative, strand displacement negative DNA polymerase is unable to continue extending a primer when it encounters the 5′ end of another primer. This may occur after a short amount of extension, such as up to 50 nucleotides, e.g., up to 40, 30, 20, 10, 5, 4, 3, 2, or 1 nucleotides. The amount of permitted extension before becoming blocked will depend on the distance between the primers when annealed to the template sequence. In some embodiments, at least 60%, 70%, 80%, 90%, or 95% of the the plurality of first primers and the plurality of second primers are blocked this way when annealed to wild-type sequence. In some embodiments, the sample comprises a mixture of DNA having a wild-type sequence and a structural variation mutant sequence, and the extension of at least one primer is blocked on the wild-type sequence but not on the structural variation mutant sequence. Thus, such methods can preferentially generate products in an amplified (primer-extended) sample complementary to the structural variation mutant sequence. This can facilitate the detection of such sequences because they are more common in the amplified sample than in the initial sample.

In some embodiments, a method of analyzing DNA in an amplified, adapted library is provided. Methods disclosed herein include preparing the amplified, adapted library by ligating adaptors to cfDNA from a subject having or suspected of having a cancer, thereby producing adapted cfDNA, followed by amplifying (e.g., LP-PCR) the adapted cfDNA. The amplified, adapted library may be partitioned into at least a first subsample comprising sequence-variable target regions, a second subsample comprising recombined CDR3 sequences, and an optional third subsample that can be retained as a test backup. A plurality of target regions comprising sequence-variable target regions from the first subsample can be captured, thereby providing captured regions. Segments comprising recombined CDR3 sequences from the second subsample can be amplified by multiplex amplification using a plurality of first primers that bind V regions and a plurality of second primers that bind J regions thereby providing CDR3-enriched DNA. The multiplex amplification can be exponential. The plurality of first primers that bind V regions can be oppositely oriented relative to the plurality of second primers that bind J regions (e.g., the plurality of first primers that bind V regions can be forward or sense primers and the plurality of second primers that bind J regions can be reverse or antisense primers). The captured regions and the CDR3-enriched DNA can be sequenced. In some embodiments, the captured regions and the CDR3-enriched DNA can be pooled and sequenced together. In other embodiments, the captured regions and the CDR3-enriched DNA can be sequenced separately.

In some embodiments, the amplified, adapted library can be prepared from cfDNA, and the amplified, adapted library can be prepared by ligating adaptors to cfDNA, thereby producing adapted cfDNA, and the adapted cfDNA can be then amplified. In some embodiments, the captured regions can be amplified prior to sequencing using a plurality of primers that anneal to the captured regions. In some embodiments, the method can further include capturing epigenetic target regions from the adapter-ligated DNA and amplifying and sequencing the epigenetic target regions, for example, hypermethylation variable target regions, hypomethylation variable target regions, methylation control target regions, or fragmentation variable target regions. In some embodiments, the method can further include quantifying a somatic mutation load using a plurality of captured regions comprising the sequence-variable target regions. In some embodiments, the method can further include detecting a presence or absence of a DNA molecule that comprises a structural variation, for example, from the first subsample or the second subsample that may comprise a structural variation.

In some embodiments, the primers used herein can be shorter than 100 nucleosides in length and at least 20 nucleotides in length. In some embodiments, the first or second primers can be 20-60, 25-60, or 30-40 nucleotides in length. In some embodiments, a majority (e.g., at least 60%, 70%, 80%, 90%, or all) of the primers used in primer extension anneal to their corresponding target regions in a parallel orientation. In such embodiments, the 3′ end of each primer points toward the 5′ end of another primer and/or the 5′ end of each primer points toward the 3′ end of another primer when they are annealed to the same DNA strand. Thus, in such embodiments, only one strand of DNA in target region anneals to any of the plurality of primers. In some embodiments, a majority (e.g., at least 60%, 70%, 80%, 90%, or all) of the primers anneal to a site separated from the site to which another primer anneals by less than or equal to about 50 nucleotides (e.g., less than or equal to 40, 30, 20, 10, 5, 4, 3, 2, or 1 nucleotides).

In some embodiments, the primers used have approximately uniform melting temperatures with respect to annealing to their complementary sequences, e.g., so that substantially all of the primers are capable of annealing under the same condition. For example, the plurality of primers may each anneal to their respective complementary sequence with a melting temperature within a range of 10° C., 9° C., 8° C., 7° C., 6° C., 5° C., 4° C., 3° C., 2° C., or 1° C. Variation of parameters such as primer length, GC content, and nucleotide modifications (e.g., methylation, base analogs, or LNA modifications) are known approaches for adjusting primer melting temperatures to desired values.

In some embodiments, the amplified products are enriched and/or captured using a solid support linked to a binding partner of the capture moiety, thereby also enriching and/or capturing the DNA molecules isolated from the sample that are hybridized to the amplified products. In some embodiments, the DNA molecules are denatured from their corresponding amplified products and sequenced in order to determine if they comprise a rearrangement and, if so, the breakpoint(s) of the rearrangement and further analysis as described herein. In some embodiments, the DNA molecules isolated from the sample that are hybridized to the amplified products comprise adapters and/or barcodes. In some embodiments, the adapters are used to amplify such molecules after denaturation. The barcodes can be used to identify sequence reads originating from the same molecule.

In some embodiments, the first or second primers that anneal to the target region of the DNA do not comprise a tail that does not bind to a target region. In some embodiments, the first or second primers that anneal to the target region of the DNA do comprise a tail that does not bind to a target region but is short enough to allow the primers to anneal to the target region. In some such embodiments, the primer tail is at the 5′ end of the primer. In such embodiments, the primer tail binds to the 5′ end of the adapter ligated to the 3′ end of the DNA molecule. In some embodiments, the primer tail comprises a capture moiety and the deoxynucleoside triphosphates used in primer extension may not comprise a capture moiety. In other embodiments, the primer tail does not comprise a capture moiety and the deoxynucleoside triphosphates used in primer extension comprise a capture moiety. The primer-extended products are captured using a solid support linked to a binding partner of the capture moiety and amplified on the solid support using PCR primers that anneal to a sequence within the primer tail and to the adapter ligated to the 5′ end of the DNA. Alternatively, the primer tail comprises a modification at the 5′ end that protects the primer from exonuclease activity, such as phosphorothioate internucleoside linkages. In some such embodiments, primer-extended products are enriched by contacting the primer-extended sample with a 5′ to 3′ exonuclease to degrade wild type sequences. The remaining sequences are amplified by PCR. In some embodiments, TdT ddATP tailing is performed prior to amplification in order to prevent truncated sequences from acting as primers in the PCR.

The disclosed methods can be combined with analysis of one or more additional biomarkers. In some embodiments, the disclosed methods are combined with one or more methods, such as but not limited to, methods for assessing DNA methylation patterns, DNA mutations (such as somatic mutations), nucleic acid fragmentation patterns, non-coding RNA (such as micro RNAs (miRNAs), ribosomal RNAs, transfer RNAs, small nucleolar RNAs (snow RNAs), and/or small nuclear RNAs (snRNAs)) levels, and/or cell type proportions/levels, cellular locations, extracellular vesicular surface proteins, intravesicular nucleic acids, histone modifications and/or structural modifications of one or more proteins (such as in a sample from a subject). In some embodiments, the disclosed methods are combined with one or more analyses of genetic variations including mutations, rare mutations, indels, rearrangements, copy number variations, transversions, translocations, recombinations, inversion, deletions, aneuploidy, partial aneuploidy, polyploidy, chromosomal instability, chromosomal structure alterations, gene fusions, chromosome fusions, gene truncations, gene amplification, gene duplications, chromosomal lesions, DNA lesions, abnormal changes in nucleic acid chemical modifications, abnormal changes in epigenetic patterns, and/or abnormal changes in nucleic acid 5-methylcytosine.

### B. Adapter Ligation or Addition; Tagging

In some embodiments, the methods comprise ligating adapters to DNA. In some embodiments, the ligating adapters to DNA produces adapter-ligated DNA. In some embodiments, DNA molecules can be subjected to blunt-end ligation with blunt-ended adapters. In some embodiments, DNA molecules can be subjected to sticky-end ligation with sticky-ended adapters. DNA molecules can be ligated to adapters at either one end or both ends. DNA molecules can be ligated with at least partially double stranded adapter (e.g., a Y shaped or bell-shaped adapter).

DNA ligase and adapters are added to ligate DNA molecules in the sample with an adapter on one or both ends, i.e. to form adapted DNA. As used herein, “adapter” refers to short nucleic acids (e.g., less than about 500, less than about 100 or less than about 50 nucleotides in length, or be 20-30, 20-40, 30-50, 30-60, 40-60, 40-70, 50-60, 50-70, 20-500, or 30-100 bases from end to end) that are typically at least partially double-stranded and can be ligated to the end of a given sample DNA molecule. In some instances, two adapters can be ligated to a single sample DNA molecule, with one adapter ligated to each end of the sample nucleic acid molecule.

In some embodiments, the ligase used in ligation reactions can act on both single strand DNA nicks and double stranded DNA ends. In some cases, the ligase is T4 DNA ligase or T3 DNA ligase. Adapters can include nucleic acid primer binding sites to permit amplification of a sample DNA molecule flanked by adapters at both ends, and/or a sequencing primer binding site, including primer binding sites for sequencing applications, such as various next generation sequencing (NGS) applications. Adapters can include a sequence for hybridizing to a solid support, e.g., a flow cell sequence. Adapters can also include binding sites for capture probes, such as an oligonucleotide attached to a flow cell support or the like. Adapters can also include sample indexes and/or molecular barcodes. These are typically positioned relative to amplification primer and sequencing primer binding sites, such that the sample index and/or molecular barcode is included in amplicons and sequencing reads of a given DNA molecule. Adapters of the same or different sequence can be linked to the respective ends of a sample DNA molecule. In some cases, adapters of the same or different sequence are linked to the respective ends of the DNA molecule except that the sample index and/or molecular barcode differs in its sequence. In some embodiments, the adapter is an asymmetric adapter, such as a Y-shaped adapter in which one end is blunt ended or tailed as described herein, for joining to a nucleic acid molecule, which is also blunt ended or tailed with one or more complementary nucleotides to those in the tail of the adapter. In another exemplary embodiment, an adapter is a bell-shaped adapter that includes a blunt or tailed end for joining to a DNA molecule to be analyzed. Other exemplary adapters include T-tailed, C-tailed or hairpin shaped adapters and bubble adapters. For example, a hairpin shaped adapter can comprise a complementary double stranded portion and a loop portion, where the double stranded portion can be attached (e.g. ligated) to a double-stranded polynucleotide. Hairpin shaped sequencing adapters can be attached to both ends of a polynucleotide fragment to generate a circular molecule, which can be sequenced multiple times.

The adapters used in the methods of the present disclosure can comprise one or more known modified nucleosides, such as methylated nucleosides. In some embodiments, the modified nucleosides comprise modification resistant cytosines. In some embodiments, each cytosine in each adapter is a modification resistant cytosine. In some embodiments, the modification resistant cytosine is a deamination resistant cytosine. In some embodiments, the deamination resistant cytosine comprises 5-propynylC (5pyC), 5-pyrrolo-dC (5pyrC), 5-hydroxymethylcytosine (5hmC), glucosylated5-hydroxymethylcytosine (5ghmC), cytosine 5-methylenesulfonate (CMS), or N4-modified cytosine.

In some embodiments, a procedure that affects a first nucleobase in the DNA differently from a second nucleobase, e.g., a conversion step, is performed. In some embodiments, the ligation step can take place before or after the conversion step. In general, “conversion step” or “conversion procedure” refers to any step or procedure that changes the base pairing specificity of one or more nucleotides. In some embodiments, the conversion step comprises contacting DNA with a deaminase.

In instances where two adapters are ligated to a sample nucleic acid (one at each end), either or both of the adapters may comprise one or more known modified nucleosides. Typically, the primer binding site(s), sequencing primer binding site(s), sample index(es) and/or molecular barcode(s), if present, do not comprise the known modified nucleosides that change base pairing specificity as a result of the conversion procedure.

In some embodiments, adapters may be added to the DNA or a subsample thereof. Adapters can be ligated to DNA at any point in the methods herein. In some embodiments, adapters are ligated to the DNA in a sample. In some embodiments, adapters are ligated to the DNA of a sample or subsample thereof prior to annealing primers to the DNA for capture probe generation. In some such embodiments, the adapter-ligated DNA is amplified prior to annealing primers to the DNA for capture probe generation. In some embodiments, adapters are ligated to the DNA of a sample or subsample thereof before the DNA is contacted with the capture probes. In some embodiments, the DNA to which the adapters are ligated is in the same sample or subsample as the DNA used as a template to generate capture probes. In some embodiments, the DNA to which the adapters are ligated is in a different sample or subsample, e.g., a second sample or a second subsample of a first sample, than the DNA used as a template to generate capture probes. In some embodiments, the adapters ligated to DNA captured by the capture probes.

In some embodiments, the primers used to generate capture probes are not complementary to adapters, and the resulting capture probes therefore do not comprise adapters. Adapter-ligated DNA can therefore be selectively amplified in the presence of capture probes that do not comprise adapters. Similarly, adapter-ligated DNA can be separated from DNA that does not comprise adapters.

In some embodiments, the disclosed methods further comprise adding adapters to the DNA. In some embodiments, the adapters may be added before the partitioning and capturing steps. This may be done concurrently with an amplification procedure, e.g., by providing the adapters in a 5′ portion of a primer (where PCR is used, this can be referred to as library prep-PCR or LP-PCR), or before or after an amplification step. In some embodiments, adapters are added by other approaches, such as ligation. In some such methods, prior to partitioning or prior to capturing, first adapters are added to the nucleic acids by ligation to the 3′ ends thereof, which may include ligation to single-stranded DNA. In some embodiments, prior to any partitioning or capturing steps, first adapters are added to the nucleic acids by ligation, which may include ligation to single-stranded DNA (e.g., to the 3′ ends thereof). In some embodiments, the capture probes can be isolated after partitioning and ligation. For example, the hypomethylated partition can be ligated with adapters and a portion of the ligated hypomethylated partition can then be used to generate the capture probes for rearrangements. The adapter can be used as a priming site for second-strand synthesis, e.g., using a universal primer and a DNA polymerase. A second adapter can then be ligated to at least the 3′ end of the second strand of the now double-stranded molecule. In some embodiments, the first adapter comprises an affinity tag, such as biotin, and nucleic acid ligated to the first adapter is bound to a solid support (e.g., bead), which may comprise a binding partner for the affinity tag, such as streptavidin. For further discussion of a related procedure, see Gansauge et al., Nature Protocols 8:737-748 (2013). Commercial kits for sequencing library preparation compatible with single-stranded nucleic acids are available, e.g., the Accel-NGS® Methyl-Seq DNA Library Kit from Swift Biosciences. In some embodiments, after adapter ligation, nucleic acids are amplified.

In some embodiments, single-stranded DNA library preparation is performed in a one-step combined phosphorylation/ligation reaction, e.g., as described in Troll et al., BMC Genomics, 20:1023 (2019), available at https://doi.org/10.1186/s12864-019-6355-0. This method, called Single Reaction Single-stranded LibrarY (“SRSLY,”) can be performed without end-polishing. SRSLY may be useful for converting short and fragmented DNA molecules, e.g., cfDNA fragments, into sequencing libraries while retaining native lengths and ends. The SRSLY method can create sequencing libraries (e.g., Illumina sequencing libraries) from fragmented or degraded template (input) DNA. In particular embodiments, template DNA is first heat denatured and then immediately cold shocked to render the template DNA molecules single-stranded. The DNA can be maintained as single-stranded throughout the ligation reaction by the inclusion of a thermostable single-stranded binding protein (SSB). Next, the template DNA, which at this point can be single-stranded and coated with SSB, is placed in a phosphorylation/ligation dual reaction with directional dsDNA NGS adapters that contain single-stranded overhangs. Both the forward and reverse sequencing adapters can share similar structures but differ in which termini is unblocked in order to facilitate proper ligations. Both sequencing adapters can comprise a dsDNA portion and a single-stranded splint overhang of random nucleotides that occurs on the 3-prime terminus of the bottom strand of the forward adapter and the 5-prime terminus of the bottom strand of the reverse adapter. In this way, the forward adapter (e.g., (P5) Illumina adapter) can be delivered to the 5-prime end of template molecules and the reverse adapter (e.g., (P7) Illumina adapter) is delivered to the 3-prime end of template molecules. Thus, the native polarity of input DNA molecules can be retained.

During the dual phosphorylation/ligation reaction, T4 Polynucleotide Kinase (PNK) can be used to prepare template DNA termini for ligation by phosphorylating 5-prime termini and dephosphorylating 3-prime termini. T4 PNK works on both ssDNA and dsDNA molecules and has no activity on the phosphorylation state of proteins. Simultaneously, the random nucleotides of the splint adapter can be annealed to the single-stranded template molecule. This creates a short, localized dsDNA molecule, enabling ligation of template to adapter with a ligase such as T4 DNA ligase, which has high ligation efficiency on dsDNA templates but low efficiency on ssDNA. After the single phosphorylation/ligation reaction is complete, the library DNA can be, e.g., purified and placed directly into standard NGS indexing PCR, compatible with both traditional single or dual index primers.

In some embodiments, the adapters include different tags of sufficient numbers that the number of combinations of tags results in a low probability e.g., 95, 99 or 99.9% of two nucleic acids with the same start and stop points receiving the same combination of tags. Adapters, whether bearing the same or different tags, can include the same or different primer binding sites, but preferably adapters include the same primer binding site.

In some embodiments, following attachment of adapters, the nucleic acids are subject to amplification. The amplification can use, e.g., universal primers that recognize primer binding sites in the adapters.

In some embodiments, following attachment of adapters, the DNA or a subsample or portion of the DNA is partitioned, comprising contacting the DNA with an agent that preferentially binds to nucleic acids bearing a sequence-variable target region or an epigenetic modification. The nucleic acids are partitioned into at least two partitioned subsamples differing in the extent to which the nucleic acids bear the modification from binding to the agents. For example, if the agent has affinity for nucleic acids bearing the modification, nucleic acids overrepresented in the modification (compared with median representation in the population) preferentially bind to the agent, whereas nucleic acids underrepresented for the modification do not bind or are more easily eluted from the agent. The nucleic acids can then be amplified from primers binding to the primer binding sites within the adapters. Partitioning may be performed instead before adapter attachment, in which case the adapters may comprise differential tags that include a component that identifies which partition a molecule occurred in.

In some embodiments, the nucleic acids are linked at both ends to Y-shaped adapters including primer binding sites and tags. The molecules are amplified.

In some embodiments, the DNA molecules of the adapted library may be tagged with sample indexes and/or molecular barcodes (referred to generally as “tags”), which may be present in the adapters. In some embodiments, the DNA molecules of the sample comprise barcodes, e.g., in the adapters. In some embodiments, the adapted DNA comprises barcodes. Tagging DNA molecules is a procedure in which a tag is attached to or associated with the DNA molecules. Such tags can be molecules, such as nucleic acids, containing information that indicates a feature of the molecule with which the tag is associated; tags, such as tags contained in adapters, can be attached to sample DNA molecules, e.g., by ligation, such as during library preparation. For example, DNA molecules can bear a sample tag or sample index (which distinguishes molecules in one sample from those in a different sample), a partition tag (which distinguishes molecules in one partition from those in a different partition) and/or a molecular tag/molecular barcode (which distinguishes different molecules from one another (in both unique and non-unique tagging scenarios).

Tagging strategies can be divided into unique tagging and non-unique tagging strategies. In unique tagging, all or substantially all of the molecules in a sample bear a different tag, so that reads can be assigned to original molecules based on tag information alone. Tags used in such methods are sometimes referred to as “unique tags”. In non-unique tagging, different molecules in the same sample can bear the same tag, so that other information in addition to tag information is used to assign a sequence read to an original molecule. Such information may include start and stop coordinate, coordinate to which the molecule maps, start or stop coordinate alone, etc. Tags used in such methods are sometimes referred to as “non-unique tags”. Accordingly, it is not necessary to uniquely tag every molecule in a sample. It suffices to uniquely tag molecules falling within an identifiable class within a sample. Thus, molecules in different identifiable families can bear the same tag without loss of information about the identity of the tagged molecule.

For methods that involve a partitioning step, a partition tag (which distinguishes molecules in one partition from those in a different partition) may be included. In some embodiments, adapters added to DNA molecules comprise tags. In certain embodiments, a tag can comprise one or a combination of barcodes. As used herein, the term “barcode” refers to a nucleic acid molecule having a particular nucleotide sequence, or to the nucleotide sequence, itself, depending on context. A barcode can have, for example, between 10 and 100 nucleotides. A collection of barcodes can have degenerate sequences or can have sequences having a certain Hamming distance, as desired for the specific purpose. So, for example, a molecular barcode can be comprised of one barcode or a combination of two barcodes, each attached to different ends of a molecule. Additionally or alternatively, for different partitions and/or samples, different sets of molecular barcodes, molecular tags, or molecular indexes can be used such that the barcodes serve as a molecular tag through their individual sequences and also serve to identify the partition and/or sample to which they correspond based the set of which they are a member.

In some embodiments, two or more partitions, e.g., each partition, is/are differentially tagged. For example, barcodes can be used to allow the origin of the DNA (e.g., the subject, biological sample (e.g., samples collected at various time points), enriched DNA sample (e.g., enriched DNA comprising an epigenetic target region set or enriched DNA comprising a sequence-variable target region set), partition, or similar) to be identified, e.g., following pooling of a plurality of samples for parallel sequencing. Tags comprising barcodes can be incorporated into or otherwise joined to adapters. Tags can be incorporated by ligation, overlap extension PCR among other methods. Tags can be used to label the individual polynucleotide population partitions so as to correlate the tag (or tags) with a specific partition. Alternatively, tags can be used in embodiments of the disclosure that do not employ a partitioning step. In some embodiments, a single tag can be used to label a specific partition. In some embodiments, multiple different tags can be used to label a specific partition. In embodiments employing multiple different tags to label a specific partition, the set of tags used to label one partition can be readily differentiated for the set of tags used to label other partitions. In some embodiments, the tags may have additional functions; for example, the tags can be used to index sample sources or used as unique molecular identifiers (which can be used to improve the quality of sequencing data by differentiating sequencing errors from mutations, for example as in Kinde et al., Proc Nat'l Acad Sci USA 108: 9530-9535 (2011), Kou et al., PLoS ONE, 11: e0146638 (2016)) or used as non-unique molecule identifiers, for example as described in U.S. Pat. No. 9,598,731. Similarly, in some embodiments, the tags may have additional functions, for example, the tags can be used to index sample sources or used as non-unique molecular identifiers (which can be used to improve the quality of sequencing data by differentiating sequencing errors from mutations).

Tags may be incorporated into or otherwise joined to adapters by chemical synthesis, ligation (e.g., as described above, e.g. by blunt-end ligation or sticky-end ligation), or overlap extension polymerase chain reaction (PCR), among other methods. Such adapters are ultimately joined to the sample DNA molecule. In other embodiments, one or more rounds of amplification cycles (e.g., PCR amplification) may be applied to introduce sample indexes to a nucleic acid molecule using conventional nucleic acid amplification methods. The amplifications may be conducted in one or more reaction mixtures (e.g., a plurality of microwells in an array). Molecular barcodes and/or sample indexes may be introduced simultaneously, or in any sequential order. In some embodiments, molecular barcodes and/or sample indexes are introduced prior to and/or after any conversion procedure. In the case of molecular barcodes and/or sample indexes being introduced through amplification processes, the conversion step will occur before the molecular barcodes and/or sample indexes are introduced. In some embodiments, molecular barcodes and/or sample indexes are introduced prior to and/or after sequence capturing steps, if present, are performed. In some embodiments, only the molecular barcodes are introduced prior to probe capturing and the sample indexes are introduced after sequence capturing steps are performed. In some embodiments, both the molecular barcodes and the sample indexes are introduced prior to performing probe-based capturing steps, if present. In some embodiments, the sample indexes are introduced after sequence capturing steps are performed, if present. In some embodiments, sample indexes are incorporated through overlap extension polymerase chain reaction (PCR).

In some embodiments, the tags may be located at one end or at both ends of the sample DNA molecule. In some embodiments, tags are predetermined or random or semi-random sequence oligonucleotides. In some embodiments, the tag(s) may together be less than about 500, 200, 100, 50, 20, 10, 9, 8, 7, 6, 5, 4, 3, 2, or 1 nucleotides in length. Typically tags are about 5 to 20 or 6 to 15 nucleotides in length. The tags may be linked to sample DNA molecules randomly or non-randomly.

In some embodiments, each sample or partition (discussed below) is uniquely tagged with a sample index or a combination of sample indexes. In some embodiments, each nucleic acid molecule of a sample or sub-sample is uniquely tagged with a molecular barcode or a combination of molecular barcodes. In other embodiments, a plurality of molecular barcodes may be used such that molecular barcodes are not necessarily unique to one another in the plurality (e.g., non-unique molecular barcodes). In these embodiments, molecular barcodes are generally attached (e.g., by ligation as part of an adapter) to individual molecules such that the combination of the molecular barcode and the sequence it may be attached to creates a unique sequence that may be individually tracked. Detection of non-unique molecular barcodes in combination with endogenous sequence information (e.g., the beginning (start) and/or end (stop) genomic location/position corresponding to the sequence of the original DNA molecule in the sample, start and stop genomic positions corresponding to the sequence of the original DNA molecule in the sample, the beginning (start) and/or end (stop) genomic location/position of the sequence read that is mapped to the reference sequence, start and stop genomic positions of the sequence read that is mapped to the reference sequence, sub-sequences of sequence reads at one or both ends, length of sequence reads, and/or length of the original DNA molecule in the sample) typically allows for the assignment of a unique identity to a particular molecule. In some embodiments, beginning region comprises the first 1, first 2, the first 5, the first 10, the first 15, the first 20, the first 25, the first 30 or at least the first 30 base positions at the 5′ end of the sequencing read that align to the reference sequence. In some embodiments, the end region comprises the last 1, last 2, the last 5, the last 10, the last 15, the last 20, the last 25, the last 30 or at least the last 30 base positions at the 3′ end of the sequencing read that align to the reference sequence. The length, or number of base pairs, of an individual sequence read are also optionally used to assign a unique identity to a given molecule. As described herein, fragments from a single strand of nucleic acid having been assigned a unique identity, may thereby permit subsequent identification of fragments from the parent strand, and/or a complementary strand.

In certain embodiments of non-unique tagging, the number of different tags used can be sufficient that there is a very high likelihood (e.g., at least 99%, at least 99.9%, at least 99.99% or at least 99.999% that all DNA molecules of a particular group bear a different tag. It is to be noted that when barcodes are used as tags, and when barcodes are attached, e.g., randomly, to both ends of a molecule, the combination of barcodes, together, can constitute a tag. This number, in term, is a function of the number of molecules falling into the calls. For example, the class may be all molecules mapping to the same start-stop position on a reference genome. The class may be all molecules mapping across a particular genetic locus, e.g., a particular base or a particular region (e.g., up to 100 bases or a gene or an exon of a gene). In certain embodiments, the number of different tags used to uniquely identify a number of molecules, z, in a class can be between any of2*z, 3*z, 4*z, 5*z, 6*z, 7*z, 8*z, 9*z, 10*z, 11*z, 12*z, 13*z, 14*z, 15*z, 16*z, 17*z, 18*z, 19*z, 20*z or 100*z (e.g., lower limit) and any of 100,000*z, 10,000*z, 1000*z or 100*z (e.g., upper limit).

In some embodiments, molecular barcodes are introduced at an expected ratio of a set of identifiers (e.g., a combination of unique or non-unique molecular barcodes) to molecules in a sample. One example format uses from about 2 to about 1,000,000 different molecular barcode sequences, or from about 5 to about 150 different molecular barcode sequences, or from about 20 to about 50 different molecular barcode sequences, ligated to both ends of a target molecule. Alternatively, from about 25 to about 1,000,000 different molecular barcode sequences may be used. For example, 20-50×20-50 molecular barcode sequences (i.e., one of the 20-50 different molecular barcode sequences can be attached to each end of the target molecule) can be used. Such numbers of identifiers are typically sufficient for different molecules having the same start and stop points to have a high probability (e.g., at least 94%, 99.5%, 99.99%, or 99.999%) of receiving different combinations of identifiers. In some embodiments, about 80%, about 90%, about 95%, or about 99% of molecules have the same combinations of molecular barcodes. For example, in a sample of about 5 ng to 30 ng of cell free DNA, one expects around 3000 molecules to map to a particular nucleotide coordinate, and between about 3 and 10 molecules having any start coordinate to share the same stop coordinate. Accordingly, about 50 to about 50,000 different tags (e.g., between about 6 and 220 barcode combinations) can suffice to uniquely tag all such molecules. To uniquely tag all 3000 molecules mapping across a nucleotide coordinate, about 1 million to about 20 million different tags would be required.

In some embodiments, the assignment of unique or non-unique molecular barcodes in reactions is performed using methods and systems described by, for example, U.S. Patent Application Nos. 20010053519, 20030152490, and 20110160078, and U.S. Pat. Nos. 6,582,908 and 7,537,898 and 9,598,731, and 9,902,992, each of which is hereby incorporated by reference in its entirety. Alternatively, in some embodiments, different nucleic acid molecules of a sample may be identified using only endogenous sequence information (e.g., start and/or stop positions, sub-sequences of one or both ends of a sequence, and/or lengths. Tags can be linked to sample nucleic acids randomly or non-randomly.

In some embodiments, the tagged nucleic acids are sequenced after loading into a microwell plate. The microwell plate can have 96, 384, or 1536 microwells. In some cases, they are introduced at an expected ratio of unique tags to microwells. For example, the unique tags may be loaded so that more than about 1, 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 unique tags are loaded per genome sample. In some cases, the unique tags may be loaded so that less than about 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 unique tags are loaded per genome sample. In some cases, the average number of unique tags loaded per sample genome is less than, or greater than, about 1, 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 unique tags per genome sample.

In some embodiments, a format uses 20-50 different tags (e.g., barcodes) ligated to both ends of target nucleic acids. For example, 35 different tags (e.g., barcodes) ligated to both ends of target molecules creating 35×35 permutations, which equals 1225 for 35 tags. Such numbers of tags are sufficient so that different molecules having the same start and stop points have a high probability (e.g., at least 94%, 99.5%, 99.99%, 99.999%) of receiving different combinations of tags. Other barcode combinations include any number between 10 and 500, e.g., about 15×15, about 35×35, about 75×75, about 100×100, about 250×250, about 500×500.

In some cases, unique tags may be predetermined or random or semi-random sequence oligonucleotides. In other cases, a plurality of barcodes may be used such that barcodes are not necessarily unique to one another in the plurality. In this example, barcodes may be ligated to individual molecules such that the combination of the barcode and the sequence it may be ligated to creates a unique sequence that may be individually tracked. As described herein, detection of non-unique barcodes in combination with sequence data of beginning (start) and end (stop) portions of sequence reads may allow assignment of a unique identity to a particular molecule. The length or number of base pairs, of an individual sequence read may also be used to assign a unique identity to such a molecule. As described herein, fragments from a single strand of nucleic acid having been assigned a unique identity, may thereby permit subsequent identification of fragments from the parent strand.

In some embodiments, the method includes adding one or more internal control DNAs and forward and reverse primers for amplifying the internal control DNAs. The internal control DNAs may be added before amplification using the primers that anneal upstream and downstream of the rearrangement breakpoints. The forward and reverse primers for amplifying the internal control DNAs may be included with, or added at the same time as, the primers that anneal upstream and downstream of the rearrangement breakpoints. The internal control DNAs may comprise or consist of sequences that do not occur in the genome of the subject, or that do not occur in the genome of the species of which the subject is a member (e.g., the human genome). The forward and/or reverse primers for amplifying the internal control DNAs may comprise sequences that are not complementary to any sequence in the genome of the subject, e.g., the human genome. The internal control DNAs may be used to ensure that the amplification process proceeded as designed. As such, the method may comprise detecting (e.g., sequencing) molecules amplified from and/or captured by the one or more internal control DNAs. The method can comprise comparing an amount of internal control DNAs (e.g., number of molecules or reads detected that correspond to an internal control DNA sequence) to a predetermined threshold, and either rejecting sequencing results if the predetermined threshold is not met or accepting sequencing results if the predetermined threshold is met. The predetermined threshold may be established, e.g., based on historical data or by testing the method on samples of DNA from test subjects, such as healthy volunteers. For example, amplification and detection of the one or more internal control DNAs provides confirmation that the amplification process proceeded properly, thus reducing the likelihood of a false negative.

C. Partitioning the Sample into a Plurality of Subsamples

In some embodiments, the amplified, adapted library is partitioned into at least a first subsample and a second subsample. This may be accomplished simply by dividing the library into identical or substantially identical subsamples. Alternatively, in some methods, different DNA (e.g., sequence-variable target regions, recombined CDR3 sequences, and epigenetic target regions) can be partitioned based on one or more characteristics of the DNA. Detecting aberrant features in DNA (whether sequence-based, epigenetic, or both) while also detecting recombined CDR3 sequences and/or target regions comprising structural variations may provide greater specificity and/or sensitivity for identifying an abnormal state than detecting the DNA features alone or levels of one or more post-translationally modified proteins alone.

Disclosed methods herein comprise analyzing DNA in a sample. In some embodiments described herein, the disclosed methods comprise partitioning DNA. In such methods, different forms of DNA (e.g., hypermethylated and hypomethylated DNA) can be physically partitioned based on one or more characteristics of the DNA. This approach can be used to determine, for example, whether certain sequences are hypermethylated or hypomethylated.

In some embodiments, a first subsample or aliquot of a sample is subjected to steps for making capture probes as described elsewhere herein and a second subsample or aliquot of a sample is subjected to partitioning. In some embodiments, a sample or subsample or aliquot thereof is subjected to partitioning and differential tagging, followed by a capture step using capture probes for rearranged sequences and optionally additional capture probes, e.g., for sequence-variable and/or epigenetic target regions.

Methylation profiling can involve determining methylation patterns across different regions of the genome. For example, after partitioning molecules based on extent of methylation (e.g., relative number of methylated nucleobases per molecule) and sequencing, the sequences of molecules in the different partitions can be mapped to a reference genome. This can show regions of the genome that, compared with other regions, are more highly methylated or are less highly methylated. In this way, genomic regions, in contrast to individual molecules, may differ in their extent of methylation.

In some embodiments, the partitioning comprises contacting the DNA with an agent that recognizes a modification associated with (e.g., in) the DNA. In some embodiments, the agent that recognizes the modification is an antibody or a methyl binding domain (MBD) protein. In some embodiments, the agent is immobilized on a solid support. In some embodiments, the solid support comprises a bead. In some embodiments, the partitioning comprises immunoprecipitation, e.g., using the agent that recognizes the modification, such as an antibody or an MBD protein, immobilized on solid support.

In some embodiments, the partitioning comprises precipitating the methylated DNA. In some embodiments, the partitioning comprises precipitating the methylated DNA to separate it from the unmethylated DNA. In some embodiments, the precipitating the methylated DNA can be performed using any pair of binding partners. In some embodiments, one of the binding partners may be linked to the MBD protein or antibody, and the other binding partner may be linked to a solid support. In some embodiments, the binding partner comprises biotin and streptavidin. In some embodiments, the biotin may be linked to the MBD protein, and the streptavidin may be linked to a solid support. In some embodiments, the MBD protein is linked to a solid support, optionally using any pair of binding partners. In some embodiments, the partitioning comprises immunoprecipitating the methylated DNA. In some embodiments, the partitioning comprises immunoprecipitating the methylated DNA separately from the unmethylated DNA.

In some embodiments, the modification is methylation, and in some such embodiments, the partitioning comprises partitioning on the basis of methylation level. In some such embodiments, the agent is a methyl binding reagent. In some embodiments, the methyl binding reagent specifically recognizes 5-methylcytosine. In some such embodiments, the agent is a hydroxymethyl binding reagent. In some embodiments, the methyl binding reagent specifically recognizes 5-hydroxymethylcytosine, biotinylated 5-hydroxymethylcytosine, glucosylated 5-hydroxymethylcytosine, or sulfonylated 5-hydroxymethylcytosine. In some embodiments, the partitioning comprises partitioning on the basis of binding to a protein comprising contacting the sample comprising the DNA with a binding reagent specific for the protein. In some such embodiments, binding reagent specifically binds a methylated protein or an acetylated protein, such as a methylated or acetylated histone, or an unmethylated protein or an unacetylated protein such as an unmethylated or unacetylated histone. In some embodiments, the binding reagent specifically binds an unmethylated or unacetylated protein epitope.

In some embodiments, the modification is hydroxymethylation, and in some such embodiments, the partitioning comprises partitioning on the basis of hydroxymethylation level. In some such embodiments, the agent is a hydroxymethyl binding reagent, such as an antibody. In some embodiments, the hydroxymethyl binding reagent (e.g., antibody) specifically recognizes 5-hydroxymethylcytosine (5-hmC). In some embodiments, a modification such as hydroxymethylation is labeled (e.g., biotinylated, glucosylated, or sulfonated) before being contacted with an agent that recognizes the labeled form of the modification. For example, 5-hmC can be enzymatically glucosylated and then partitioned based on binding to J-binding protein 1. Exemplary methods of labeling and/or partitioning 5-hmC are provided, e.g., in Song et al., Nat. Biotech. 29:68-72 (2010); Ko et al., Nature 468:839-843 (2010); and Robertson et al., Nucleic Acids Res. 39:e55 (2011).

Where immunoprecipitation is used and involves an antibody that recognizes single-stranded DNA, the DNA may be converted to double-stranded form by complementary strand synthesis before a subsequent step. Such synthesis may use an adapter as a primer binding site, or can use random priming.

Partitioning nucleic acid molecules in a sample can increase a rare signal, e.g., by enriching rare nucleic acid molecules that are more prevalent in one partition of the sample. For example, a genetic variation present in epigenetic target regions, sequence-variable target regions, and/or recombined CDR3 sequences, e.g. in a TCR or BCR or immunoglobulin, can be more easily detected by partitioning a sample into a subsample comprising epigenetic target regions or sequence-variable target regions. By analyzing multiple partitions of a sample, a multi-dimensional analysis of a single molecule can be performed, and hence, greater sensitivity can be achieved. Partitioning may include physically partitioning nucleic acid molecules into partitions or subsamples based on the presence or absence of one or more methylated nucleobases. A sample may be partitioned into partitions or subsamples based on a characteristic that is indicative of differential gene expression or a disease state. A sample may be partitioned based on a characteristic, or combination thereof that provides a difference in signal between a normal and diseased state during analysis of nucleic acids, e.g., cell free DNA (cfDNA), non-cfDNA, tumor DNA, circulating tumor DNA (ctDNA) and cell free nucleic acids (cfNA).

In some embodiments, hypermethylation and/or hypomethylation variable epigenetic target regions are analyzed to determine whether they show differential methylation characteristic of tumor cells or cells of a type that does not normally contribute to the DNA sample being analyzed (such as cfDNA), and/or particular immune cell types.

In some instances, heterogeneous DNA in a sample can be partitioned into two or more partitions (e.g., at least 3, 4, 5, 6 or 7 partitions). In some embodiments, each partition is differentially tagged. Tagged partitions can then be pooled together for collective sample prep and/or sequencing. The partitioning-tagging-pooling steps can occur more than once, with each round of partitioning occurring based on a different characteristic (examples provided herein) and tagged using differential tags that are distinguished from other partitions and partitioning means. In other instances, the differentially tagged partitions are separately sequenced.

In some embodiments, sequence reads from differentially tagged and pooled DNA are obtained and analyzed in silico. After sequencing, analysis of reads can be performed on a partition-by-partition level, as well as a whole DNA population level. Tags are used to sort reads from different partitions. Analysis to detect genetic variants can be performed on a partition-by-partition level, as well as whole nucleic acid population level. For example, analysis can include in silico analysis to determine genetic variants, such as copy number variations (CNVs), single nucleotide variations (SNVs), insertions/deletions (indels), and/or fusions in nucleic acids in each partition. In some instances, in silico analysis can include determining chromatin structure or analysis to determine epigenetic variation (one or more of methylation chromatin structure, etc.). Analysis can include in silico using sequence information, genomic coordinates length, coverage, and/or copy number. For example, coverage of sequence reads can be used to determine nucleosome positioning in chromatin. Tags are used to sort reads from different partitions. Higher coverage can correlate with higher nucleosome occupancy in genomic region while lower coverage can correlate with lower nucleosome occupancy or nucleosome depleted region (NDR).

Examples of characteristics that can be used for partitioning include sequence length, methylation level, nucleosome binding, sequence mismatch, immunoprecipitation, and/or proteins that bind to DNA. Resulting partitions can include one or more of the following nucleic acid forms: single-stranded DNA (ssDNA), double-stranded DNA (dsDNA), shorter DNA fragments and longer DNA fragments. In some embodiments, partitioning based on a cytosine modification (e.g., cytosine methylation) or methylation generally is performed and is optionally combined with at least one additional partitioning step, which may be based on any of the foregoing characteristics or forms of DNA. In some embodiments, a heterogeneous population of nucleic acids is partitioned into nucleic acids with base modification and without one or more base modifications, including e.g., one or more sequence-variable target regions or one or more epigenetic modifications and nucleic acids with recombined CDR3 sequences or target regions that may comprise a structural variation. Examples of epigenetic modifications include presence or absence of methylation; level of methylation; type of methylation (e.g., 5-methylcytosine versus other types of methylation, such as adenine methylation and/or cytosine hydroxymethylation); and association and level of association with one or more proteins, such as histones. Additional examples of base modifications are described elsewhere herein. Alternatively or additionally, a heterogeneous population of nucleic acids can be partitioned into nucleic acid molecules associated with nucleosomes and nucleic acid molecules devoid of nucleosomes. Alternatively or additionally, a heterogeneous population of nucleic acids may be partitioned into single-stranded DNA (ssDNA) and double-stranded DNA (dsDNA). Alternatively, or additionally, a heterogeneous population of nucleic acids may be partitioned based on nucleic acid length (e.g., molecules of up to 160 bp and molecules having a length of greater than 160 bp).

In some cases, different procedures are applied to different partitions to determine different characteristics of the initial sample. In some embodiments, the DNA of at least one partition is subjected to an end repair and sequencing procedure described herein. In some embodiments at least one partition is not subjected to the end repair and sequencing procedure described herein. In cases where the method comprises a conversion procedure, corresponding sequences from the converted and non-converted partitions can be compared to identify single nucleotides that have undergone conversion and therefore identify corresponding modified nucleosides in the initial sample.

In some embodiments, a method described herein comprising partitioning further comprises tagging molecules in each partition with a partition tag. After re-combining partitions (e.g., to reduce the number of sequencing runs needed and avoid unnecessary cost) and sequencing molecules, the partition tags identify the source partition. In another embodiment, different partitions are tagged with different sets of molecular tags, e.g., comprised of a pair of barcodes. In this way, each molecular barcode indicates the source partition as well as being useful to distinguish molecules within a partition. For example, a first set of 35 barcodes can be used to tag molecules in a first partition, while a second set of 35 barcodes can be used tag molecules in a second partition.

In some embodiments, after partitioning and tagging with partition tags, the molecules may be pooled for sequencing in a single run. In some embodiments, a sample tag is added to the molecules, e.g., in a step subsequent to addition of partition tags and pooling. Sample tags can facilitate pooling material generated from multiple samples for sequencing in a single sequencing run.

Alternatively, in some embodiments, partition tags may be correlated to the sample as well as the partition. As a simple example, a first tag can indicate a first partition of a first sample; a second tag can indicate a second partition of the first sample; a third tag can indicate a first partition of a second sample; and a fourth tag can indicate a second partition of the second sample.

While tags may be attached to molecules already partitioned based on one or more characteristics, the final tagged molecules in the library may no longer possess that characteristic. For example, while single stranded DNA molecules may be partitioned and tagged, the final tagged molecules in the library are likely to be double stranded. Similarly, while DNA may be subject to partition based on different levels of methylation, in the final library, tagged molecules derived from these molecules are likely to be unmethylated. Accordingly, the tag attached to a molecule in the library typically indicates the characteristic of the “parent molecule” from which the ultimate tagged molecule is derived, not necessarily to characteristic of the tagged molecule, itself.

As an example, barcodes 1, 2, 3, 4, etc. are used to tag and label molecules in the first partition; barcodes A, B, C, D, etc. are used to tag and label molecules in the second partition; and barcodes a, b, c, d, etc. are used to tag and label molecules in the third partition. Differentially tagged partitions can be pooled prior to sequencing. Differentially tagged partitions can be separately sequenced or sequenced together concurrently, e.g., in the same flow cell of an Illumina sequencer.

After sequencing, analysis of reads can be performed on a partition-by-partition level, as well as a whole DNA population level. Tags are used to sort reads from different partitions. Analysis can include in silico analysis to determine genetic and epigenetic variation (one or more of methylation, chromatin structure, etc.) using sequence information, genomic coordinates length, coverage, and/or copy number. In some embodiments, higher coverage can correlate with higher nucleosome occupancy in a genomic region, while lower coverage can correlate with lower nucleosome occupancy or a nucleosome depleted region (NDR).

The agents used to partition populations of nucleic acids within a sample can be affinity agents, such as antibodies with the desired specificity, natural binding partners or variants thereof (Bock et al., Nat Biotech 28: 1106-1114 (2010); Song et al., Nat Biotech 29: 68-72 (2011)), or artificial peptides selected e.g., by phage display to have specificity to a given target. In some embodiments, the agent used in the partitioning is an agent that recognizes a modified nucleobase. In some embodiments, the modified nucleobase recognized by the agent is a modified cytosine, such as a methylcytosine (e.g., 5-methylcytosine). In some embodiments, the modified nucleobase recognized by the agent is a product of a procedure that affects the first nucleobase in the DNA differently from the second nucleobase in the DNA of the sample. In some embodiments, the modified nucleobase may be a “converted nucleobase,” meaning that its base pairing specificity was changed by a procedure. For example, certain procedures convert unmethylated or unmodified cytosine to dihydrouracil, or more generally, at least one modified or unmodified form of cytosine undergoes deamination, resulting in uracil (considered a modified nucleobase in the context of DNA) or a further modified form of uracil. Examples of partitioning agents include antibodies, such as antibodies that recognize a modified nucleobase, which may be a modified cytosine, such as a methylcytosine (e.g., 5-methylcytosine). In some embodiments, the partitioning agent is an antibody that recognizes a modified cytosine other than 5-methylcytosine, such as 5-carboxylcytosine (5caC). Alternative partitioning agents include methyl binding domain (MBDs) and methyl binding proteins (MBPs) as described herein, including proteins such as MeCP2, MBD2, and antibodies preferentially binding to 5-methylcytosine. Where an antibody is used to immunoprecipitate methylated DNA, the methylated DNA may be recovered in single-stranded form. In such embodiments, a second strand can be synthesized. Hypermethylated (and optionally intermediately methylated) subsamples may then be contacted with a methylation sensitive nuclease that does not cleave hemi-methylated DNA, such as HpaII, BstUI, or Hin6i. Alternatively or in addition, hypomethylated (and optionally intermediately methylated) subsamples may then be contacted with a methylation dependent nuclease that cleaves hemi-methylated DNA.

Additional, non-limiting examples of partitioning agents are histone binding proteins which can separate nucleic acids bound to histones from free or unbound nucleic acids. Examples of histone binding proteins that can be used in the methods disclosed herein include RBBP4, RbAp48 and SANT domain peptides.

In some embodiments, partitioning can comprise both binary partitioning and partitioning based on degree/level of modifications. For example, methylated fragments can be partitioned by methylated DNA immunoprecipitation (MeDIP), or all methylated fragments can be partitioned from unmethylated fragments using methyl binding domain proteins (e.g., MethylMiner™ Methylated DNA Enrichment Kit (ThermoFisher Scientific). Subsequently, additional partitioning may involve eluting fragments having different levels of methylation by adjusting the salt concentration in a solution with the methyl binding domain and bound fragments. As salt concentration increases, fragments having greater methylation levels are eluted.

In some embodiments, methylation levels can be determined using partitioning, modification-sensitive conversion such as bisulfite conversion, direct detection during sequencing, methylation-sensitive restriction enzyme digestion, methylation-dependent restriction enzyme digestion, or any other suitable approach. For example, different forms of DNA (e.g., hypermethylated and hypomethylated DNA) can be physically partitioned based on one or more characteristics of the DNA. For example, a methylated DNA binding protein (e.g., an MBD such as MBD2, MBD4, or MeCP2) or an antibody specific for 5-methylcytosine (as in MeDIP) can be used to partition the DNA. In some embodiments, a DNA fragmentation pattern can be determined based on endpoints and/or centerpoints of DNA molecules, such as cfDNA molecules.

In some instances, the final partitions are enriched in nucleic acids having different extents of modifications (overrepresentative or underrepresentative of modifications). Overrepresentation and underrepresentation can be defined by the number of modifications born by a nucleic acid relative to the median number of modifications per strand in a population. For example, if the median number of 5-methylcytosine residues in nucleic acid in a sample is 2, a nucleic acid including more than two 5-methylcytosine residues is overrepresented in this modification and a nucleic acid with 1 or zero 5-methylcytosine residues is underrepresented. The effect of the affinity separation is to enrich for nucleic acids overrepresented in a modification in a bound phase and for nucleic acids underrepresented in a modification in an unbound phase (i.e. in solution). The nucleic acids in the bound phase can be eluted before subsequent processing.

When using MeDIP or MethylMiner™ Methylated DNA Enrichment Kit (ThermoFisher Scientific) various levels of methylation can be partitioned using sequential elutions. For example, a hypomethylated partition (no methylation) can be separated from a methylated partition by contacting the nucleic acid population with the MBD from the kit, which is attached to magnetic beads. The beads are used to separate out the methylated nucleic acids from the non-methylated nucleic acids. Subsequently, one or more elution steps are performed sequentially to elute nucleic acids having different levels of methylation. For example, a first set of methylated nucleic acids can be eluted at a salt concentration of 160 mM or higher, e.g., at least 150 mM, at least 200 mM, 300 mM, 400 mM, 500 mM, 600 mM, 700 mM, 800 mM, 900 mM, 1000 mM, or 2000 mM. After such methylated nucleic acids are eluted, magnetic separation is once again used to separate higher level of methylated nucleic acids from those with lower level of methylation. The elution and magnetic separation steps can be repeated to create various partitions such as a hypomethylated partition (enriched in nucleic acids comprising no methylation), a methylated partition (enriched in nucleic acids comprising low levels of methylation), and a hyper methylated partition (enriched in nucleic acids comprising high levels of methylation).

In some methods, nucleic acids bound to an agent used for affinity separation based partitioning are subjected to a wash step. The wash step washes off nucleic acids weakly bound to the affinity agent. Such nucleic acids can be enriched in nucleic acids having the modification to an extent close to the mean or median (i.e., intermediate between nucleic acids remaining bound to the solid phase and nucleic acids not binding to the solid phase on initial contacting of the sample with the agent).

The affinity separation results in at least two, and sometimes three or more partitions of nucleic acids with different extents of a modification. While the partitions are still separate, the nucleic acids of at least one partition, and usually two or three (or more) partitions are linked to nucleic acid tags, usually provided as components of adapters, with the nucleic acids in different partitions receiving different tags that distinguish members of one partition from another. The tags linked to nucleic acid molecules of the same partition can be the same or different from one another. But if different from one another, the tags may have part of their code in common so as to identify the molecules to which they are attached as being of a particular partition.

For further details regarding portioning nucleic acid samples based on characteristics such as methylation, see WO2018/119452, which is incorporated herein by reference.

In some embodiments, the nucleic acid molecules can be partitioned into different partitions based on the nucleic acid molecules that are bound to a specific protein or a fragment thereof and those that are not bound to that specific protein or fragment thereof.

Nucleic acid molecules can be partitioned based on DNA-protein binding. Protein-DNA complexes can be partitioned based on a specific property of a protein. Examples of such properties include various epitopes, modifications (e.g., histone methylation or acetylation) or enzymatic activity. Examples of proteins which may bind to DNA and serve as a basis for fractionation may include, but are not limited to, protein A and protein G. Any suitable method can be used to partition the nucleic acid molecules based on protein bound regions. Examples of methods used to partition nucleic acid molecules based on protein bound regions include, but are not limited to, SDS-PAGE, chromatin-immuno-precipitation (ChIP), heparin chromatography, and asymmetrical field flow fractionation (AF4).

In some embodiments, the partitioning is performed by contacting the nucleic acids with a methyl binding domain (“MBD”) of a methyl binding protein (“MBP”). In some such embodiments, the nucleic acids are contacted with an entire MBP. In some embodiments, an MBD binds to 5-methylcytosine (5mC), and an MBP comprises an MBD and is referred to interchangeably herein as a methyl binding protein or a methyl binding domain protein. In some embodiments, MBD is coupled to paramagnetic beads, such as Dynabeads® M-280 Streptavidin via a biotin linker. Partitioning into fractions with different extents of methylation can be performed by eluting fractions by increasing the NaCl concentration.

In some embodiments, bound DNA is eluted by contacting the antibody or MBD with a protease, such as proteinase K. This may be performed instead of or in addition to elution steps using NaCl as discussed above.

Examples of agents that recognize a modified nucleobase contemplated herein include, but are not limited to:
(a) MeCP2 is a protein that preferentially binds to 5-methyl-cytosine over unmodified cytosine.
(b) RPL26, PRP8 and the DNA mismatch repair protein MHS6 preferentially bind to 5-hydroxymethyl-cytosine over unmodified cytosine.
(c) FOXK1, FOXK2, FOXP1, FOXP4 and FOXI3 preferably bind to 5-formyl-cytosine over unmodified cytosine (Iurlaro et al., Genome Biol. 14: R119 (2013)).
(d) Antibodies specific to one or more methylated or modified nucleobases or conversion products thereof, such as 5mC, 5caC, or DHU.

In general, elution is a function of the number of modifications, such as the number of methylated sites per molecule, with molecules having more methylation eluting under increased salt concentrations. To elute the DNA into distinct populations based on the extent of methylation, one can use a series of elution buffers of increasing NaCl concentration. Salt concentration can range from about 100 nm to about 2500 mM NaCl. In one embodiment, the process results in three (3) partitions. Molecules are contacted with a solution at a first salt concentration and comprising a molecule comprising an agent that recognizes a modified nucleobase, which molecule can be attached to a capture moiety, such as streptavidin. At the first salt concentration a population of molecules will bind to the agent and a population will remain unbound. The unbound population can be separated as a “hypomethylated” population. For example, a first partition enriched in hypomethylated form of DNA is that which remains unbound at a low salt concentration, e.g., 100 mM or 160 mM. A second partition enriched in intermediate methylated DNA is eluted using an intermediate salt concentration, e.g., between 100 mM and 2000 mM concentration. This is also separated from the sample. A third partition enriched in hypermethylated form of DNA is eluted using a high salt concentration, e.g., at least about 2000 mM.

In some embodiments, a monoclonal antibody raised against 5-methylcytidine (5mC) is used to purify methylated DNA. DNA is denatured, e.g., at 95° C. in order to yield single-stranded DNA fragments. Protein G coupled to standard or magnetic beads as well as washes following incubation with the anti-5mC antibody are used to immunoprecipitate DNA bound to the antibody. Such DNA may then be eluted. Partitions may comprise unprecipitated DNA and one or more partitions eluted from the beads.

In some embodiments, the partitions of DNA are desalted and concentrated in preparation for enzymatic steps of library preparation.

Sequences that comprise aberrantly high copy numbers may tend to be hypermethylated. Accordingly, in some embodiments, the DNA contacted with target-specific probes specific for members of an epigenetic target region set comprising a plurality of target regions that are both type-specific differentially methylated regions and copy number variants comprises at least a portion of a hypermethylated partition. The DNA from or comprising at least a portion of the hypermethylated partition may or may not be combined with DNA from or comprising at least a portion of one or more other partitions, such as an intermediate partition or a hypomethylated partition.

In some cases, different procedures are applied to different partitions to determine different characteristics of the initial sample. In some embodiments, the DNA of at least one partition is subjected to an end repair and sequencing procedure described herein. In some embodiments at least one partition is not subjected to the end repair and sequencing procedure according to the methods of the disclosure described herein. In cases where the sequencing procedure comprises a conversion procedure, corresponding sequences from the converted and non-converted partitions can be compared to identify single nucleotides that have undergone conversion and therefore identify corresponding modified nucleosides in the initial sample.

Disclosed methods herein can comprise analyzing DNA in a sample. In some embodiments described herein, the disclosed methods comprise partitioning DNA. In such methods, different forms of DNA (e.g., hypermethylated and hypomethylated DNA) can be physically partitioned based on one or more characteristics of the DNA. This approach can be used to determine, for example, whether certain sequences are hypermethylated or hypomethylated and whether certain hypermethylated regions overlap with regions with copy number variants. In some embodiments, a first subsample or aliquot of a sample is subjected to steps for making capture probes as described elsewhere herein and a second subsample or aliquot of a sample is subjected to partitioning. In some embodiments, a sample or subsample or aliquot thereof is subjected to partitioning and differential tagging, followed by a capture step using capture probes for rearranged sequences and optionally additional capture probes, e.g., for sequence-variable and/or epigenetic target regions.

Methylation profiling can involve determining methylation patterns across different regions of the genome. For example, after partitioning molecules based on extent of methylation (e.g., relative number of methylated nucleobases per molecule) and sequencing, the sequences of molecules in the different partitions can be mapped to a reference genome. This can show regions of the genome that, compared with other regions, are more highly methylated or are less highly methylated. In this way, genomic regions, in contrast to individual molecules, may differ in their extent of methylation.

### D. Enriching, Capturing, and Using Capture Probes

DNA molecules in a sample or subsample can be subjected to a capture step (also referred to herein as a “enriching” or “enrichment” step), in which molecules having target sequences are captured for subsequent analysis.

In some embodiments, methods disclosed herein can comprise a step of capturing (i.e., enriching) DNA, such as type-specific cfDNA target regions. In some embodiments, the capturing step comprises contacting the DNA with probes specific for the target regions. Methods disclosed herein can comprise enriching, capturing, or isolating target regions and/or segments comprising recombined CDR3 sequences from DNA, such as cfDNA, e.g., from the first subsample. In some embodiments, e.g., to capture target regions such as sequence-variable target regions, the capturing comprises contacting the DNA with probes specific for the target regions. In some embodiments, e.g., to enrich segments comprising recombined CDR3 sequences, the segments can be amplified by performing multiplex amplification, e.g., from the second subsample. Exemplary primers for such multiplex amplification are provided elsewhere herein. In some embodiments, the capturing step comprises enriching for or capturing one or more primer-extended products and/or target regions hybridized to the one or more primer-extended products. Enrichment or capture may be performed on any sample or subsample described herein using any suitable approach known in the art.

In some embodiments, the CDR3 of the recombined CDR3 sequences can be a part of a T cell receptor (TCR), TCR beta chain, B cell receptor, immunoglobulin, B cell receptor heavy chain, or immunoglobulin heavy chain. In some embodiments, the CDR3 can be part of TCR CDR3 repertoire. A TCR repertoire, for example, generated by the process of V(D)J recombination, encompasses the T cell clones within a given individual or sample, and TCRs can be indicative of disease (e.g., cancer) status, prior infections or immunizations, and individual-specific attributes of epitope selection.

In some embodiments, the probes specific for DNA target regions comprise a capture moiety that facilitates the enrichment or capture of the DNA hybridized to the probes, respectively. In some embodiments, the amplified products comprise a capture moiety that facilitates the enrichment or capture of the primer-extended products and DNA hybridized to the primer-extended products. The capture moiety may be provided as part of the primer or incorporated during extension as part of a modified deoxyribonucleotide triphosphate as discussed in detail elsewhere herein.

As discussed above, nucleic acids in a sample can be subject to a capture step, in which molecules having certain characteristics are captured and analyzed. Target capture can involve use of a bait set comprising oligonucleotide baits labeled with a capture moiety, such as biotin or the other examples noted below. The probes can have sequences selected to tile across a panel of regions, such as genes. In some embodiments, a bait set can have higher and lower capture yields for sets of target regions such as those of the sequence-variable target region set and the epigenetic target region set, respectively, as discussed elsewhere herein. Such bait sets are combined with a sample under conditions that allow hybridization of the target molecules with the baits. Then, captured molecules are isolated using the capture moiety. DNA capture can involve use of oligonucleotides labeled with a capture moiety, such as target-specific probes labeled with biotin, and a second moiety or binding partner that binds to the capture moiety, such as streptavidin. In some embodiments, a capture moiety and binding partner can have higher and lower capture yields for different sets of probes, such as those used to capture a sequence-variable target region set, recombined CDR3 sequences, target regions that may comprise a structural variation, and an epigenetic target region set, respectively, as discussed elsewhere herein.

Capture may be performed using any suitable approach known in the art. Target capture can involve use of a bait set comprising oligonucleotide baits (a type of probe useful herein) labeled with a capture moiety, such as biotin or the other examples noted below. The probes can have sequences selected to tile across a panel of regions, such as genes. Such bait sets are combined with a sample under conditions that allow hybridization of the target molecules with the baits. Then, captured molecules are isolated using the capture moiety. For example, a biotin capture moiety by bead-based streptavidin. Such methods are further described in, for example, U.S. Pat. No. 9,850,523, issuing Dec. 26, 2017, which is incorporated herein by reference.

Capture moieties include, without limitation, biotin, avidin, streptavidin, a nucleic acid comprising a particular nucleotide sequence, digoxygenin, a histidine tag, an affinity tag, an immunoglobulin constant domain, a hapten recognized by an antibody, and magnetically attractable particles. In some embodiments, the immunoglobulin constant domain may be bound using protein A, protein G, or a secondary antibody. In some embodiments, the secondary antibody comprises an anti-mouse secondary antibody. In some embodiments, the anti-mouse secondary antibody is a goat anti-mouse secondary antibody, rabbit anti-mouse secondary antibody, or a donkey anti-mouse secondary antibody. The extraction moiety can be a member of a binding pair, such as biotin/streptavidin or hapten/antibody. In some embodiments, a capture moiety that is attached to an analyte is captured by its binding pair which is attached to an isolatable moiety, such as a magnetically attractable particle or a large particle that can be sedimented through centrifugation. The capture moiety can be any type of molecule that allows affinity separation of nucleic acids bearing the capture moiety from nucleic acids lacking the capture moiety. Exemplary capture moieties are biotin that allows affinity separation by binding to streptavidin linked or linkable to a solid phase or an oligonucleotide, which allows affinity separation through binding to a complementary oligonucleotide linked or linkable to a solid phase.

In some embodiments, the probes specific for the target regions (i.e., target-specific probes) comprise a capture moiety that facilitates the enrichment or capture of the DNA hybridized to the probes. In some embodiments, the capture moiety is biotin. In some such embodiments, streptavidin attached to a solid support, such as magnetic beads, is used to bind to the biotin. Nonspecifically bound DNA that does not comprise a target region is washed away from the captured DNA. In some embodiments, DNA is then dissociated from the probes and eluted from the solid support using salt washes or buffers comprising another DNA denaturing agent. In some embodiments, the probes are also eluted from the solid support by, e.g., disrupting the biotin-streptavidin interaction. In some embodiments, captured DNA is amplified following elution from the solid support. In some such embodiments, DNA comprising adapters is amplified using PCR primers that anneal to the adapters. In some embodiments, captured DNA is amplified while attached to the solid support. In some such embodiments, the amplification comprises the use of a PCR primer that anneals to a sequence within an adapter and a PCR primer that anneals to a sequence within a probe annealed to the target region of the DNA. In some embodiments, e.g., where the capture moiety is provided as part of the primer, the presence of short primer-extended products and unextended primers can be minimized by performing an amplification subsequent to capture that is dependent on the presence of adapter sequences at both ends of the DNA for exponential amplification, e.g., such that short primer-extended products (which resulted from extension that was blocked before reaching an adapter sequence in the template molecule) are not exponentially amplified or are amplified to a lesser extent than primer-extended products that comprise adapter sequences at both ends.

In some embodiments, the methods herein comprise enriching for or capturing DNA comprising sequence-variable target region set, recombined CDR3 sequences, target regions that may comprise a structural variation, and/or the epigenetic target region set. Such regions may be captured from an aliquot of a sample (e.g., a sample that has undergone attachment of adapters and amplification). Enriching for or capturing DNA comprising sequence-variable target region set, recombined CDR3 sequences, target regions that may comprise a structural variation, and the epigenetic target region set may comprise contacting the DNA with a different set of target-specific probes.

A panel of regions targeted for enrichment can be selected such that they do not contain regions known to include the base modification used in the end repair reaction. When the end repair is performed with dNTPs comprising 5mC or 5hmC, a panel of regions targeted for enrichment may be selected such that they do not contain CpH dinucleotides which are known to be naturally methylated in the subject (e.g. humans). Such CpH dinucleotides can be identified through the use of publicly available resources (e.g. MethBank3.0: a database of DNA methylomes across a variety of species Nucleic Acids Res 2018). Such an approach has the advantage that any detected methylated CpH dinucleotides can unambiguously be attributed to regions synthesized in the end repair.

In some embodiments, capturing comprises contacting the DNA to be captured with a set of target-specific probes. Such target-specific probes may have any of the features described herein for sets of target-specific probes, including but not limited to in the embodiments set forth above and the sections relating to probes below. Capturing may be performed on one or more subsamples prepared during methods disclosed herein. In some embodiments, DNA is captured from at least the first subsample or the second subsample, e.g., at least the first subsample and the second subsample. In some embodiments, the subsamples are differentially tagged (e.g., as described herein) and then pooled before undergoing capture.

The capturing step may be performed using conditions suitable for specific nucleic acid hybridization, which generally depend to some extent on features of the probes such as length, base composition, etc. Those skilled in the art will be familiar with appropriate conditions given general knowledge in the art regarding nucleic acid hybridization. In some embodiments, complexes of target-specific probes and DNA are formed.

In some embodiments, a method described herein comprises capturing cfDNA obtained from a subject for a plurality of sets of target regions. The target regions comprise epigenetic target regions, which may show differences in methylation levels and/or fragmentation patterns depending on whether they originated from a tumor or from healthy cells. The target regions also comprise sequence-variable target regions, which may show differences in sequence depending on whether they originated from a tumor or from healthy cells. The capturing step produces a captured set of cfDNA molecules and the cfDNA molecules corresponding to the sequence-variable target region set are captured at a greater capture yield in the captured set of cfDNA molecules than cfDNA molecules corresponding to the epigenetic target region set.

In some embodiments, a method described herein comprises contacting cfDNA obtained from a subject with a set of target-specific probes, wherein the set of target-specific probes is configured to capture cfDNA corresponding to the sequence-variable target region set at a greater capture yield than cfDNA corresponding to the epigenetic target region set. In some embodiments, a method described herein comprises contacting cfDNA obtained from a subject with a set of target-specific probes, wherein the set of target-specific probes is configured to capture cfDNA corresponding to the recombined CDR3 sequences at a greater capture yield than cfDNA corresponding to other target region set. For additional discussion of capturing steps, capture yields, and related aspects, see WO2020/160414, which is incorporated herein by reference for all purposes.

In some embodiments, it can be beneficial to capture cfDNA corresponding to the recombined CDR3 sequences or cfDNA corresponding to the target regions that may comprise a structural variation at a greater capture yield than cfDNA corresponding to the sequence-variable target region set or cfDNA corresponding to the epigenetic target region set because a greater depth of sequencing may be necessary to analyze the recombined CDR3 sequences with sufficient confidence or accuracy and optimized sensitivity. In some embodiments, it can be beneficial to capture cfDNA corresponding to the sequence-variable target region set at a greater capture yield than cfDNA corresponding to the epigenetic target region set because a greater depth of sequencing may be necessary to analyze the sequence-variable target regions with sufficient confidence or accuracy than may be necessary to analyze the epigenetic target regions. The volume of data needed to determine fragmentation patterns (e.g., to test for perturbation of transcription start sites or CTCF binding sites) or fragment abundance (e.g., in hypermethylated and hypomethylated partitions) is generally less than the volume of data needed to determine the presence or absence of cancer-related sequence mutations. Capturing the target region sets at different yields can facilitate sequencing the target regions to different depths of sequencing in the same sequencing run (e.g., using a pooled mixture and/or in the same sequencing cell). Although copy number variations such as focal amplifications are somatic mutations, they can be detected by sequencing based on read frequency in a manner analogous to approaches for detecting certain epigenetic changes such as changes in methylation. Thus, they can be considered epigenetic target regions for functional reasons. Additionally, regions showing copy number variation that are also hypermethylation-variable or fragmentation-variable target regions are considered epigenetic target regions because they may show epigenetic variation.

In some embodiments, the DNA is amplified. In some embodiments, amplification is performed before the capturing step. In some embodiments, amplification is performed after the capturing step. In some embodiments, amplification is performed before and after the capturing step. In various embodiments, the methods further comprise sequencing the captured DNA, e.g., to different degrees of sequencing depth for the epigenetic and sequence-variable target region sets, consistent with the discussion herein.

In some embodiments, a capturing step is performed with probes for a sequence-variable target region set, probes for an epigenetic target region set, or probes for recombined CDR3 sequences in the same vessel at the same time, e.g., the probes for the sequence-variable, epigenetic target region sets, or probes for recombined CDR3 sequences are in the same composition. This approach provides a relatively streamlined workflow. In some embodiments, complexes of target-specific probes and DNA are separated from DNA not bound to target-specific probes. For example, where target-specific probes are bound covalently or noncovalently to a solid support, a washing or aspiration step can be used to separate unbound material. Alternatively, where the complexes have chromatographic properties distinct from unbound material (e.g., where the probes comprise a ligand that binds a chromatographic resin), chromatography can be used.

As discussed in detail elsewhere herein, the set of target-specific probes may comprise a plurality of sets such as probes for a sequence-variable target region set and probes for an epigenetic target region set. In some embodiments, a capturing step is performed with the probes for a sequence-variable target region set and the probes for an epigenetic target region set in the same vessel at the same time, e.g., the probes for the sequence-variable and epigenetic target region sets and capture probes are in the same composition. This approach provides a relatively streamlined workflow. In some embodiments, the concentration of the probes for the sequence-variable target region set is greater than the concentration of the probes for the epigenetic target region set.

Alternatively, the capturing step is performed with the sequence-variable target region probe set in a first vessel and with a recombined CDR3 sequence probe or probes for target regions that may comprise a structural variation in a second vessel, and/or the epigenetic target region probe set in a third vessel. In some embodiments, the contacting step is performed with the sequence-variable target region probe set at a first time, a recombined CDR3 sequence probe or probes for target regions that may comprise a structural variation at a second time before or after the first time, and an epigenetic target region probe set at a third time different than the first or second time. This approach allows for preparation of separate first and second compositions and/or third compositions comprising captured DNA corresponding to the sequence-variable target region set and captured or enriched DNA corresponding to a recombined CDR3 sequence or target regions that may comprise a structural variation, and the epigenetic target region set. The compositions can be processed separately as desired (e.g., to fractionate based on methylation as described elsewhere herein) and pooled (e.g., recombined) in appropriate proportions to provide material for further processing and analysis, such as sequencing.

In some embodiments, a captured set of DNA (e.g., cfDNA) is provided. With respect to the disclosed methods, the captured set of DNA may be provided, e.g., by performing a capturing step prior to a sequencing step as described herein. The captured set may comprise DNA corresponding to a sequence-variable target region set, an epigenetic target region set, or a combination thereof. In some embodiments, a capture step is performed prior to a conversion step or after a conversion step.

In some embodiments, a first target region set is captured (e.g., from a sample or a first subsample), comprising at least epigenetic target regions. The epigenetic target regions captured from the first subsample may comprise hypermethylation variable target regions. In some embodiments, the hypermethylation variable target regions are CpG-containing regions that are unmethylated or have low methylation in cfDNA from healthy subjects (e.g., below-average methylation relative to bulk cfDNA). In some embodiments, the hypermethylation variable target regions are regions that are regions that show lower methylation in healthy cfDNA than in at least one other tissue type. Without wishing to be bound by any particular theory, cancer cells may shed more DNA into the bloodstream that healthy cells of the same tissue type. As such, the distribution of tissue of origin of cfDNA may change upon carcinogenesis. Thus, an increase in the level of hypermethylation variable target regions in the first subsample can be an indicator of the presence (or recurrence, depending on the history of the subject) of cancer.

In some embodiments, a second target region set is captured from the second subsample comprising at least epigenetic target regions. The epigenetic target regions may comprise hypomethylation variable target regions. In some embodiments, the hypomethylation variable target regions are CpG-containing regions that are methylated or have high methylation in cfDNA from healthy subjects (e.g., above-average methylation relative to bulk cfDNA). In some embodiments, the hypomethylation variable target regions are regions that show higher methylation in healthy cfDNA than in at least one other tissue type. Without wishing to be bound by any particular theory, cancer cells may shed more DNA into the bloodstream than healthy cells of the same tissue type. As such, the distribution of tissue of origin of cfDNA may change upon carcinogenesis. Thus, an increase in the level of hypomethylation variable target regions in the second subsample can be an indicator of the presence (or recurrence, depending on the history of the subject) of cancer.

In some embodiments the quantity of captured sequence-variable target region DNA is greater than the quantity of the captured epigenetic target region DNA, when normalized for the difference in the size of the targeted regions (footprint size).

Alternatively, first and second captured sets may be provided, comprising, respectively, DNA corresponding to a sequence-variable target region set and DNA corresponding to an epigenetic target region set. The captured sets may be combined to provide a combined captured set.

In some embodiments in which a captured set comprising DNA corresponding to the sequence-variable target region set and the epigenetic target region set includes a combined captured set as discussed above, the DNA corresponding to the sequence-variable target region set may be present at a greater concentration than the DNA corresponding to the epigenetic target region set, e.g., a 1.1 to 1.2-fold greater concentration, a 1.2- to 1.4-fold greater concentration, a 1.4- to 1.6-fold greater concentration, a 1.6- to 1.8-fold greater concentration, a 1.8- to 2.0-fold greater concentration, a 2.0- to 2.2-fold greater concentration, a 2.2- to 2.4-fold greater concentration a 2.4- to 2.6-fold greater concentration, a 2.6- to 2.8-fold greater concentration, a 2.8- to 3.0-fold greater concentration, a 3.0- to 3.5-fold greater concentration, a 3.5- to 4.0, a 4.0- to 4.5-fold greater concentration, a 4.5- to 5.0-fold greater concentration, a 5.0-to 5.5-fold greater concentration, a 5.5- to 6.0-fold greater concentration, a 6.0- to 6.5-fold greater concentration, a 6.5- to 7.0-fold greater, a 7.0- to 7.5-fold greater concentration, a 7.5- to 8.0-fold greater concentration, an 8.0- to 8.5-fold greater concentration, an 8.5- to 9.0-fold greater concentration, a 9.0- to 9.5-fold greater concentration, 9.5- to 10.0-fold greater concentration, a 10- to 11-fold greater concentration, an 11- to 12-fold greater concentration a 12- to 13-fold greater concentration, a 13- to 14-fold greater concentration, a 14- to 15-fold greater concentration, a 15- to 16-fold greater concentration, a 16- to 17-fold greater concentration, a 17- to 18-fold greater concentration, an 18- to 19-fold greater concentration, a 19- to 20-fold greater concentration, a 20- to 30-fold greater concentration, a 30- to 40-fold greater concentration, a 40- to 50-fold greater concentration, a 50- to 60-fold greater concentration, a 60- to 70-fold greater concentration, a 70- to 80-fold greater concentration, a 80-to 90-fold greater concentration, or a 90- to 100-fold greater concentration. The degree of difference in concentrations accounts for normalization for the footprint sizes of the target regions, as discussed in the definition section.

In some embodiments, the DNA that is captured comprises intronic regions. In some embodiments, the intronic regions comprise one or more introns likely to differentiate DNA from neoplastic (e.g., tumor or cancer) cells and from healthy cells, e.g., non-neoplastic circulating cells. For example, an intron comprising a rearrangement known to be present in some neoplastic cells and absent from healthy cells can be used to differentiate DNA from neoplastic (e.g., tumor or cancer) cells and from healthy cells. In some embodiments, the rearrangement is a translocation.

In some embodiments, captured intronic regions have a footprint of at least 30 bp, e.g., at least 100 bp, at least 200 bp, at least 500 bp, at least 1 kb, at least 2 kb, at least 5 kb, at least 10 kb, at least 20 kb, at least 50 kb, at least 200 kb, at least 300 kb, or at least 400 kb. In some embodiments, the intronic target region set has a footprint in the range of 30 bp-1000 kb, e.g., 30 bp-100 bp, 100 bp-200 bp, 200 bp-500 bp, 500 bp-lkb, 1 kb-2 kb, 2 kb-5 kb, 5 kb-10 kb, 10 kb-20 kb, 20 kb-50 kb, 50 kb-100 kb, 100-200 kb, 200-300 kb, 300-400 kb, 400-500 kb, 500-600 kb, 600-700 kb, 700-800 kb, 800-900 kb, and 900-1,000 kb.

Exemplary rearrangements, such as intronic translocations that can be detected using the methods described herein include but are not limited to translocations wherein at least one of the two genes involved in the translocation is a receptor tyrosine kinase. Exemplary translocation products are the BCR-ABL fusion. and fusions comprising any of ALK, FGFR2, FGFR3, NTRK1, RET, or ROS1.

In some embodiments, adapters are included in the DNA as described herein. In some embodiments, tags, which may be or include barcodes, are included in the DNA. In some embodiments, such tags are included in adapters. Tags can facilitate identification of the origin of a nucleic acid. For example, barcodes can be used to allow the origin (e.g., subject) whence the DNA came to be identified following pooling of a plurality of samples for parallel sequencing. This may be done concurrently with an amplification procedure, e.g., by providing the barcodes in a 5′ portion of a primer, e.g., as described herein. In some embodiments, adapters and tags/barcodes are provided by the same primer or primer set. For example, the barcode may be located 3′ of the adapter and 5′ of the target-hybridizing portion of the primer. Alternatively, barcodes can be added by other approaches, such as ligation, optionally together with adapters in the same ligation substrate.

Additional details regarding amplification, tags, and barcodes are discussed herein, which can be combined to the extent practicable with any of these embodiments.

In some embodiments, the DNA that is captured comprises target regions having a type-specific epigenetic variation and/or a copy number variation. In some embodiments, an epigenetic target region set consists of target regions having a type-specific epigenetic variation and/or a copy number variation. In some embodiments, the type-specific epigenetic variations, e.g., differential methylation or a type-specific fragmentation pattern, are likely to differentiate DNA from one or more related cell or tissue types cells from DNA from other cell or tissue types present in a sample or in a subject.

In some embodiments, nucleic acids captured or enriched using a method described herein comprise captured DNA, such as one or more captured sets of DNA, e.g., from the first subsample. In some embodiments, the captured DNA comprise target regions that sequence-variable target regions, target regions that may comprise a structural variation, and epigenetic target regions. In some embodiments, the captured DNA comprise target regions that are differentially methylated in different immune cell types. In some embodiments, the immune cell types comprise rare or closely related immune cell types, such as activated and naïve lymphocytes or myeloid cells at different stages of differentiation.

In some embodiments, a captured epigenetic target region set captured from a sample or first subsample comprises hypermethylation variable target regions. In some embodiments, the hypermethylation variable target regions are differentially or exclusively hypermethylated in one or more related cell or tissue types. In some embodiments, the hypermethylation variable target regions are differentially or exclusively hypermethylated in one cell type or in one immune cell type, or in one immune cell type within a cluster. In some embodiments, the hypermethylation variable target regions are hypermethylated to an extent that is distinguishably higher or exclusively present in one cell type or one immune cell type or one immune cell type within a cluster. Such hypermethylation variable target regions may be hypermethylated in other cell or tissue types but not to the extent observed in the one or more related cell or tissue types. In some embodiments, the hypermethylation variable target regions show lower methylation in healthy cfDNA than in at least one other tissue type. In some embodiments, the hypermethylation variable target regions show even higher methylation in cfDNA from a diseased cell of the one or more related cell or tissue types. In some embodiments, target regions comprise hypermethylated regions with aberrantly high copy number. In some such embodiments, the target regions are hypermethylated in healthy and diseased colon tissue and have aberrantly high copy number in precancerous or cancerous colon tissue. Examples of such target regions are shown in Table 5 below. A gene is considered to comprise a DMR when the DMR is located within an untranslated region (UTR), intron, or exon of the gene, or within 5000 nucleotides of either the 5′ end of the sense strand of the 5′ UTR or the 3′ end of the sense strand of the 3′ UTR.

TABLE 5
Hypermethylated target regions with aberrantly
high copy number in colon cancer or precancer

| Chromosomal | |
|---|---|
| region with | |
| copy number | Genes comprising DMRs within |
| gain | the chromosomal region |
| 7p21.3-15.1 | VWDE, TWIST1, DNAH1, GPNMB, NPY, |
| | GSDME, NFE2L3, HOXA1, HOXA7, EVX1, |
| | CREB5 |
| 8q13.3-24.3 | PRDM14, MSC, TRPA1, SBSPON, CRISPLD1, |
| | ZFHX4, RALYL, MMP16, C8orf88, RUNX1T1, |
| | CDH17, GDF6, SDC2, OSR2, PABPC1, ZNF706, |
| | RIMS2, ZFPM2, SYBU, CSMD3, TRPS1, |
| | AARD, TNFRSF11B, MAL2, COL14A1, |
| | POU5F1B, ADCY8, ST3GAL1, TRAPPC9, |
| | AGO2, ADGRB1, LY6E, LY6H, GLI4, MAFA, |
| | TSTA3, PLEC, OPLAH, FOXH1 |
| 13q14.1-33.3 | ELF1, RGCC, TNFSF11, SERP2, RB1, CNMD, |
| | PCDH17, EDNRB, POU4F1, SLITRK5, GPC6, |
| | SOX21, CLDN10, STK24, ZIC2, NALCN, |
| | FGF14, MYO16, COL4A1, COL4A2, SOX1, F7 |
| 20q12-13.33 | MAFB, LPIN3, EMILIN3, PTPRT, TOX2, |
| | GDAP1L1, HNF4A, MMP9, CDH22, PREX1, |
| | ZNFX1, PARD6B, ZFP64, DOK5, FAM210B, |
| | TFAP2C, RBM38, CTCFL, GNAS, PHACTR3, |
| | CDH4, GATA5, BHLHE23, YTHDF1, NKAIN4, |
| | CHRNA4, KCNQ2, GMEB2 |

TABLE 6
Exemplary Hypermethylation Target Regions
based on Lung Cancer studies

| Gene Name | Chromosome |
|---|---|
| MARCH11 | chr5 |
| TAC1 | chr7 |
| TCF21 | chr6 |
| SHOX2 | chr3 |
| p16 | chr3 |
| Casp8 | chr2 |
| CDH13 | chr16 |
| MGMT | chr10 |
| MLH1 | chr3 |
| MSH2 | chr2 |
| TSLC1 | chr11 |
| APC | chr5 |
| DKK1 | chr10 |
| DKK3 | chr11 |
| LKB1 | chr11 |
| WIF1 | chr12 |
| RUNX3 | chr1 |
| GATA4 | chr8 |
| GATA5 | chr20 |
| PAX5 | chr9 |
| E-Cadherin | chr16 |
| H-Cadherin | chr16 |

In some embodiments, a captured epigenetic target region set captured from a sample or subsample comprises hypomethylation variable target regions. In some embodiments, the hypomethylation variable target regions are exclusively hypomethylated in one or more related cell or tissue types. In some embodiments, the hypomethylation variable target regions are exclusively hypomethylated in one cell type or in one immune cell type or in one immune cell type within a cluster. In some embodiments, the hypomethylation variable target regions are hypomethylated to an extent that is exclusively present in one cell type or one immune cell type or in one immune cell type within a cluster. Such hypomethylation variable target regions may be hypomethylated in other cell or tissue types but not to the extent observed in the one or more cell or tissue types. In some embodiments, the hypomethylation variable target regions show higher methylation in healthy cfDNA than in at least one other tissue type.

Without wishing to be bound by any particular theory, in an individual with cancer, proliferating or activated immune cells and/or dying cancer cells may shed more DNA into the bloodstream than cells (e.g., immune cells) in a healthy individual and/or healthy cells of the same tissue type, respectively. As such, the distribution of cell type and/or tissue of origin of cfDNA may change upon carcinogenesis. Variations in hypermethylation and/or hypomethylation can be an indicator of disease. Thus, the presence and/or levels of cfDNA originating from certain cell or tissue types can be an indicator of disease. For example, an increase in the level of hypermethylation variable target regions and/or hypomethylation variable target regions in a subsample following a partitioning step can be an indicator of the presence (or recurrence, depending on the history of the subject) of cancer.

Exemplary hypermethylation variable target regions and hypomethylation variable target regions useful for distinguishing between various cell types, including but not limited to immune cell types, have been identified by analyzing DNA obtained from various cell types via whole genome bisulfite sequencing, as described, e.g., in Scott, C. A., Duryea, J. D., MacKay, H. et al., “Identification of cell type-specific methylation signals in bulk whole genome bisulfite sequencing data,” Genome Biol 21, 156 (2020) (doi.org/10.1186/si3059-020-02065-5). Whole-genome bisulfite sequencing data is available from the Blueprint consortium, available on the internet at dcc.blueprint-epigenome.eu.

In some embodiments, first and second captured target region sets comprise, respectively, DNA corresponding to a sequence-variable target region set and DNA corresponding to an epigenetic target region set, and DNA comprising a structural variation, for example, as described in WO 2020/160414. The first and second captured sets may be combined to provide a combined captured set. In some embodiments, the sequence-variable target region set, structural variation, and epigenetic target region set may have any of the features described for such sets in WO 2020/160414, which is incorporated by reference herein in its entirety. In some embodiments, the epigenetic target region set comprises a hypermethylation variable target region set. In some embodiments, the epigenetic target region set comprises a hypomethylation variable target region set. In some embodiments, the epigenetic target region set comprises CTCF binding regions. In some embodiments, the epigenetic target region set comprises fragmentation variable target regions. In some embodiments, the epigenetic target region set comprises transcriptional start sites. In some embodiments, the epigenetic target region set comprises regions that may show focal amplifications in cancer, e.g., one or more of AR, BRAF, CCND1, CCND2, CCNE1, CDK4, CDK6, EGFR, ERBB2, FGFR1, FGFR2, KIT, KRAS, MET, MYC, PDGFRA, PIK3CA, and RAF1. For example, in some embodiments, the epigenetic target region set comprises at least 2, 3, 4, 5, 6, 7, 8, 9, 10, 11, 12, 13, 14, 15, 16, 17, or 18 of the foregoing targets.

In some embodiments, the sequence-variable target region set comprises a plurality of regions known to undergo somatic mutations in cancer. In some aspects, the sequence-variable target region set targets a plurality of different genes or genomic regions (“panel”) selected such that a determined proportion of subjects having a cancer exhibits a genetic variant or tumor marker in one or more different genes or genomic regions in the panel. The panel may be selected to limit a region for sequencing to a fixed number of base pairs. The panel may be selected to sequence a desired amount of DNA, e.g., by adjusting the affinity and/or amount of the probes as described elsewhere herein. The panel may be further selected to achieve a desired sequence read depth. The panel may be selected to achieve a desired sequence read depth or sequence read coverage for an amount of sequenced base pairs. The panel may be selected to achieve a theoretical sensitivity, a theoretical specificity, and/or a theoretical accuracy for detecting one or more genetic variants in a sample.

In some embodiments, DNA structural variations comprise rearrangements, such as translocations, insertions, deletions, duplications, copy-number variants, and inversions. In some embodiments, a rearrangement is a translocation, gene fusion, insertion, deletion, or inversion. Exemplary rearrangements include products of a translocation, gene fusion, and VDJ recombination. In some embodiments, the rearrangement is the product of a translocation comprising fusion of two intronic regions. In some embodiments, the rearrangement is a product of VDJ recombination comprising adjacent J exonic regions. In some embodiments, an insertion is an insertion of at least 5, 10, 20, 30, 40, 50, 60, 70, 80, 90, or 100 nucleotides. In some embodiments, a deletion affects sequence spanning the end of the target region, so as to result in a primer being unblocked and undergoing extension in a method described herein.

Probes for detecting the panel of regions can include those for detecting genomic regions of interest (hotspot regions). Information about chromatin structure can be taken into account in designing probes, and/or probes can be designed to maximize the likelihood that particular sites (e.g., KRAS codons 12 and 13) can be captured, and may be designed to optimize capture based on analysis of cfDNA coverage and fragment size variation impacted by nucleosome binding patterns and GC sequence composition. Regions used herein can also include non-hotspot regions optimized based on nucleosome positions and GC models.

Examples of listings of genomic locations of interest may be found in Table 3 and Table 4 of WO 2020/160414. In some embodiments, a sequence-variable target region set used in the methods of the present disclosure comprises at least a portion of at least 5, at least 10, at least 15, at least 20, at least 25, at least 30, at least 35, at least 40, at least 45, at least 50, at least 55, at least 60, at least 65, or 70 of the genes of Table 3 of WO 2020/160414. In some embodiments, a sequence-variable target region set used in the methods of the present disclosure comprises at least a portion of at least 5, at least 10, at least 15, at least 20, at least 25, at least 30, at least 35, at least 40, at least 45, at least 50, at least 55, at least 60, at least 65, at least 70, or 73 of the genes of Table 4 of WO 2020/160414. Additionally or alternatively, suitable target region sets are available from the literature. For example, Gale et al., PLoS One 13: e0194630 (2018), which is incorporated herein by reference, describes a panel of 35 cancer-related gene targets that can be used as part or all of a sequence-variable target region set. These 35 targets are AKT1, ALK, BRAF, CCND1, CDK2A, CTNNB1, EGFR, ERBB2, ESR1, FGFR1, FGFR2, FGFR3, FOXL2, GATA3, GNA11, GNAQ, GNAS, HRAS, IDH1, IDH2, KIT, KRAS, MED12, MET, MYC, NFE2L2, NRAS, PDGFRA, PIK3CA, PPP2R1A, PTEN, RET, STK11, TP53, and U2AF1.

In some embodiments, the sequence-variable target region set comprises target regions from at least 10, 20, 30, or 35 cancer-related genes, such as the cancer-related genes listed above and in WO 2020/160414.

In some embodiments, a collection of capture probes is used in methods described herein, e.g., comprising capture probes prepared by any method disclosed herein for doing so. In some embodiments, the collection of capture probes further comprises target-binding probes specific for a sequence-variable target region set and/or target-binding probes specific for a sequence-variable target region set and/or target-binding probes specific an epigenetic target region set. In some embodiments, the capture yield of the capture probes specific for the sequence-variable target region set is higher (e.g., at least 2-fold higher) than the capture yield of the target-binding probes specific for the epigenetic target region set. In some embodiments, the collection of capture probes is configured to have a capture yield specific for the sequence-variable target region set higher (e.g., at least 2-fold higher) than its capture yield specific for the epigenetic target region set.

In some embodiments, the capture yield of the target-binding probes specific for the sequence-variable target region set is at least 1.25-, 1.5-, 1.75-, 2-, 2.25-, 2.5-, 2.75-, 3-, 3.5-, 4-, 4.5-, 5-, 6-, 7-, 8-, 9-, 10-, 11-, 12-, 13-, 14-, or 15-fold higher than the capture yield of the target-binding probes specific for the epigenetic target region set. In some embodiments, the capture yield of the target-binding probes specific for the sequence-variable target region set is 1.25- to 1.5-, 1.5- to 1.75-, 1.75- to 2-, 2- to 2.25-, 2.25- to 2.5-, 2.5- to 2.75-, 2.75- to 3-, 3- to 3.5-, 3.5- to 4-, 4- to 4.5-, 4.5- to 5-, 5- to 5.5-, 5.5- to 6-, 6- to 7-, 7- to 8-, 8- to 9-, 9- to 10-, 10- to 11-, 11- to 12-, 13- to 14-, or 14- to 15-fold higher than the capture yield of the target-binding probes specific for the epigenetic target region set.

In some embodiments, the collection of capture probes is configured to have a capture yield specific for the sequence-variable target region set at least 1.25-, 1.5-, 1.75-, 2-, 2.25-, 2.5-, 2.75-, 3-, 3.5-, 4-, 4.5-, 5-, 6-, 7-, 8-, 9-, 10-, 11-, 12-, 13-, 14-, or 15-fold higher than its capture yield for the epigenetic target region set. In some embodiments, the collection of capture probes is configured to have a capture yield specific for the sequence-variable target region set is 1.25- to 1.5-, 1.5- to 1.75-, 1.75- to 2-, 2- to 2.25-, 2.25- to 2.5-, 2.5- to 2.75-, 2.75- to 3-, 3- to 3.5-, 3.5- to 4-, 4- to 4.5-, 4.5- to 5-, 5- to 5.5-, 5.5- to 6-, 6- to 7-, 7- to 8-, 8- to 9-, 9- to 10-, 10- to 11-, 11- to 12-, 13- to 14-, or 14- to 15-fold higher than its capture yield specific for the epigenetic target region set.

The collection of probes can be configured to provide higher capture yields for the sequence-variable target region set in various ways, including concentration, different lengths and/or chemistries (e.g., that affect affinity), and combinations thereof. Affinity can be modulated by adjusting probe length and/or including nucleotide modifications as discussed below.

In some embodiments, the capture probes specific for the sequence-variable target region set are present at a higher concentration than the capture probes specific for the epigenetic target region set. In some embodiments, concentration of the target-binding probes specific for the sequence-variable target region set is at least 1.25-, 1.5-, 1.75-, 2-, 2.25-, 2.5-, 2.75-, 3-, 3.5-, 4-, 4.5-, 5-, 6-, 7-, 8-, 9-, 10-, 11-, 12-, 13-, 14-, or 15-fold higher than the concentration of the target-binding probes specific for the epigenetic target region set. In some embodiments, the concentration of the target-binding probes specific for the sequence-variable target region set is 1.25- to 1.5-, 1.5- to 1.75-, 1.75- to 2-, 2- to 2.25-, 2.25- to 2.5-, 2.5- to 2.75-, 2.75- to 3-, 3- to 3.5-, 3.5- to 4-, 4- to 4.5-, 4.5- to 5-, 5- to 5.5-, 5.5- to 6-, 6- to 7-, 7- to 8-, 8- to 9-, 9- to 10-, 10- to 11-, 11- to 12-, 13- to 14-, or 14- to 15-fold higher than the concentration of the target-binding probes specific for the epigenetic target region set. In such embodiments, concentration may refer to the average mass per volume concentration of individual probes in each set.

In some embodiments, the capture probes specific for the sequence-variable target region set have a higher affinity for their targets than the capture probes specific for the epigenetic target region set. Affinity can be modulated in any way known to those skilled in the art, including by using different probe chemistries. For example, certain nucleotide modifications, such as cytosine 5-methylation (in certain sequence contexts), modifications that provide a heteroatom at the 2′ sugar position, and LNA nucleotides, can increase stability of double-stranded nucleic acids, indicating that oligonucleotides with such modifications have relatively higher affinity for their complementary sequences. See, e.g., Severin et al., Nucleic Acids Res. 39: 8740-8751 (2011); Freier et al., Nucleic Acids Res. 25: 4429-4443 (1997); U.S. Pat. No. 9,738,894. Also, longer sequence lengths will generally provide increased affinity. Other nucleotide modifications, such as the substitution of the nucleobase hypoxanthine for guanine, reduce affinity by reducing the amount of hydrogen bonding between the oligonucleotide and its complementary sequence. In some embodiments, the capture probes specific for the sequence-variable target region set have modifications that increase their affinity for their targets. In some embodiments, alternatively or additionally, the capture probes specific for the epigenetic target region set have modifications that decrease their affinity for their targets. In some embodiments, the capture probes specific for the sequence-variable target region set have longer average lengths and/or higher average melting temperatures than the capture probes specific for the epigenetic target region set. These embodiments may be combined with each other and/or with differences in concentration as discussed above to achieve a desired fold difference in capture yield, such as any fold difference or range thereof described above.

In some embodiments, the capture probes comprise a capture moiety. The capture moiety may be any of the capture moieties described herein, e.g., biotin. In some embodiments, the target-specific probes are linked to a solid support, e.g., covalently or non-covalently such as through the interaction of a binding pair of capture moieties. In some embodiments, the solid support is a bead, such as a magnetic bead.

In some embodiments, the capture probes specific for the sequence-variable target region set and/or the capture probes specific for the epigenetic target region set are a capture probe set as discussed above, e.g., probes comprising capture moieties and sequences selected to tile across a panel of regions, such as genes.

In some embodiments, the capture probes are provided in a single composition. The single composition may be a solution (liquid or frozen). Alternatively, it may be a lyophilizate.

Alternatively, the capture probes may be provided as a plurality of compositions, e.g., comprising a first composition comprising probes specific for the epigenetic target region set and a second composition comprising probes specific for the sequence-variable target region set. These probes may be mixed in appropriate proportions to provide a combined probe composition with any of the foregoing fold differences in concentration and/or capture yield. Alternatively, they may be used in separate capture procedures (e.g., with aliquots of a sample or sequentially with the same sample) to provide first and second compositions comprising captured epigenetic target regions and sequence-variable target regions, respectively.

### 1. Probes Specific for Epigenetic Target Regions

The probes for the epigenetic target region set may comprise probes specific for one or more types of target regions likely to differentiate DNA from neoplastic (e.g., tumor or cancer) cells from healthy cells, e.g., non-neoplastic circulating cells. Exemplary types of such regions are discussed in detail herein, e.g., in the sections above concerning captured sets. The probes for the epigenetic target region set may also comprise probes for one or more control regions, e.g., as described herein.

In some embodiments, the probes for the epigenetic target region set have a footprint of at least 100 kbp, e.g., at least 200 kbp, at least 300 kbp, or at least 400 kbp. In some embodiments, the epigenetic target region set has a footprint in the range of 100-20 Mbp, e.g., 100-200 kbp, 200-300 kbp, 300-400 kbp, 400-500 kbp, 500-600 kbp, 600-700 kbp, 700-800 kbp, 800-900 kbp, 900-1,000 kbp, 1-1.5 Mbp, 1.5-2 Mbp, 2-3 Mbp, 3-4 Mbp, 4-5 Mbp, 5-6 Mbp, 6-7 Mbp, 7-8 Mbp, 8-9 Mbp, 9-10 Mbp, or 10-20 Mbp. In some embodiments, the epigenetic target region set has a footprint of at least 20 Mbp.

a. Hypermethylation Variable Target Regions

In some embodiments, the probes for the epigenetic target region set comprise probes specific for one or more hypermethylation variable target regions. Hypermethylation variable target regions may also be referred to herein as hypermethylated DMRs (differentially methylated regions). The hypermethylation variable target regions may be any of those set forth above. For example, in some embodiments, the probes specific for hypermethylation variable target regions comprise probes specific for a plurality of loci listed in Table 5, e.g., at least 10%, 20%, 30%, 40%, 50%, 60%, 70%, 80%, 90%, or 100% of the loci listed in Table 5. In some embodiments, the probes specific for hypermethylation variable target regions comprise probes specific for a plurality of loci listed in Table 6, e.g., at least 10%, 20%, 30%, 40%, 50%, 60%, 70%, 80%, 90%, or 100% of the loci listed in Table 6. In some embodiments, the probes specific for hypermethylation variable target regions comprise probes specific for a plurality of loci listed in Table 5 or Table 6, e.g., at least 10%, 20%, 30%, 40%, 50%, 60%, 70%, 80%, 90%, or 100% of the loci listed in Table 5 or Table 6. In some embodiments, for each locus included as a target region, there may be one or more probes with a hybridization site that binds between the transcription start site and the stop codon (the last stop codon for genes that are alternatively spliced) of the gene. In some embodiments, the one or more probes bind within 300 bp of the listed position, e.g., within 200 or 100 bp. In some embodiments, a probe has a hybridization site overlapping the position listed above. In some embodiments, the probes specific for the hypermethylation target regions include probes specific for one, two, three, four, or five subsets of hypermethylation target regions that collectively show hypermethylation in one, two, three, four, or five of breast, colon, kidney, liver, and lung cancers.

b. Hypomethylation Variable Target Regions

In some embodiments, the probes for the epigenetic target region set comprise probes specific for one or more hypomethylation variable target regions. Hypomethylation variable target regions may also be referred to herein as hypomethylated DMRs (differentially methylated regions). The hypomethylation variable target regions may be any of those set forth above. For example, the probes specific for one or more hypomethylation variable target regions may include probes for regions such as repeated elements, e.g., LINE1 elements, Alu elements, centromeric tandem repeats, pericentromeric tandem repeats, and satellite DNA, and intergenic regions that are ordinarily methylated in healthy cells may show reduced methylation in tumor cells.

In some embodiments, probes specific for hypomethylation variable target regions include probes specific for repeated elements and/or intergenic regions. In some embodiments, probes specific for repeated elements include probes specific for one, two, three, four, or five of LINE1 elements, Alu elements, centromeric tandem repeats, pericentromeric tandem repeats, and/or satellite DNA.

Exemplary probes specific for genomic regions that show cancer-associated hypomethylation include probes specific for nucleotides 8403565-8953708 and/or 151104701-151106035 of human chromosome 1. In some embodiments, the probes specific for hypomethylation variable target regions include probes specific for regions overlapping or comprising nucleotides 8403565-8953708 and/or 151104701-151106035 of human chromosome 1.

c. CTCF Binding Regions

In some embodiments, the probes for the epigenetic target region set include probes specific for CTCF binding regions. In some embodiments, the probes specific for CTCF binding regions comprise probes specific for at least 10, 20, 50, 100, 200, or 500 CTCF binding regions, or 10-20, 20-50, 50-100, 100-200, 200-500, or 500-1000 CTCF binding regions, e.g., such as CTCF binding regions described above or in one or more of CTCFBSDB or the Cuddapah et al., Martin et al., or Rhee et al. articles cited above. In some embodiments, the probes for the epigenetic target region set comprise at least 100 bp, at least 200 bp at least 300 bp, at least 400 bp, at least 500 bp, at least 750 bp, or at least 1000 bp upstream and downstream regions of the CTCF binding sites.

d. Transcription Start Sites

In some embodiments, the probes for the epigenetic target region set include probes specific for transcriptional start sites. In some embodiments, the probes specific for transcriptional start sites comprise probes specific for at least 10, 20, 50, 100, 200, or 500 transcriptional start sites, or 10-20, 20-50, 50-100, 100-200, 200-500, or 500-1000 transcriptional start sites, e.g., such as transcriptional start sites listed in DBTSS. In some embodiments, the probes for the epigenetic target region set comprise probes for sequences at least 100 bp, at least 200 bp, at least 300 bp, at least 400 bp, at least 500 bp, at least 750 bp, or at least 1000 bp upstream and downstream of the transcriptional start sites.

e. Focal Amplifications

As noted above, although focal amplifications are somatic mutations, they can be detected by sequencing based on read frequency in a manner analogous to approaches for detecting certain epigenetic changes such as changes in methylation. As such, regions that may show focal amplifications in cancer can be included in the epigenetic target region set, as discussed above. In some embodiments, the probes specific for the epigenetic target region set include probes specific for focal amplifications. In some embodiments, the probes specific for focal amplifications include probes specific for one or more of AR, BRAF, CCND1, CCND2, CCNE1, CDK4, CDK6, EGFR, ERBB2, FGFR1, FGFR2, KIT, KRAS, MET, MYC, PDGFRA, PIK3CA, and RAF1. For example, in some embodiments, the probes specific for focal amplifications include probes specific for one or more of at least 2, 3, 4, 5, 6, 7, 8, 9, 10, 11, 12, 13, 14, 15, 16, 17, or 18 of the foregoing targets.

f. Control Regions

It can be useful to include control regions to facilitate data validation. In some embodiments, the probes specific for the epigenetic target region set include probes specific for control methylated regions that are expected to be methylated in essentially all samples. In some embodiments, the probes specific for the epigenetic target region set include probes specific for control hypomethylated regions that are expected to be hypomethylated in essentially all samples.

### 2. Probes Specific for Sequence-Variable Target Regions

The probes for the sequence-variable target region set may comprise probes specific for a plurality of regions known to undergo somatic mutations in cancer. The probes may be specific for any sequence-variable target region set described herein. Exemplary sequence-variable target region sets are discussed in detail herein, e.g., in the sections above concerning captured sets.

In some embodiments, the sequence-variable target region probe set has a footprint of at least 0.5 kb, e.g., at least 1 kb, at least 2 kb, at least 5 kb, at least 10 kb, at least 20 kb, at least 30 kb, or at least 40 kb. In some embodiments, the epigenetic target region probe set has a footprint in the range of 0.5-100 kb, e.g., 0.5-2 kb, 2-10 kb, 10-20 kb, 20-30 kb, 30-40 kb, 40-50 kb, 50-60 kb, 60-70 kb, 70-80 kb, 80-90 kb, and 90-100 kb. In some embodiments, the sequence-variable target region probe set has a footprint of at least 50 kbp, e.g., at least 100 kbp, at least 200 kbp, at least 300 kbp, or at least 400 kbp. In some embodiments, the sequence-variable target region probe set has a footprint in the range of 100-2000 kbp, e.g., 100-200 kbp, 200-300 kbp, 300-400 kbp, 400-500 kbp, 500-600 kbp, 600-700 kbp, 700-800 kbp, 800-900 kbp, 900-1,000 kbp, 1-1.5 Mbp or 1.5-2 Mbp. In some embodiments, the sequence-variable target region set has a footprint of at least 2 Mbp.

In some embodiments, probes specific for the sequence-variable target region set comprise probes specific for at least a portion of at least 5, at least 10, at least 15, at least 20, at least 25, at least 30, at least 35, at least 40, at least 45, at least 50, at least 55, at least 60, at least 65, or at 70 of the genes of Table 7. In some embodiments, probes specific for the sequence-variable target region set comprise probes specific for the at least 5, at least 10, at least 15, at least 20, at least 25, at least 30, at least 35, at least 40, at least 45, at least 50, at least 55, at least 60, at least 65, or 70 of the SNVs of Table 7. In some embodiments, probes specific for the sequence-variable target region set comprise probes specific for at least 1, at least 2, at least 3, at least 4, at least 5, or 6 of the fusions of Table 7. In some embodiments, probes specific for the sequence-variable target region set comprise probes specific for at least a portion of at least 1, at least 2, or 3 of the indels of Table 7. In some embodiments, probes specific for the sequence-variable target region set comprise probes specific for at least a portion of at least 5, at least 10, at least 15, at least 20, at least 25, at least 30, at least 35, at least 40, at least 45, at least 50, at least 55, at least 60, at least 65, at least 70, or 73 of the genes of Table 8. In some embodiments, probes specific for the sequence-variable target region set comprise probes specific for at least 5, at least 10, at least 15, at least 20, at least 25, at least 30, at least 35, at least 40, at least 45, at least 50, at least 55, at least 60, at least 65, at least 70, or 73 of the SNVs of Table 8. In some embodiments, probes specific for the sequence-variable target region set comprise probes specific for at least 1, at least 2, at least 3, at least 4, at least 5, or 6 of the fusions of Table 8. In some embodiments, probes specific for the sequence-variable target region set comprise probes specific for at least a portion of at least 1, at least 2, at least 3, at least 4, at least 5, at least 6, at least 7, at least 8, at least 9, at least 10, at least 11, at least 12, at least 13, at least 14, at least 15, at least 16, at least 17, or 18 of the indels of Table 8. In some embodiments, probes specific for the sequence-variable target region set comprise probes specific for at least a portion of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 of the genes of Table 9.

In some embodiments, the probes specific for the sequence-variable target region set comprise probes specific for target regions from at least 10, 20, 30, or 35 cancer-related genes, such as AKT1, ALK, BRAF, CCND1, CDK2A, CTNNB1, EGFR, ERBB2, ESR1, FGFR1, FGFR2, FGFR3, FOXL2, GATA3, GNA11, GNAQ, GNAS, HRAS, IDH1, IDH2, KIT, KRAS, MED12, MET, MYC, NFE2L2, NRAS, PDGFRA, PIK3CA, PPP2R1A, PTEN, RET, STK11, TP53, and U2AF1.

TABLE 7

| Point Mutations (SNVs) | | | | | | Fusions |
|---|---|---|---|---|---|---|
| AKT1 | ALK | APC | AR | ARAF | ARID1A | ALK |
| ATM | BRAF | BRCA1 | BRCA2 | CCND1 | CCND2 | FGFR2 |
| CCNE1 | CDH1 | CDK4 | CDK6 | CDKN2A | CDKN2B | FGFR3 |
| CTNNB1 | EGFR | ERBB2 | ESR1 | EZH2 | FBXW7 | NTRK1 |
| FGFR1 | FGFR2 | FGFR3 | GATA3 | GNA11 | GNAQ | RET |
| GNAS | HNF1A | HRAS | IDH1 | IDH2 | JAK2 | ROS1 |
| JAK3 | KIT | KRAS | MAP2K1 | MAP2K2 | MET | |
| MLH1 | MPL | MYC | NF1 | NFE2L2 | NOTCH1 | |
| NPM1 | NRAS | NTRK1 | PDGFRA | PIK3CA | PTEN | |
| PTPN11 | RAF1 | RB1 | RET | RHEB | RHOA | |
| RIT1 | ROS1 | SMAD4 | SMO | SRC | STK11 | |
| TERT | TP53 | TSC1 | VHL | | | |

TABLE 8

| Point Mutations (SNVs) | | | | | | Fusions |
|---|---|---|---|---|---|---|
| AKT1 | ALK | APC | AR | ARAF | ARID1A | ALK |
| ATM | BRAF | BRCA1 | BRCA2 | CCND1 | CCND2 | FGFR2 |
| CCNE1 | CDH1 | CDK4 | CDK6 | CDKN2A | DDR2 | FGFR3 |
| CTNNB1 | EGFR | ERBB2 | ESR1 | EZH2 | FBXW7 | NTRK1 |
| FGFR1 | FGFR2 | FGFR3 | GATA3 | GNA11 | GNAQ | RET |
| GNAS | HNF1A | HRAS | IDH1 | IDH2 | JAK2 | ROS1 |
| JAK3 | KIT | KRAS | MAP2K1 | MAP2K2 | MET | |
| MLH1 | MPL | MYC | NF1 | NFE2L2 | NOTCH1 | |
| NPM1 | NRAS | NTRK1 | PDGFRA | PIK3CA | PTEN | |
| PTPN11 | RAF1 | RB1 | RET | RHEB | RHOA | |
| RIT1 | ROS1 | SMAD4 | SMO | MAPK1 | STK11 | |
| TERT | TP53 | TSC1 | VHL | MAPK3 | MTOR | |
| NTRK3 | | | | | | |

TABLE 9

| | | Start | Stop | Length | Exons | Critical |
|---|---|---|---|---|---|---|
| Gene | Chromosome | Position | Position | (bp) | Covered | Feature |
| ALK | chr2 | 29446405 | 29446655 | 250 | intron 19 | Fusion |
| ALK | chr2 | 29446062 | 29446197 | 135 | intron 20 | Fusion |
| ALK | chr2 | 29446198 | 29446404 | 206 | 20 | Fusion |
| ALK | chr2 | 29447353 | 29447473 | 120 | intron 19 | Fusion |
| ALK | chr2 | 29447614 | 29448316 | 702 | intron 19 | Fusion |
| ALK | chr2 | 29448317 | 29448441 | 124 | 19 | Fusion |
| ALK | chr2 | 29449366 | 29449777 | 411 | intron 18 | Fusion |
| ALK | chr2 | 29449778 | 29449950 | 172 | 18 | Fusion |
| BRAF | chr7 | 140453064 | 140453203 | 139 | 15 | BRAF V600 |
| CTNNB1 | chr3 | 41266007 | 41266254 | 247 | 3 | S37 |
| EGFR | chr7 | 55240528 | 55240827 | 299 | 18 and 19 | G719 and |
| | | | | | | deletions |
| EGFR | chr7 | 55241603 | 55241746 | 143 | 20 | Insertions/T790M |
| EGFR | chr7 | 55242404 | 55242523 | 119 | 21 | L858R |
| ERBB2 | chr17 | 37880952 | 37881174 | 222 | 20 | Insertions |
| ESR1 | chr6 | 152419857 | 152420111 | 254 | 10 | V534, P535, |
| | | | | | | L536, Y537, |
| | | | | | | D538 |
| FGFR2 | chr10 | 123279482 | 123279693 | 211 | 6 | S252 |
| GATA3 | chr10 | 8111426 | 8111571 | 145 | 5 | SS/Indels |
| GATA3 | chr10 | 8115692 | 8116002 | 310 | 6 | SS/Indels |
| GNAS | chr20 | 57484395 | 57484488 | 93 | 8 | R844 |
| IDH1 | chr2 | 209113083 | 209113394 | 311 | 4 | R132 |
| IDH2 | chr15 | 90631809 | 90631989 | 180 | 4 | R140, R172 |
| KIT | chr4 | 55524171 | 55524258 | 87 | 1 | |
| KIT | chr4 | 55561667 | 55561957 | 290 | 2 | |
| KIT | chr4 | 55564439 | 55564741 | 302 | 3 | |
| KIT | chr4 | 55565785 | 55565942 | 157 | 4 | |
| KIT | chr4 | 55569879 | 55570068 | 189 | 5 | |
| KIT | chr4 | 55573253 | 55573463 | 210 | 6 | |
| KIT | chr4 | 55575579 | 55575719 | 140 | 7 | |
| KIT | chr4 | 55589739 | 55589874 | 135 | 8 | |
| KIT | chr4 | 55592012 | 55592226 | 214 | 9 | |
| KIT | chr4 | 55593373 | 55593718 | 345 | 10 and 11 | 557, 559, |
| | | | | | | 560, 576 |
| KIT | chr4 | 55593978 | 55594297 | 319 | 12 and 13 | V654 |
| KIT | chr4 | 55595490 | 55595661 | 171 | 14 | T670, S709 |
| KIT | chr4 | 55597483 | 55597595 | 112 | 15 | D716 |
| KIT | chr4 | 55598026 | 55598174 | 148 | 16 | L783 |
| KIT | chr4 | 55599225 | 55599368 | 143 | 17 | C809, R815, |
| | | | | | | D816, L818, |
| | | | | | | D820, S821F, |
| | | | | | | N822, Y823 |
| KIT | chr4 | 55602653 | 55602785 | 132 | 18 | A829P |
| KIT | chr4 | 55602876 | 55602996 | 120 | 19 | |
| KIT | chr4 | 55603330 | 55603456 | 126 | 20 | |
| KIT | chr4 | 55604584 | 55604733 | 149 | 21 | |
| KRAS | chr12 | 25378537 | 25378717 | 180 | 4 | A146 |
| KRAS | chr12 | 25380157 | 25380356 | 199 | 3 | Q61 |
| KRAS | chr12 | 25398197 | 25398328 | 131 | 2 | G12/G13 |
| MET | chr7 | 116411535 | 116412255 | 720 | 13, 14, | MET exon |
| | | | | | intron 13, | 14 SS |
| | | | | | intron 14 | |
| NRAS | chr1 | 115256410 | 115256609 | 199 | 3 | Q61 |
| NRAS | chr1 | 115258660 | 115258791 | 131 | 2 | G12/G13 |
| PIK3CA | chr3 | 178935987 | 178936132 | 145 | 10 | E545K |
| PIK3CA | chr3 | 178951871 | 178952162 | 291 | 21 | H1047R |
| PTEN | chr10 | 89692759 | 89693018 | 259 | 5 | R130 |
| SMAD4 | chr18 | 48604616 | 48604849 | 233 | 12 | D537 |
| TERT | chr5 | 1294841 | 1295512 | 671 | promoter | chr5: 1295228 |
| TP53 | chr17 | 7573916 | 7574043 | 127 | 11 | Q331, R337, R342 |
| TP53 | chr17 | 7577008 | 7577165 | 157 | 8 | R273 |
| TP53 | chr17 | 7577488 | 7577618 | 130 | 7 | R248 |
| TP53 | chr17 | 7578127 | 7578299 | 172 | 6 | R213/Y220 |
| TP53 | chr17 | 7578360 | 7578564 | 204 | 5 | R175/Deletions |
| TP53 | chr17 | 7579301 | 7579600 | 299 | 4 | |
| | | | | 12574 (total | | |
| | | | | target region) | | |
| | | | | 16330 (total | | |
| | | | | probe coverage) | | |

### E. Amplification

In some embodiments, DNA is amplified. In some embodiments, adapted DNA is amplified. In some embodiments, multiplex amplification of segments comprising recombined CDR3 sequences from a sample or subsample using a plurality of first primers that bind V regions and a plurality of second primers that bind J regions is performed. In some embodiments, multiplex amplification of a plurality of target regions that may comprise a structural variation from a sample or subsample using a plurality of first primers and a plurality of second primers that anneal to the second plurality of target regions is performed.

Amplification is typically primed by primers binding to primer binding sites in adapters flanking a DNA molecule to be amplified. Amplification methods can involve cycles of denaturation, annealing and extension, resulting from thermocycling or can be isothermal as in transcription-mediated amplification. For example, sample nucleic acids flanked by adapters can be amplified by PCR and other amplification methods. Amplification methods of use herein can include any suitable methods, such as known to those of ordinary skill in the art. In some embodiments, amplification is primed by primers binding to primer binding sites in adapters flanking a DNA molecule to be amplified. Amplification methods can involve cycles of denaturation, annealing and extension, resulting from thermocycling, such as polymerase chain reaction (PCR), or can be isothermal, such as in linear amplification methods, transcription-mediated amplification, recombinase polymerase amplification (RPA), helices dependent amplification (HDA), loop-mediated isothermal amplification (LAMP) (Notomi et al., Nuc. Acids Res., 28, e63, 2000), rolling-circle amplification (RCA) (Blanco et al., J. Biol. Chem., 264, 8935-8940, 1989), or hyperbranched rolling circle amplification (Lizard et al., Nat. Genetics, 19, 225-232, 1998). Other amplification methods include the ligase chain reaction, strand displacement amplification, nucleic acid sequence based amplification, and self-sustained sequence based replication.

In some embodiments, the present methods perform dsDNA ligations with T-tailed and C-tailed adapters. The addition of C-tailed adapters can increase ligation efficiency because the A-tailing reaction can also add G-tails to a small portion of the DNA molecules, when the A tailing is performed in the presence of dGTP, such as when the A-tailing is performed in the same reaction as the end repair. The use of T-tailed and C-tailed adapters can result in amplification of at least 50, 60, 70 or 80% of double stranded nucleic acids before. The present methods can increase the amount or number of amplified molecules relative to control methods performed with T-tailed adapters alone by at least 10, 15 or 20%.

In some embodiments, adapted DNA is amplified before sequencing. Amplification may in some cases be before one or more capture steps. In some embodiments, the ligation step occurs after the conversion step. In some embodiments, the ligation occurs before or simultaneously with amplification.

In some embodiments, the amplification of the DNA (e.g., adapter ligated DNA) comprises using a DNA polymerase. In some embodiments, the DNA polymerase may be Q5® High-Fidelity DNA Polymerase, Q5U® Hot Start High-Fidelity DNA Polymerase, Phusion® High-Fidelity DNA Polymerase, OneTaq® DNA Polymerase, Taq DNA Polymerase, LongAmp® Taq DNA Polymerase, Hemo KlenTaq, Epimark® Hot Start Taq DNA Polymerase, Bst DNA Polymerase, Full Length, Bst DNA Polymerase, Large Fragment, Bst 2.0 DNA Polymerase, Bst 3.0 DNA Polymerase, Bsu DNA Polymerase, Large Fragment, phi29 DNA Polymerase, phi29-XT DNA Polymerase, Sulfolobus DNA Polymerase IV, Therminator™ DNA Polymerase, T7 DNA Polymerase, DNA Polymerase I (E. coli), DNA Polymerase I, Large (Klenow) Fragment (“Klenow fragment”), Klenow Fragment (3′→5′ exo-), T4 DNA Polymerase, Vent® DNA Polymerase, Vent® (exo-) DNA Polymerase, Deep Vent® DNA Polymerase, Deep Vent® (exo-) DNA Polymerase, or any combination thereof.

In some embodiments, DNA can be amplified by methylation-preserving amplification. In some embodiments, the methylation-preserving amplification can occur before the contacting the DNA in a sample with an mCpG-binding protein. For an exemplary description of mCpG binding domain proteins, see, e.g., Du et al., Methyl-CpG-binding domain proteins: readers of the epigenome. Epigenomics. 2015; 7(6):1051-73.

Amplification, including methylation-preserving amplification, is typically primed by primers binding to primer binding sites in adapters flanking a DNA molecule to be amplified. Amplification methods can involve cycles of denaturation, annealing and extension, resulting from thermocycling or can be isothermal as in transcription-mediated amplification. For example, DNA flanked by adapters added to the DNA as described herein can be amplified by PCR or other amplification methods. Amplification methods of use herein, including methylation-preserving amplification, can include any suitable methods, such as known to those of ordinary skill in the art. In some embodiments, amplification is primed by primers binding to primer binding sites in adapters flanking a DNA molecule to be amplified. Amplification methods can involve cycles of denaturation, annealing and extension, resulting from thermocycling, such as polymerase chain reaction (PCR), or can be isothermal, such as in linear amplification methods, transcription-mediated amplification, recombinase polymerase amplification (RPA), helices dependent amplification (HDA), loop-mediated isothermal amplification (LAMP) (Notomi et al., Nuc. Acids Res., 28, e63, 2000), rolling-circle amplification (RCA) (Blanco et al., J. Biol. Chem., 264, 8935-8940, 1989), or hyperbranched rolling circle amplification (Lizard et al., Nat. Genetics, 19, 225-232, 1998). Other amplification methods include the ligase chain reaction, strand displacement amplification, nucleic acid sequence-based amplification, and self-sustained sequence based replication. In some embodiments, the methylation-preserving amplification comprises linear amplification with thermocycling.

In some embodiments, methylation-preserving amplification comprises amplification performed in the presence of a methyltransferase. Methylating agents of use in methylation-preserving amplification methods described herein are known to those of ordinary skill in the art, and can include, for example, any suitable methyltransferase. In some embodiments, the methylating agent is DNMT1. DNMT1 is the most abundant DNA methyltransferase in mammalian cells and predominantly methylates hemimethylated CpG di-nucleotides in the mammalian genome. For example, DNA molecules replicated using PCR amplification with DNMT1 incubation will maintain their methylation status post-amplification, for use in further analyses, such as those described herein (such as an epigenetic base conversion step and/or an enrichment step).

Additional methylating agents useful herein include the mammalian methyltransferases, DNMT3a and DNMT3b, the plant methyltransferases, MET1, and CMT3. In some embodiments, DNMT1 or another suitable methyltransferase is used with a methyl donor and may be used with or without cofactors known to those of ordinary skill in the art. DNMT1 works in vitro at 95% efficiency without a cofactor; however, DNMT1 may be used with a cofactor such as NP95(Uhrfl), such as described in Bashtrykov P I, et al. “The UHRF1 protein stimulates the activity and specificity of the maintenance DNA methyltransferase DNMT1 by an allosteric mechanism.” J Biol Chem. 2014. In some embodiments, DNMT1 is used at a concentration of about 50-10000 U/mL, such as about 50-2000, about 50-5000, about 2500-7500, or about 5000-10000 U/mL. In some embodiments, DNMT1 is used at a concentration of about 100-500, about 500-1000, about 100-1000, about 1000-1500, about 500-1500, about 600-1400, about 700-1300, about 800-1200, about 900-1100, or about 950-1050 U/mL. In some embodiments, DNMT1 is used at a concentration of about 50, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, or about 2000 U/mL. In some embodiments, DNMT1 is used at a concentration of about 1,000 U/ml.

In some embodiments, enriching methylated DNA in a sample comprises amplification, such as embodiments comprising quantitative PCR (qPCR) or digital PCR. Some such embodiments comprising targeted detection of DNA sequences using qPCR or digital PCR do not comprise standard DNA library preparation steps, such as adapter ligation or tagging.

In some embodiments, the present methods perform dsDNA ligations with T-tailed and C-tailed adapters. The addition of C-tailed adapters can increase ligation efficiency because the A-tailing reaction can also add G-tails to a small portion of the DNA molecules, when the A tailing is performed in the presence of dGTP, such as when the A-tailing is performed in the same reaction as the end repair. The use of T-tailed and C-tailed adapters can result in amplification of at least 50, 60, 70 or 80% of double stranded nucleic acids. The present methods can increase the amount or number of amplified molecules relative to control methods performed with T-tailed adapters alone by at least 10, 15, or 20%.

In some embodiments, adapted DNA is amplified before sequencing. Amplification may in some cases be before one or more capture steps. In some embodiments, the ligation step occurs after the conversion step. In some embodiments, the ligation occurs before or simultaneously with amplification.

F. Subjecting the Sample or Treated Sample to a Procedure that Affects a First Nucleobase in the DNA Differently from a Second Nucleobase

Methods disclosed herein may comprise a step of subjecting a sample or a subsample thereof (e.g., resulting from partitioning, e.g., on the basis of methylation) to a procedure that affects a first nucleobase in the DNA differently from a second nucleobase.

In some embodiments, the first nucleobase is a modified or unmodified nucleobase, the second nucleobase is a modified or unmodified nucleobase different from the first nucleobase, and the first nucleobase and the second nucleobase have the same base pairing specificity. In some embodiments, if the first nucleobase is a modified or unmodified adenine, then the second nucleobase is a modified or unmodified adenine; if the first nucleobase is a modified or unmodified cytosine, then the second nucleobase is a modified or unmodified cytosine; if the first nucleobase is a modified or unmodified guanine, then the second nucleobase is a modified or unmodified guanine; and if the first nucleobase is a modified or unmodified thymine, then the second nucleobase is a modified or unmodified thymine (where modified and unmodified uracil are encompassed within modified thymine for the purpose of this step). Such a procedure can be used to identify nucleotides in the subsample that have or lack certain modifications, such as methylation.

In some embodiments, the first nucleobase is a modified or unmodified cytosine, then the second nucleobase is a modified or unmodified cytosine. In some embodiments, the first nucleobase is a modified cytosine, then the second nucleobase is an unmodified cytosine. In some embodiments, the first nucleobase is an unmodified cytosine, then the second nucleobase is a modified cytosine. For example, first nucleobase may comprise unmodified cytosine (C) and the second nucleobase may comprise one or more of 5-methylcytosine (mC) and 5-hydroxymethylcytosine (hmC). Alternatively, the second nucleobase may comprise C and the first nucleobase may comprise one or more of mC and hmC. Other combinations are also possible, as indicated, e.g., in the Summary above and the following discussion, such as where one of the first and second nucleobases comprises mC and the other comprises hmC.

In some embodiments, the procedure that affects a first nucleobase of the DNA differently from a second nucleobase of the DNA is a conversion. In some embodiments, the procedure that affects a first nucleobase of the DNA differently from a second nucleobase of the DNA is methylation-sensitive conversion. The methods disclosed herein can comprise contacting DNA in a sample with a deaminase, thereby providing a converted sample. In some embodiments, the deaminase is a methyl-sensitive deaminase or a methyl-insensitive deaminase. In some embodiments, the deaminase is a dsDNA deaminase and/or a ssDNA deaminase. This step of contacting the DNA in the sample with a deaminase can be referred to as, or be included in, a conversion procedure, such as any of the conversion procedures described elsewhere herein. For an exemplary description of conversion using a deaminase, see, e.g., Schutsky et al., Nature Biotechnology 2018; 36: 1083-1090. In some embodiments, the DNA in the converted sample is then sequenced, and a level or methylation at one or more differentially methylated regions of the DNA is quantified, or a variation of the copy number at one or more regions of the DNA is quantified.

Table 4 summarizes exemplary methods of deamination with the type of modified bases detectable with these methods. These are described in more detail below.

| Conversion method | Base modification detectable |
|---|---|
| Bisulfite conversion | 5mC and 5hmC distinguishable from C |
| Oxidative bisulfite conversion | 5mC distinguishable from 5hmC and C |
| Tet-assisted bisulfite (TAB) conversion | 5hmC distinguishable from 5mC and C |
| APOBEC-coupled epigenetic (ACE) | 5hmC distinguishable from 5mC and C |
| conversion | |
| Enzymatic methyl-seq (EM-seq). | 5mC and 5hmC distinguishable from C |
| Single-enzyme 5-methylctyosine | 5mC and 5hmC distinguishable from C |
| sequencing (SEM-seq) | |
| Tet-assisted pyridine borane | 5mC and 5hmC distinguishable from C |
| sequencing (TAPS) | |

As outlined below, there are various methods of detecting and/or identifying modified nucleosides that rely on a conversion procedure that changes the base-pairing specificity of a nucleoside, based on the modification status of the nucleosides. These changes of base-pairing specificity can then be detected, and thus the modification status of the nucleoside inferred, by sequencing.

In some embodiments, the conversion procedure used in the methods of the disclosure is one that changes the base pairing specificity of a modified nucleoside (e.g. methylated cytosine) but does not change the base pairing specificity of the corresponding unmodified nucleoside (e.g. cytosine) or does not change the base pairing specificity of any un-modified nucleoside (e.g. cytosine, adenosine, guanosine and thymidine (or uracil)). Advantages of methods that do not convert the base-pairing specificity of unmodified nucleosides include reduced loss of sequence complexity, higher sequencing efficiency and reduced alignment losses. Additionally, methods such as TAPS may in some cases be preferred over methods such as bisulfite sequencing and EM-seq because they are less destructive (especially important for low yield samples such as cfDNA or FFPE samples) and do not require denaturation, meaning that non-conversion errors are theoretically more likely to be random. In methods that require denaturation for conversion, failure to denature a DNA molecule will result in non-conversion of all bases in the DNA molecule. As biological changes in methylation are predominantly concerted to a localized regions of interest, these non-random (localized) non-conversion events can appear as false negatives (non-methylated regions). Random non-conversion methods can maximally affect a low percent of bases within a region, and thus the specificity of methylation change detection can be maximized (reduce false positives) by placing a threshold on percentage of bases within a region that are methylated/non-methylated. Hence, in some cases, a conversion procedure that does not involve denaturation can be preferred.

In other cases, the conversion procedure that can be used in the methods of the disclosure is one that changes the base pairing specificity of an unmodified nucleoside (e.g. cytosine) but does not change the base pairing specificity of the corresponding modified nucleoside (e.g. methylated cytosine such as 5hmC and/or 5mC). Such methods include, for example, bisulfite sequencing.

The skilled person can select a suitable method according to their needs, including which nucleoside modifications are to be detected and/or identified and which type of modified base is used in the end repair reaction.

In some embodiments, the conversion procedure converts modified nucleosides. In some embodiments, the conversion procedure which converts modified nucleosides comprises Tet-assisted conversion with a substituted borane reducing agent, optionally wherein the substituted borane reducing agent is 2-picoline borane, borane pyridine, tert-butylamine borane, ammonia borane or pyridine borane. In Tet-assisted pic-borane conversion with a substituted borane reducing agent conversion, a TET protein is used to convert 5mC and 5hmC to 5caC, without affecting unmodified C. 5caC, and 5fC if present, are then converted to dihydrouracil (DHU) by treatment with 2-picoline borane (pic-borane) or another substituted borane reducing agent such as borane pyridine, tert-butylamine borane, or ammonia borane, also without affecting unmodified C. See, e.g., Liu et al., Nature Biotechnology 2019; 37:424-429 (e.g., at Supplementary FIG. 1 and Supplementary Note 7). Thus, when this type of conversion is used, the first nucleobase comprises one or more of 5mC, 5fC, 5caC, or 5hmC, and the second nucleobase comprises unmodified cytosine. DHU is read as a T in sequencing. Sequencing of the converted DNA identifies positions that are read as cytosine as being unmodified C positions. Meanwhile, positions that are read as T are identified as being T, 5mC, 5fC, 5caC, or 5hmC. Performing TAP conversion, such as on a DNA sample as described herein, thus facilitates identifying positions containing unmodified C using the sequence reads obtained.

Hence, in these embodiments, the end repair reaction can be performed with dNTPs, wherein the at least one type of dNTP comprises a 5mC or 5hmC, and regions of the end-repaired DNA synthesized during the end repair reaction can be identified as those regions comprising 5mC or 5hmC (via T being called at positions which are C in the reference) at non-CpG positions. This procedure encompasses Tet-assisted pyridine borane sequencing (TAPS), described in further detail in Liu et al. 2019, supra. In this method Tet enzyme is used to progressively oxidize 5mC and 5hmC to 5fC or 5caC, then pyridine borane deaminates 5fC, 5CaC to DHU, amplified as T.

Alternatively, protection of 5hmC (e.g., using βGT or 5-hydroxymethylcytosine carbamoyltransferase) can be combined with Tet-assisted conversion with a substituted borane reducing agent, e.g. as described above. In this method (TAPS-β), 5hmC can be protected from conversion, for example through glucosylation using β-glucosyl transferase (βGT), forming (forming 5-glucosylhydroxymethylcytosine) 5ghmC, or through carbamoylation using 5-hydroxymethylcytosine carbamoyltransferase, forming 5cmC. This is described in Yu et al., Cell 2012; 149: 1368-80. Treatment with a TET protein such as mTetl then converts 5mC to 5caC but does not convert C, 5ghmC, or 5cmC. 5caC is then converted to DHU by treatment with pic-borane or another substituted borane reducing agent such as borane pyridine, tert-butylamine borane, or ammonia borane, also without affecting ghmC, 5cmC, or unmodified C. Thus, when Tet-assisted conversion with a substituted borane reducing agent is used, the first nucleobase comprises mC, and the second nucleobase comprises one or more of unmodified cytosine or hmC, such as unmodified cytosine and optionally hmC, fC, and/or caC. Sequencing of the converted DNA identifies positions that are read as cytosine as being either 5hmC or unmodified C positions. Meanwhile, positions that are read as T are identified as being T, 5fC, 5caC, or 5mC. Performing TAPSO conversion on a sample as described herein thus facilitates distinguishing positions containing unmodified C or 5hmC on the one hand from positions containing 5mC using the sequence reads obtained. Hence, in these embodiments, the end repair reaction can be performed with dNTPs, wherein the at least one type of dNTP comprises a 5mC, and regions synthesized during the end repair reaction can be identified as those regions comprising 5mC (via T being called at positions which are C in the reference) at non-CpG positions. For an exemplary description of this type of conversion, see, e.g., Liu et al., Nature Biotechnology 2019; 37:424-429. 5-hydroxymethylcytosine carbamoyltransferase is described in Yang et al., Bio-protocol, 2023; 12(17): e4496.

In some embodiments, the conversion procedure converts modified nucleosides. In some embodiments, the conversion procedure which converts modified nucleosides comprises chemical-assisted conversion with a substituted borane reducing agent, optionally wherein the substituted borane reducing agent is 2-picoline borane, borane pyridine, tert-butylamine borane, borane pyridine or ammonia borane. In chemical-assisted conversion with a substituted borane reducing agent, an oxidizing agent such as potassium perruthenate (KRuO₄) (also suitable for use in ox-BS conversion) is used to specifically oxidize 5hmC to 5fC. Treatment with pic-borane or another substituted borane reducing agent such as borane pyridine, tert-butylamine borane, or ammonia borane converts 5fC and 5caC to DHU but does not affect 5mC or unmodified C. Thus, when this type of conversion is used, the first nucleobase comprises one or more of hmC, fC, and caC, and the second nucleobase comprises one or more of unmodified cytosine or mC, such as unmodified cytosine and optionally mC. Sequencing of the converted DNA identifies positions that are read as cytosine as being either 5mC or unmodified C positions. Meanwhile, positions that are read as T are identified as being T, 5fC, 5caC, or 5hmC. Performing this type of conversion as described herein thus facilitates distinguishing positions containing unmodified C or 5mC on the one hand from positions containing 5hmC using the sequence reads obtained. Hence, in these embodiments, the end repair reaction can be performed with dNTPs, wherein at least one type of dNTP comprises a 5hmC, and regions synthesized during the end repair reaction can be identified as those regions comprising 5hmC (via T being called at positions which are C in the reference) at non-CpG positions. For an exemplary description of this type of conversion, see, e.g., Liu et al., Nature Biotechnology 2019; 37:424-429.

Exemplary conversion procedures that change the base-pairing specificity of modified cytosines have been described. However, the methods described herein could in principle use any modified nucleoside and suitable conversion procedure (i.e. single-base epigenetic conversion assay) that changes the base-pairing specificity of the modified nucleoside and thereby allows the modified base to be distinguished from the corresponding unmodified nucleoside and/or other types of modification when sequenced. For example, any conversion procedure could be used allowing any one of N⁶-methyladenine (6 mA), N⁶-hydroxymethyladenine (6hmA), or N⁶-formyladenine (6fA) to be distinguished from unmodified adenosine.

In some embodiments, the conversion procedure converts unmodified nucleosides. In some embodiments, the conversion procedure which converts unmodified nucleosides comprises bisulfite conversion. Treatment with bisulfite converts unmodified cytosine and certain modified cytosine nucleotides (e.g. 5-formyl cytosine (5fC) or 5-carboxylcytosine (5caC)) to uracil whereas other modified cytosines (e.g., 5mC and 5hmC) are not converted. Thus, where bisulfite conversion is used, the first nucleobase comprises one or more of unmodified cytosine, 5fC, 5caC, or other cytosine forms affected by bisulfite, and the second nucleobase may comprise one or more of 5mC and 5hmC, such as 5mC and optionally 5hmC. Sequencing of bisulfite-treated DNA identifies positions that are read as cytosine as being 5mC or 5hmC positions. Meanwhile, positions that are read as T are identified as being T or a bisulfite-susceptible form of C, such as unmodified cytosine, 5fC, or 5caC. Thus, performing bisulfite conversion, such as on a DNA sample as described herein facilitates identifying positions containing 5mC or 5hmC. Hence, in these embodiments, the end repair reaction can be performed with dNTPs, wherein at least one type of dNTP comprises a 5mC and/or a 5hmC, and regions synthesized during the end repair reaction can be identified as those regions comprising 5mC or a 5hmC (via C being called at these positions) at non-CpG positions. For an exemplary description of bisulfite conversion, see, e.g., Moss et al., Nat Commun. 2018; 9: 5068.

In some embodiments, the procedure which converts unmodified nucleosides comprises oxidative bisulfite (Ox-BS) conversion. This procedure first converts 5hmC to 5fC, which is bisulfite susceptible, followed by bisulfite conversion. Thus, when oxidative bisulfite conversion is used, the first nucleobase comprises one or more of unmodified cytosine, 5fC, 5caC, 5hmC, or other cytosine forms affected by bisulfite, and the second nucleobase comprises 5mC. Sequencing of Ox-BS converted DNA identifies positions that are read as cytosine as being 5mC positions. Meanwhile, positions that are read as T are identified as being T or a bisulfite-susceptible form of C, such as unmodified cytosine, 5fC, or 5hmC. Hence, in these embodiments, the end repair reaction can be performed with dNTPs, wherein at least one type of dNTP comprises a 5mC, and regions synthesized during the end repair reaction can be identified as those regions comprising 5mC (via C being called at these positions) at non-CpG positions. Performing Ox-BS conversion thus facilitates identifying positions containing mC. For an exemplary description of oxidative bisulfite conversion, see, e.g., Booth et al., Science 2012; 336: 934-937.

In some embodiments, the procedure which converts unmodified nucleosides comprises Tet-assisted bisulfite (TAB) conversion. In TAB conversion, 5hmC is protected from conversion and 5mC is oxidized in advance of bisulfite treatment, so that positions originally occupied by 5mC are converted to U while positions originally occupied by 5hmC remain as a protected form of cytosine. For example, as described in Yu et al., Cell 2012; 149: 1368-80, β-glucosyl transferase can be used to protect 5hmC (forming 5-glucosylhydroxymethylcytosine (5ghmC)), then a TET protein such as mTet1 can be used to convert 5mC to 5caC, and then bisulfite treatment can be used to convert C and 5caC to U while 5ghmC remains unaffected.

Alternatively, a carbamoyltransferase enzyme, such as 5-hydroxymethylcytosine carbamoyltransferase as described in Yang et al., Bio-protocol, 2023; 12(17): e4496, can be used to protect hmC (by converting hmC to 5-carbamoyloxymethylcytosine (5cmC)), then a TET protein such as mTetl can be used to convert mC to caC, and then bisulfite treatment can be used to convert C and caC to U while 5cmC remains unaffected. Thus, when TAB conversion is used, the first nucleobase comprises one or more of unmodified cytosine, 5fC, 5caC, 5mC, or other cytosine forms affected by bisulfite, and the second nucleobase comprises 5hmC. Sequencing of TAB-converted DNA identifies positions that are read as cytosine as being 5hmC positions. Meanwhile, positions that are read as T are identified as being T, or a bisulfite-susceptible form of C, such as unmodified cytosine, 5mC, 5fC, or 5caC. Performing TAB conversion on a first subsample as described herein thus facilitates identifying positions containing 5hmC. Hence, in these embodiments, the end repair reaction can be performed with dNTPs, wherein at least one type of dNTP comprises a 5hmC, and regions synthesized during the end repair reaction can be identified as those regions comprising 5hmC (via C being called at these positions) at non-CpG positions.

In some embodiments, the conversion procedure which converts unmodified cytosines comprises APOBEC-coupled epigenetic (ACE) conversion. In ACE conversion, an AID/APOBEC family DNA deaminase enzyme such as APOBEC3A (A3A) is used to deaminate an unmodified cytosine and 5mC without deaminating 5hmC, 5fC, or 5-caC. Thus, when ACE conversion is used, the first nucleobase comprises unmodified C and/or mC (e.g., unmodified C and optionally mC), and the second nucleobase comprises hmC. Sequencing of ACE-converted DNA identifies positions that are read as cytosine as being 5hmC, 5fC, or 5-caC positions. Meanwhile, positions that are read as T are identified as being T, unmodified C, or 5mC. Performing ACE conversion as described herein thus facilitates distinguishing positions containing 5hmC from positions containing 5mC or unmodified C using the sequence reads obtained from the first subsample. In some embodiments, the end repair reaction can be performed with dNTPs, wherein at least one type of dNTP comprises a 5hmC, and regions synthesized during the end repair reaction can be identified as those regions comprising 5hmC (via C being called at these positions) at non-CpG positions. For an exemplary description of ACE conversion, see, e.g., Schutsky et al., Nature Biotechnology 2018; 36: 1083-1090.

In some embodiments, the procedure that affects a first nucleobase in the DNA differently from a second nucleobase in the DNA of the first subsample comprises enzymatic conversion of the first nucleobase, e.g., as in EM-Seq. See, e.g., Vaisvila R, et al. (2019) EM-seq: Detection of DNA methylation at single base resolution from picograms of DNA. bioRxiv; DOI: 10.1101/2019.12.20.884692, available at www.biorxiv.org/content/10.1101/2019.12.20.884692v1. For example, TET2 and T4-PGT or 5-hydroxymethylcytosine carbamoyltransferase (described in Yang et al., Bio-protocol, 2023; 12(17): e4496) can be used to convert 5mC and 5hmC into substrates that cannot be deaminated by a deaminase (e.g., APOBEC3A), and then a deaminase (e.g., APOBEC3A) can be used to deaminate unmodified cytosines, converting them to uracils.

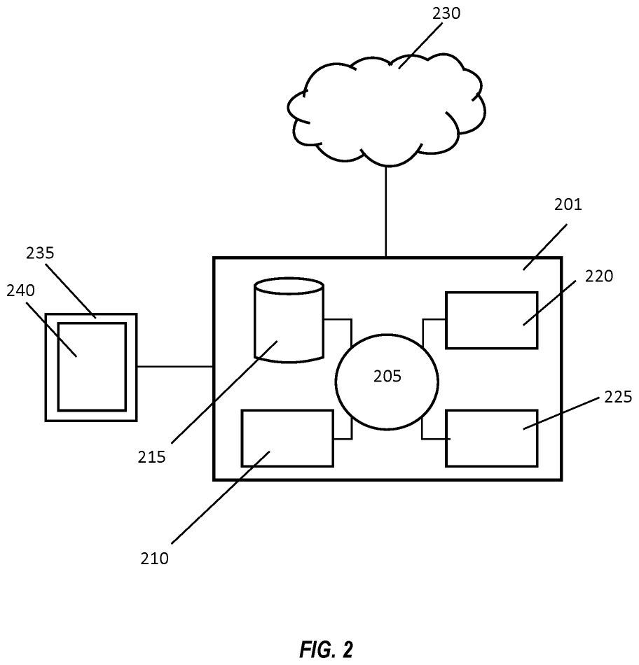

In some embodiments, the procedure that affects a first nucleobase in the DNA differently from a second nucleobase in the DNA comprises enzymatic conversion of the first nucleobase using a non-specific, modification-sensitive double-stranded DNA deaminase, e.g., as in SEM-seq. See, e.g., Vaisvila et al. (2023) Discovery of novel DNA cytosine deaminase activities enables a nondestructive single-enzyme methylation sequencing method for base resolution high-coverage methylome mapping of cell-free and ultra-low input DNA. bioRxiv; DOI: 10.1101/2023.06.29.547047, available at https://www.biorxiv.org/content/10.1101/2023.06.29.547047v1. SEM-Seq employs a non-specific, modification-sensitive double-stranded DNA deaminase (MsddA) in a nondestructive single-enzyme 5-methylctyosine sequencing (SEM-seq) method that deaminates unmodified cytosines. Accordingly, SEM-seq does not require the TET2 and T4-OGT or 5-hydroxymethylcytosine carbamoyltransferase protection and denaturing steps that are of use, e.g., in APOEC3A-based protocols. Additionally, MsddA does not deaminate 5-formylated cytosines (5fC) or 5-carboxylated cytosines (5-caC). In SEM-seq, unmodified cytosines in the DNA are deaminated to uracil and is read as “T” during sequencing. Modified cytosines (e.g., 5mC) are not converted and are read as “C” during sequencing. Cytosines that are read as thymines are identified as unmodified (e.g., unmethylated) cytosines or as thymines in the DNA. Performing SEM-seq conversion thus facilitates identifying positions containing 5mC using the sequence reads obtained. In some embodiments, the procedure that affects a first nucleobase in the DNA differently from a second nucleobase in the DNA comprises enzymatic conversion of unmodified cytosine using MsddA or a modification-sensitive DNA deaminase A (MsddA)-like deaminase. For an exemplary description of MsddA and MsddA-like deaminases, see, e.g., Vaisvila et al. Mol Cell. 2024 Mar. 7; 84(5):854-866.e7, which illustrates in FIG. 2A-C that MsddA-like deaminases have reduced activity on each of 5mC, 5hmC, and 5gmC relative to unmodified cytosine in dsDNA, e.g., a reduction of about 75%, 80%, or more on each of 5mC, 5hmC, and 5gmC relative to unmodified cytosine (e.g., using assay conditions as described in Vaisvila et al., such as analysis of deamination of C in E. coli or lambda dcm−DNA, deamination of 5mC in XP12 phage DNA, deamination of 5hmC in a C-hydroxymethylated adenovirus PCR fragment or fully C-hydroxymethylated T4147 phage DNA, and deamination of 5gmC in alpha-glucosyltransferase knockout (AGT-) T4 phage DNA. Deamination can be performed by contacting substrate DNA with deaminase and analyzed using NGS as follows: 50 ng of unmodified E. coli C2566 genomic DNA can be combined with the control DNAs (about 1 ng of Lambda, XP12, and T4147, and 0.1 ng of the 5hmC Adenovirus PCR fragment), sheared to about 300 bp and ligated to pyrrolo-dC adapters with 1 uL of in vitro synthesized deaminase (e.g., synthesized using the PURExpress In Vitro Protein Synthesis kit (NEB, Ipswich, MA) following manufacturer's recommendations with 100-400 ng of PCR fragment template DNA containing codon-optimized deaminase coding sequence and T7 promoter and terminator). Exemplary deamination reaction conditions are 50 mM Bis-Tris pH 6.0, 0.1% Triton X-100 for 1 hour at 37 degrees C. After the deamination reaction, 1 uL of Thermolabile Proteinase K (NEB, Ipswich, MA) can be added and incubated for 30 min at 37 degrees C. and then the Proteinase K can be heat inactivated at 60 degrees C. for 10 minutes. The deaminated product can then be used for library amplification using the NEBNext Q5U Master Mix (New England Biolabs, Ipswich, MA, USA) with 5 mM of NEBNext Unique Dual Index Primers. The resulting library can be purified using 1×NEBNext Sample Purification Beads according to the manufacturer's instructions and the purified library can be analyzed and quantified by an Agilent Bioanalyzer 2100 DNA Highsensitivity chip. The libraries can be sequenced using the Illumina NextSeq and NovaSeq platforms. Paired-end sequencing of 75 cycles (2×75 bp) can be performed for all the sequencing runs. Base calling and demultiplexing can be carried out with the standard Illumina pipeline.

In some embodiments, the conversion procedure converts modified nucleosides. In some embodiments, the conversion procedure which converts modified nucleosides comprises enzymatic conversion, such as DM-seq, for example, as described in WO2023/288222A1. In DM-seq, unmodified cytosines in the DNA are enzymatically protected from a subsequent deamination step wherein 5mC in 5mCpG is converted to T. The enzymatically protected unmodified (e.g., unmethylated) cytosines are not converted and are read as “C” during sequencing. Cytosines that are read as thymines (in a CpG context) are identified as methylated cytosines in the DNA. For an exemplary description of mCpG binding domain proteins, see, e.g., Du et al., Methyl-CpG-binding domain proteins: readers of the epigenome. Epigenomics. 2015; 7(6):1051-73.

Thus, when this type of conversion is used, the first nucleobase comprises unmodified (such as unmethylated) cytosine, and the second nucleobase comprises modified (such as methylated) cytosine. Sequencing of the converted DNA identifies positions that are read as cytosine as being unmodified C positions. Meanwhile, positions that are read as T are identified as being T or 5mC. Performing DM-seq conversion thus facilitates identifying positions containing 5mC using the sequence reads obtained.

Exemplary cytosine deaminases for use herein include APOBEC enzymes, for example, APOBEC3A. Generally, AID/APOBEC family DNA deaminase enzymes such as APOBEC3A (A3A) are used to deaminate (unprotected) unmodified cytosine and 5mC. For an exemplary description of APOBEC enzymes, see, e.g., Gajula et al., Nucleic Acids Res. 2014 September; 42(15):9964-75 and Schutsky et al., Nucleic Acids Res. 2017 Jul. 27; 45(13):7655-7665. For an exemplary description of APOBEC conversion, see, e.g., Schutsky et al., Nature Biotechnology 2018; 36: 1083-1090.

The enzymatic protection of unmodified cytosines in the DNA comprises addition of a protective group to the unmodified cytosines. Such protective groups can comprise an alkyl group, an alkyne group, a carboxyl group, a carboxyalkyl group, an amino group, a hydroxymethyl group, a glucosyl group, a glucosylhydroxymethyl group, an isopropyl group, or a dye. For example, DNA can be treated with a methyltransferase, such as a CpG-specific methyltransferase, which adds the protective group to unmodified cytosines. The term methyltransferase is used broadly herein to refer to enzymes capable of transferring a methyl or substituted methyl (e.g.,carboxymethyl) to a substrate (e.g., a cytosine in a nucleic acid). In some embodiments, the DNA is contacted with a CpG-specific DNA methyltransferase (MTase), such as a CpG-specific carboxymethyltransferase (CxMTase), and a substituted methyl donor, such as a carboxymethyl donor (e.g., carboxymethyl-S-adenosyl-L-methionine). See, e.g., WO2021/236778A2. In particular embodiments, the CxMTase can facilitate the addition of a protective carboxymethyl group to an unmethylated cytosine. In some embodiments, the unmethylated cytosine is unmodified cytosine. The carboxymethyl group can prevent deamination of the cytosine during a deamination step (such as a deamination step using an APOBEC enzyme, such as A3A). Substituted methyl or carboxymethyl donors useful in the disclosed methods include but are not limited to, S-adenosyl-L-methionine (SAM) analogs, optionally wherein the SAM analog is carboxy-S-adenosyl-L-methionine (CxSAM). SAM analogs are described, for example, in WO2022/197593A1. The MTase may be, for example, a CpG methyltransferase from Spiroplasma sp. strain MQ1 (M.SssI), DNA-methyltransferase 1 (DNMT1), DNA-methyltransferase 3 alpha (DNMT3A), DNA-methyltransferase 3 beta (DNMT3B), or DNA adenine methyltransferase (Dam). The CxMTase may be a CpG methyltransferase from Mycoplasma penetrans (M.MpeI). In a particular embodiment, the methyltransferase enzyme is a variant of M.MpeI having SEQ ID NO: 1 or SEQ ID NO: 2, or a sequence at least 90%, at least 92%, at least 94%, at least 96%, at least 97%, at least 98%, or at least 99% identical thereto, optionally wherein the amino acid corresponding to position 374 is R or K.

In one embodiment, the methyltransferase enzyme is a variant of M.MpeI having an N374R substitution or an N374K substitution. The methyltransferase of SEQ ID NO: 1 or SEQ ID NO: 2 can further comprise one or more amino acid substitutions selected from a) substitution of one or both residues T300 and E305 with S, A, G, Q, D, or N; b) substitution of one or more residues A323, N306, and Y299 with a positively charged amino acid selected from K, R or H; and/or c) substitution of S323 with A, G, K, R or H, which may enhance the activity of the enzyme.

Optionally, the conversion procedure further includes enzymatic protection of 5hmCs, such as by glucosylation of the 5hmCs (e.g., using βGT) or by carbamoylation of the 5hmCs (e.g., using 5-hydroxymethylcytosine carbamoyltransferase), in the DNA prior to the deamination of unprotected modified cytosines. In this method, 5hmC can be protected from conversion, for example through glucosylation using β-glucosyl transferase (βGT), forming (5-glucosylhydroxymethylcytosine) 5ghmC, or through carbamoylation using 5-hydroxymethylcytosine carbamoyltransferase, forming 5cmC. This is described, for example, in Yu et al., Cell 2012; 149: 1368-80, and in Yang et al., Bio-protocol, 2023; 12(17): e4496. Glucosylation or carbamoylation of 5hmC can reduce or eliminate deamination of 5hmC by a deaminase such as APOBEC3A. Treatment with an MTase or CxMTase then adds a protecting group to unmodified (unmethylated) cytosines in the DNA. 5mC (but not protected, unmodified cytosine and not 5ghmC or 5cmC) is then deaminated (converted to T in the case of 5mC) by treatment with a deaminase, for example, an APOBEC enzyme (such as APOBEC3A). Sequencing of the converted DNA identifies positions that are read as cytosine as being either 5hmC or unmodified C positions. Meanwhile, positions that are read as T are identified as being T or 5mC. Performing DM-seq conversion with glucosylation of 5hmC on a sample as described herein thus facilitates distinguishing positions containing unmodified C or 5hmC on the one hand from positions containing 5mC using the sequence reads obtained.

Also provided herein are methods in which alternative base conversion schemes can be used. For example, unmethylated cytosines can be left intact while methylated cytosines and hydroxymethylcytosines are converted to a base read as a thymine (e.g., uracil, thymine, or dihydrouracil).

In some embodiments, methylating a cytosine in at least one first complementary strand or second complementary strand comprises contacting the cytosine with a methyltransferase such as DNMT1 or DNMT5. In such embodiments, the step of oxidizing a 5-hydroxymethylated cytosine to 5-formylcytosine (such as by contacting the 5-hydroxymethyl cytosine in a first strand and a second strand with KRuO₄) can be optional.

In some embodiments, converting the modified cytosine in at least one first or second strand to a thymine or a base read as thymine comprises oxidizing a hydroxymethyl cytosine, e.g., the hydroxymethyl cytosine is oxidized to formylcytosine. In some embodiments, oxidizing the hydroxymethyl cytosine to formylcytosine comprises contacting the hydroxymethyl cytosine with a ruthenate, such as potassium ruthenate (KRuO₄).

In some embodiments, the modified cytosine is converted to thymine, uracil, or dihydrouracil. In any such embodiments, amplification methods may comprise uracil- and/or dihydrouracil-tolerant amplification methods, such as PCR using a uracil- and/or dihydrouracil-tolerant DNA polymerase.

In some embodiments, the method comprises converting a formylcytosine and/or a methylcytosine to carboxylcytosine as part of converting the modified cytosine in at least one first or second strand to a thymine or a base read as thymine. For example, converting the formylcytosine and/or the methylcytosine to carboxylcytosine can comprise contacting the formylcytosine and/or the methylcytosine with a TET enzyme, such as TET1, TET2, or TET3. In some embodiments, the method comprises reducing the carboxylcytosine as part of converting the modified cytosine in at least one first or second strand to a thymine or a base read as thymine, and/or the carboxylcytosine is reduced to dihydrouracil. In some embodiments, reducing the carboxylcytosine comprises contacting the carboxylcytosine with a borane or borohydride reducing agent.

In some embodiments, the borane or borohydride reducing agent comprises pyridine borane, 2-picoline borane, borane, tert-butylamine borane, ammonia borane, sodium borohydride, sodium cyanoborohydride (NaBH₃CN), lithium borohydride (LiBH₄), ethylenediamine borane, dimethylamine borane, sodium triacetoxyborohydride, morpholine borane, 4-methylmorpholine borane, trimethylamine borane, dicyclohexylamine borane, or a salt thereof. In other embodiments, the reducing agent comprises lithium aluminum hydride, sodium amalgam, amalgam, sulfur dioxide, dithionate, thiosulfate, iodide, hydrogen peroxide, hydrazine, diisobutylaluminum hydride, oxalic acid, carbon monoxide, cyanide, ascorbic acid, formic acid, dithiothreitol, beta-mercaptoethanol, or any combination thereof.

As discussed above, in some embodiments, a TET protein can be used to convert 5mC and optionally 5hmC (but not unmodified C) into substrates (e.g., 5caC) that cannot be deaminated by a deaminase, and then a deaminase (e.g., APOBEC3A) can be used to deaminate unmodified cytosines, converting them to uracils. Various TET enzymes may be used in the disclosed methods as appropriate. In some embodiments, the one or more TET enzymes comprise TETv. TETv is described in U.S. Pat. No. 10,260,088 and its sequence is SEQ ID NO: 1 therein (SEQ ID NO: 3 in the present application). In some embodiments, the one or more TET enzymes comprise TETcd. TETcd is described in U.S. Pat. No. 10,260,088 and its sequence is SEQ ID NO: 3 therein (SEQ ID NO: 4 in the present application). In some embodiments, the one or more TET enzymes comprise TET1. In some embodiments, the one or more TET enzymes comprise TET2. TET2 may be expressed and used as a fragment comprising TET2 residues 1129-1480 joined to TET2 residues 1844-1936 by a linker (SEQ ID NO: 5 of the present application) as described, e.g., in U.S. Pat. No. 10,961,525. In some embodiments, the one or more TET enzymes comprise TETl and TET2. In some embodiments, the one or more TET enzymes comprise a T1372 TET mutant, such as T1372S. In some embodiments, the one or more TET enzymes comprise a V1900 TET mutant, such as a V1900A, V1900C, V1900G, V19001, or V1900P TET mutant. In some embodiments, the one or more TET enzymes comprise a V1900 TET2 mutant, such as a V1900A, V1900C, V1900G, V19001, or V1900P TET2 mutant. Examples of V1900A, V1900C, V1900G, V19001, and V1900P TET2 mutants are provided as SEQ ID NOs: 6-10. In some embodiments, the V1900 TET mutant has at least 80%, 85%, 90%, 95%, 96%, 97%, 98%, or 99% sequence identity to SEQ ID NO: 6, 7, 8, 9, or 10. Position 1900 of the wild-type TET2 sequence corresponds to position 438 in each of SEQ ID NOs: 5-10. It can be beneficial to use a TET enzyme that maximizes formation of 5-carboxylcytosine (5-caC) relative to less oxidized modified cytosines, particularly 5-formylcytosine, because 5-caC is not a substrate for enzymatic deamination, e.g., by APOBEC enzymes such as APOBEC3A. Maximizing formation of 5-caC thus reduces the risk of false calls in which a base is identified as unmethylated because it underwent deamination even though it was methylated (or hydroxymethylated) in the original sample. Accordingly, in some embodiments, the TET enzyme comprises a mutation that increases formation of 5-caC. Exemplary mutations are set forth above. “A mutation that increases formation of 5-caC” means that the TET enzyme having the mutation produces more 5-caC than a TET enzyme that lacks the mutation but is otherwise identical. 5-caC production can be measured as described, e.g., in Liu et al., Nat Chem Biol 13:181-187 (2017) (see Online Methods section, TET reactions in vitro subsection, “driving” conditions). Any variants and/or mutants described in Liu et al. (2017) can be used in the disclosed methods as appropriate.

In some embodiments, the one or more TET enzymes comprise a TET2 enzyme comprising a T1372S mutation, such as TET2-CS-T1372S and TET2-CD-T1372S. Examples of TET2-CS-T1372S and TET2-CD-T1372S are provided as SEQ ID NOs: 11 and 12. A TET2 comprising a T1372S mutation is described in U.S. Pat. No. 10,961,525 and may be expressed and used as a fragment comprising TET2 residues 1129-1480 joined to TET2 residues 1844-1936 by a linker. Position 1372 of TET2 corresponds to position 258 of SEQ ID NO: 21 (wild type TET2 catalytic domain) of U.S. Pat. No. 10,961,525. Thus, the sequence of a T1372S TET2 catalytic domain may be obtained by changing the threonine at position 258 of SEQ ID NO: 21 of U.S. Pat. No. 10,961,525 to serine. TET2 comprising a T1372S mutation is also described in Liu et al., Nat Chem Biol. 2017 February; 13(2): 181-187. As demonstrated in Liu et al., TET2 comprising a T1372S mutation can more efficiently oxidize 5mC to produce 5-carboxylcytosine (5-caC) than other versions of TET2 such as TET2 lacking a T1372S mutation. In some embodiments, the TET2 enzyme comprises SEQ ID NO: 14 or optionally a variant of SEQ ID NO: 14 in which at least 5, 6, 7, or 8 positions match SEQ ID NO: 14 including position 5 of SEQ ID NO: 14. In some embodiments, the TET2 enzyme is a human TET2 enzyme comprising a T1372S mutation. In some embodiments, the TET2 enzyme comprises the sequence of SEQ ID NO: 11. In some embodiments, the TET2 enzyme comprises a sequence having at least 80%, 85%, 90%, 95%, 96%, 97%, 98%, or 99% identity to SEQ ID NO: 11. In some embodiments, the TET2 enzyme comprises a sequence having at least 80%, 85%, 90%, 95%, 96%, 97%, 98%, or 99% identity to SEQ ID NO: 12. In some embodiments, the TET2 enzyme comprises the sequence of SEQ ID NO: 12. The sequences of SEQ ID NOs: 11 and 12 are shown below.

In some embodiments, the deaminase is thermally inactivated after contacting DNA with the deaminase. In some embodiments, the thermal inactivation comprises heating or cooling of the deaminase to a temperature at which the deaminase has reduced or inhibited activity relative to a deaminase that has not been subjected to heating or cooling. In some embodiments, the thermal inactivation completely inhibits the activity of the deaminase or reduces the activity of the deaminase by at least about 5%, about 10%, about 15%, about 20%, about 25%, about 50%, about 75%, about 90%, about 95%, about 98%, about 99%, or 100% relative to a deaminase that has not been subjected to heating or cooling.

G. Pooling of DNA from Samples or Subsamples or Portions Thereof.

In some embodiments, the methods herein comprise preparing a pool comprising at least a portion of the DNA of a second partitioned subsample (such as a recombined CDR3 sequence partition or a structural variation target regions partition) and at least a portion of the DNA of a first partitioned subsample (such as sequence-variable target regions partition). Target regions, e.g., including intronic target regions, exonic VDJ target regions, epigenetic target regions and/or sequence-variable target regions, may be captured from a pool. The steps of capturing a target region set from at least an aliquot or portion of a sample or subsample described elsewhere herein encompass capture steps performed on a pool comprising DNA from first and second subsamples. A step of amplifying DNA in a pool may be performed before capturing target regions from the pool. The capturing step may have any of the features described for capturing steps elsewhere herein.

In some embodiments, sequence-variable target regions or epigenetic target regions can be captured from the first subsample. The first subsample may include some, a majority, substantially all, or all of the DNA of the subsample that was not included in the pool. The regions captured from the pool and from the subsample may be combined and analyzed in parallel. In some embodiments, the pool comprises a minority of the DNA of the first subsample, e.g., less than about 50% of the DNA of the recombined CDR3 sequences partition, such as less than or equal to about 45%, 40%, 35%, 30%, 25%, 20%, 15%, 10%, or 5% of the DNA of the first subsample. In some embodiments, the pool comprises about 5%-25% of the DNA of the first subsample. In some embodiments, the pool comprises about 10%-20% of the DNA of the first subsample. In some embodiments, the pool comprises about 10% of the DNA of the first subsample. In some embodiments, the pool comprises about 15% of the DNA of the first subsample. In some embodiments, the pool comprises about 20% of the DNA of the first subsample.

In some embodiments, the pool comprises a portion of the first subsample, which may be at least about 50% of the DNA of the first subsample. For example, the pool may comprise at least about 55%, 60%, 65%, 70%, 75%, 80%, 85%, 90%, or 95% of the DNA of the first subsample. In some embodiments, the pool comprises 50-55%, 55-60%, 60-65%, 65-70%, 70-75%, 75-80%, 80-85%, 85-90%, 90-95%, or 95-100% of the DNA of the first subsample. In some embodiments, the second pool comprises all or substantially all of the first subsample.

The epigenetic target regions may show differences in methylation levels and/or fragmentation patterns depending on whether they originated from a tumor or from healthy cells, or what type of tissue they originated from, as discussed elsewhere herein. The sequence-variable target regions may show differences in sequence depending on whether they originated from a tumor or from healthy cells.

Analysis of epigenetic target regions from the hypomethylated partition may be less informative in some applications than analysis of sequence-variable target regions from the hypermethylated and hypomethylated partitions and epigenetic target regions from the hypermethylated partition. As such, in methods where sequence-variable target regions and epigenetic target regions are being captured, the latter may be captured to a lesser extent than one or more of the sequence-variable target regions from the hypermethylated and hypomethylated partitions and epigenetic target regions from the hypermethylated partition. For example, sequence-variable target regions can be captured from the portion of the hypomethylated partition not pooled with the hypermethylated partition, and the pool can be prepared with some (e.g., a majority, substantially all, or all) of the DNA from the hypermethylated partition and none or some (e.g., a minority) of the DNA from the hypomethylated partition. Such approaches can reduce or eliminate sequencing of epigenetic target regions from the hypomethylated partition, thereby reducing the amount of sequencing data that suffices for further analysis.

In some embodiments, including a minority of the DNA of the first subsample in the pool facilitates quantification of one or more epigenetic features (e.g., methylation or other epigenetic feature(s) discussed in detail elsewhere herein), e.g., on a relative basis.

In some embodiments, recombined CDR3 sequences or structural variation target regions can be captured from the second subsample. In some embodiments, the second subsample may include some, a majority, substantially all, or all of the DNA of the subsample that was not included in the pool. The regions captured from the pool and from the subsample may be combined and analyzed in parallel. In some embodiments, the pool comprises a minority of the DNA of the second subsample, e.g., less than about 50% of the DNA of the recombined CDR3 sequences partition, such as less than or equal to about 45%, 40%, 35%, 30%, 25%, 20%, 15%, 10%, or 5% of the DNA of the second subsample. In some embodiments, the pool comprises about 5%-25% of the DNA of the second subsample. In some embodiments, the pool comprises about 10%-20% of the DNA of the second subsample. In some embodiments, the pool comprises about 10% of the DNA of the second subsample. In some embodiments, the pool comprises about 15% of the DNA of the second subsample. In some embodiments, the pool comprises about 20% of the DNA of the second subsample.

In some embodiments, the pool comprises a portion of the second subsample, which may be at least about 50% of the DNA of the second subsample. For example, the pool may comprise at least about 55%, 60%, 65%, 70%, 75%, 80%, 85%, 90%, or 95% of the DNA of the second subsample. In some embodiments, the pool comprises 50-55%, 55-60%, 60-65%, 65-70%, 70-75%, 75-80%, 80-85%, 85-90%, 90-95%, or 95-100% of the DNA of the second subsample. In some embodiments, the second pool comprises all or substantially all of the second subsample.

### H. Sequencing

In general, sample nucleic acids being analyzed according to the disclosed methods, such as captured regions and CDR3-enriched DNA and/or structural variation-enriched DNA. In general, sample nucleic acids, including nucleic acids flanked by adapters, with or without prior amplification can be subject to sequencing. Sequencing methods include, for example, Sanger sequencing, high-throughput sequencing, pyrosequencing, sequencing-by-synthesis, long-read sequencing (also known as single-molecule sequencing or third generation sequencing), nanopore sequencing (a type of long-read sequencing), 5-letter sequencing or 6-letter sequencing, semiconductor sequencing, sequencing-by-ligation, sequencing-by-hybridization, Digital Gene Expression (Helicos), Next generation sequencing (NGS), Single Molecule Sequencing by Synthesis (SMSS) (Helicos), massively-parallel sequencing, Clonal Single Molecule Array (Solexa), shotgun sequencing, Ion Torrent, Oxford Nanopore, Roche Genia, Maxim-Gilbert sequencing, primer walking, and sequencing using PacBio, SOLiD, Ion Torrent, or Nanopore platforms. Sequencing reactions can be performed in a variety of sample processing units, which may multiple lanes, multiple channels, multiple wells, or other means of processing multiple sample sets substantially simultaneously. Sample processing unit can also include multiple sample chambers to enable processing of multiple runs simultaneously.

In some embodiments, sequencing comprises detecting and/or distinguishing unmodified and modified nucleobases. For example, long-read sequencing (also referred to herein as third generation sequencing) methods include those that can generate longer sequencing reads, such as reads in excess of 10 kilobases, as compared to short-read sequencing methods, which generally produce reads of up to about 600 bases in length. Compared to short reads, long reads can improve de novo assembly, transcript isoform identification, and detection and/or mapping of structural variants. Furthermore, long-read sequencing of native DNA or RNA molecules reduces amplification bias and preserves base modifications, such as methylation status. Long-read sequencing technologies useful herein can include any suitable long-read sequencing methods, including, but not limited to, Pacific Biosciences (PacBio) single-molecule real-time (SMRT) sequencing, Oxford Nanopore Technologies (ONT) nanopore sequencing, and synthetic long-read sequencing approaches, such as linked reads, proximity ligation strategies, and optical mapping. Synthetic long-read approaches comprise assembly of short reads from the same DNA molecule to generate synthetic long reads, and may be used in conjunction with “true” long-read sequencing technologies, such as SMRT and nanopore sequencing methods.

Single-molecule real-time (SMRT) sequencing can facilitate direct detection of, e.g., 5-methylcytosine and 5-hydroxymethylcytosine as well as unmodified cytosine. (Weirather J L, et al., “Comprehensive comparison of Pacific Biosciences and Oxford Nanopore Technologies and their applications to transcriptome analysis,” F1000Research, 6:100, 2017). Whereas next-generation sequencing methods detect augmented signals from a clonal population of amplified DNA fragments, SMRT sequencing captures a single DNA molecule, maintaining base modification during sequencing. The error rate of raw PacBio SMRT sequencing-generated data is about 13-15%, as the signal-to-noise ratio from single DNA molecules not high. To increase accuracy, this platform uses a circular DNA template by ligating hairpin adapters to both ends of target double-stranded DNA. As the polymerase repeatedly traverses and replicates the circular molecule, the DNA template is sequenced multiple times to generate a continuous long read (CLR). The CLR can be split into multiple reads (“subreads”) by removing adapter sequences, and multiple subreads generate circular consensus sequence (“CCS”) reads with higher accuracy. The average length of a CLR is >10 kb and up to 60 kb, with length depending on the polymerase lifetime. Thus, the length and accuracy of CCS reads depends on the fragment sizes. PacBio sequencing has been utilized for genome (e.g., de novo assembly, detection of structural variants and haplotyping) and transcriptome (e.g., gene isoform reconstruction and novel gene/isoform discovery) studies.

SMRT sequencing relies on sequencing-by-synthesis, where the sequence of a circular DNA template is determined from the succession of fluorescence pulses, each resulting from the addition of one labelled nucleotide by a polymerase fixed to the bottom of a well. Base modifications do not affect the base-called sequence, but they affect the kinetics of the polymerase. By considering the inter-pulse duration (IPD), base modifications can be inferred from the comparison of a modified template to an in silico model or an unmodified template. Such methods can therefore use the pulse width of a signal from sequencing bases, the interpulse duration (IPD) of bases, and the identity of the bases in order to detect a modification in a base or in a neighboring base. (See e.g., Weirather et al., F1000Research, 6:100, 2017.) SMRT sequencing can thus be used to detect base modifications such as 5-caC, 4mC, 5mC, 5hmC, 6 mA, and 8oxoG (Gouil & Keniry Essays in Biochemistry (2019) 63 639-648). Accordingly, in some embodiments, the sequencing comprises SMRT sequencing.

Some sequencing reactions involve use of an enzyme to control passage of a nucleic acid through a nanopore, and in such cases reaction data can include both kinetics and other behavior of the enzyme and fluctuations in current through the nanopore. For example, ratchet proteins, helicases, or motor proteins can be used to push or pull a nucleic acid molecule through a hole in a biological or synthetic membrane. The kinetics of these proteins can vary depending on the sequence context of a nucleic acid on which they are acting. For example, they may slow down or pause at a modified base, and this behavior, captured as a part of the reaction data, is indicative of the presence of the modified base even where the modified base is not within the sensing portion of the nanopore.

One example of a nanopore-based single molecule sequencing system is that commercialized by Oxford Nanopore Technologies (ONT). (Weirather J L, et al., F1000Research, 6:100, 2017). ONT directly sequences a native single-stranded DNA (ssDNA) molecule by measuring characteristic current changes as the bases are threaded through the nanopore by a molecular motor protein. ONT uses a hairpin library structure similar to the PacBio circular DNA template: the DNA template and its complement are bound by a hairpin adapter. Therefore, the DNA template passes through the nanopore, followed by a hairpin and finally the complement. The raw read can be split into two “lD” reads (“template” and “complement”) by removing the adapter. The consensus sequence of two “1D” reads is a “2D” read with a higher accuracy.

Nanopore sequencing can be used to detect base modifications including 5-caC, 5mC, 5hmC, 6 mA, BrdU, FldU, IdU, and EdU (see e.g., Gouil & Keniry Essays in Biochemistry (2019) 63 639-648; Kutyavin, Biochemistry (2008), 47, 51, 13666-1367; Müller et al., Nature Methods (2019), volume 16, pages 429-436; Hennion et al., Genome Biology (2020), volume 21, Article number: 125). Accordingly, in some embodiments, the sequencing comprises nanopore sequencing.

5-letter and 6-letter sequencing methods include whole genome sequencing methods capable of sequencing A, C, T, and G in addition to 5mC and 5hmC to provide a 5-letter (A, C, T, G, and either 5mC or 5hmC) or 6-letter (A, C, T, G, 5mC, and 5hmC) digital readout in a single workflow. The processing of the DNA sample is entirely enzymatic and avoids the DNA degradation and genome coverage biases of bisulfite treatment. In an exemplary 5-letter sequencing method developed by Cambridge Epigenetix, the sample DNA is first fragmented via sonication and then ligated to short, synthetic DNA hairpin adapters at both ends (Füllgrabe, et al. 2022, bioRxiv doi: https://doi.org/10.1101/2022.07.08.499285). The construct is then split to separate the sense and antisense sample strands. For each original sample strand a complementary copy strand is synthesized by DNA polymerase extension of the 3′-end to generate a hairpin construct with the original sample DNA strand connected to its complementary strand, lacking epigenetic modifications, via a synthetic loop. Sequencing adapters are then ligated to the end. Modified cytosines are enzymatically protected. The unprotected Cs are then deaminated to uracil, which is subsequently read as thymine. In any such embodiments, amplification methods may comprise uracil- and/or dihydrouracil-tolerant amplification methods, such as PCR using a uracil- and/or dihydrouracil-tolerant DNA polymerase (i.e., a DNA polymerase that can read and amplify templates comprising uracil and/or dihydrouracil bases). The deaminated constructs are no longer fully complementary and have substantially reduced duplex stability, thus the hairpins can be readily opened and amplified by PCR. The constructs can be sequenced in paired-end format whereby read 1 (P1 primed) is the original stand and read 2 (P2 primed) is the copy stand. The read data is pairwise aligned so read 1 is aligned to its complementary read 2. Cognate residues from both reads are computationally resolved to produce a single genetic or epigenetic letter. Pairings of cognate bases that differ from the permissible five are the result of incomplete fidelity at some stage(s) comprising sample preparation, amplification, or erroneous base calling during sequencing. As these errors occur independently to cognate bases on each strand, substitutions result in a non-permissible pair. Non-permissible pairs are masked (marked as N) within the resolved read and the read itself is retained, leading to minimal information loss and high accuracy at read-level. The resolved read is aligned to the reference genome. Genetic variants and methylation counts are produced by read-counting at base-level.

5hmC has been shown to have value as a marker of biological states and disease which includes early cancer detection from cell-free DNA. In adapting 5-letter to 6-letter sequencing, 5mC is disambiguated from 5hmC without compromising genetic base calling within the same sample fragment. The first three steps of the workflow are identical to 5-letter sequencing described above, to generate the adapter ligated sample fragment with the synthetic copy strand. Methylation at 5mC is enzymatically copied across the CpG unit to the C on the copy strand, whilst 5hmC is enzymatically protected from such a copy. Thus, unmodified C, 5mC and 5hmC in each of the original CpG units are distinguished by unique 2-base combinations. The unmodified cytosines are then deaminated to uracil, which is subsequently read as thymine. The DNA is subjected to PCR amplification and sequencing as described earlier. The reads are pairwise aligned and resolved using a 2-base code. Each of unmodified C, 5mC, and 5hmC can be resolved as the three CpG units are distinct sequencing environments of the 2-base code.

The sequencing reactions can be performed on one or more forms of nucleic acids, such as those known to contain markers of cancer or of other disease. The sequencing reactions can also be performed on any nucleic acid fragments present in the sample.

In some embodiments, sequence coverage of the genome may be, for example, less than 5%, 10%, 15%, 20%, 25%, 30%, 40%, 50%, 60%, 70%, 80%, 90%, 95%, 99%, 99.9% or 100%. In some embodiments, the sequence reactions may provide for sequence coverage of at least 5%, 10%, 15%, 20%, 25%, 30%, 40%, 50%, 60%, 70%, or 80% of the genome. Sequence coverage can be performed on at least 5, 10, 20, 70, 100, 200 or 500 different genes, or up to, for example, 5000, 2500, 1000, 500 or 100 different genes.

Simultaneous sequencing reactions may be performed using multiplex sequencing. In some cases, cell-free nucleic acids may be sequenced with at least, for example, 1000, 2000, 3000, 4000, 5000, 6000, 7000, 8000, 9000, 10000, 50000, 100,000 sequencing reactions. In other embodiments, cell-free nucleic acids may be sequenced with less than, for example, 1000, 2000, 3000, 4000, 5000, 6000, 7000, 8000, 9000, 10000, 50000, 100,000 sequencing reactions. Sequencing reactions may be performed sequentially or simultaneously. Subsequent data analysis may be performed on all or part of the sequencing reactions. In some cases, data analysis may be performed on at least, for example, 1000, 2000, 3000, 4000, 5000, 6000, 7000, 8000, 9000, 10000, 50000, 100,000 sequencing reactions. In other cases, data analysis may be performed on less than, for example, 1000, 2000, 3000, 4000, 5000, 6000, 7000, 8000, 9000, 10000, 50000, 100,000 sequencing reactions. An exemplary read depth is 1000-50000, 1000-10000, or 1000-20000 reads per locus (base).

### 1. Differential Depth of Sequencing

In some embodiments, where nucleic acids corresponding to a sequence-variable target region set and nucleic acids corresponding to an epigenetic target region set are sequenced, the nucleic acids corresponding to the sequence-variable target region set are sequenced to a greater depth of sequencing than nucleic acids corresponding to the epigenetic target region set. In some embodiments, nucleic acids corresponding to the hydroxymethylation-variable target region set are sequenced to a greater depth of sequencing than nucleic acids corresponding to at least one other target region set. For example, the depth of sequencing for nucleic acids corresponding to the sequence-variable and/or hydroxymethylation-variable target region sets may be at least 1.25-, 1.5-, 1.75-, 2-, 2.25-, 2.5-, 2.75-, 3-, 3.5-, 4-, 4.5-, 5-, 6-, 7-, 8-, 9-, 10-, 11-, 12-, 13-, 14-, or 15-fold greater, or 1.25- to 1.5-, 1.5- to 1.75-, 1.75- to 2-, 2- to 2.25-, 2.25- to 2.5-, 2.5- to 2.75-, 2.75- to 3-, 3- to 3.5-, 3.5- to 4-, 4- to 4.5-, 4.5- to 5-, 5- to 5.5-, 5.5- to 6-, 6- to 7-, 7- to 8-, 8- to 9-, 9- to 10-, 10- to 11-, 11- to 12-, 13- to 14-, 14- to 15-fold, or 15- to 100-fold greater, than the depth of sequencing for nucleic acids corresponding to the epigenetic target region set or to at least one other target region set. In some embodiments, said depth of sequencing is at least 2-fold greater. In some embodiments, said depth of sequencing is at least 5-fold greater. In some embodiments, said depth of sequencing is at least 10-fold greater. In some embodiments, said depth of sequencing is 4- to 10-fold greater. In some embodiments, said depth of sequencing is 4- to 100-fold greater. Each of these embodiments refer to the extent to which nucleic acids corresponding to the sequence-variable target region set are sequenced to a greater depth of sequencing than nucleic acids corresponding to the epigenetic target region set.

In some embodiments, the captured cfDNA corresponding to the sequence-variable target region set and the captured cfDNA corresponding to the epigenetic target region set are sequenced concurrently, e.g., in the same sequencing cell (such as the flow cell of an Illumina sequencer) and/or in the same composition, which may be a pooled composition resulting from recombining separately captured sets or a composition obtained by capturing the cfDNA corresponding to the sequence-variable target region set and the captured cfDNA corresponding to the epigenetic target region set in the same vessel.

In some embodiments, the captured cfDNA corresponding to the hydroxymethylation variable target region set and the captured cfDNA corresponding to the at least one other target region set are sequenced concurrently, e.g., in the same sequencing cell (such as the flow cell of an Illumina sequencer) and/or in the same composition, which may be a pooled composition resulting from recombining separately captured sets or a composition obtained by capturing the cfDNA corresponding to the hydroxymethylation variable target region set and the captured cfDNA corresponding to the at least one other target region set in the same vessel.

### I. Samples and Subjects

The disclosure relates to methods of analyzing the modified nucleoside profile of DNA in a sample, e.g., an amplified, adapted library. In some cases, the DNA sample used in a method disclosed herein is obtained or has been obtained from a subject. In some embodiments, the DNA sample may comprise or consist of DNA from a biological sample obtained from a subject. The subject may be a human, a mammal, an animal, a primate, rodent (including mice and rats), or other common laboratory, domestic, companion, service or agricultural animal, for example a rabbit, dog, cat, horse, cow, sheep, goat or pig. Preferably, the DNA sample is from a human. The subject may in some cases have or be suspected of having a cancer, tumor or neoplasm. In other cases, the subject may not have cancer or a detectable cancer symptom. The subject may have been treated with one or more cancer therapy, e.g., any one or more of chemotherapies, antibodies, vaccines or biologics. The subject may be in remission, e.g. from a tumor, cancer, or neoplasia (e.g., following treatment such as chemotherapy, surgical resection, radiation, or a combination thereof). The subject may or may not be diagnosed as being susceptible to cancer or any cancer-associated genetic mutations/disorders. In some embodiments, the sample is a DNA sample obtained from a tumor tissue biopsy. The cancer, tumor, or neoplasm may generally be of any type, for example a cancer tumor or neoplasm of the lung, colon, rectum (or colorectum), kidney, breast, prostate, or liver, or other type of cancer as described herein. In some embodiments, the sample is obtained from a subject in remission from a tumor, cancer, or neoplasia (e.g., following chemotherapy, surgical resection, radiation, or a combination thereof). In any of the foregoing embodiments, the precancer, cancer, tumor, or neoplasia or suspected precancer, cancer, tumor, or neoplasia may be of the bladder, head and neck, lung, colon, rectum, kidney, breast, prostate, skin, or liver. In some embodiments, the precancer, cancer, tumor, or neoplasia or suspected precancer, cancer, tumor, or neoplasia is of the lung. In some embodiments, the precancer, cancer, tumor, or neoplasia or suspected precancer, cancer, tumor, or neoplasia is of the colon or rectum. In some embodiments, the precancer, cancer, tumor, or neoplasia or suspected precancer, cancer, tumor, or neoplasia is of the breast. In some embodiments, the precancer, cancer, tumor, or neoplasia or suspected precancer, cancer, tumor, or neoplasia is of the prostate. In any of the foregoing embodiments, the subject may be a human subject. In some embodiments, the sample is obtained from a subject having a stage I cancer, stage II cancer, stage III cancer or stage IV cancer.

The biological sample can be any biological sample isolated from a subject. Biological samples can include body tissues, such as known or suspected solid tumors (such as carcinomas, adenocarcinomas, or carcinomas), whole blood, platelets, serum, plasma, stool, red blood cells, white blood cells or leucocytes, endothelial cells, tissue biopsies, cerebrospinal fluid synovial fluid, lymphatic fluid, ascites fluid, interstitial or extracellular fluid, the fluid in spaces between cells, including gingival crevicular fluid, bone marrow, pleural effusions, cerebrospinal fluid, saliva, mucous, sputum, semen, sweat, and urine. In some embodiments, biological samples are preferably body fluids, particularly blood and fractions thereof (e.g., plasma and/or serum) or urine. A sample can be in the form originally isolated from a subject or can have been subjected to further processing to remove or add components, such as cells, or enrich for one component relative to another.

In some embodiments, a population of nucleic acids is obtained from a serum, plasma or blood sample from a subject suspected of having neoplasia, a tumor, precancer, or cancer or previously diagnosed with neoplasia, a tumor, precancer, or cancer. The population includes nucleic acids having varying levels of sequence variation, epigenetic variation, and/or post-replication or transcriptional modifications. Post-replication modifications include modifications of cytosine, particularly at the 5-position of the nucleobase, e.g., 5-methylcytosine, 5-hydroxymethylcytosine, 5-formylcytosine and 5-carboxylcytosine.

A sample can be isolated or obtained from a subject and transported to a site of sample analysis. The sample may be preserved and shipped at a desirable temperature, e.g., room temperature, 4° C., −20° C., and/or −80° C.

In a particular embodiment, the DNA sample comprises cell-free DNA. In another particular embodiment the DNA sample is a DNA sample from a formalin fixed paraffin embedded (FFPE) sample.

A sample can be isolated or obtained from a subject at the site of the sample analysis. The subject can be a human, a mammal, an animal, a companion animal, a service animal, or a pet. In some embodiments, the subject may have a cancer, precancer, infection, transplant rejection, or other disease or disorder related to changes in the immune system. The subject may not have cancer or a detectable cancer symptom. The subject may have been treated with one or more cancer therapy, e.g., any one or more of chemotherapies, antibodies, vaccines or biologics. The subject may be in remission. The subject may or may not be diagnosed as being susceptible to cancer or any cancer-associated genetic mutations/disorders.

In some embodiments, the sample comprises plasma. The volume of plasma used to obtain the DNA sample can depend on the desired read depth for sequenced regions. Exemplary volumes are 0.4-40 ml, 5-20 ml, 10-20 ml. For example, the volume can be 0.5 mL, 1 mL, 2 mL, 3 mL, 4 mL, 5 mL, 6 mL, 7 mL, 8 mL, 9 mL, 10 mL, 20 mL, 30 mL, or 40 mL. A volume of sampled plasma may be 5 to 20 mL. In some embodiments, the sample volume is 3-5 mL of plasma, such as 4 mL of plasma, per 10 mL whole blood.

In some embodiments, the sample comprises whole blood. Exemplary volumes of sampled whole blood are 0.4-40 mL, 5-20 mL, 10-20 mL, 1-6 mL, 1-3 mL, and 3-5 mL. For example, the volume can be 0.5 mL, 1 mL, 2 mL, 3 mL, 4 mL, 5 mL, 6 mL, 7 mL, 8 mL, 9 mL, 10 mL, 20 mL, 30 mL, or 40 mL. A volume of sampled whole blood may be 5 to 20 mL. In some embodiments, the sample volume is 1-5 mL of whole blood, such as 2.5 mL of whole blood.

In some embodiments, the sample comprises buffy coat separated from whole blood. Exemplary volumes of sampled buffy coat are 0.1-20 mL, 1-10 mL, 1-5 mL, 0.2-0.6 mL, and 0.3-0.5 mL. For example, the volume can be 0.1 mL, 0.2 mL, 0.3 mL, 0.4 mL, 0.5 mL, 0.6 mL, 0.7 mL, 0.8 mL, 0.9 mL, 1 mL, 2 mL, 3 mL, 4 mL, 5 mL 10 mL, or 20 mL. A volume of sampled buffy coat may be 1 to 10 mL. In some embodiments, the sample volume is 0.1-0.5 mL of buffy coat, such as 0.3 mL of buffy coat, per 10 mL whole blood.

In some embodiments, the sample comprises PBMCs separated from whole blood. Exemplary volumes of sampled PBMCs are 0.1-20 mL, 1-10 mL, 1-5 mL, 0.2-0.6 mL, and 0.3-0.5 mL. For example, the volume can be 0.1 mL, 0.2 mL, 0.3 mL, 0.4 mL, 0.5 mL, 0.6 mL, 0.7 mL, 0.8 mL, 0.9 mL, 1 mL, 2 mL, 3 mL, 4 mL, 5 mL 10 mL, or 20 mL. A volume of sampled PBMCs may be 1 to 10 mL. In some embodiments, the sample volume is 0.1-0.5 mL of PBMCs, such as 0.3 mL of PBMCs, per 10 mL whole blood.

In some embodiments, the sample comprises leukocytes separated from subject blood using leukapheresis. Exemplary volumes of sampled leukocytes from leukapheresis are 0.1-20 mL, 1-10 mL, 1-5 mL, 0.2-0.6 mL, and 0.3-0.5 mL. For example, the volume can be 0.1 mL, 0.2 mL, 0.3 mL, 0.4 mL, 0.5 mL, 0.6 mL, 0.7 mL, 0.8 mL, 0.9 mL, 1 mL, 2 mL, 3 mL, 4 mL, 5 mL, 10 mL, or 20 mL. A volume of sampled leukocytes from leukapheresis may be I to 10 mL. In some embodiments, the sample volume is 0.1-0.6 mL of leukocytes from leukapheresis, such as 0.4 mL of leukocytes, per 10 mL whole blood.

A sample can comprise various amounts of nucleic acid that contain genome equivalents. For example, a sample of about 30 ng DNA can contain about 10,000 (104) haploid human genome equivalents and, in the case of cell free DNA (cfDNA), about 200 billion (2×10¹¹) individual polynucleotide molecules. Similarly, a sample of about 100 ng of DNA can contain about 30,000 haploid human genome equivalents and, in the case of cfDNA, about 600 billion individual molecules.

A sample can comprise nucleic acids from different sources, e.g., nucleic acids and nucleic acids from cells and cell-free nucleic acids of the same subject, from cells and cell-free of different subjects. In some embodiments, the nucleic acid may be DNA. A sample can comprise nucleic acids (e.g., DNA) carrying mutations. For example, a sample can comprise DNA carrying germline mutations and/or somatic mutations. Germline mutations refer to mutations existing in germline DNA of a subject. Somatic mutations refer to mutations originating in somatic cells of a subject, e.g., cancer cells. A sample can comprise DNA carrying cancer-associated mutations (e.g., cancer-associated somatic mutations). A sample can comprise an epigenetic variant (i.e., a chemical or protein modification), wherein the epigenetic variant associated with the presence of a genetic variant such as a cancer-associated mutation. In some embodiments, the sample comprises an epigenetic variant associated with the presence of a genetic variant, wherein the sample does not comprise the genetic variant.

The DNA sample may be or comprise cell free nucleic acids or cfDNA. The cfDNA may be obtained from a test subject, for example as described above. For example, the sample for analysis may be plasma or serum containing cell-free nucleic acids. “Cell-free DNA” “cfDNA molecules,” or “cfDNA”, for example, include DNA molecules that naturally occur in a subject in extracellular form (e.g., in blood, serum, plasma, or other bodily fluids such as lymph, cerebrospinal fluid, urine, or sputum). While the cfDNA originally existed in a cell or cells in a large complex biological organism, e.g., a mammal, it has undergone release from the cell(s) in vivo into a fluid found in the organism, and may be obtained by obtaining a sample of the fluid without the need to perform an in vitro cell lysis step. In other words, cell-free nucleic acids or cfDNA are nucleic acids or DNA not contained within or otherwise bound to a cell, or the nucleic acids or DNA remaining in a sample after removing intact cells. Cell-free nucleic acids include DNA, RNA, and hybrids thereof, including genomic DNA, mitochondrial DNA, siRNA, miRNA, circulating RNA (cRNA), tRNA, rRNA, small nucleolar RNA (snoRNA), Piwi-interacting RNA (piRNA), long non-coding RNA (long ncRNA), or fragments of any of these. Cell-free nucleic acids can be double-stranded, single-stranded, or a hybrid thereof. A cell-free nucleic acid can be released into bodily fluid through secretion or cell death processes, e.g., cellular necrosis and apoptosis. Some cell-free nucleic acids are released into bodily fluid from cancer cells e.g., circulating tumor DNA, (ctDNA). Others are released from healthy cells. In some embodiments, cfDNA is cell-free fetal DNA (cffDNA). In some embodiments, cell free nucleic acids are produced by tumor cells. In some embodiments, cell free nucleic acids are produced by a mixture of tumor cells and non-tumor cells.

Exemplary amounts of cell-free nucleic acids in a sample before amplification range from about 1 fg to about 1 μg, e.g., 1 pg to 200 ng, 1 ng to 100 ng, 10 ng to 1000 ng. For example, the amount can be up to about 600 ng, up to about 500 ng, up to about 400 ng, up to about 300 ng, up to about 200 ng, up to about 100 ng, up to about 50 ng, or up to about 20 ng of cell-free nucleic acid molecules. The amount can be at least 1 fg, at least 10 fg, at least 100 fg, at least 1 pg, at least 10 pg, at least 100 pg, at least 1 ng, at least 10 ng, at least 100 ng, at least 150 ng, or at least 200 ng of cell-free nucleic acid molecules. The amount can be up to 1 femtogram (fg), 10 fg, 100 fg, 1 picogram (pg), 10 pg, 100 pg, 1 ng, 10 ng, 100 ng, 150 ng, or 200 ng of cell-free nucleic acid molecules. The method can comprise obtaining 1 femtogram (fg) to 200 ng of cell-free nucleic acid molecules from samples.

Cell-free DNA refers to DNA not contained within a cell at the time of its isolation from a subject. For example, cfDNA can be isolated from a sample as the DNA remaining in the sample after removing intact cells, without lysing the cells or otherwise extracting intracellular DNA. Cell-free nucleic acids include DNA, RNA, and hybrids thereof, including genomic DNA, mitochondrial DNA, siRNA, miRNA, circulating RNA (cRNA), tRNA, rRNA, small nucleolar RNA (snoRNA), Piwi-interacting RNA (piRNA), long non-coding RNA (long ncRNA), or fragments of any of these. Cell-free nucleic acids can be double-stranded, single-stranded, or a hybrid thereof. A cell-free nucleic acid can be released into bodily fluid through secretion or cell death processes, e.g., cellular necrosis and apoptosis. Some cell-free nucleic acids are released into bodily fluid from cancer cells e.g., circulating tumor DNA, (ctDNA). Others are released from healthy cells. In some embodiments, cfDNA is cell-free fetal DNA (cffDNA) In some embodiments, cell free nucleic acids are produced by tumor cells. In some embodiments, cell free nucleic acids are produced by a mixture of tumor cells and non-tumor cells.

Cell-free nucleic acids have an exemplary size distribution of about 100-500 nucleotides, with molecules of 110 to about 230 nucleotides representing about 90% of molecules, with a mode of about 168 nucleotides and a second minor peak in a range between 240 to 440 nucleotides.

Cell-free nucleic acids can be isolated from bodily fluids through a fractionation or partitioning step in which cell-free nucleic acids, as found in solution, are separated from intact cells and other non-soluble components of the bodily fluid. In some embodiments, a blood sample is fractionated prior to capturing at least an epigenetic target region set of DNA. Partitioning may include techniques such as centrifugation or filtration. Alternatively, cells in bodily fluids can be lysed and cell-free and cellular nucleic acids processed together. Generally, after addition of buffers and wash steps, nucleic acids can be precipitated with an alcohol. Further clean up steps may be used such as silica-based columns to remove contaminants or salts. Non-specific bulk carrier nucleic acids, DNA or protein for bisulfite sequencing, hybridization, and/or ligation, may be added throughout the reaction to optimize certain aspects of the procedure such as yield.

After such processing, samples can include various forms of nucleic acid including double stranded DNA, single stranded DNA and single stranded RNA. In some embodiments, single stranded DNA and RNA can be converted to double stranded forms so they are included in subsequent processing and analysis steps.

Double-stranded DNA molecules in a sample and single stranded nucleic acid molecules converted to double stranded DNA molecules can be linked to adapters at either one end or both ends. Typically, double stranded molecules are blunt ended by treatment with a polymerase with a 5′-3′ polymerase and a 3 ′-5′ exonuclease (or proof reading function), in the presence of all four standard nucleotides. Klenow large fragment and T4 polymerase are examples of suitable polymerase. The blunt ended DNA molecules can be ligated with at least partially double stranded adapter (e.g., a Y shaped or bell-shaped adapter). Alternatively, complementary nucleotides can be added to blunt ends of sample nucleic acids and adapters to facilitate ligation. Contemplated herein are both blunt end ligation and sticky end ligation. In blunt end ligation, both the nucleic acid molecules and the adapter tags have blunt ends. In sticky-end ligation, typically, the nucleic acid molecules bear an “A” overhang and the adapters bear a “T” overhang.

A sample can be, or can be prepared from, any biological sample isolated from a subject. In some embodiments, the amplified, adapted library is or has been prepared from a sample, which may be any of the samples described herein. A sample can be a bodily sample. Samples can include body tissues or fluids, such as known or suspected solid tumors, whole blood, platelets, serum, plasma, stool, red blood cells, white blood cells or leucocytes, endothelial cells, tissue biopsies, cerebrospinal fluid synovial fluid, lymphatic fluid, ascites fluid, interstitial or extracellular fluid, the fluid in spaces between cells, gingival crevicular fluid, bone marrow, pleural effusions, pleura fluid, cerebrospinal fluid, saliva, mucous, sputum, semen, sweat, and urine. Samples are preferably body fluids, particularly blood and fractions thereof, cerebrospinal fluid, pleura fluid, saliva, sputum, or urine. A sample can be in the form originally isolated from a subject or can have been subjected to further processing to remove or add components, such as cells, or enrich for one component relative to another. Thus, a preferred body fluid for analysis is plasma or serum containing cell-free nucleic acids.

In some embodiments, a population of nucleic acids is obtained from a serum, plasma or blood sample from a subject suspected of having neoplasia, a tumor, precancer, or cancer or previously diagnosed with neoplasia, a tumor, precancer, or cancer. The population includes nucleic acids having varying levels of sequence variation, epigenetic variation, and/or post-replication or transcriptional modifications. Post-replication modifications include modifications of cytosine, particularly at the 5-position of the nucleobase, e.g., 5-methylcytosine, 5-hydroxymethylcytosine, 5-formylcytosine and 5-carboxylcytosine.

A sample can be isolated or obtained from a subject and transported to a site of sample analysis. The sample may be preserved and shipped at a desirable temperature, e.g., room temperature, 4° C., −20° C., and/or −80° C. A sample can be isolated or obtained from a subject at the site of the sample analysis. The subject can be a human, a mammal, an animal, a companion animal, a service animal, or a pet. The subject may have a cancer, precancer, infection, transplant rejection, or other disease or disorder related to changes in the immune system. The subject may not have cancer or a detectable cancer symptom. The subject may have been treated with one or more cancer therapy, e.g., any one or more of chemotherapies, antibodies, vaccines or biologics. The subject may be in remission. The subject may be in remission. The subject may or may not be diagnosed of being susceptible to cancer or any cancer-associated genetic mutations/disorders.

The methods disclosed herein are also particularly suited for the analysis of DNA from formalin-fixed paraffin-embedded (FFPE) tissue samples. While the formalin fixation process adequately preserves the ultrastructure of the tissues, it results in various types of damage to the DNA within the tissues, such as nicks in the DNA. As explained elsewhere herein, these nicks can lead to synthesis of regions of the DNA molecule in the end repair process. The methods disclosed herein allow for these regions to be identified and the sequence data to be interpreted accordingly.

Reference or control molecules can be added to or spiked into a sample as a control or normalization standard. For example, a certain amount of modified DNA from a species other than the species of the subject from which the sample was obtained or synthetic nucleic acids comprising certain modifications may be added to the sample. In some embodiments, the reference or control molecules are distinguishable from the molecules originally present in the sample. In some embodiments, the detected DNA sequences are normalized to the reference or control molecules.

In some embodiments, the sample comprises plasma. The volume of plasma obtained can depend on the desired read depth for sequenced regions. Exemplary volumes are 0.4-40 ml, 5-20 ml, 10-20 ml. For examples, the volume can be 0.5 mL, 1 mL, 5 mL 10 mL, 20 mL, 30 mL, or 40 mL. A volume of sampled plasma may be 5 to 20 mL.

### J. End Repair and A-Tailing

In some embodiments, the disclosed methods comprise subjecting the DNA from a sample to end repair to generate end-repaired DNA molecules, e.g., prior to preparing the amplified, adapted library.

In some embodiments, the end repair is performed using deoxynucleotide triphosphates (dNTPs). In some embodiments, at least one type of dNTP comprises a modified base, and the at least one dNTP comprising a modified base is incorporated into repaired regions of the end-repaired DNA molecules at one or more locations.

End repair refers to methods for repairing DNA by the conversion of non-blunt ended DNA into blunt ended DNA. Sequencing workflows typically use end repair to make ends of DNA molecules compatible with adapters, which are subsequently ligated onto the DNA. Fragmented and/or damaged DNA (e.g. cfDNA or DNA from FFPE samples) often contain non-blunt ends, which contain 3′overhangs and/or 5′overhangs. A 3′overhang refers to the 3′ end of a DNA strand which extends beyond the 5′end of the paired strand, resulting in one or more unpaired nucleotides at the 3′end of the DNA strand. Conversely, a 5′overhang refers to the 5′ end of a DNA strand which extends beyond the 3′end of the paired strand, resulting in one or more unpaired nucleotides at the 5′end of the DNA strand.

The process of end repair involves the conversion of double-stranded DNA with 3′overhangs and/or 5′overhangs to double-stranded DNA without overhangs. This can be done using one or more enzymes such as T4 DNA polymerase and/or Klenow fragment. The 3′ to 5′ exonuclease activity of these enzymes removes the 3′ends at 3′overhangs and the 5′ to 3′ polymerase activity of these enzymes extends the 3′ ends at 5′ overhangs to remove the 5′ overhang, thereby generating a blunt-ended DNA molecule. In order to fill in these 5′ overhangs, end repair is conducted in the presence of dATP, dCTP, dGTP and dTTP. End repair can also include a second step, which involves the addition of a phosphate group to the 5′ ends of DNA, by an enzyme such as polynucleotide kinase. This makes the 5′ends of the end-repaired DNA molecules compatible with the subsequent action of DNA polymerases and DNA ligases.

As used herein, the term “A-tailing” refers to the addition of a single deoxyadenosine residue to the end of a blunt-ended double-stranded DNA fragment to form a 3′ deoxyadenosine single-base overhang. Such A tailing reactions are conducted with polymerases which have the ability to add a non-templated A to the 3′ end of a blunt, double-stranded DNA molecule. Polymerases capable of A-tailing typically do not possess 3′-5′ exonuclease activity. When A-tailing is performed as a separate reaction to end repair, it is typically conducted in the presence of dATP, but the absence of dCTP, dTTP and dGTP. A-tailed fragments are not compatible for self-ligation (i.e., self-circularizatian and concantenation of the DNA), but they are compatible with 3′ T-overhangs, which can be used on adapters. Methods comprising end repair, A-tailing and ligation to adapters with 3′ T-overhangs can result in higher efficiency ligation, compared to blunt ended ligation, as blunt ligation can lead to self-ligation of the adapters and/or DNA molecules.

In some embodiments, the methods disclosed herein comprise end repair of the DNA molecules followed by blunt end ligation of adapters. In other embodiments, the methods disclosed herein comprise end repair of the DNA molecules followed by A-tailing and sticky-end ligation of T-tailed adapters. When the methods disclosed herein comprise an A-tailing step, it may be performed separately from the end repair with an intervening reaction clean-up step or it may be performed in the same reaction as the end repair (e.g. using NEBNext® Ultra™ II End Repair/dA-Tailing Module (E7546)). In some embodiments, the reaction clean-up step removes unincorporated dNTPs. In instances wherein the A-tailing reaction is performed in the same reaction as end repair, the sticky-end ligation may be performed with a mixture of T-tailed adapters and C-tailed adapters.

End repair and A tailing reactions can have varying impacts on the composition of the DNA molecule, dependent on the exact workflow and reaction components used. These reactions can lead to the synthesis of regions at the 3′ends of DNA strands, but also the synthesis of internal regions through nick translation and through gap filling followed by ligation.

In some embodiments, end repair can lead to 3′fill in with unmethylated cytosines, which may not reflect the true methylation status of that position in the DNA molecule prior to the generation of the 5′overhang. In nicked DNA, polymerases which contain 5′ to 3′ exonuclease activity and/or strand displacement activity can lead to the synthesis of internal regions of the DNA molecule through nick translation. If the end repair reaction is conducted with non-methylated deoxycytidine triphosphate (dCTP), the synthesized regions will incorporate the non-methylated dCTP, potentially at positions which initially comprised methylated cytosines. In gapped DNA, both the DNA polymerases used in end repair and A tailing can lead to the generation of synthesized regions. The gaps can be filled in with DNA polymerases used in the end repair reaction, regardless of whether they possess 5′ to 3′ exonuclease activity or strand displacement activity. After this gap filling, a nick will still exist between the synthesized region and the region of the original DNA molecule 3′ of the gap. The A-tailing enzymes may then introduce further synthesized regions through nick translation, as described for the nicked DNA. This synthesized region may extend to the 3′end of the DNA molecule.

In some embodiments, the end-repair and the A-tailing reactions are performed in a single tube. In such cases, the A tailing reaction can be performed at a higher temperature than the end repair. Optionally, end repair is performed at ambient temperature (e.g. 15-35° C.) and A tailing is performed at a temperature over 60° C., including e.g., about 60° C.-75° C. The A tailing reaction can be performed using a thermostable polymerase (e.g. Taq DNA polymerase, Tfl DNA polymerase, Bst DNA Polymerase, Large Fragment or Tth DNA polymerase) and the method further comprises increasing temperature of the sample after the end repair to inactivate the polymerase used in end repair (e.g. T4 DNA polymerase or Klenow fragment). In some embodiments the A-tailing is performed using a DNA polymerase that: (i) does not possess 5′-3′ exonuclease activity; and/or (ii) is not a strand displacing DNA polymerase. These properties reduce the ability of the DNA polymerase to extend from nick. This reduces the level of synthesis which may occur during the end repair and A-tailing reactions thus reducing the proportion of sequencing data that may be filtered out as potentially containing artifactual data. Accordingly, in some embodiments, the A-tailing is performed using a DNA polymerase that cannot extend from a nick in the DNA such as HemoKlen Taq. In other embodiments, the A-tailing is performed using Taq DNA polymerase. In other embodiments, the A-tailing is performed using Tfl polymerase, Bst DNA Polymerase, Large Fragment or Tth polymerase.

In order to reduce the level of synthesized regions, the end repair reaction can be performed using DNA polymerases can be used which lack 5′ to 3′ exonuclease activity and/or strand displacement activity (e.g. T4 DNA polymerase or Klenow fragment).

In nicked DNA and gapped DNA, nick translation is reduced in end repair through the use of polymerases which lack 5′ to 3′ exonuclease activity and/or strand displacement activity. The separation of the end repair and A tailing reaction by a reaction clean-up means that only dATP (not dCTP, dTTP or dGTP) is present in the A tailing reaction. This means that efficient nick translation cannot occur in the A tailing reaction because the three of the four nucleotide components are not present in the reaction mixture. In gapped DNA, the gaps can be filled in with DNA polymerases used in the end repair reaction, regardless of whether they possess 5′ to 3′ exonuclease activity or strand displacement activity. These filled gaps thereby generate synthesized regions.

In some embodiments, the end-repair is performed with a polymerase which lacks 5′ to 3′ exonuclease activity and/or strand displacement activity. In some cases, the polymerase used in the end repair reaction may be Q5® High-Fidelity DNA Polymerase, Q5U® Hot Start High-Fidelity DNA Polymerase, Phusion® High-Fidelity DNA Polymerase, Hemo KlenTaq, phi29 DNA Polymerase, T7 DNA Polymerase, DNA Polymerase I (E. coli), DNA Polymerase I, Large (Klenow) Fragment (“Klenow fragment”) or T4 DNA Polymerase. In some embodiments, the polymerase used in the end repair is T4 DNA Polymerase or Klenow fragment. In some embodiments, the end repair is performed with a DNA polymerase which has 5′-3′ exonuclease activity and/or is a strand displacing DNA polymerase.

In some embodiments, the methods disclosed herein comprise an A tailing reaction after the end repair and before the ligation reaction, wherein the end repair and A tailing reactions are separated by a reaction cleanup. The A tailing reaction is typically performed in the presence of dATP, but in the absence of dCTP, dTTP and dGTP. In some embodiments, the A tailing reaction is performed using Klenow Fragment lacking 3′-5′ exonuclease activity.

In some embodiments, a dNTP that comprises a modified base is used in end repair, which may be any modified base wherein the presence or the absence of the modification can be detected by a type of sequencing. In some embodiments, a dNTP comprising a modified base can be used in a combined end repair and A-tailing reaction. In some embodiments, the modified base is incorporated in the synthesized regions at both CpG sites and CpH sites (i.e. CpA, CpC and CpT sites). While methylation of cytosines in non-CpG contexts has been described, it is thought to comprise 0.02% of total methyl-cytosine in differentiated somatic cells (Jang et al. Genes (Basel). 2017 June; 8(6): 148). The presence of methylated cytosine in non-CpG contexts in the end-repaired DNA can therefore be interpreted as being introduced during the end repair and/or A-tailing reactions. Using a dNTP comprising a modified base can therefore be used to effectively label the synthesized regions of the end repaired DNA.

In some embodiments, a dNTP that comprises a modified base may comprise any modified base wherein the presence or the absence of the modification can be detected by a type of sequencing. The modified base may be 5-caryboxylcytosine (5-caC), 4-methylcytosine (4mC), 5-methylcytosine (5mC), 5-hydroxymethyl-cytosine (5hmC), N6-methyladenosine (6 mA), bromodeoxyuridine (BrdU), 5-fluorodeoxyuridine (FldU), 5-iododeoxyuridine (IdU), 5-ethynyldeoxyuridine (EdU) and/or 8-oxoguanine (8oxoG).

When a dNTP comprising a modified base is used, it may be used in place of the equivalent unmodified base in the end repair reaction. For instance, if a dCTP comprising 5mC is used in the end repair reaction, there may be no dCTP comprising an unmodified cytosine. This would ensure that dCTPs incorporated into the DNA molecule during the end repair reaction contain 5mC. In some embodiments, multiple types of dNTP comprising a modified base are used in the end repair. For example, dATP comprising 6 mA and dCTP comprising 5mC can be used in the end repair reaction in place of dATP comprising unmodified adenine and dCTP comprising unmodified cytosine. The use of multiple types of dNTP comprising a modified base is advantageous because it provides increased resolution in defining the regions of the end-repaired DNA molecule which have been synthesized during the end repair reaction. This is because, in this example, the end of a synthesized region can be defined as the first unmodified adenine or unmodified cytosine after a stretch of containing 6mAs and/or 5mCs, rather than relying on the detection of solely an unmodified adenine or solely an unmodified cytosine.

The sequencing method used will depend on the type of modified base used in the end-repair reaction such that the specific modification can be detected. Exemplary conversion-based methods are described above alongside the base modification which they can detect. Moreover, nanopore-based sequencing can be used to detect 5-caC, 4mC, 5mC, 5hmC, 6 mA, BrdU, FldU, IdU, and EdU, and single-molecule real time (SMRT) sequencing from Pacific Biosciences can be used to detect 5-caC, 4mC, 5mC, 5hmC, 6 mA, and 8oxoG.

The disclosed methods use at least one type of dNTP which comprises a modified base (e.g. a methylated deoxycytidine triphosphate, such as deoxycytidine triphosphate comprising 5-methylcytosine (5mC) and/or 5-hydroxymethyl-cytosine (5hmC)) in the end repair reaction. In particular embodiments, the dNTP is 5mC. During end repair reactions, the methylated deoxycytidine triphosphate will be incorporated into the synthesized regions regardless of the sequence context. This will result in methylated cytosines in non-CpG positions (i.e., methylated cytosines in a CpH context), which are very rare in nature. These methylated non-CpG cytosines can therefore be used as labels for identifying synthesized regions in the end repaired DNA molecule. Similarly, other types of dNTP which comprise a modified base uncommon or absent in nature can be used. The identification of such modified bases can be performed using sequencing, and regions comprising these modifications can be interpreted as defining regions which were synthesized in the end repair reaction.

In other embodiments of the disclosed methods, the modified base is a methylated cytosine, such as 5mC or 5hmC. In some embodiments, the methylated cytosine is 5mCpH (where H=A, T, or C). In other embodiments, the modified base is other than 5mC or 5hmC. In other embodiments, the modified base is other than 5mC or 5hmC and the at least one type of dNTP comprising a modified base is incorporated into a repaired region of the end-repaired DNA molecules at one or more locations. In some embodiments wherein the modified base is other than 5mC or 5hmC, a repaired region is defined as (i) the sequence between two non-modified bases spanning a modified base, wherein the bases are of the same identity to the modified bases present in the at least one type of dNTP; and/or (ii) the sequence between a non-modified base and the end of a sequence read, wherein there is no additional non-modified bases between the non-modified base and the end of the sequence read, where the non-modified bases are the same identity as the modified base present in the at least one type of dNTP. In some embodiments wherein the modified base is a methylated cytosine, such as 5mC or 5hmC, a repaired region is defined as (i) the sequence between two non-methylated cytosines which span one or more methylated CpH cytosines; and/or (ii) the sequence between a methylated CpH cytosine and an end of a sequence read, wherein the methylated CpH cytosine is the CpH cytosine most distant from the end of the sequence read, or a subsequence thereof comprising one or more methylated CpH cytosines.

### K. Analysis

Analyzing DNA may comprise detecting or quantifying DNA of interest. Analyzing DNA can comprise detecting genetic variants and/or epigenetic features (e.g., DNA methylation and/or DNA fragmentation). In some embodiments, the DNA of interest is one or more differentially methylated regions of the DNA. In some embodiments, the detecting or quantifying the DNA of interest comprises quantifying and/or detecting a level of methylation at one or more differentially methylated regions of the DNA. In some embodiments, quantifying and/or detecting the level of methylation at one or more differentially methylated regions of the DNA comprises sequencing at least a portion of the amplified DNA or quantitative PCR (qPCR). In some embodiments, the DNA of interest is a differentially methylated region. In some embodiments, the detecting or quantifying the DNA of interest comprises quantifying and/or detecting a level of a differentially methylated region of the DNA. In some embodiments, quantifying and/or detecting the level of a differentially methylated region of the DNA comprises quantitative PCR (qPCR).

The present methods can be used to diagnose presence of conditions, particularly cancer or precancer, in a subject, to characterize conditions (e.g., staging cancer or determining heterogeneity of a cancer), monitor response to treatment of a condition, effect prognosis risk of developing a condition or subsequent course of a condition. The present disclosure can also be useful in determining the efficacy of a particular treatment option. Successful treatment options may increase the amount of copy number variation, rare mutations, or target proteins detected in a subject's blood if the treatment is successful as more cancers may die and shed DNA and proteins, among other things. In other examples, this may not occur. In another example, perhaps certain treatment options may be correlated with genetic profiles of cancers over time. This correlation may be useful in selecting a therapy.

Additionally, if a cancer is observed to be in remission after treatment, the present methods can be used to monitor residual disease or recurrence of disease.

The types and number of cancers that may be detected may include blood cancers, brain cancers, lung cancers, skin cancers, nose cancers, throat cancers, liver cancers, bone cancers, lymphomas, pancreatic cancers, skin cancers, bowel cancers, rectal cancers, colon cancers, prostate cancers, thyroid cancers, bladder cancers, head and neck cancers, kidney cancers, mouth cancers, stomach cancers, solid state tumors, heterogeneous tumors, homogenous tumors and the like. Type and/or stage of cancer can be detected from genetic variations including mutations, rare mutations, indels, copy number variations, transversions, translocations, recombination, inversion, deletions, aneuploidy, partial aneuploidy, polyploidy, chromosomal instability, chromosomal structure alterations, gene fusions, chromosome fusions, gene truncations, gene amplification, gene duplications, chromosomal lesions, DNA lesions, abnormal changes in nucleic acid chemical modifications, abnormal changes in epigenetic patterns, and abnormal changes in nucleic acid 5-methylcytosine.

In some embodiments, a method described herein comprises identifying the presence of nucleic acids, such as DNA produced by a tumor (or neoplastic cells, or cancer cells) or by precancer cells.

Genetic data can be used for characterizing a specific form of cancer. Cancers are often heterogeneous in both composition and staging. Genetic profile data may allow characterization of specific sub-types of cancer that may be useful in the diagnosis or treatment of that specific sub-type. This information may also provide a subject or practitioner clues regarding the prognosis of a specific type of cancer and allow either a subject or practitioner to adapt treatment options in accord with the progress of the disease. Some cancers progress, becoming more aggressive and genetically unstable. Other cancers may remain benign, inactive or dormant. The system and methods of this disclosure may be useful in determining disease progression.

The present methods are useful in determining the efficacy of a particular treatment option. The present methods can also be used for detecting epigenetic variations in conditions other than cancer. Further, the methods of the disclosure may be used to characterize the heterogeneity of an abnormal condition in a subject, the method comprising generating a genetic profile of extracellular polynucleotides in the subject, wherein the genetic profile comprises a plurality of data resulting from epigenetic information (such as methylation profiling), and optionally copy number variation and rare mutation analyses. In some cases, including but not limited to cancer, a disease may be heterogeneous. Disease cells may not be identical. In the example of cancer, some tumors are known to comprise different types of tumor cells, some cells in different stages of the cancer. In other examples, heterogeneity may comprise multiple foci of disease. Again, in the example of cancer, there may be multiple tumor foci, perhaps where one or more foci are the result of metastases that have spread from a primary site.

The present methods can thus be used to generate or profile, fingerprint or set of data that is a summation of epigenetic, and optionally genetic, information derived from different cells in a heterogeneous disease. This set of data may comprise epigenetic information, copy number variation, and/or rare mutation analyses alone or in combination.

The present disclosure provides methods of analyzing DNA. In some embodiments, the disclosed methods comprise analyzing DNA (such as DNA from a subject) to identify at least one cell type, cell cluster type, tissue type, and/or cancer type from which one or more type-specific epigenetic target regions and/or type-specific sequence-variable target regions originated. In some embodiments, methods comprise determining the level of one or more type-specific epigenetic target regions and/or type-specific sequence-variable target regions that originated from the at least one cell type, cell cluster type, tissue type, and/or cancer type.

In some embodiments, detecting the presence, levels, or absence of DNA sequences and/or modifications facilitates disease diagnosis or identification of appropriate treatments. In some embodiments, the presence of or a change in the levels of one or more sequences and/or modifications is indicative of the presence or absence of a disease or disorder in a subject, such as cancer or precancer, or other disorder that causes changes in nucleic acids relative to a healthy subject.

Information and data generated by the methods disclosed herein can also be used for characterizing a specific form of cancer. The methods disclosed herein may allow characterization of specific sub-types of cancer that may be important in the diagnosis or treatment of that specific sub-type. This information may also provide a subject or practitioner clues regarding the prognosis of a specific type of cancer and allow either a subject or practitioner to adapt treatment options in accord with the progress of the disease. Some cancers can progress to become more aggressive and genetically unstable. Other cancers may remain benign, inactive or dormant. The system and methods of this disclosure may be useful in determining disease progression.

Further, the methods of the disclosure may be used to characterize the heterogeneity of a condition in a subject. Such methods can include, e.g., generating an aggregate profile of extracellular nucleic acids derived from the subject, wherein the aggregate profile comprises a plurality of data resulting from various nucleic acid analyses. In some embodiments, the aggregate profile comprises epigenetic and mutation analyses.

In some embodiments, an aggregate profile comprises a summation of information derived from different cells in a heterogeneous disease. This summation may comprise structural variation identities and levels, copy number variation, epigenetic variation, or other mutation analyses.

The present methods can be used to diagnose, prognose, monitor or observe cancers, or other diseases. In some embodiments, the methods herein do not involve the diagnosing, prognosing or monitoring a fetus and as such are not directed to non-invasive prenatal testing. In other embodiments, these methodologies may be employed in a pregnant subject to diagnose, prognose, monitor or observe cancers or other diseases in an unborn subject whose DNA and other polynucleotides may co-circulate with maternal molecules.

### L. Computer Systems

Methods of the present disclosure can be implemented using, or with the aid of, computer systems. FIG. 2 shows a computer system 201 that is programmed or otherwise configured to implement the methods of the present disclosure. The computer system 201 can regulate various aspects sample preparation, sequencing, and/or analysis. In some examples, the computer system 201 is configured to perform sample preparation and sample analysis, including (where applicable) nucleic acid sequencing, e.g., according to any of the methods disclosed herein.

The computer system 201 includes a central processing unit (CPU, also “processor” and “computer processor” herein) 205, which can be a single core or multi core processor, or a plurality of processors for parallel processing. The computer system 201 also includes memory or memory location 210 (e.g., random-access memory, read-only memory, flash memory), electronic storage unit 215 (e.g., hard disk), communication interface 220 (e.g., network adapter) for communicating with one or more other systems, and peripheral devices 225, such as cache, other memory, data storage, and/or electronic display adapters. The memory 210, storage unit 215, interface 220, and peripheral devices 225 are in communication with the CPU 205 through a communication network or bus (solid lines), such as a motherboard. The storage unit 215 can be a data storage unit (or data repository) for storing data. The computer system 201 can be operatively coupled to a computer network 230 with the aid of the communication interface 220. The computer network 230 can be the Internet, an internet and/or extranet, or an intranet and/or extranet that is in communication with the Internet. The computer network 230 in some cases is a telecommunication and/or data network. The computer network 230 can include one or more computer servers, which can enable distributed computing, such as cloud computing. The computer network 230, in some cases with the aid of the computer system 201, can implement a peer-to-peer network, which may enable devices coupled to the computer system 201 to behave as a client or a server.

The CPU 205 can execute a sequence of machine-readable instructions, which can be embodied in a program or software. The instructions may be stored in a memory location, such as the memory 210. Examples of operations performed by the CPU 205 can include fetch, decode, execute, and writeback.

The storage unit 215 can store files, such as drivers, libraries, and saved programs. The storage unit 215 can store programs generated by users and recorded sessions, as well as output(s) associated with the programs. The storage unit 215 can store user data, e.g., user preferences and user programs. The computer system 201 in some cases can include one or more additional data storage units that are external to the computer system 201, such as located on a remote server that is in communication with the computer system 201 through an intranet or the Internet. Data may be transferred from one location to another using, for example, a communication network or physical data transfer (e.g., using a hard drive, thumb drive, or other data storage mechanism).

The computer system 201 can communicate with one or more remote computer systems through the network 230. For embodiment, the computer system 201 can communicate with a remote computer system of a user (e.g., operator). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 201 via the network 230.

Methods as described herein can be implemented by way of machine (e.g., computer processor) executable code stored on an electronic storage location of the computer system 201, such as, for example, on the memory 210 or electronic storage unit 215. The machine executable or machine-readable code can be provided in the form of software. During use, the code can be executed by the processor 205. In some cases, the code can be retrieved from the storage unit 215 and stored on the memory 210 for ready access by the processor 205. In some situations, the electronic storage unit 215 can be precluded, and machine-executable instructions are stored on memory 210.

In an aspect, the present disclosure provides a non-transitory computer-readable medium comprising computer-executable instructions which, when executed by at least one electronic processor, perform at least a portion of a method described herein. For example, the method may comprise: collecting a sample from a subject and, optionally, fractionating the sample into subsamples; pre-enrichment of post-translationally modified proteins comprising contacting the sample or a subsample thereof with a first lectin that specifically binds a first saccharide present in a post-translational modification (PTM) on one or more target proteins in the sample, thereby producing first complexes comprising the first lectin and a target protein; and separating the first complexes from other components of the sample or subsample thereof, thereby obtaining a first pre-enriched subsample; determining the presence or level of at least one of the post-translationally modified target proteins comprising contacting the first pre-enriched subsample with a plurality of binding molecules comprising a first binding molecule that specifically binds a first epitope of a first target protein and a second binding molecule that specifically binds a second epitope of the first target protein, wherein the first and second binding molecules each comprise a label; and detecting the labels of the first and second binding molecules.

The code can be pre-compiled and configured for use with a machine have a processer adapted to execute the code or can be compiled during runtime. The code can be supplied in a programming language that can be selected to enable the code to execute in a pre-compiled or as-compiled fashion.

Aspects of the systems and methods provided herein, such as the computer system 201, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming.

All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as those used across physical interfaces between local devices, through wired and optical landline networks, and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Hence, a machine-readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards, paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 201 can include or be in communication with an electronic display 235 that comprises a user interface (UI) 240 for providing, for example, one or more results of sample analysis. Examples of UIs include, without limitation, a graphical user interface (GUI) and web-based user interface.

Additional details relating to computer systems and networks, databases, and computer program products are also provided in, for example, Peterson, Computer Networks: A Systems Approach, Morgan Kaufmann, 5th Ed. (2011), Kurose, Computer Networking: A Top-Down Approach, Pearson, 7^{th} Ed. (2016), Elmasri, Fundamentals ofDatabase Systems, Addison Wesley, 6th Ed. (2010), Coronel, Database Systems: Design, Implementation, & Management, Cengage Learning, 11^{th} Ed. (2014), Tucker, Programming Languages, McGraw-Hill Science/Engineering/Math, 2nd Ed. (2006), and Rhoton, Cloud Computing Architected: Solution Design Handbook, Recursive Press (2011), each of which is hereby incorporated by reference in its entirety.

### M. Applications

### 1. Cancer and Other Diseases; Cell Type Quantification

The present methods can be used to diagnose the presence of a condition, e.g., cancer or precancer, in a subject, to characterize a condition (such as to determine a cancer stage or determining heterogeneity of a cancer), to monitor a subject's response to receiving a treatment for a condition (such as a response to a chemotherapeutic or immunotherapeutic), assess prognosis of a subject (such as to predict a survival outcome in a subject having a cancer), to determine a subject's risk of developing a condition, to predict a subsequent course of a condition in a subject, to determine metastasis or recurrence of a cancer in a subject (or a risk of cancer metastasis or recurrence), and/or to monitor a subject's health as part of a preventative health monitoring program (such as to determine whether and/or when a subject is in need of further diagnostic screening). The present disclosure can also be useful in determining the efficacy of a particular treatment option. Successful treatment options may increase the amount of rare mutations detected in a subject's blood if the treatment is successful as more cancers may die and shed nucleic acids (e.g., DNA). In other examples, this may not occur. In another example, certain treatment options may be correlated with genetic profiles of cancers over time. This correlation may be useful in selecting a therapy. In some embodiments, target regions are analyzed to determine whether they show methylation characteristics of tumor cells or cells that do not ordinarily contribute significantly to cfDNA and/or target regions are analyzed to determine whether they show methylation characteristic of tumor cells or cells that do not ordinarily contribute significantly to cfDNA. In some embodiments, successful treatment options may result in changes in levels of different immune cell types (including rare immune cell types), and/or increases in the amount of target proteins, copy number variation, rare mutations, and/or cancer-related epigenetic signatures (such as hypermethylated regions or hypomethylated regions) detected in, e.g., a sample from a subject, such as detected in a subject's blood (such as in DNA isolated from a buffy coat sample or any other sample comprising cells, such as in a blood sample (e.g., a whole blood sample, a plasma sample, a buffy coat sample, a leukapheresis sample, or a PBMC sample) from the subject) if the treatment is successful as more cancer cells may die and shed DNA, or, e.g., if a successful treatment results in an increase or decrease in the quantity of a specific immune cell type in the blood and an unsuccessful treatment results in no change. In other examples, this may not occur. In another example, certain treatment options may be correlated with genetic profiles of cancers over time.

Additionally, if a cancer is observed to be in remission after treatment, the present methods can be used to monitor the likelihood of residual disease or the likelihood of recurrence of disease.

In some embodiments, the present methods are used for screening for a cancer, such as a metastasis, or in a method for screening cancer, such as in a method of detecting the presence or absence of a metastasis. For example, the sample can be a sample from a subject who has or has not been previously diagnosed with cancer. In some embodiments, one or more, such as 1, 2, 3, 4, 5, 6, 7, 8, 9, 10, or more samples are collected from a subject as described herein, such as before and/or after the subject is diagnosed with a cancer. In some embodiments, the subject may or may not have cancer. In some embodiments, the subject may or may not have an early-stage cancer. In some embodiments, the subject has one or more risk factors for cancer, such as tobacco use (e.g., smoking), being overweight or obese, having a high body mass index (BMI), being of advanced age, poor nutrition, high alcohol consumption, or a family history of cancer. Furthermore, in some embodiments, the one or more methods described in the present disclosure may be used to assist in the treatment of a type of cancer.

In some embodiments, the subject has used tobacco, e.g., for at least 1, 5, 10, or 15 years. In some embodiments, the subject has a high BMI, e.g., a BMI of 25 or greater, 26 or greater, 27 or greater, 28 or greater, 29 or greater, or 30 or greater. In some embodiments, the subject is at least 40, 45, 50, 55, 60, 65, 70, 75, or 80 years old. In some embodiments, the subject has poor nutrition, e.g., high consumption of one or more of red meat and/or processed meat, trans fat, saturated fat, and refined sugars, and/or low consumption of fruits and vegetables, complex carbohydrates, and/or unsaturated fats. High and low consumption can be defined, e.g., as exceeding or falling below, respectively, recommendations in Dietary Guidelines for Americans 2020-2025, available at dietaryguidelines.gov/sites/default/files/2021-03/Dietary_Guidelines_for_Americans-2020-2025.pdf. In some embodiments, the subject has high alcohol consumption, e.g., at least three, four, or five drinks per day on average (where a drink is about one ounce or 30 mL of 80-proof hard liquor or the equivalent). In some embodiments, the subject has a family history of cancer, e.g., at least one, two, or three blood relatives were previously diagnosed with cancer. In some embodiments, the relatives are at least third-degree relatives (e.g., great-grandparent, great aunt or uncle, first cousin), at least second-degree relatives (e.g., grandparent, aunt or uncle, or half-sibling), or first-degree relatives (e.g., parent or full sibling).

Typically, the disease under consideration is a type of cancer. Non-limiting examples of such cancers include biliary tract cancer, bladder cancer, transitional cell carcinoma, urothelial carcinoma, brain cancer, gliomas, astrocytomas, breast cancer, metaplastic carcinoma, cervical cancer, cervical squamous cell carcinoma, rectal cancer, colorectal carcinoma, colon cancer, hereditary nonpolyposis colorectal cancer, colorectal adenocarcinomas, gastrointestinal stromal tumors (GISTs), endometrial carcinoma, endometrial stromal sarcomas, esophageal cancer, esophageal squamous cell carcinoma, esophageal adenocarcinoma, ocular melanoma, uveal melanoma, gallbladder carcinomas, gallbladder adenocarcinoma, renal cell carcinoma, clear cell renal cell carcinoma, transitional cell carcinoma, urothelial carcinomas, Wilms tumor, leukemia, lymphocytic cancer, acute lymphocytic leukemia (ALL), acute myeloid leukemia (AML), chronic lymphocytic leukemia (CLL), chronic myeloid leukemia (CML), chronic myelomonocytic leukemia (CMML), liver cancer, liver carcinoma, hepatoma, hepatocellular carcinoma, cholangiocarcinoma, hepatoblastoma, Lung cancer, non-small cell lung cancer (NSCLC), mesothelioma, lymphoma, B-cell lymphomas, non-Hodgkin lymphoma, diffuse large B-cell lymphoma, Mantle cell lymphoma, T cell lymphomas, non-Hodgkin lymphoma, precursor T-lymphoblastic lymphoma/leukemia, peripheral T cell lymphomas, myeloma, multiple myeloma, nasopharyngeal carcinoma (NPC), neuroblastoma, oropharyngeal cancer, oral cavity squamous cell carcinomas, osteosarcoma, ovarian carcinoma, pancreatic cancer, pancreatic ductal adenocarcinoma, pseudopapillary neoplasms, acinar cell carcinomas. Prostate cancer, prostate adenocarcinoma, skin cancer, melanoma, malignant melanoma, cutaneous melanoma, small intestine carcinomas, stomach cancer, gastric carcinoma, gastrointestinal stromal tumor (GIST), uterine cancer, or uterine sarcoma.

In some embodiments, the cancer is a type of cancer that is not a hematological cancer, e.g., a solid tumor cancer such as a carcinoma, adenocarcinoma, or sarcoma. Type and/or stage of cancer can be detected from genetic variations including mutations, rare mutations, indels, rearrangements, copy number variations, transversions, translocations, recombinations, inversion, deletions, aneuploidy, partial aneuploidy, polyploidy, chromosomal instability, chromosomal structure alterations, gene fusions, chromosome fusions, gene truncations, gene amplification, gene duplications, chromosomal lesions, DNA lesions, abnormal changes in nucleic acid chemical modifications, abnormal changes in epigenetic patterns, such as 5mC and 5mC profiles. Hence, the present methods can in some cases be used in combination with methods used to detect other genetic/epigenetic variations, e.g. in a method of detecting or characterizing a cancer or other methods described herein. In some embodiments, a method described herein comprises identifying the presence of target regions and/or DNA produced by a tumor (or neoplastic cells, or cancer cells) or by precancer cells. In some embodiments, a method described herein comprises determining the level of target regions and/or identifying the presence of DNA produced by a tumor (or neoplastic cells, or cancer cells) or by precancer cells. In some embodiments, determining the level of target regions comprises determining either an increased level or decreased level of target regions, wherein the increased or decreased level of target regions is determined by comparing the level of target regions with a threshold level/value.

Genetic and/or epigenetic data can also be used for characterizing a specific form of cancer. Cancers are often heterogeneous in both composition and staging. Genetic and/or epigenetic profile data may allow characterization of specific sub-types of cancer that may be important in the diagnosis or treatment of that specific sub-type. This information may also provide a subject or practitioner clues regarding the prognosis of a specific type of cancer and allow either a subject or practitioner to adapt treatment options in accord with the progress of the disease. Some cancers can progress to become more aggressive and genetically unstable. Other cancers may remain benign, inactive or dormant. The system and methods of this disclosure may be useful in determining disease progression.

Further, the methods of the disclosure may be used to characterize the heterogeneity of an abnormal condition in a subject. Such methods can include, e.g., generating a genetic and/or epigenetic profile of nucleic acids (e.g., cfDNA) derived from the subject, wherein the genetic and/or epigenetic profile comprises a plurality of data resulting from copy number variation and rare mutation analyses. In some embodiments, an abnormal condition is cancer, e.g., as described herein. In some embodiments, the abnormal condition may be one resulting in a heterogeneous genomic population. In the example of cancer, some tumors are known to comprise tumor cells in different stages of the cancer. In other examples, heterogeneity may comprise multiple foci of disease, such as where one or more foci (such as one or more tumor foci) are the result of metastases that have spread from a primary site of a cancer. The tissue(s) of origin can be useful for identifying organs affected by the cancer, including the primary cancer and/or metastatic tumors.

The present methods can also be used to quantify levels of different cell types, such as immune cell types, including rare immune cell types, such as activated lymphocytes and myeloid cells at particular stages of differentiation. Such quantification can be based on the numbers of molecules corresponding to a given cell type in a sample. In some embodiments, the sequencing comprises generating a plurality of sequencing reads. Sequence information obtained in the present methods may comprise sequence reads of the nucleic acids generated by a nucleic acid sequencer. In some embodiments, the nucleic acid sequencer performs pyrosequencing, single-molecule sequencing, nanopore sequencing, semiconductor sequencing, sequencing-by-synthesis, 5-letter sequencing, 6-letter sequencing, sequencing-by-ligation or sequencing-by-hybridization on the nucleic acids to generate sequencing reads.

In some embodiments, the method further comprises mapping the plurality of sequence reads to one or more reference sequences to generate mapped sequence reads. In some embodiments, the method further comprises grouping the sequence reads into families of sequence reads, each family comprising sequence reads generated from a nucleic acid in the sample. In some embodiments, the methods comprise determining the likelihood that the subject from which the sample was obtained has cancer or precancer, or has a metastasis, that is related to changes in proportions of types of immune cells. In some embodiments, the methods comprise processing the mapped sequence reads to determine the likelihood that the subject has cancer or precancer. In some embodiments, the detecting a presence or absence of DNA originating or derived from a tumor cell using the mapped sequence reads. In some embodiments, the methods comprise detecting a presence or absence of DNA originating or derived from a tumor cell using the mapped sequence reads.

The present methods can be used to generate or profile, fingerprint or set of data that is a summation of genetic and/or epigenetic information derived from different cells in a heterogeneous disease. This set of data may comprise copy number variation, epigenetic variation, and mutation analyses alone or in combination.

The present methods can be used to diagnose, prognose, monitor or observe cancers, or other diseases. In some embodiments, the methods herein do not involve the diagnosing, prognosing or monitoring a fetus and as such are not directed to non-invasive prenatal testing. In other embodiments, these methodologies may be employed in a pregnant subject to diagnose, prognose, monitor or observe cancers or other diseases in an unborn subject whose DNA and other polynucleotides may co-circulate with maternal molecules.

In some embodiments, the methods can provide a measure of the extent of DNA damage through the quantification of the methylated cytosines of CpG sites, the methods disclosed herein can also be used to quantify the level of DNA damage present in the original DNA sample.

In some embodiments, the method further comprises calculating a synthesis index which is a quantitative measure of the regions synthesized. The synthesis index may be on a molecule level and/or a sample level. The synthesis index may be the proportion of sequencing data which corresponds to synthesized regions. In some embodiments, the method further comprises comparing the synthesis index to one or more reference values to classify the DNA sample. The classification may be whether the DNA sample derives from a subject with or without cancer.

The reference values may be derived from one or more control DNA samples which are known to have specific properties, such as being derived from a subject known to have cancer, e.g. a specific type of cancer. The reference values may be obtained by performing the method used to obtain the synthesis index on control samples (i.e., using the same deamination, amplification, ligation and/or sequencing methods).

In some embodiments, the subject method can comprise detecting the single nucleotide variants (SNVs) in the DNA sample, and further comprises analyzing the sequence data, wherein the analyzing the sequence data comprises classifying all base calls within the one or more end repaired regions as not having double stranded support.

In some embodiments, the subject method can comprise comprising analyzing the sequence data to determine a level of measured artifacts in the DNA of the sample.

In some embodiments, the sample is obtained from a subject who was previously diagnosed with a cancer and received one or more previous cancer treatments. In some embodiments, the sample is obtained at one or more preselected time points following the one or more previous cancer treatments. In some embodiments, a method described herein comprises detecting a presence or absence of DNA originating or derived from a tumor cell at a preselected timepoint following a previous cancer treatment of a subject previously diagnosed with cancer using a set of sequence information obtained as described herein. The method may further comprise determining a cancer recurrence score that is indicative of the presence or absence of the DNA originating or derived from the tumor cell for the subject.

Where a cancer recurrence score is determined, it may further be used to determine a cancer recurrence status. The cancer recurrence status may be at risk for cancer recurrence, e.g., when the cancer recurrence score is above a predetermined threshold. The cancer recurrence status may be at low or lower risk for cancer recurrence, e.g., when the cancer recurrence score is below a predetermined threshold. In particular embodiments, a cancer recurrence score equal to the predetermined threshold may result in a cancer recurrence status of either at risk for cancer recurrence or at low or lower risk for cancer recurrence.

In some embodiments, a cancer recurrence score is compared with a predetermined cancer recurrence threshold, and the subject is classified as a candidate for a subsequent cancer treatment when the cancer recurrence score is above the cancer recurrence threshold or not a candidate for therapy when the cancer recurrence score is below the cancer recurrence threshold.

In particular embodiments, a cancer recurrence score equal to the cancer recurrence threshold may result in classification as either a candidate for a subsequent cancer treatment or not a candidate for therapy.

In some embodiments, the methods herein do not involve the diagnosing, prognosing or monitoring a fetus and as such are not directed to non-invasive prenatal testing. The present methods can be used to diagnose, prognose, monitor or observe cancers, or other diseases. In other embodiments, these methodologies may be employed in a pregnant subject to diagnose, prognose, monitor or observe cancers or other diseases in an unborn subject whose DNA and other polynucleotides may co-circulate with maternal molecules.

The present methods can also be used to quantify levels of different cell types, such as cancer cell types and/or immune cell types, including rare immune cell types, such as activated lymphocytes and myeloid cells at particular stages of differentiation. Such quantification can be based on the numbers of molecules corresponding to a given cell type in a sample.

Non-limiting examples of other genetic-based diseases, disorders, or conditions that are optionally evaluated using the methods and systems disclosed herein include achondroplasia, alpha-1 antitrypsin deficiency, antiphospholipid syndrome, autism, autosomal dominant polycystic kidney disease, Charcot-Marie-Tooth (CMT), cri du chat, Crohn's disease, cystic fibrosis, Dercum disease, down syndrome, Duane syndrome, Duchenne muscular dystrophy, Factor V Leiden thrombophilia, familial hypercholesterolemia, familial mediterranean fever, fragile X syndrome, Gaucher disease, hemochromatosis, hemophilia, holoprosencephaly, Huntington's disease, Klinefelter syndrome, Marfan syndrome, myotonic dystrophy, neurofibromatosis, Noonan syndrome, osteogenesis imperfecta, Parkinson's disease, phenylketonuria, Poland anomaly, porphyria, progeria, retinitis pigmentosa, severe combined immunodeficiency (SCID), sickle cell disease, spinal muscular atrophy, Tay-Sachs, thalassemia, trimethylaminuria, Turner syndrome, velocardiofacial syndrome, WAGR syndrome, Wilson disease, or the like.

In some embodiments, quantities of each of a plurality of cell types, such as cancer cell types and/or immune cell types, are determined based on sequencing and analysis (such as determination of epigenetic and/or genomic signatures) of DNA isolated from at least one sample, e.g., cfDNA, or DNA from a sample comprising cells (such as a buffy coat sample or another type of blood sample (e.g., a whole blood sample, a leukapheresis sample, or a PBMC sample) from a subject. For example, a plurality of immune cell types can include, but is not limited to, macrophages (including M1 macrophages and M2 macrophages), activated B cells (including regulatory B cells, memory B cells and plasma cells); T cell subsets, such as central memory T cells, naïve-like T cells, and activated T cells (including cytotoxic T cells, regulatory T cells (Tregs), CD4 effector memory T cells, CD4 central memory T cells, CD8 effector memory T cells, and CD8 central memory T cells); immature myeloid cells (including myeloid-derived suppressor cells (MDSCs), low-density neutrophils, immature neutrophils, and immature granulocytes); and natural killer (NK) cells. As disclosed herein, differences in levels and/or presence of particular genetic and/or epigenetic signatures in DNA isolated from blood samples from a subject can be used to quantify cell types, such as cancer cell types and/or immune cell types, within the sample.

Sequence information obtained in the present methods may comprise sequence reads of the nucleic acids generated by a nucleic acid sequencer. In some embodiments, the nucleic acid sequencer performs pyrosequencing, single-molecule sequencing, nanopore sequencing, semiconductor sequencing, sequencing-by-synthesis, 5-letter sequencing, 6-letter sequencing, sequencing-by-ligation or sequencing-by-hybridization on the nucleic acids to generate sequencing reads. In some embodiments, the method further comprises grouping the sequence reads into families of sequence reads, each family comprising sequence reads generated from a nucleic acid in the sample. In some embodiments, the methods comprise determining the likelihood that the subject from which the sample was obtained has cancer, precancer, an infection, transplant rejection, or other diseases or disorder that is related to changes in proportions of types of immune cells. Comparisons of immune cell identities and/or immune cell quantities/proportions between two or more samples collected from a subject at two different time points can allow for monitoring of one or more aspects of a condition in the subject over time, such as a response of the subject to a treatment, the severity of the condition (such as a cancer stage) in the subject, a recurrence of the condition (such as a cancer), and/or the subject's risk of developing the condition (such as a cancer).

The methods discussed above may further comprise any compatible feature or features set forth elsewhere herein, including in the section regarding methods of determining a risk of cancer recurrence in a subject and/or classifying a subject as being a candidate for a subsequent cancer treatment.

2. Methods of Determining a Risk of Cancer Recurrence in a Subject and/or Classifying a Test Subject as being a Candidate for a Subsequent Cancer Treatment

In some embodiments, a method provided herein is or comprises a method of determining a risk of cancer recurrence in a subject. In some embodiments, a method provided herein is or comprises a method of detecting the presence of absence of a metastasis in a subject. In some embodiments, a method provided herein is or comprises a method of classifying a subject as being a candidate for a subsequent cancer treatment.

Any of such methods may comprise collecting a sample (such as DNA, such as DNA originating or derived from a tumor cell) from the subject diagnosed with the cancer at one or more preselected timepoints following one or more previous cancer treatments to the subject. The subject may be any of the subjects described herein. The sample may comprise chromatin, cfDNA, or other cell materials. The sample, such as the DNA sample, may be a tissue sample. The DNA may be DNA, such as cfDNA, from a blood sample (e.g., a whole blood sample, a buffy coat sample, a leukapheresis sample, or a PBMC sample). The DNA may comprise DNA obtained from a tissue sample or a liquid sample.

Any of such methods may comprise capturing for a plurality of sets of target regions from DNA from the subject, wherein the plurality of target region sets comprises a sequence-variable target region set and an epigenetic target region set, whereby a captured set of DNA molecules is produced. The capturing step or steps may be performed according to any of the embodiments described elsewhere herein.

In any of such methods, the previous cancer treatment may comprise surgery, administration of a therapeutic composition, and/or chemotherapy.

Any of such methods may comprise sequencing the captured DNA molecules, whereby a set of sequence information is produced. The captured DNA molecules of the sequence-variable target region set may be sequenced to a greater depth of sequencing than the captured DNA molecules of the epigenetic target region set.

Any of such methods may comprise detecting a presence or absence of DNA originating or derived from a tumor cell at a preselected timepoint using the set of sequence information. The detection of the presence or absence of DNA, such as cfDNA originating or derived from a tumor cell may be performed according to any of the embodiments thereof described elsewhere herein.

Methods of determining a risk of cancer recurrence in a subject may comprise determining a cancer recurrence score that is indicative of the presence or absence, or amount, of the DNA, such as genomic regions of interest and target regions, originating or derived from the tumor cell for the subject. The cancer recurrence score may further be used to determine a cancer recurrence status. The cancer recurrence status may be at risk for cancer recurrence, e.g., when the cancer recurrence score is above a predetermined threshold. The cancer recurrence status may be at low or lower risk for cancer recurrence, e.g., when the cancer recurrence score is above a predetermined threshold. In particular embodiments, a cancer recurrence score equal to the predetermined threshold may result in a cancer recurrence status of either at risk for cancer recurrence or at low or lower risk for cancer recurrence.

Methods of detecting the presence or absence of metastasis in a subject may comprise comparing the presence or level of a tissue-specific cell material to the presence or level of the tissue-specific cell material obtained from the subject at a different time, a reference level of the tissue-specific cell material, or to a comparator cell material. Methods herein may comprise additional steps to determine whether a metastasis is present.

Methods of classifying a subject as being a candidate for a subsequent cancer treatment may comprise comparing the cancer recurrence score of the subject with a predetermined cancer recurrence threshold, thereby classifying the subject as a candidate for the subsequent cancer treatment when the cancer recurrence score is above the cancer recurrence threshold or not a candidate for therapy when the cancer recurrence score is below the cancer recurrence threshold. In particular embodiments, a cancer recurrence score equal to the cancer recurrence threshold may result in classification as either a candidate for a subsequent cancer treatment or not a candidate for therapy. In some embodiments, the subsequent cancer treatment comprises chemotherapy or administration of a therapeutic composition.

Any of such methods may comprise determining a disease-free survival (DFS) period for the subject based on the cancer recurrence score; for example, the DFS period may be 1 year, 2 years, 3, years, 4 years, 5 years, or 10 years.

In some embodiments, sequence-variable target region sequences are obtained, and determining the cancer recurrence score may comprise determining at least a first subscore indicative of the amount of the levels of particular immune cell types, SNVs, insertions/deletions, CNVs and/or fusions present in sequence-variable target region sequences.

In some embodiments, a number of mutations in the sequence-variable target regions chosen from 1, 2, 3, 4, or 5 is sufficient for the first subscore to result in a cancer recurrence score classified as positive for cancer recurrence. In some embodiments, the number of mutations is chosen from 1, 2, or 3.

In some embodiments, epigenetic target region sequences are obtained, and determining the cancer recurrence score comprises determining a second subscore indicative of the amount of molecules (obtained from the epigenetic target region sequences) that represent an epigenetic state different from DNA found in a corresponding sample from a healthy subject (e.g., DNA, such as cfDNA, found in a blood sample (e.g., a whole blood sample, a buffy coat sample, a leukapheresis sample, or a PBMC sample) from a healthy subject) from a health subject, or DNA found in a tissue sample from a healthy subject where the tissue sample is of the same type of tissue as was obtained from the subject). These abnormal molecules (i.e., molecules with an epigenetic state different from DNA found in a corresponding sample from a healthy subject) may be consistent with epigenetic changes associated with cancer (such as with a metastasis), e.g., methylation of hypermethylation variable target regions and/or perturbed fragmentation of fragmentation variable target regions, where “perturbed” means different from DNA found in a corresponding sample from a healthy subject.

In some embodiments, a proportion of molecules corresponding to the hypermethylation variable target region set and/or fragmentation variable target region set that indicate hypermethylation in the hypermethylation variable target region set and/or abnormal fragmentation in the fragmentation variable target region set greater than or equal to a value in the range of 0.001%-10% is sufficient for the second subscore to be classified as positive for cancer recurrence. The range may be 0.001%-1%, 0.005%-1%, 0.01%-5%, 0.01%-2%, or 0.01%-1%.

In some embodiments, any of such methods may comprise determining a fraction of tumor DNA from the fraction of molecules in the set of sequence information that indicate one or more features indicative of origination from a tumor cell. This may be done for molecules corresponding to some or all of the target regions, e.g., including one or more of hypermethylation variable target regions, hypomethylation variable target regions, and fragmentation variable target regions (hypermethylation of a hypermethylation variable target region and/or abnormal fragmentation of a fragmentation variable target region may be considered indicative of origination from a tumor cell). This may be done for molecules corresponding to sequence-variable target regions, e.g., molecules comprising alterations consistent with cancer, such as SNVs, indels, CNVs, and/or fusions. The fraction of tumor DNA may be determined based on a combination of molecules corresponding to epigenetic target regions and molecules corresponding to sequence-variable target regions.

Determination of a cancer recurrence score may be based at least in part on the fraction of tumor DNA, wherein a fraction of tumor DNA greater than a threshold in the range of 10⁻¹¹ to 1 or 10⁻¹⁰ to 1 is sufficient for the cancer recurrence score to be classified as positive for cancer recurrence. In some embodiments, a fraction of tumor DNA greater than or equal to a threshold in the range of 10⁻¹⁰ to 10⁻⁹, 10⁻⁹ to 10⁻⁸, 10⁻⁸ to 10⁻⁷, 10⁻⁷ to 10⁻⁶, 10⁻⁶ to 10⁻¹, 10⁻⁵ to 10⁻⁴, 10⁻⁴ to 10⁻³, 10⁻³ to 10⁻², or 10⁻² to 10⁻¹ is sufficient for the cancer recurrence score to be classified as positive for cancer recurrence. In some embodiments, the fraction of tumor DNA greater than a threshold of at least 10⁻⁷ is sufficient for the cancer recurrence score to be classified as positive for cancer recurrence. A determination that a fraction of tumor DNA is greater than a threshold, such as a threshold corresponding to any of the foregoing embodiments, may be made based on a cumulative probability. For example, the sample was considered positive if the cumulative probability that the tumor fraction was greater than a threshold in any of the foregoing ranges exceeds a probability threshold of at least 0.5, 0.75, 0.9, 0.95, 0.98, 0.99, 0.995, or 0.999. In some embodiments, the probability threshold is at least 0.95, such as 0.99.

In some embodiments, the set of sequence information comprises sequence-variable target region sequences and epigenetic target region sequences, and determining the cancer recurrence score comprises determining a subscore indicative of the amount of SNVs, insertions/deletions, CNVs and/or fusions present in sequence-variable target region sequences and a subscore indicative of the amount of abnormal molecules in epigenetic target region sequences, and combining the subscores to provide the cancer recurrence score. Where the subscores are combined, they may be combined by applying a threshold to each subscore independently (e.g., greater than a predetermined number of mutations (e.g., >1) in sequence-variable target regions, and greater than a predetermined fraction of abnormal molecules (i.e., molecules with an epigenetic state different from the DNA found in a corresponding sample from a healthy subject; e.g., tumor) in epigenetic target regions), or training a machine learning classifier to determine status based on a plurality of positive and negative training samples.

In some embodiments, the set of sequence information comprises sequence-variable target region sequences and epigenetic target region sequences, and determining the cancer recurrence score comprises determining a first subscore indicative of the levels of particular immune cell types, a second subscore indicative of the amount of SNVs, insertions/deletions, CNVs and/or fusions present in sequence-variable target region sequences and a third subscore indicative of the amount of abnormal molecules in epigenetic target region sequences, and combining the first, second, and third subscores to provide the cancer recurrence score. Where the subscores are combined, they may be combined by applying a threshold to each subscore independently in sequence-variable target regions, respectively, and greater than a predetermined fraction of abnormal molecules (i.e., molecules with an epigenetic state different from the DNA found in a corresponding sample from a healthy subject; e.g., tumor) in epigenetic target regions), or training a machine learning classifier to determine status based on a plurality of positive and negative training samples.

In some embodiments, a value for the combined score in the range of −4 to 2 or −3 to 1 is sufficient for the cancer recurrence score to be classified as positive for cancer recurrence.

In any embodiment where a cancer recurrence score is classified as positive for cancer recurrence, the cancer recurrence status of the subject may be at risk for cancer recurrence and/or the subject may be classified as a candidate for a subsequent cancer treatment.

In some embodiments, the cancer is any one of the types of cancer described elsewhere herein, e.g., colorectal cancer.

### 3. Methods of Monitoring a Cancer in a Subject Over Time; Sample Collection at Two or More Time Points

In some embodiments, the present methods can be used to monitor one or more aspects of a condition in a subject over time, such as a subject's response to receiving a treatment for a condition (such as a response to a chemotherapeutic or immunotherapeutic), the severity of the condition (such as a cancer stage) in the subject, a recurrence of the condition (such as a cancer), and/or the subject's risk of developing the condition (such as a cancer) and/or to monitor a subject's health as part of a preventative health monitoring program (such as to determine whether and/or when a subject is in need of further diagnostic screening). In some embodiments, monitoring comprises analysis of at least two samples collected from a subject at at least two different time points as described herein.

The methods according to the present disclosure can be useful in predicting a subject's response to a particular treatment option, such as over a period of time. As described elsewhere herein, successful treatment options may increase the amount of cancer associated DNA sequences detected in a subject's blood, such as if the treatment is successful as more cancers may die and shed DNA. In such examples, certain treatment options may be correlated with genetic profiles of cancers over time. This correlation may be useful in selecting a therapy. In some embodiments, successful treatment options may result in an increase or decrease in the levels of different immune cell types (including rare immune cell types), and/or an increase or decrease in the levels of a specific protein or proteins and/or a specific DNA sequence (e.g., of a CDR3), such as in the blood, and an unsuccessful treatment may result in no change. In other examples, this may not occur.

As disclosed herein, in some embodiments, quantities of each of a plurality of cell types, such as immune cell types, are determined based on sequencing and analysis (such as determination of epigenetic and/or genomic signatures) of DNA isolated from at least one sample comprising cells (such as a tissue sample or a blood sample (e.g., a whole blood sample, a buffy coat sample, a leukapheresis sample, or a PBMC sample) from a subject. In some embodiments, differences in levels and/or presence of particular genetic and/or epigenetic signatures in DNA isolated from blood samples from a subject can be used to quantify cell types, such as immune cell types, within the sample. Thus, a comparison of the disclosed genetic and/or epigenetic signatures in DNA isolated from blood samples collected from a subject at two or more time points can be used to monitor changes in cell type quantities in the subject under different conditions (such as prior to and after a treatment), or over time (e.g., as part of a preventative health monitoring program).

The disclosed methods can include evaluating (such as quantifying) and/or interpreting cell types (such as immune cell types) present in one or more samples (such as a tissue sample or a blood sample, e.g., a whole blood sample, a buffy coat sample, a leukapheresis sample, or a PBMC sample) collected from a subject at one or more timepoints in comparison to a selected baseline value or reference standard (or a selected set of baseline values or reference standards). A baseline value or reference standard may be a quantity of cell types measured in one or more samples (such as an average quantity or range of quantities of cell types present in at least two samples) collected from the subject at one or more time points, such as prior to receiving a treatment, prior to diagnosis of a condition (such as a cancer), or as part of a preventative health monitoring program. A baseline value or reference standard may be a quantity of cell types measured in one or more samples (such as an average quantity or range of quantities of cell types present in at least two samples) collected at one or more timepoints from one or more subjects that do not have the condition (such as a healthy subject that does not have a cancer), one or more subjects that responded favorably to the treatment, or one or more subjects that have not received the treatment. In certain embodiments, the baseline value or reference standard utilized is a standard or profile derived from a single reference subject. In other embodiments, the baseline value or reference standard utilized is a standard or profile derived from averaged data from multiple reference subjects. The reference standard, in various embodiments, can be a single value, a mean, an average, a numerical mean or range of numerical means, a numerical pattern, or a graphical pattern created from the cell type quantity data derived from a single reference subject or from multiple reference subjects. Selection of the particular baseline values or reference standards, or selection of the one or more reference subjects, depends upon the use to which the methods described herein are to be put by, for example, a research scientist or a clinician (such as a physician).

In some embodiments, one or more samples (such as a tissue sample or a blood sample, e.g., a whole blood sample, a buffy coat sample, a leukapheresis sample, or a PBMC sample) may be collected from a subject at two or more timepoints, to assess changes in cell types (such as changes in quantities of cell types) between the two or more timepoints. In some embodiments, a sample collected at a first time point is a tissue sample or a blood sample, and a sample collected at a subsequent time point (such as a second time point) is a blood sample. In some embodiments, a sample collected at a first time point is a tissue sample and a sample collected at a subsequent time point (such as a second time point) is a blood sample. By monitoring cell types and identifying differences between cell types in samples collected from a subject at two or more timepoints, the present methods can be used, for example, to determine the presence or absence of a condition (such as a cancer), a response of the subject to a treatment, one or more characteristic of a condition (such as a cancer stage) in the subject, recurrence of a condition (such as a cancer), and/or a subject's risk of developing a condition (such as a cancer). Thus, in some embodiments, methods are provided wherein quantities of cell types present in at least one sample (such as at least one tissue sample and/or at least one blood sample, e.g., a whole blood sample, buffy coat sample, leukapheresis sample, or PBMC sample) collected from a subject at one or more timepoints (such as prior to receiving a treatment) are compared to quantities of cell types present in at least one sample collected from the subject at one or more different time points (such as after receiving the treatment). The disclosed methods can allow for patient-specific monitoring, such that, for example, differences in cell type quantities between samples collected from the subject at different timepoints may indicate changes (such as presence or absence of a condition, response to a treatment, a prognosis, or the like) that are significant with respect to the subject but may yet fall within a normal range of a general healthy population.

As disclosed herein, methods are provided for monitoring one or more aspects of a condition in a subject over time, such as but not limited to, a subject's response to receiving a treatment for a condition (such as a response to a chemotherapeutic or immunotherapeutic). In certain embodiments, one or more samples is collected from the subject at at least 1-10, at least 1-5, at least 2-5, or at least 1, at least 2, least 3, at least 4, at least 5, at least 6, at least 7, at least 8, at least 9, at least 10, at least 15, or at least 20 time points prior to the subject receiving the treatment. In certain embodiments, one or more samples is collected from the subject at at least 1-10, at least 1-5, at least 2-5, or at least 1, at least 2, least 3, at least 4, at least 5, at least 6, at least 7, at least 8, at least 9, at least 10, at least 15, or at least 20 time points after the subject has received the treatment. Sample collection from a subject can be ongoing during and/or after treatment to monitor the subject's response to the treatment.

In some embodiments, samples are not collected from a subject prior to diagnosis of a condition (such as a cancer) or prior to receiving a treatment. In such embodiments, wherein the response of a subject to a treatment, or the course or stage of a condition (such as a cancer) in the subject is being monitored over time, cell types are compared between samples taken at at least 2-10, at least 2-5, at least 3-6, or at least 2, such as at least 3, at least 4, at least 5, at least 6, at least 7, at least 8, at least 9, at least 10, at least 15, or at least 20 time points collected after the subject has been diagnosed and/or after the subject has received the treatment. Sample collection from a subject can be ongoing during and/or after treatment to monitor the subject's response to the treatment.

In some embodiments of the disclosed methods, one or more samples (such as one or more tissue, whole blood, buffy coat, leukapheresis, or PBMC samples) is collected from a subject at least once per year, such as about 1-12 times or about 2-6 times, such as about 1, 2, 3, 4, 5, 6, 7, 8, 9, 10, 11, or 12 times per year. In other embodiments, one or more samples is collected from the subject less than once per year, such as about once every 13, 14, 15, 16, 17, 18, 19, 20, 21, 22, 23, or 24 months. In some embodiments, one or more samples is collected from the subject about once every 1-5 years or about once every 1-2 years, such as about every 1, 1.5, 2, 2.5, 3, 3.5, 4, 4.5, or 5 years.

In other embodiments of the disclosed methods, one or more samples (such as one or more tissue samples or blood samples, e.g., or one or more buffy coat samples, whole blood samples, leukapheresis samples, or PBMC samples) are collected from a subject at least once per week, such as on 1-4 days, 1-2 days, or on 1, 2, 3, 4, 5, 6, or 7 days per week. In certain embodiments, one or more samples is collected from the subject at least once per month, such as 1-15 times, 1-10 times, 2-5 times, or 1, 2, 3, 4, 5, 6, 7, 8, 9, 10, 11, 12, 13, 14, or 15 times per month. In other embodiments, one or more samples is collected from the subject every month, every 2 months, every 3 months, every 4 months, every 5 months, every 6 months, every 7 months, every 8 months, every 9 months, every 10 months, every 11 months, or every 12 months. In some embodiments, one or more samples is collected from the subject at least once per day, such as 1, 2, 3, 4, 5, or 6 times per day. Selection of the one or more sample collection timepoints (e.g., the frequency of sample collection), or of the number of samples to be collected at each timepoint, depends upon the use to which the methods described herein are to be put by, for example, a research scientist or a clinician (such as a physician).

### 4. Therapies and Related Administration

In certain embodiments, the methods disclosed herein relate to identifying and administering therapies, such as customized therapies, to patients. In some embodiments, determination of the levels of particular immune cell types, including rare immune cell types, facilitates selection of appropriate treatment. In some embodiments, the patient or subject has a given disease, disorder or condition, e.g., any of the cancers or other conditions described elsewhere herein. Essentially any cancer therapy (e.g., surgical therapy, radiation therapy, chemotherapy, immunotherapy, and/or the like) may be included as part of these methods. In certain embodiments, the therapy administered to a subject comprises at least one chemotherapy drug. In some embodiments, the chemotherapy drug may comprise alkylating agents (for example, but not limited to, Chlorambucil, Cyclophosphamide, Cisplatin and Carboplatin), nitrosoureas (for example, but not limited to, Carmustine and Lomustine), anti-metabolites (for example, but not limited to, Fluorauracil, Methotrexate and Fludarabine), plant alkaloids and natural products (for example, but not limited to, Vincristine, Paclitaxel and Topotecan), anti-tumor antibiotics (for example, but not limited to, Bleomycin, Doxorubicin and Mitoxantrone), hormonal agents (for example, but not limited to, Prednisone, Dexamethasone, Tamoxifen and Leuprolide) and biological response modifiers (for example, but not limited to, Herceptin and Avastin, Erbitux and Rituxan). In some embodiments, the chemotherapy administered to a subject may comprise FOLFOX or FOLFIRI. In certain embodiments, a therapy may be administered to a subject that comprises at least one PARP inhibitor. In some embodiments, the therapies are PARP inhibitors, such as Olaparib (LYNPARZA®), Rucaparib (RUBRACA®), Niraparib (ZEJULA®), and Talazoparib (TALZENNA®). These may be used for treating mutations in BRCA1, BRCA2, ATM, BARD1, BRIP1, CDK12, CHEK1, CHEK2, FANCL, PALB2, RAD51B, RAD51C, RAD51D and RAD54L alterations, and/or for genes associated Homologous Recombination Repair (HRR). Typically, therapies include at least one immunotherapy (or an immunotherapeutic agent). Immunotherapy refers generally to methods of enhancing an immune response against a given cancer type. In certain embodiments, immunotherapy refers to methods of enhancing a T cell response against a tumor or cancer.

In some embodiments, therapy is customized based on the status of a nucleic acid variant as being of somatic or germline origin. In some embodiments, essentially any cancer therapy (e.g., surgical therapy, radiation therapy, chemotherapy, immunotherapy, and/or the like) may be included as part of these methods. Customized therapies can include at least one immunotherapy (or an immunotherapeutic agent). Immunotherapy refers generally to methods of enhancing an immune response against a given cancer type. In certain embodiments, immunotherapy refers to methods of enhancing a T cell response against a tumor or cancer.

In some embodiments, the immunotherapy or immunotherapeutic agent targets an immune checkpoint molecule. Certain tumors are able to evade the immune system by co-opting an immune checkpoint pathway. Thus, targeting immune checkpoints has emerged as an effective approach for countering a tumor's ability to evade the immune system and activating anti-tumor immunity against certain cancers. Pardoll, Nature Reviews Cancer, 2012, 12:252-264.

In some embodiments the treatment comprises immunotherapies and/or immune checkpoint inhibitors (ICIS). Immunotherapies are treatments with one or more agents that act to stimulate the immune system so as to kill or at least to inhibit growth of cancer cells, and preferably to reduce further growth of the cancer, reduce the size of the cancer and/or eliminate the cancer. Some such agents bind to a target present on cancer cells; some bind to a target present on immune cells and not on cancer cells; some bind to a target present on both cancer cells and immune cells. Such agents include, but are not limited to, checkpoint inhibitors and/or antibodies. Checkpoint inhibitors are inhibitors of pathways of the immune system that maintain self-tolerance and modulate the duration and amplitude of physiological immune responses in peripheral tissues to minimize collateral tissue damage (see, e.g., Pardoll, Nature Reviews Cancer 12, 252-264 (2012)). Exemplary agents include antibodies against any of PD-1, PD-2, PD-L1, PD-L2, CTLA-4, OX40, B7.1, B7He, LAG3, CD137, KIR, CCR5, CD27, CD40, or CD47. Other exemplary agents include proinflammatory cytokines, such as IL-1, IL-6, and TNF-α. Other exemplary agents are T-cells activated against a tumor, such as T-cells activated by expressing a chimeric antigen targeting a tumor antigen recognized by the T-cell. In some embodiments, anti-PD-1 or anti-PD-L1 therapies comprise pembrolizumab (KEYTRUDA®), nivolumab (OPDIVO®), and cemiplimab (LIBTAYO®), atezolizumab (TECENTRIQ®), durvalumab (INFINZI®), and avelumab (BAVENCIO®). These therapies may be used to treat patients identified as having high microsatellite instability (MSI) status or high tumor mutational burden (TMB).

In certain embodiments, the immune checkpoint molecule is an inhibitory molecule that reduces a signal involved in the T cell response to antigen. For example, CTLA4 is expressed on T cells and plays a role in downregulating T cell activation by binding to CD80 (aka B7.1) or CD86 (aka B7.2) on antigen presenting cells. PD-1 is another inhibitory checkpoint molecule that is expressed on T cells. PD-1 limits the activity of T cells in peripheral tissues during an inflammatory response. In addition, the ligand for PD-1 (PD-L1 or PD-L2) is commonly upregulated on the surface of many different tumors, resulting in the downregulation of anti-tumor immune responses in the tumor microenvironment. In certain embodiments, the inhibitory immune checkpoint molecule is CTLA4 or PD-1. In other embodiments, the inhibitory immune checkpoint molecule is a ligand for PD-1, such as PD-L1 or PD-L2. In other embodiments, the inhibitory immune checkpoint molecule is a ligand for CTLA4, such as CD80 or CD86. In other embodiments, the inhibitory immune checkpoint molecule is lymphocyte activation gene 3 (LAG3), killer cell immunoglobulin like receptor (KIR), T cell membrane protein 3 (TIM3), galectin 9 (GAL9), or adenosine A2a receptor (A2aR).

Antagonists that target these immune checkpoint molecules can be used to enhance antigen-specific T cell responses against certain cancers. Accordingly, in certain embodiments, the immunotherapy or immunotherapeutic agent is an antagonist of an inhibitory immune checkpoint molecule. In certain embodiments, the inhibitory immune checkpoint molecule is PD-1. In certain embodiments, the inhibitory immune checkpoint molecule is PD-L1. In certain embodiments, the antagonist of the inhibitory immune checkpoint molecule is an antibody (e.g., a monoclonal antibody). In certain embodiments, the antibody or monoclonal antibody is an anti-CTLA4, anti-PD-1, anti-PD-L1, or anti-PD-L2 antibody. In certain embodiments, the antibody is a monoclonal anti-PD-1 antibody. In some embodiments, the antibody is a monoclonal anti-PD-L1 antibody. In certain embodiments, the monoclonal antibody is a combination of an anti-CTLA4 antibody and an anti-PD-1 antibody, an anti-CTLA4 antibody and an anti-PD-L1 antibody, or an anti-PD-L1 antibody and an anti-PD-1 antibody. In certain embodiments, the anti-PD-1 antibody is one or more of pembrolizumab (Keytruda®) or nivolumab (Opdivo®). In certain embodiments, the anti-CTLA4 antibody is ipilimumab (Yervoy®). In certain embodiments, the anti-PD-L1 antibody is one or more of atezolizumab (Tecentriq®), avelumab (Bavencio®), or durvalumab (Imfinzi®).

In certain embodiments, the immunotherapy or immunotherapeutic agent is an antagonist (e.g. antibody) against CD80, CD86, LAG3, KIR, TIM3, GAL9, or A2aR. In other embodiments, the antagonist is a soluble version of the inhibitory immune checkpoint molecule, such as a soluble fusion protein comprising the extracellular domain of the inhibitory immune checkpoint molecule and an Fc domain of an antibody. In certain embodiments, the soluble fusion protein comprises the extracellular domain of CTLA4, PD-1, PD-L1, or PD-L2. In some embodiments, the soluble fusion protein comprises the extracellular domain of CD80, CD86, LAG3, KIR, TIM3, GAL9, or A2aR. In one embodiment, the soluble fusion protein comprises the extracellular domain of PD-L2 or LAG3.

In some embodiments, the therapies target mutated forms of the EGFR protein. Such therapies can include osimertinib (TAGRISSO®), erlotinib (TARCEVA®), and gefinitib (IRESSA®).

Therapies can include one or more of treatments for target therapies, including abemaciclib (VERZENIO®), abiraterone acetate (ZYTIGA®), acalabrutinib (CALQUENCE®), adagrasib (KRAZATI®), ado-trastuzumab emtansine (KADCYLA®), afatinib dimaleate (GILOTRIF®), alectinib (ALCENSA®), alemtuzumab (CAMPATH®), alitretinoin (PANRETIN®), alpelisib (PIQRAY®), amivantamab-vmjw (RYBREVANT®), anastrozole (ARIMIDEX®), apalutamide (ERLEADA®), asciminib hydrochloride (SCEMBLIX®), atezolizumab (TECENTRIQ®), avapritinib (AYVAKIT®), avelumab (BAVENCIO®), axicabtagene ciloleucel (YESCARTA®), axitinib (INLYTA®), belinostat (BELEODAQ®), belzutifan (WELIREG®), bevacizumab (AVASTIN®), bexarotene (TARGRETIN®), binimetinib (MEKTOVI®), blinatumomab (BLINCYTO®), bortezomib (VELCADE®), bosutinib (BOSULIF®), brentuximab vedotin (ADCETRIS®), brexucabtagene autoleucel (TECARTUS®), brigatinib (ALUNBRIG®), cabazitaxel (JEVTANA), cabozantinib-s-malate (CABOMETYX®), cabozantinib-s-malate (COMETRIQ®), capmatinib hydrochloride (TABRECTA®), carfilzomib (KYPROLIS®), cemiplimab-rwlc (LIBTAYO®), ceritinib (ZYKADIA®), cetuximab (ERBITUX®), ciltacabtagene autoleucel (CARVYKTI®), cobimetinib fumarate (COTELLIC®), copanlisib hydrochloride (ALIQUOPA®), crizotinib (XALKORI®), dabrafenib (TAFMLAR®), dabrafenib mesylate (TAFMLAR®), dacomitinib (VIZIMPRO®), daratumumab (DARZALEX®), daratumumab and hyaluronidase-fihj (DARZALEX FASPRO®), darolutamide (NUBEQA®), dasatinib (SPRYCEL®), denileukin diftitox (ONTAK®), denosumab (XGEVA®), dinutuximab (UNITUXIN®), dostarlimab-gxly (JEMPERLI®), durvalumab (IMFINZI®), duvelisib (COPIKTRA®), elacestrant dihydrochloride (ORSERDU®), elotuzumab (EMPLICITI®), enasidenib mesylate (IDHIFA®), encorafenib (BRAFTOVI®), enfortumab vedotin-ejfv (PADCEV®), entrectinib (ROZLYTREK®), enzalutamide (XTANDI®), erdafitinib (BALVERSA®), erlotinib hydrochloride (TARCEVA®), everolimus (AFINITOR®), exemestane (AROMASIN®), fam-trastuzumab deruxtecan-nxki (ENHERTU®), fedratinib hydrochloride (INREBIC®), fulvestrant (FASLODEX®), futibatinib (LYTGOBI®), gefitinib (IRESSA®), gemtuzumab ozogamicin (MYLOTARG®), gilteritinib fumarate (XOSPATA®), glasdegib maleate (DAURISMO®), ibritumomab tiuxetan (ZEVALIN®), ibrutinib (IMBRUVICA®), idecabtagene vicleucel (ABECMA®), idelalisib (ZYDELIG®), imatinib mesylate (GLEEVEC®), infigratinib phosphate (TRUSELTIQ®), inotuzumab ozogamicin (BESPONSA®), iobenguane 1 131 (AZEDRA®), ipilimumab (YERVOY®), isatuximab-irfc (SARCLISA®), ivosidenib (TIBSOVO®), ixazomib citrate (NINLARO®), lanreotide acetate (SOMATULINE DEPOT®), lapatinib ditosylate (TYKERB®), larotrectinib sulfate (VITRAKVI®), lenvatinib mesylate (LENVIMA®), letrozole (FEMARA®), lisocabtagene maraleucel (BREYANZI®), loncastuximab tesirine-lpyl (ZYNLONTA®), lorlatinib (LORBRENA®), lutetium Lu 177 vipivotide tetraxetan (PLUVICTO®), lutetium Lu 177-dotatate (LUTATHRA®), margetuximab-cmkb (MARGENZA®), midostaurin (RYDAPT®), mirvetuximab soravtansine-gynx (ELAHERE®), mobocertinib succinate (EXKIVITY®), mogamulizumab-kpkc (POTELIGEO®), mosunetuzumab-axgb (LUNSUMIO®), moxetumomab pasudotox-tdfk (LUMOXITI®), naxitamab-gqgk (DANYELZA®), necitumumab (PORTRAZZA®), neratinib maleate (NERLYNX®), nilotinib (TASIGNA®), niraparib tosylate monohydrate (ZEJULA®), nivolumab (OPDIVO®), nivolumab and relatlimab-rmbw (OPDUALAG®), obinutuzumab (GAZYVA®), ofatumumab (ARZERRA®), olaparib (LYNPARZA®), olutasidenib (REZLHIDIA®), osimertinib mesylate (TAGRISSO®), pacritinib citrate (VONJO®), palbociclib (IBRANCE®), panitumumab (VECTIBIX®), pazopanib hydrochloride (VOTRIENT®), pembrolizumab (KEYTRUDA®), pemigatinib (PEMAZYRE®), pertuzumab (PERJETA®), pertuzumab, trastuzumab, and hyaluronidase-zzxf (PHESGO®), pexidartinib hydrochloride (TURALIO®), pirtobrutinib (JAYPIRCA®), polatuzumab vedotin-piiq (POLIVY®), ponatinib hydrochloride (ICLUSIG®), pralatrexate (FOLOTYN®), pralsetinib (GAVRETO®), radium 223 dichloride (XOFIGO®), ramucirumab (CYRAMZA®), regorafenib (STIVARGA®), retifanlimab-dlwr (ZYNYZ®), ribociclib (KISQALI®), ripretinib (QINLOCK®), rituximab (RITUXAN®), rituximab and hyaluronidase human (RITUXAN HYCELA®), romidepsin (ISTODAX®), rucaparib camsylate (RUBRACA®), ruxolitinib phosphate (JAKAFI®), sacituzumab govitecan-hziy (TRODELVY®), selinexor (XPOVIO®), selpercatinib (RETEVMO®), selumetinib sulfate (KOSELUGO®), siltuximab (SYLVANT®), sirolimus protein-bound particles (FYARRO®), sonidegib (ODOMZO®), sorafenib tosylate (NEXAVAR®), sotorasib (LUMAKRAS®), sunitinib malate (SUTENT®), tafasitamab-cxix (MONJUVI®), tagraxofusp-erzs (ELZONRIS®), talazoparib tosylate (TALZENNA®), tamoxifen citrate (SOLTAMOX®), tazemetostat hydrobromide (TAZVERIK®), tebentafusp-tebn (KIMMTRAK®), teclistamab-cqyv (TECVAYLI®), temsirolimus (TORISEL®), tepotinib hydrochloride (TEPMETKO®), tisagenlecleucel (KYMRIAH®), tisotumab vedotin-tftv (TIVDAK®), tivozanib hydrochloride (FOTIVDA®), toremifene (FARESTON®), trametinib (MEKINIST®), trametinib dimethyl sulfoxide (MEKINIST®), trastuzumab (HERCEPTIN®), tremelimumab-actl (IMJUDO®), tretinoin (VESANOID®), tucatinib (TUKYSA®), vandetanib (CAPRELSA®), vemurafenib (ZELBORAF®), venetoclax (VENCLEXTA®), vismodegib (ERIVEDGE®), vorinostat (ZOLINZA®), zanubrutinib (BRUKINSA®), and/or ziv-aflibercept (ZALTRAP®).

Table 10 provides an exemplary list of drugs used to treat cancers with mutations observed in target genes associated with certain cancer types. In certain embodiments, the subject has a cancer of a type listed in Table 10 including a mutation in one or more target genes listed in Table 10 for that cancer type, and the therapy administered to the subject comprises the drug listed in Table 10 for that cancer type and mutation.

TABLE 10
Exemplary drugs

| Cancer type | Drug | Target genes |
|---|---|---|
| Breast | abemaciclib (VERZENIO ®) | CDK4, CDK6 |
| prostate | abiraterone acetate (ZYTIGA ®) | CYP17A1 |
| leukemia | acalabrutinib (CALQUENCE ®) | BTK |
| lymphoma | acalabrutinib (CALQUENCE ®) | BTK |
| lung | adagrasib (KRAZATI ®) | KRAS G12C |
| Breast | ado-trastuzumab emtansine | Tubulin, Her2 |
| | (KADCYLA ®) | |
| lung | afatinib dimaleate (GILOTRIF ®) | EGFR + EGFR exon 21 L858R |
| | | mutation + EGFR-Ex19del + HER2 + |
| | | HER4 |
| lung | alectinib (ALECENSA ®) | ALK, RET |
| leukemia | alemtuzumab (CAMPATH ®) | CD52 |
| skin | alitretinoin (PANRENTIN ®) | RARα + RARβ2 + RARγ |
| soft tissue | alitretinoin (PANRENTIN ®) | RARα + RARβ2 + RARγ |
| sarcoma | | |
| Breast | alpelisib (PIQRAY ®) | PI3Kα |
| lung | amivantamab-vmjw (RYVEBRANT ®) | EGFR+c-MET |
| Breast | anastrozole (ARIMIDEX ®) | Aromatase |
| prostate | apalutamide (ERLEADA ®) | AR |
| leukemia | asciminib hydrochloride (SCEMBLIX ®) | Bcr-Abl |
| bladder | atezolizumab (TECENTRIQ ®) | PDL1 |
| liver and bile | atezolizumab (TECENTRIQ ®) | PDL1 |
| duct | | |
| lung | atezolizumab (TECENTRIQ ®) | PDL1 |
| skin | atezolizumab (TECENTRIQ ®) | PDL1 |
| soft tissue | atezolizumab (TECENTRIQ ®) | PDL1 |
| sarcoma | | |
| Gastrointestinal | avapritinib (AYVAKIT ®) | PDGFRα + c-Kit |
| systemic | avapritinib (AYVAKIT ®) | PDGFRα + c-Kit |
| mastocytosis | | |
| bladder | avelumab (BAVENCIO ®) | PDL1 |
| endocrine and | avelumab (BAVENCIO ®) | PDL1 |
| neuroendocrine | | |
| tumors | | |
| kidney | avelumab (BAVENCIO ®) | PDL1 |
| skin | avelumab (BAVENCIO ®) | PDL1 |
| lymphoma | axicabtagene ciloleucel (YESCARTA ®) | CD19 |
| kidney | axitinib (INLYTA ®) | VEGFR1 + VEGFR2 + VEGFR3 |
| lymphoma | belinostat (BELEODAQ ®) | HDAC |
| Brain | belzutifan (WELIREG ®) | HIF-2A |
| kidney | belzutifan (WELIREGR) | HIF-2A |
| pancreatic | belzutifan (WELIREG ®) | HIF-2A |
| ovarian | bevacizumab (AVASTIN ®) | VEGF-A |
| epithelial, | | |
| fallopian tube, | | |
| and primary | | |
| peritoneal | | |
| Brain | bevacizumab (AVASTIN ®) | VEGF-A |
| Cervical | bevacizumab (AVASTIN ®) | VEGF-A |
| colorectal | bevacizumab (AVASTIN ®) | VEGF-A |
| kidney | bevacizumab (AVASTIN ®) | VEGF-A |
| liver and bile | bevacizumab (AVASTIN ®) | VEGF-A |
| duct | | |
| lung | bevacizumab (AVASTIN ®) | VEGF-A |
| lymphoma | bexarotene (TARGRETIN ®) | RXRs |
| skin | binimetinib (MEKTOVI ®) | MEK1, MEK2 |
| leukemia | blinatumomab (BLINCYTO ®) | CD19, CD3 |
| lymphoma | bortezomib (VELCADE ®) | Proteasome |
| multiple | bortezomib (VELCADE ®) | Proteasome |
| myeloma | | |
| leukemia | bosutinib (BOSULIF ®) | BCR-ABL, SRC |
| lymphoma | brentuximab vedotin (ADCETRIS ®) | CD130, Tubulin |
| leukemia | brexucabtagene autoleucel | CD19 |
| | (TECARTUS ®) | |
| lymphoma | brexucabtagene autoleucel | CD19 |
| | (TECARTUS ®) | |
| lung | brigatinib (ALUNBRIG ®) | ALK, EGFR, FLT3, IGF-1R, ROS1 |
| prostate | cabazitaxel (JEVTANA ®) | Tubulin |
| kidney | cabozantinib-s-malate (CABOMETYX ®) | AXL + RET + ROSI + TYRO3 + |
| | | Tie-2 + TrkB + VEGFR1 + |
| | | VEGFR2 + VEGFR3 + c-Kit + c-Met |
| liver and bile | cabozantinib-s-malate (CABOMETYX ®) | AXL + RET + ROS1 + TYRO3 + |
| duct | | Tie-2 + TrkB + VEGFR1 + |
| | | VEGFR2 + VEGFR3 + c-Kit + c-Met |
| thyroid | cabozantinib-s-malate (COMETRIQ ®) | AXL + RET + ROS1 + TYRO3 + |
| | | Tie-2 + TrkB + VEGFR1 + |
| | | VEGFR2 + VEGFR3 + c-Kit + c-Met |
| lung | capmatinib hydrochloride (TABRECTA ®) | c-Met |
| multiple | carfilzomib (KYPROLIS ®) | protoasome |
| myeloma | | |
| lung | cemiplimab-rwlc (LIBTAYO ®) | PD-1 |
| skin | cemiplimab-rwlc (LIBTAYO ®) | PD-1 |
| lung | ceritinib (ZYKADIA ®) | ALK + IGF-1R + INSR + ROS1 |
| colorectal | cetuximab (ERBITUX ®) | EGFR |
| multiple | ciltacabtagene autoleucel (CARVYKTI ®) | BCMA |
| myeloma | | |
| skin | cobimetinib fumarate (COTELLIC ®) | MEK1, MEK2 |
| lymphoma | copanlisib hydrochloride (ALIQOPA ®) | PI3Kα, PI3Kδ |
| lung | crizotinib (XALKORI ®) | MTH1 |
| lymphoma | crizotinib (XALKORI ®) | MTH1 |
| Myofibroblastic | crizotinib (XALKORI ®) | MTH1 |
| Brain | dabrafenib (TAFINLAR ®) | BRAF, CRAF |
| lung | dabrafenib mesylate (TAFINLAR ®) | BRAF, CRAF |
| skin | dabrafenib mesylate (TAFINLAR ®) | BRAF, CRAF |
| solid tumors | dabrafenib mesylate (TAFINLAR ®) | BRAF, CRAF |
| anywhere in the | | |
| body | | |
| thyroid | dabrafenib mesylate (TAFINLAR ®) | BRAF, CRAF |
| lung | dacomitinib (VIZIMPRO ®) | EGFR + EGFR exon 21 L858R |
| | | mutation + EGFR-Ex19del + HER2 + |
| | | HER4 |
| multiple | daratumumab (DARZALEX ®) | CD38 + Hyaluronic acid |
| myeloma | | |
| multiple | daratumumab and hyaluronidase-fihj | CD38 + Hyaluronic acid |
| myeloma | (DARZALEX FASPRO ®) | |
| prostate | darolutamide (NUBEQAR) | AR |
| leukemia | dasatinib (SPRYCEL ®) | Bcr-Abl + EphA2 + FYN + LCK + |
| | | PDGFRβ + Protein-tyrosine kinases + |
| | | SRC + YES1 + c-Kit |
| neuroblastoma | dinutuximab (UNITUXIN ®) | GD2 |
| endometrial | dostarlimab-gxly (JEMPERLI ®) | PD-1 |
| solid tumors | dostarlimab-gxly (JEMPERLI ®) | PD-1 |
| anywhere in the | | |
| body | | |
| liver and bile | durvalumab (IMFINZI ®) | PDL1 |
| duct | | |
| lung | durvalumab (IMFINZI ®) | PDL1 |
| leukemia | duvelisib (COPIKTRA ®) | PI3Kγ + PI3Kδ |
| Breast | elacestrant dihydrochloride (ORSERDU ®) | Erα/ESR1 |
| multiple | elotuzumab (EMPLICITIR) | SLAMF7 |
| myeloma | | |
| leukemia | enasidenib mesylate (IDHIFAR) | IDH2 |
| colorectal | encorafenib (BRAFTOVI ®) | BRAF, BRAF V600E |
| skin | encorafenib (BRAFTOVI ®) | BRAF V600E, V600K mutation |
| bladder | enfortumab vedotin-ejfv (PADCEVR) | Nectin-4, Tubulins |
| lung | entrectinib (ROZLYTREK ®) | ALK + ROS1 + TRKA + TrkB + |
| | | TrkC |
| solid tumors | entrectinib (ROZLYTREK ®) | ALK + ROS1 + TRKA + TrkB + |
| anywhere in the | | TrkC |
| body | | |
| prostate | enzalutamide (XTANDI ®) | AR |
| bladder | erdafitinib (BALVERSA ®) | FGFR1, FGFR2, FGFR3, FGFR4 |
| lung | erlotinib hydrochloride (TARCEVA ®) | EGFR antagonists, EGFR exon 21 |
| | | L858R mutation inhibitors, EGFR- |
| | | Ex19del inhibitors |
| pancreatic | erlotinib hydrochloride (TARCEVA ®) | EGFR + EGFR exon 21 L858R |
| | | mutation + EGFR-Ex19del |
| Breast | everolimus (AFINITOR ®) | mTORC1, mTORC2 |
| kidney | everolimus (AFINITOR ®) | mTORC1, mTORC2 |
| pancreatic | everolimus (AFINITOR ®) | mTORC1, mTORC2 |
| Breast | exemestane (AROMASIN ®) | Aromatase |
| Breast | fam-trastuzumab deruxtecan-nxki | HER2, TOP1 |
| | (ENHERTU ®) | |
| Gastric | fam-trastuzumab deruxtecan-nxki | HER2, TOP1 |
| | (ENHERTU ®) | |
| lung | fam-trastuzumab deruxtecan-nxki | HER2, TOP1 |
| | (ENHERTU ®) | |
| myelodysplastic | fedratinib hydrochloride (INREBIC ®) | JAK2, FLT3 |
| and | | |
| myeloproliferative | | |
| disorders | | |
| Breast | fulvestrant (FASLODEX ®) | ER |
| liver and bile | futibatinib (LYTGOBI ®) | FGFRs |
| duct | | |
| lung | gefitinib (IRESSA ®) | EGFR + EGFR exon 21 L858R |
| | | mutation + EGFR-Ex19del |
| leukemia | gemtuzumab ozogamicin (MYLOTARG ®) | CD33, DNA |
| leukemia | gilteritinib fumarate (XOSPATA ®) | AXL + FLT3 |
| leukemia | glasdegib maleate (DAURISMO ®) | SMO |
| lymphoma | ibritumomab tiuxetan (ZEVALIN ®) | CD20 |
| leukemia | ibrutinib (IMBRUVICA ®) | BTK |
| lymphoma | ibrutinib (IMBRUVICA ®) | BTK |
| multiple | idecabtagene vicleucel (ABECMA ®) | BCMA |
| myeloma | | |
| leukemia | idelalisib (ZYDELIG ®) | PI3Kδ |
| dermatofibrosar | imatinib mesylate (GLEEVEC ®) | BCR-ABL, PDGFR, C-Kit |
| coma | | |
| protuberans | | |
| Gastrointestinal | imatinib mesylate (GLEEVEC ®) | BCR-ABL, PDGFR, C-Kit |
| leukemia | imatinib mesylate (GLEEVEC ®) | BCR-ABL, PDGFR, C-Kit |
| myelodysplastic | imatinib mesylate (GLEEVEC ®) | BCR-ABL, PDGFR, C-Kit |
| and | | |
| myeloproliferat | | |
| ive disorders | | |
| systemic | imatinib mesylate (GLEEVEC ®) | BCR-ABL, PDGFR, C-Kit |
| mastocytosis | | |
| liver and bile | infigratinib phosphate (TRUSELTIQ ®) | FGFR1 + FGFR2 + FGFR3 + FGFR4 |
| duct | | |
| leukemia | inotuzumab ozogamicin (BESPONSA ®) | CD22 + DNA |
| endocrine and | iobenguane I 131 (AZEDRA ®) | NET |
| neuroendocrine | | |
| tumors | | |
| colorectal | ipilimumab (YERVOY ®) | CTLA4 |
| esophageal | ipilimumab (YERVOY ®) | CTLA4 |
| kidney | ipilimumab (YERVOY ®) | CTLA4 |
| liver and bile | ipilimumab (YERVOY ®) | CTLA4 |
| duct | | |
| lung | ipilimumab (YERVOY ®) | CTLA4 |
| malignant | ipilimumab (YERVOY ®) | CTLA4 |
| mesothelioma | | |
| skin | ipilimumab (YERVOY ®) | CTLA4 |
| multiple | isatuximab-irfc (SARCLISA ®) | CD38 |
| myeloma | | |
| leukemia | ivosidenib (TIBSOVOR) | IDH1 |
| multiple | ixazomib citrate (NINLAROR) | proteasome |
| myeloma | | |
| endocrine and | lanreotide acetate (SOMATULINE | SSTR |
| neuroendocrine | DEPOT ®) | |
| tumors | | |
| Breast | lapatinib ditosylate (TYKERB ®) | EGFR |
| solid tumors | larotrectinib sulfate (VITRAKVI ®) | TRKA + TrkB + TrkC |
| anywhere in the | | |
| body | | |
| endometrial | lenvatinib mesylate (LENVIMA ®) | FGFR1 + FGFR2 + FGFR3 + |
| | | FGFR4 + PDGFRα + RET + |
| | | VEGFR1 + VEGFR2 + VEGFR3 + c-Kit |
| kidney | lenvatinib mesylate (LENVIMA ®) | FGFR1 + FGFR2 + FGFR3 + |
| | | FGFR4 + PDGFRα + RET + |
| | | VEGFR1 + VEGFR2 + VEGFR3 + c-Kit |
| liver and bile | lenvatinib mesylate (LENVIMA ®) | FGFR1 + FGFR2 + FGFR3 + |
| duct | | FGFR4 + PDGFRα + RET + |
| | | VEGFR1 + VEGFR2 + VEGFR3 + c-Kit |
| thyroid | lenvatinib mesylate (LENVIMA ®) | FGFR1 + FGFR2 + FGFR3 + |
| | | FGFR4 + PDGFRα + RET + |
| | | VEGFR1 + VEGFR2 + VEGFR3 + c-Kit |
| Breast | letrozole (FEMARA ®)/Ribociclib | CDK4/ER |
| | Succinate | |
| lymphoma | lisocabtagene maraleucel (BREYANZI ®) | CD19 |
| lymphoma | loncastuximab tesirine-lpyl | CD19 + DNA |
| | (ZYNLONTAR) | |
| lung | lorlatinib (LORBRENA ®) | ALK + ROS1 |
| prostate | lutetium Lu 177 vipivotide tetraxetan | PSMA |
| | (PLUVICTOR) | |
| endocrine and | lutetium Lu 177-dotatate | SSTR2 |
| neuroendocrine | (LUTATHERA ®) | |
| tumors | | |
| Breast | margetuximab-cmkb (MARGENZA ®) | HER2 |
| leukemia | midostaurin (RYDAPT ®) | FLT3 + PDGFR + PKC + Syk + |
| | | VEGFR2 + c-Kit |
| systemic | midostaurin (RYDAPT ®) | FLT3 + PDGFR + PKC + Syk + |
| mastocytosis | | VEGFR2 + c-Kit |
| ovarian | mirvetuximab soravtansine-gynx | FOLR1 + Tubulin |
| epithelial, | (ELAHERE ®) | |
| fallopian tube, | | |
| and primary | | |
| peritoneal | | |
| lung | mobocertinib succinate (EXKIVITY ®) | EGFR exon 20 + HER2 exon 20 |
| lymphoma | mogamulizumab-kpkc (POTELIGEOR) | CCR4 |
| lymphoma | mosunetuzumab-axgb (LUNSUMIO ®) | CD20, CD3 |
| leukemia | moxetumomab pasudotox- | CD22 |
| | tdfk (LUMOXITI ®) | |
| neuroblastoma | naxitamab-gqgk (DANYELZAR) | GD2 |
| lung | necitumumab (PORTRAZZA ®) | EGFR |
| Breast | neratinib maleate (NERLYNX ®) | EGFR, HER2, HER4 |
| leukemia | nilotinib (TASIGNA ®) | Bcr-Abl + CSF-1R + DDR1 + |
| | | PDGFR + c-Kit |
| ovarian | niraparib tosylate monohydrate | PARP1 + PARP2 |
| epithelial, | (ZEJULA ®) | |
| fallopian tube, | | |
| and primary | | |
| peritoneal | | |
| bladder | nivolumab (OPDIVO ®) | PD-1 |
| colorectal | nivolumab (OPDIVO ®) | PD-1 |
| esophageal | nivolumab (OPDIVO ®) | PD-1 |
| kidney | nivolumab (OPDIVO ®) | PD-1 |
| liver and bile | nivolumab (OPDIVO ®) | PD-1 |
| duct | | |
| lung | nivolumab (OPDIVO ®) | PD-1 |
| lymphoma | nivolumab (OPDIVO ®) | PD-1 |
| malignant | nivolumab (OPDIVO ®) | PD-1 |
| mesothelioma | | |
| skin | nivolumab (OPDIVO ®) | PD-1 |
| stomach | nivolumab (OPDIVO ®) | PD-1 |
| (gastric) | | |
| skin | nivolumab and relatlimab-rmbw | LAG3 + PD-1 |
| | (OPDUALAGR) | |
| leukemia | obinutuzumab (GAZYVAR) | CD20 |
| lymphoma | obinutuzumab (GAZYVA ®) | CD20 |
| leukemia | ofatumumab (ARZERRA ®) | CD20 |
| ovarian | olaparib (LYNPARZA ®) | PARP1, PARP2, PARP3 |
| epithelial, | | |
| fallopian tube, | | |
| and primary | | |
| peritoneal | | |
| Breast | olaparib (LYNPARZA ®) | PARP1, PARP2, PARP3 |
| pancreatic | olaparib (LYNPARZA ®) | PARP1, PARP2, PARP3 |
| prostate | olaparib (LYNPARZAR) | PARP1, PARP2, PARP3 |
| leukemia | olutasidenib (REZLIDHIA ®) | IDH1 |
| lung | osimertinib mesylate (TAGRISSO ®) | EGFR + EGFR T790M + EGFR exon |
| | | 21 L858R mutation + EGFR-Ex19del |
| myelodysplasti | pacritinib citrate (VONJO ®) | CSF-1R + FLT3 + IRAK1 + JAK2 |
| c and | | |
| myeloproliferat | | |
| ive disorders | | |
| Breast | palbociclib (IBRANCE ®) | CDK4, CDK6 |
| colorectal | panitumumab (VECTIBIX ®) | EGFR |
| kidney | pazopanib hydrochloride (VOTRIENT ®) | FGFR1 + FGFR3 + Flt3L + ITK + |
| | | LCK + PDGFRα + PDGFRβ + |
| | | VEGFR1 + VEGFR2 + VEGFR3 + c- |
| | | Kit |
| soft tissue | pazopanib hydrochloride (VOTRIENT ®) | FGFR1 + FGFR3 + Flt3L + ITK + |
| sarcoma | | LCK + PDGFRα + PDGFRβ + |
| | | VEGFR1 + VEGFR2 + VEGFR3 + c- |
| | | Kit |
| Breast | pembrolizumab (KEYTRUDA ®) | PD-1 |
| Cervical | pembrolizumab (KEYTRUDA ®) | PD-1 |
| colorectal | pembrolizumab (KEYTRUDA ®) | PD-1 |
| endometrial | pembrolizumab (KEYTRUDA ®) | PD-1 |
| esophageal | pembrolizumab (KEYTRUDA ®) | PD-1 |
| kidney | pembrolizumab (KEYTRUDA ®) | PD-1 |
| liver and bile | pembrolizumab (KEYTRUDA ®) | PD-1 |
| duct | | |
| lung | pembrolizumab (KEYTRUDA ®) | PD-1 |
| lymphoma | pembrolizumab (KEYTRUDA ®) | PD-1 |
| skin | pembrolizumab (KEYTRUDA ®) | PD-1 |
| solid tumors | pembrolizumab (KEYTRUDA ®) | PD-1 |
| anywhere in the | | |
| body | | |
| stomach | pembrolizumab (KEYTRUDA ®) | PD-1 |
| (gastric) | | |
| leukemia | pemigatinib (PEMAZYRER) | FGFR1, FGFR2, FGFR3, FGFR4 |
| liver and bile | pemigatinib (PEMAZYRER) | FGFR1, FGFR2, FGFR3, FGFR4 |
| duct | | |
| lymphoma | pemigatinib (PEMAZYRER) | FGFR1, FGFR2, FGFR3, FGFR4 |
| myelodysplasti | pemigatinib (PEMAZYRER) | FGFR1, FGFR2, FGFR3, FGFR4 |
| c and | | |
| myeloproliferat | | |
| ive disorders | | |
| Breast | pertuzumab (PERJETA ®) | HER2 |
| Breast | pertuzumab, trastuzumab, and | HER2, Hyaluronic acid |
| | hyaluronidase-zzxf (PHESGO ®) | |
| giant cell tumor | pexidartinib hydrochloride (TURALIOR) | CSF-1R + FLT3 + c-Kit |
| lymphoma | pirtobrutinib (JAYPIRCAR) | BTK C481S |
| lymphoma | polatuzumab vedotin-piiq (POLIVY ®) | CD79B + Tubulin |
| leukemia | ponatinib hydrochloride (ICLUSIG ®) | Bcr-Abl + Protein-tyrosine kinases |
| lymphoma | pralatrexate (FOLOTYN ®) | Bcr-Abl + Protein-tyrosine kinases |
| lung | pralsetinib (GAVRETO ®) | RET |
| thyroid | pralsetinib (GAVRETO ®) | RET |
| prostate | radium 223 dichloride (XOFIGO ®) | DNA |
| colorectal | ramucirumab (CYRAMZA ®) | VEGFR2 |
| Gastric | ramucirumab (CYRAMZA ®) | VEGFR2 |
| liver and bile | ramucirumab (CYRAMZA ®) | VEGFR2 |
| duct | | |
| lung | ramucirumab (CYRAMZA ®) | VEGFR2 |
| stomach | ramucirumab (CYRAMZA ®) | VEGFR2 |
| (gastric) | | |
| colorectal | regorafenib (STIVARGA ®) | Abl family, BRAF, BRAF V600E, |
| | | CRAF, CSF-1R, DDR2, EphA2, |
| | | FGFR1, FGFR2, FRK, |
| | | MAPK11,PDGFRa, PDGFRb, |
| | | RET,TRKA, Tie-2, VEGFR1, |
| | | VEGFR2, VEGFR3, c-kit |
| Gastrointestinal | regorafenib (STIVARGA ®) | Abl family, BRAF, BRAF V600E, |
| | | CRAF, CSF-1R, DDR2, EphA2, |
| | | FGFR1, FGFR2, FRK, |
| | | MAPK11,PDGFRa, PDGFRb, |
| | | RET,TRKA, Tie-2, VEGFR1, |
| | | VEGFR2, VEGFR3, c-kit |
| liver and bile | regorafenib (STIVARGA ®) | Abl family, BRAF, BRAF V600E, |
| duct | | CRAF, CSF-1R, DDR2, EphA2, |
| | | FGFR1, FGFR2, FRK, |
| | | MAPK11,PDGFRa, PDGFRb, |
| | | RET,TRKA, Tie-2, VEGFR1, |
| | | VEGFR2, VEGFR3, c-kit |
| skin | retifanlimab-dlwr (ZYNYZ ®) | PD-1 |
| Breast | ribociclib (KISQALI ®) | CDK4, CDK6 |
| gastrointestinal | ripretinib (QINLOCK ®) | EGFR + PDGFRα + c-Kit |
| leukemia | rituximab (RITUXAN ®) | CD20 + Hyaluronic acid |
| lymphoma | rituximab (RITUXAN ®) | CD20 + Hyaluronic acid |
| leukemia | rituximab and hyaluronidase human | CD20 + Hyaluronic acid |
| | (RITUXAN HYCELA ®) | |
| lymphoma | rituximab and hyaluronidase human | CD20 + Hyaluronic acid |
| | (RITUXAN HYCELA ®) | |
| lymphoma | romidepsin (ISTODAX ®) | HDAC |
| ovarian | rucaparib camsylate (RUBRACA ®) | PARP1 + PARP2 + PARP3 |
| epithelial, | | |
| fallopian tube, | | |
| and primary | | |
| peritoneal | | |
| prostate | rucaparib camsylate (RUBRACA ®) | PARP1 + PARP2 + PARP3 |
| myelodysplasti | ruxolitinib phosphate (JAKAFI ®) | JAK1 + JAK2 |
| c and | | |
| myeloproliferat | | |
| ive disorders | | |
| Breast | sacituzumab govitecan-hziy | TOP1 |
| | (TRODELVY ®) | |
| lymphoma | selinexor (XPOVIO ®) | XPO1 |
| multiple | selinexor (XPOVIO ®) | XPO1 |
| myeloma | | |
| lung | selpercatinib (RETEVMOR) | RET |
| solid tumors | selpercatinib (RETEVMO ®) | RET |
| anywhere in the | | |
| body | | |
| thyroid | selpercatinib (RETEVMO ®) | RET |
| plexiform | selumetinib sulfate (KOSELUGO ®) | MEK1 + MEK2 |
| neurofibroma | | |
| soft tissue | sirolimus protein-bound particles | MUT + mTOR |
| sarcoma | (FYARRO ®) | |
| skin | sonidegib (ODOMZO ®) | SMO |
| kidney | sorafenib tosylate (NEXAVAR ®) | BRAF inhibitors, CRAF inhibitors, |
| | | FLT3 + PDGFRβ + RET + |
| | | VEGFR1 + VEGFR2 + VEGFR3 + c-Kit |
| liver and bile | sorafenib tosylate (NEXAVAR ®) | BRAF + CRAF + FLT3 + PDGFRβ + |
| duct | | RET + VEGFR1 + VEGFR2 + |
| | | VEGFR3 + c-Kit |
| thyroid | sorafenib tosylate (NEXAVAR ®) | BRAF + CRAF + FLT3 + PDGFRβ + |
| | | RET + VEGFR1 + VEGFR2 + |
| | | VEGFR3 + c-Kit |
| lung | sotorasib (LUMAKRAS ®) | KRAS G12C |
| Gastrointestinal | sunitinib malate (SUTENT ®) | PDGFR + RTK + VEGFR |
| kidney | sunitinib malate (SUTENT ®) | PDGFR + RTK + VEGFR |
| pancreatic | sunitinib malate (SUTENT ®) | PDGFR + RTK + VEGFR |
| lymphoma | tafasitamab-cxix (MONJUVI ®) | CD19 |
| leukemia | tagraxofusp-erzs (ELZONRIS ®) | CD123 |
| Breast | talazoparib tosylate (TALZENNA ®) | PARP1, PARP2 |
| Breast | tamoxifen citrate (SOLTAMOX ®) | ER |
| lymphoma | tazemetostat hydrobromide | EZH2 |
| | (TAZVERIK ®) | |
| soft tissue | tazemetostat hydrobromide | EZH2 |
| sarcoma | (TAZVERIK ®) | |
| skin | tebentafusp-tebn (KIMMTRAK ®) | CD3 + gp100 |
| multiple | teclistamab-cqyv (TECVAYLI ®) | CD3 + gp100 |
| myeloma | | |
| kidney | temsirolimus (TORISEL ®) | CD3 + gp100 |
| lung | tepotinib hydrochloride (TEPMETKO ®) | MET |
| leukemia | tisagenlecleucel (KYMRIAH ®) | CD19 |
| lymphoma | tisagenlecleucel (KYMRIAH ®) | CD19 |
| Cervical | tisotumab vedotin-tftv (TIVDAK ®) | Tubulin, tissue factor |
| kidney | tivozanib hydrochloride (FOTIVDA ®) | (VEGFR)-1, VEGFR-2 and VEGFR- |
| | | 3, c-kit, and PDGFRβ |
| Breast | toremifene (FARESTON ®) | ER |
| Brain | trametinib (MEKINIST ®) | MEK1 and MEK2 |
| lung | trametinib dimethyl sulfoxide | MEK1 and MEK2 |
| | (MEKINIST ®) | |
| skin | trametinib dimethyl sulfoxide | MEK1 and MEK2 |
| | (MEKINIST ®) | |
| solid tumors | trametinib dimethyl sulfoxide | MEK1 and MEK2 |
| anywhere in the | (MEKINIST ®) | |
| body | | |
| thyroid | trametinib dimethyl sulfoxide | MEK1 and MEK2 |
| | (MEKINIST ®) | |
| Breast | trastuzumab (HERCEPTIN ®) | HER2 |
| esophageal | trastuzumab (HERCEPTIN ®) | HER2 |
| stomach | trastuzumab (HERCEPTIN ®) | HER2 |
| (gastric) | | |
| liver and bile | tremelimumab-actl (IMJUDO ®) | CTLA-4, CD80, and CD86 |
| duct | | |
| lung | tremelimumab-actl (IMJUDO ®) | CTLA-4, CD80, and CD86 |
| Breast | tucatinib (TUKYSA ®) | HER2 |
| colorectal | tucatinib (TUKYSA ®) | HER2 |
| thyroid | vandetanib (CAPRELSA ®) | VEGFR-2, EGFR, RET |
| skin | vemurafenib (ZELBORAFR) | BRAF, BRAF V600E, CRAF, ARAF, |
| | | SRMS, ACK1, MAP4K5, FGR |
| leukemia | venetoclax (VENCLEXTA ®) | BCL-2 |
| lymphoma | venetoclax (VENCLEXTA ®) | BCL-2 |
| lymphoma | vorinostat (ZOLINZA ®) | HDAC1, HDAC2 and HDAC3 (Class |
| | | I) and HDAC6 (Class II) |
| leukemia | zanubrutinib (BRUKINSA ®) | BTK |
| lymphoma | zanubrutinib (BRUKINSA ®) | BTK |
| colorectal | ziv-aflibercept (ZALTRAP ®) | PGF, VEGF-A |

In some embodiments, the methods described herein can be used to treat patients by (i) detecting one or more mutations in the one or more target genes listed in Table 10; and (ii) administering the corresponding one or more drugs listed in Table 10. In some embodiments, these therapies may be used alone or in combination with other therapies to treat a disease.

In certain embodiments, the immune checkpoint molecule is a co-stimulatory molecule that amplifies a signal involved in a T cell response to an antigen. For example, CD28 is a co-stimulatory receptor expressed on T cells. When a T cell binds to antigen through its T cell receptor, CD28 binds to CD80 (aka B7.1) or CD86 (aka B7.2) on antigen-presenting cells to amplify T cell receptor signaling and promote T cell activation. Because CD28 binds to the same ligands (CD80 and CD86) as CTLA4, CTLA4 is able to counteract or regulate the co-stimulatory signaling mediated by CD28. In certain embodiments, the immune checkpoint molecule is a co-stimulatory molecule selected from CD28, inducible T cell co-stimulator (ICOS), CD137, OX40, or CD27. In other embodiments, the immune checkpoint molecule is a ligand of a co-stimulatory molecule, including, for example, CD80, CD86, B7RP1, B7-H3, B7-H4, CD137L, OX40L, or CD70.

Agonists that target these co-stimulatory checkpoint molecules can be used to enhance antigen-specific T cell responses against certain cancers. Accordingly, in certain embodiments, the immunotherapy or immunotherapeutic agent is an agonist of a co-stimulatory checkpoint molecule. In certain embodiments, the agonist of the co-stimulatory checkpoint molecule is an agonist antibody and preferably is a monoclonal antibody. In certain embodiments, the agonist antibody or monoclonal antibody is an anti-CD28 antibody. In other embodiments, the agonist antibody or monoclonal antibody is an anti-ICOS, anti-CD137, anti-OX40, or anti-CD27 antibody. In other embodiments, the agonist antibody or monoclonal antibody is an anti-CD80, anti-CD86, anti-B7RP1, anti-B7-H3, anti-B7-H4, anti-CD137L, anti-OX40L, or anti-CD70 antibody.

These methods provided herein provide a deeper understanding of the changes in DNA and proteins that cause cancer, allowing the identification of biomarkers and design of treatments that target these proteins. In some embodiments, the biomarker may include an epigenetic signature, such as a methylation state, methylation score and/or DNA fragmentation pattern/score. In some embodiments, the epigenetic signature can be determined for one or more regions that include, but not limited to, transcription start sites, promoter regions, CTCF binding regions and regulatory protein binding regions. In some embodiments, the epigenetic signature is determined for one or more regions that include, but not limited to, transcription start sites, promoter regions, intergenic regions and/or intronic regions that are associated with at least one or more genes listed in Table 10. Such treatments may include small-molecule drugs or monoclonal antibodies. The methods may also improve biomarker testing in individuals suffering from disease and help determine if the individual is a candidate for a certain drug or combination of drugs based on the presence or absence of the biomarker. Additionally, the methods can improve identification of mutations that contribute to the development of resistance to targeted therapy. Consequently, the analysis techniques may reduce unnecessary or untimely therapeutic interventions, patient suffering, and patient mortality.

In some embodiments, the methods and systems disclosed herein may be used to identify customized or targeted therapies to treat a given disease or condition in patients based on the classification of a nucleic acid variant as being of somatic or germline origin. In certain embodiments, the status of a nucleic acid variant from a sample from a subject as being of somatic or germline origin may be compared with a database of comparator results from a reference population to identify customized or targeted therapies for that subject. Typically, the reference population includes patients with the same cancer or disease type as the subject and/or patients who are receiving, or who have received, the same therapy as the subject. A customized or targeted therapy (or therapies) may be identified when the nucleic variant and the comparator results satisfy certain classification criteria (e.g., are a substantial or an approximate match).

In certain embodiments, the customized therapies described herein are typically administered parenterally (e.g., intravenously or subcutaneously). Pharmaceutical compositions containing an immunotherapeutic agent are typically administered intravenously. Certain therapeutic agents are administered orally. However, customized therapies (e.g., immunotherapeutic agents, etc.) may also be administered by any method known in the art, for example, buccal, sublingual, rectal, vaginal, intraurethral, topical, intraocular, intranasal, and/or intraauricular, which administration may include tablets, capsules, granules, aqueous suspensions, gels, sprays, suppositories, salves, ointments, or the like.

In some embodiments, therapy is customized based on the status of a nucleic acid variant as being of somatic or germline origin. In some embodiments, determination of the levels of particular cell types, e.g., immune cell types, including rare immune cell types, facilitates selection of appropriate treatment.

The present methods can be used to diagnose the presence of a condition, e.g., cancer or precancer, in a subject, to characterize a condition (such as to determine a cancer stage or heterogeneity of a cancer), to monitor a subject's response to receiving a treatment for a condition (such as a response to a chemotherapeutic or immunotherapeutic), assess prognosis of a subject (such as to predict a survival outcome in a subject having a cancer), to determine a subject's risk of developing a condition, to predict a subsequent course of a condition in a subject, to determine metastasis or recurrence of a cancer in a subject (or a risk of cancer metastasis or recurrence), and/or to monitor a subject's health as part of a preventative health monitoring program (such as to determine whether and/or when a subject is in need of further diagnostic screening). The methods according to the present disclosure can also be useful in predicting a subject's response to a particular treatment option. Successful treatment options may increase the amount of copy number variation, rare mutations, and/or cancer-related epigenetic signatures (such as hypermethylated regions or hypomethylated regions) detected in a subject's blood (such as in DNA isolated from a buffy coat sample or any other sample comprising cells, such as a blood sample (e.g., a whole blood sample, a buffy coat sample, a leukapheresis sample, or a PBMC sample) from the subject) if the treatment is successful as more cancer cells may die and shed DNA, or if a successful treatment results in an increase or decrease in the quantity of a specific immune cell type in the blood and an unsuccessful treatment results in no change. In other examples, this may not occur. In another example, certain treatment options may be correlated with genetic profiles of cancers over time. This correlation may be useful in selecting a therapy for a subject. In some embodiments, determination of the metastasis site facilitates selection of appropriate treatment.

Thus, in some embodiments, quantities of each of one or more of a particular genetic and/or epigenetic signature (e.g., quantities of fusions, indels, SNPs, CNVs, and/or rare mutations, and/or cancer-related epigenetic signatures (such as specific (e.g., DMRs) or global hypermethylated or hypomethylated regions, and/or fragmentation variable regions)) in DNA from a subject's blood (such as in DNA (e.g., cfDNA) isolated from a blood sample (e.g., a whole blood sample) from the subject)) are determined based on sequencing and analysis. In some embodiments, quantities of each of a plurality of cell types, such as immune cell types, are determined based on sequencing and analysis (such as determination of epigenetic and/or genomic signatures) of DNA isolated from at least one sample comprising cells (such as blood sample (e.g., a whole blood sample, a buffy coat sample, a leukapheresis sample, or a PBMC sample) from a subject. The plurality of immune cell types can include, but is not limited to, macrophages (including M1 macrophages and M2 macrophages), activated B cells (including regulatory B cells, memory B cells and plasma cells); T cell subsets, such as central memory T cells, naïve-like T cells, and activated T cells (including cytotoxic T cells, regulatory T cells (Tregs), CD4 effector memory T cells, CD4 central memory T cells, CD8 effector memory T cells, and CD8 central memory T cells); immature myeloid cells (including myeloid-derived suppressor cells (MDSCs), low-density neutrophils, immature neutrophils, and immature granulocytes); and natural killer (NK) cells. As disclosed herein, differences in levels and/or presence of particular genetic and/or epigenetic signatures in DNA isolated from blood samples from a subject can be used to quantify cell types, such as immune cell types, within the sample. Thus, a comparison of one or more genetic and/or epigenetic signatures in DNA isolated from blood samples collected from a subject at two or more time points can be used to monitor changes in the one or more signatures and/or the one or more cell type quantities in the subject under different conditions (such as prior to and after a treatment), or over time (e.g., as part of a preventative health monitoring program).

In some embodiments, therapy is customized based on the status of a detected nucleic acid variant as being of somatic or germline origin. In some embodiments, essentially any cancer therapy (e.g., surgical therapy, radiation therapy, chemotherapy, and/or the like) may be included as part of these methods. Typically, customized therapies include at least one immunotherapy (or an immunotherapeutic agent). Immunotherapy refers generally to methods of enhancing an immune response against a given cancer type. In certain embodiments, immunotherapy refers to methods of enhancing a T cell response against a tumor or cancer.

Therapies can function by helping the immune system destroy cancer cells. For example, certain targeted therapies may mark cancer cells for the immune system to destroy them. Other targeted therapies may support the immune system to work more effectively against cancer. Yet other therapies may stop cancer cells from growing, for example, by interfering with cancer cell surface markers preventing them from dividing. Additionally, therapies can inhibit signals that promote angiogenesis. Such angiogenesis inhibitors prevent blood supply into the tumor thereby, preventing tumor growth. Other targeted therapies can deliver toxic substances to the tumor. Examples include monoclonal antibodies combined with toxins, chemotherapy, or radiation. Some targeted therapies induce apoptosis or deplete cancer of hormones.

In certain embodiments, the status of a nucleic acid variant from a sample from a subject as being of somatic or germline origin may be compared with a database of comparator results from a reference population to identify customized or targeted therapies for that subject. Typically, the reference population includes patients with the same cancer or disease type as the subject and/or patients who are receiving, or who have received, the same therapy as the subject. A customized or targeted therapy (or therapies) may be identified when the nucleic variant and the comparator results satisfy certain classification criteria (e.g., are a substantial or an approximate match).

The disclosed methods can include evaluating (such as quantifying) and/or interpreting at least one cell material released from a potential metastasis site (such as at least one cell material in a sample from a subject) and/or cell types that contribute to DNA, such as cfDNA, in one or more samples collected from a subject at one or more timepoints in comparison to a selected baseline value or reference standard (or a selected set of baseline values or reference standards). A baseline value or reference standard may be a presence or level of at least one cell material and/or a quantity of cell types measured in one or more samples (such as an average quantity or range of quantities of cell types present in at least two samples) collected from the subject at one or more time points, such as prior to receiving a treatment, prior to diagnosis of a condition (such as a cancer), or as part of a preventative health monitoring program. A baseline value or reference standard may be a presence or level of at least one cell material and/or a quantity of cell types measured with respect to one or more samples (such as an average quantity or range of quantities of cell types present in at least two samples) collected at one or more timepoints from one or more subjects that do not have the condition (such as a healthy subject that does not have a cancer), one or more subjects that responded favorably to the treatment, or one or more subjects that have not received the treatment. In certain embodiments, the baseline value or reference standard utilized is a standard or profile derived from a single reference subject. In other embodiments, the baseline value or reference standard utilized is a standard or profile derived from averaged data from multiple reference subjects. The reference standard, in various embodiments, can be a single value, a mean, an average, a numerical mean or range of numerical means, a numerical pattern, or a graphical pattern created from the cell type quantity data derived from a single reference subject or from multiple reference subjects. Selection of the particular baseline values or reference standards, or selection of the one or more reference subjects, depends upon the use to which the methods described herein are to be put by, for example, a research scientist or a clinician (such as a physician).

The disclosed methods can include evaluating (such as quantifying) and/or interpreting one or more genetic and/or epigenetic signatures, and/or one or more cell types (such as one or more immune cell types), present in one or more samples (e.g., in DNA, such as cfDNA, from a blood sample (e.g., a whole blood sample, a buffy coat sample, a leukapheresis sample, or a PBMC sample)) collected from a subject at one or more timepoints in comparison to a selected baseline value or reference standard (or a selected set of baseline values or reference standards). A baseline value or reference standard may be a quantity of copy number variation, rare mutations, cancer-related epigenetic signatures (such as hypermethylated regions or hypomethylated regions), and/or cell types measured in one or more samples (such as an average quantity or range of quantities of such signatures present in at least two samples) collected from the subject at one or more time points, such as prior to receiving a treatment, prior to diagnosis of a condition (such as a cancer), or as part of a preventative health monitoring program. A baseline value or reference standard may be a quantity of, e.g., copy number variation, rare mutations, cancer-related epigenetic signatures (such as hypermethylated regions or hypomethylated regions), and/or cell types measured in one or more samples (such as an average quantity or range of quantities of such signatures and/or cell types present in at least two samples) collected at one or more timepoints from one or more subjects that do not have the condition (such as a healthy subject that does not have a cancer), one or more subjects that responded favorably to the treatment, or one or more subjects that have not received the treatment.

In certain embodiments, the baseline value or reference standard utilized is a standard or profile derived from a single reference subject. In other embodiments, the baseline value or reference standard utilized is a standard or profile derived from averaged data from multiple reference subjects. The reference standard, in various embodiments, can be a single value, a mean, an average, a numerical mean or range of numerical means, a numerical pattern, or a graphical pattern created from the genetic and/or epigenetic signature quantity data derived from a single reference subject or from multiple reference subjects. Selection of the particular baseline values or reference standards, or selection of the one or more reference subjects, depends upon the use to which the methods described herein are to be put by, for example, a research scientist or a clinician (such as a physician).

In some embodiments, one or more samples comprising cells (such as a buffy coat sample or any other sample comprising cells, such as a blood sample (e.g., a whole blood sample, a leukapheresis sample, or a PBMC sample) may be collected from a subject at two or more timepoints, to assess changes in cell types (such as changes in quantities of cell types) between the two timepoints. By monitoring cell types and identifying differences between cell types in samples collected from a subject at two or more timepoints, the present methods can be used, for example, to determine the presence or absence of a condition (such as a cancer), a response of the subject to a treatment, one or more characteristic of a condition (such as a cancer stage) in the subject, recurrence of a condition (such as a cancer), and/or a subject's risk of developing a condition (such as a cancer). Thus, in some embodiments, methods are provided wherein quantities of cell types present in at least one sample (such as at least one whole blood sample, buffy coat sample, leukapheresis sample, or PBMC sample) collected from a subject at one or more timepoints (such as prior to receiving a treatment) are compared to quantities of cell types present in at least one sample collected from the subject at one or more different time points (such as after receiving the treatment). The disclosed methods can allow for patient-specific monitoring, such that, for example, differences in cell type quantities between samples collected from the subject at different timepoints may indicate changes (such as presence or absence of a condition, response to a treatment, a prognosis, or the like) that are significant with respect to the subject but may yet fall within a normal range of a general healthy population.

In some embodiments, methods are provided for monitoring a response (such as a change in disease state, such as a presence or absence of a metastasis in a subject, such as measured by assessing a presence or level of at least one cell material released from a potential metastasis site in a sample from the subject) of a subject to a treatment (such as a chemotherapy or an immunotherapy). In certain embodiments, one or more samples is collected from the subject at at least 1-10, at least 1-5, at least 2-5, or at least 1, at least 2, least 3, at least 4, at least 5, at least 6, at least 7, at least 8, at least 9, at least 10, at least 15, or at least 20 time points prior to the subject receiving the treatment. In certain embodiments, one or more samples is collected from the subject at at least 1-10, at least 1-5, at least 2-5, or at least 1, at least 2, least 3, at least 4, at least 5, at least 6, at least 7, at least 8, at least 9, at least 10, at least 15, or at least 20 time points after the subject has received the treatment. Sample collection from a subject can be ongoing during and/or after treatment to monitor the subject's response to the treatment.

In some embodiments, samples are not collected from a subject prior to diagnosis of a condition (such as a cancer) or prior to receiving a treatment. In such embodiments, wherein the response of a subject to a treatment or the course or stage of a condition (such as a cancer) in the subject is being monitored over time, genetic and/or epigenetic signatures, and/or cell types are compared between samples taken at at least 2-10, at least 2-5, at least 3-6, or at least 2, such as at least 3, at least 4, at least 5, at least 6, at least 7, at least 8, at least 9, at least 10, at least 15, or at least 20 time points collected after the subject has been diagnosed and/or after the subject has received the treatment. Sample collection from a subject can be ongoing during and/or after treatment to monitor the subject's response to the treatment.

In some embodiments of the disclosed methods, one or more samples is collected from a subject at least once per year, such as about 1-12 times or about 2-6 times, such as about 1, 2, 3, 4, 5, 6, 7, 8, 9, 10, 11, or 12 times per year. In other embodiments, one or more samples is collected from the subject less than once per year, such as about once every 13, 14, 15, 16, 17, 18, 19, 20, 21, 22, 23, or 24 months. In some embodiments, one or more samples is collected from the subject about once every 1-5 years or about once every 1-2 years, such as about every 1, 1.5, 2, 2.5, 3, 3.5, 4, 4.5, or 5 years.

In other embodiments of the disclosed methods, one or more samples (such as one or more whole blood, buffy coat, leukapheresis, or PBMC samples) are collected from a subject at least once per week, such as on 1-4 days, 1-2 days, or on 1, 2, 3, 4, 5, 6, or 7 days per week. In certain embodiments, one or more samples are collected from the subject at least once per month, such as 1-15 times, 1-10 times, 2-5 times, or 1, 2, 3, 4, 5, 6, 7, 8, 9, 10, 11, 12, 13, 14, or 15 times per month. In other embodiments, one or more samples is collected from the subject every month, every 2 months, every 3 months, every 4 months, every 5 months, every 6 months, every 7 months, every 8 months, every 9 months, every 10 months, every 11 months, or every 12 months. In some embodiments, one or more samples is collected from the subject at least once per day, such as 1, 2, 3, 4, 5, or 6 times per day. Selection of the one or more sample collection timepoints (e.g., the frequency of sample collection), or of the number of samples to be collected at each timepoint, depends upon the use to which the methods described herein are to be put by, for example, a research scientist or a clinician (such as a physician).

In certain embodiments, the customized therapies described herein are typically administered parenterally (e.g., intravenously or subcutaneously). Pharmaceutical compositions containing an immunotherapeutic agent are typically administered intravenously. Certain therapeutic agents are administered orally. However, customized therapies (e.g., immunotherapeutic agents, etc.) may also be administered by methods such as, for example, buccal, sublingual, rectal, vaginal, intraurethral, topical, intraocular, intranasal, and/or intraauricular, which administration may include tablets, capsules, granules, aqueous suspensions, gels, sprays, suppositories, salves, ointments, or the like.

Therapeutic options for treating specific genetic-based diseases, disorders, or conditions, other than cancer, are generally well-known to those of ordinary skill in the art and will be apparent given the particular disease, disorder, or condition under consideration.

### N. Table of Sequences

The following table shows exemplary sequences provided herein.

| SEQ ID NO | |
|---|---|
| and | |
| description | Sequence |
| SEQ ID NO: 1 | MNSNKDKIKVIKVFEAFAGIGSQFKALKNIARSKNWEIQHSGMVEWFVDAIVSYVAIHSKN |
| A variant of | FNPKIERLDRDILSISNDSKMPISEYGIKKINNTIKASYLNYAKKHFNNLFDIKKVNKDNF |
| M.MpeI | PKNIDIFTYSFPCQDLSVQGLQKGIDKELNTRSGLLWEIERILEEIKNSFSKEEMPKYLLM |
| | ENVKNLLSHKNKKNYNTWLKQLEKFGYKSKTYLLNSKNFDNCQNRERVFCLSIRDDYLEKT |
| | GFKFKELEKVKNPPKKIKDILVDSSNYKYLNLNKYETTTFRETKSNIISRPLKNYTTFNSE |
| | NYVYNINGIGPTLTASGANSRIKIETQQGVRYLTPLECFKYMQFDVNDFKKVQSTNLISEN |
| | KMIYIAGKSIPVKILEAIFNTLEFVNNEELE |
| SEQ ID NO: 2 | MNSNKDKIKVIKVFEAFAGIGSQFKALKNIARSKNWEIQHSGMVEWFVDAIVSYVAIHSKN |
| A variant of | FNPKIERLDRDILSISNDSKMPISEYGIKKINNTIKASYLNYAKKHFNNLFDIKKVNKDNF |
| M.MpeI | PKNIDIFTYSFPCQDLSVQGLQKGIDKELNTRSGLLWEIERILEEIKNSFSKEEMPKYLLM |
| | ENVKNLLSHKNKKNYNTWLKQLEKFGYKSKTYLLNSKNFDNCQNRERVFCLSIRDDYLEKT |
| | GFKFKELEKVKNPPKKIKDILVDSSNYKYLNLNKYETTTFRETKSNIISRPLKNYTTFNSE |
| | NYVYNINGIGPTLTASGANSRIKIETQQGVRYLTPLECFKYMQFDVNDFKKVQSTNLISEN |
| | KMIYIAGRSIPVKILEAIFNTLEFVNNEELE |
| SEQ ID NO: 3 | GGSQSQNGKCEGCNPDKDEAPYYTHLGAGPDVAAIRTLMEERYGEKGKAIRIEKVIYTGKE |
| TETv | GKSSQGCPIAKWVYRRSSEEEKLLCLVRVRPNHTCETAVMVIAIMLWDGIPKLLASELYSE |
| | LTDILGKCGICTNRRCSQNETRNCCCQGENPETCGASFSFGCSWSMYYNGCKFARSKKPRK |
| | FRLHGAEPKEEERLGSHLQNLATVIAPIYKKLAPDAYNNQVEFEHQAPDCCLGLKEGRPFS |
| | GVTACLDFSAHSHRDQQNMPNGSTVVVTLNREDNREVGAKPEDEQFHVLPMYIIAPEDEFG |
| | STEGQEKKIRMGSIEVLQSFRRRRVIRIGELPKSCEVSGQDAAAVQEIEYWSDSEHNFQDP |
| | CIGGVAIAPTHGSILIECAKCEVHATTKVNDPDRNHPTRISLVLYRHKNLFLPKHCLALWE |
| | AKMAEKARKEEECGKNGSDHVSQKNHGKQEKREPTGPQEPSYLRFIQSLAENTGSVTTDST |
| | VTTSPYAFTQVTGPYNTFV |
| SEQ ID NO: 4 | QSQNGKCEGCNPDKDEAPYYTHLGAGPDVAAIRTLMEERYGEKGKAIRIEKVIYTGKEGKS |
| TETcd | SQGCPIAKWVYRRSSEEEKLLCLVRVRPNHTCETAVMVIAIMLWDGIPKLLASELYSELTD |
| | ILGKCGICTNRRCSQNETRNCCCQGENPETCGASFSFGCSWSMYYNGCKFARSKKPRKFRL |
| | HGAEPKEEERLGSHLQNLATVIAPIYKKLAPDAYNNQVEFEHQAPDCCLGLKEGRPFSGVT |
| | ACLDFSAHSHRDQQNMPNGSTVVVTLNREDNREVGAKPEDEQFHVLPMYIIAPEDEFGSTE |
| | GQEKKIRMGSIEVLQSFRRRRVIRIGELPKSCKKKAEPKKAKTKKAARKRSSLENCSSRTE |
| | KGKSSSHTKLMENASHMKQMTAQPQLSGPVIRQPPTLQRHLQQGQRPQQPQPPQPQPQTTP |
| | QPQPQPQHIMPGNSQSVGSHCSGSTSVYTRQPTPHSPYPSSAHTSDIYGDTNHVNFYPTSS |
| | HASGSYLNPSNYMNPYLGLLNQNNQYAPFPYNGSVPVDNGSPFLGSYSPQAQSRDLHRYPN |
| | QDHLTNQNLPPIHTLHQQTFGDSPSKYLSYGNQNMQRDAFTTNSTLKPNVHHLATFSPYPT |
| | PKMDSHEMGAASRSPYSHPHTDYKTSEHHLPSHTIYSYTAAASGSSSSHAFHNKENDNIAN |
| | GLSRVLPGFNHDRTASAQELLYSLTGSSQEKQPEVSGQDAAAVQEIEYWSDSEHNFQDPCI |
| | GGVAIAPTHGSILIECAKCEVHATTKVNDPDRNHPTRISLVLYRHKNLFLPKHCLALWEAK |
| | MAEKARKEEECGKNGSDHVSQKNHGKQEKREPTGPQEPSYLRFIQSLAENTGSVTTDSTVT |
| | TSPYAFTQVTGPYNTFV |
| SEQ ID NO: 5 | MDYKDDDDKHMGGSDFPSCRCVEQIIEKDEGPFYTHLGAGPNVAAIREIMEERFGQKGKA |
| wild type | IRIERVIYTGKEGKSSQGCPIAKWVVRRSSSEEKLLCLVRERAGHTCEAAVIVILILVWE |
| TET2 | GIPLSLADKLYSELTETLRKYGTLTNRRCALNEERTCACQGLDPETCGASFSFGCSWSMY |
| catalytic | YNGCKFARSKIPRKFKLLGDDPKEEEKLESHLQNLSTLMAPTYKKLAPDAYNNQIEYEHR |
| domain | APECRLGLKEGRPFSGVTACLDFCAHAHRDLHNMQNGSTLVCTLTREDNREFGGKPEDEQ |
| | LHVLPLYKVSDVDEFGSVEAQEEKKRSGAIQVLSSFRRKVRMLAEPVKTCRQRKLEAKKA |
| | AAEKLSGGGGSGGGGSGGGGSDEVWSDSEQSFLDPDIGGVAVAPTHGSILIECAKRELHA |
| | TTPLKNPNRNHPTRISLVFYQHKSMNEPKHGLALWEAKMAEKAREKEEECEKYG |
| SEQ ID NO: 6 | MDYKDDDDKHMGGSDFPSCRCVEQIIEKDEGPFYTHLGAGPNVAAIREIMEERFGQKGKA |
| TET2 V1900A | IRIERVIYTGKEGKSSQGCPIAKWVVRRSSSEEKLLCLVRERAGHTCEAAVIVILILVWE |
| catalytic | GIPLSLADKLYSELTETLRKYGTLTNRRCALNEERTCACQGLDPETCGASFSFGCSWSMY |
| domain | YNGCKFARSKIPRKFKLLGDDPKEEEKLESHLQNLSTLMAPTYKKLAPDAYNNQIEYEHR |
| | APECRLGLKEGRPFSGVTACLDFCAHAHRDLHNMQNGSTLVCTLTREDNREFGGKPEDEQ |
| | LHVLPLYKVSDVDEFGSVEAQEEKKRSGAIQVLSSFRRKVRMLAEPVKTCRQRKLEAKKA |
| | AAEKLSGGGGSGGGGSGGGGSDEVWSDSEQSFLDPDIGGVAVAPTHGSILIECAKRELHA |
| | TTPLKNPNRNHPTRISLAFYQHKSMNEPKHGLALWEAKMAEKAREKEEECEKYG |
| SEQ ID NO: 7 | MDYKDDDDKHMGGSDFPSCRCVEQIIEKDEGPFYTHLGAGPNVAAIREIMEERFGQKGKA |
| TET2 V1900C | IRIERVIYTGKEGKSSQGCPIAKWVVRRSSSEEKLLCLVRERAGHTCEAAVIVILILVWE |
| catalytic | GIPLSLADKLYSELTETLRKYGTLTNRRCALNEERTCACQGLDPETCGASFSFGCSWSMY |
| domain | YNGCKFARSKIPRKFKLLGDDPKEEEKLESHLQNLSTLMAPTYKKLAPDAYNNQIEYEHR |
| | APECRLGLKEGRPFSGVTACLDFCAHAHRDLHNMQNGSTLVCTLTREDNREFGGKPEDEQ |
| | LHVLPLYKVSDVDEFGSVEAQEEKKRSGAIQVLSSFRRKVRMLAEPVKTCRQRKLEAKKA |
| | AAEKLSGGGGSGGGGSGGGGSDEVWSDSEQSFLDPDIGGVAVAPTHGSILIECAKRELHA |
| | TTPLKNPNRNHPTRISLCFYQHKSMNEPKHGLALWEAKMAEKAREKEEECEKYG |
| SEQ ID NO: 8 | MDYKDDDDKHMGGSDFPSCRCVEQIIEKDEGPFYTHLGAGPNVAAIREIMEERFGQKGKA |
| TET2 V1900G | IRIERVIYTGKEGKSSQGCPIAKWVVRRSSSEEKLLCLVRERAGHTCEAAVIVILILVWE |
| catalytic | GIPLSLADKLYSELTETLRKYGTLTNRRCALNEERTCACQGLDPETCGASESFGCSWSMY |
| domain | YNGCKFARSKIPRKFKLLGDDPKEEEKLESHLQNLSTLMAPTYKKLAPDAYNNQIEYEHR |
| | APECRLGLKEGRPFSGVTACLDFCAHAHRDLHNMQNGSTLVCTLTREDNREFGGKPEDEQ |
| | LHVLPLYKVSDVDEFGSVEAQEEKKRSGAIQVLSSFRRKVRMLAEPVKTCRQRKLEAKKA |
| | AAEKLSGGGGSGGGGSGGGGSDEVWSDSEQSFLDPDIGGVAVAPTHGSILIECAKRELHA |
| | TTPLKNPNRNHPTRISLGFYQHKSMNEPKHGLALWEAKMAEKAREKEEECEKYG |
| SEQ ID NO: 9 | MDYKDDDDKHMGGSDFPSCRCVEQIIEKDEGPFYTHLGAGPNVAAIREIMEERFGQKGKA |
| TET2 V1900I | IRIERVIYTGKEGKSSQGCPIAKWVVRRSSSEEKLLCLVRERAGHTCEAAVIVILILVWE |
| catalytic | GIPLSLADKLYSELTETLRKYGTLTNRRCALNEERTCACQGLDPETCGASESFGCSWSMY |
| domain | YNGCKFARSKIPRKFKLLGDDPKEEEKLESHLQNLSTLMAPTYKKLAPDAYNNQIEYEHR |
| | APECRLGLKEGRPFSGVTACLDFCAHAHRDLHNMQNGSTLVCTLTREDNREFGGKPEDEQ |
| | LHVLPLYKVSDVDEFGSVEAQEEKKRSGAIQVLSSFRRKVRMLAEPVKTCRQRKLEAKKA |
| | AAEKLSGGGGSGGGGSGGGGSDEVWSDSEQSFLDPDIGGVAVAPTHGSILIECAKRELHA |
| | TTPLKNPNRNHPTRISLIFYQHKSMNEPKHGLALWEAKMAEKAREKEEECEKYG |
| SEQ ID NO: | MDYKDDDDKHMGGSDFPSCRCVEQIIEKDEGPFYTHLGAGPNVAAIREIMEERFGQKGKA |
| 10 | IRIERVIYTGKEGKSSQGCPIAKWVVRRSSSEEKLLCLVRERAGHTCEAAVIVILILVWE |
| TET2 V1900P | GIPLSLADKLYSELTETLRKYGTLTNRRCALNEERTCACQGLDPETCGASESFGCSWSMY |
| catalytic | YNGCKFARSKIPRKFKLLGDDPKEEEKLESHLQNLSTLMAPTYKKLAPDAYNNQIEYEHR |
| domain | APECRLGLKEGRPFSGVTACLDFCAHAHRDLHNMQNGSTLVCTLTREDNREFGGKPEDEQ |
| | LHVLPLYKVSDVDEFGSVEAQEEKKRSGAIQVLSSFRRKVRMLAEPVKTCRQRKLEAKKA |
| | AAEKLSGGGGSGGGGSGGGGSDEVWSDSEQSFLDPDIGGVAVAPTHGSILIECAKRELHA |
| | TTPLKNPNRNHPTRISLPFYQHKSMNEPKHGLALWEAKMAEKAREKEEECEKYG |
| SEQ ID NO: | MDYKDDDDKHMGGSDFPSCRCVEQIIEKDEGPFYTHLGAGPNVAAIREIMEERFGQ |
| 11 | KGKAIRIERVIYTGKEGKSSQGCPIAKWVVRRSSSEEKLLCLVRERAGHTCEAAVI |
| TET2-CS- | VILILVWEGIPLSLADKLYSELTETLRKYGTLTNRRCALNEERTCACQGLDPETCG |
| T1372S | ASFSFGCSWSMYYNGCKFARSKIPRKFKLLGDDPKEEEKLESHLQNLSTLMAPTYK |
| | KLAPDAYNNQIEYEHRAPECRLGLKEGRPFSGVSACLDFCAHAHRDLHNMQNGSTL |
| | VCTLTREDNREFGGKPEDEQLHVLPLYKVSDVDEFGSVEAQEEKKRSGAIQVLSSF |
| | RRKVRMLAEPVKTCRQRKLEAKKAAAEKLSGGGGSGGGGSGGGGSDEVWSDSEQSF |
| | LDPDIGGVAVAPTHGSILIECAKRELHATTPLKNPNRNHPTRISLVFYQHKSMNEP |
| | KHGLALWEAKMAEKAREKEEECEKYG |
| SEQ ID NO: | MGGSDFPSCRCVEQIIEKDEGPFYTHLGAGPNVAAIREIMEERFGQKGKAIRIERV |
| 12 | IYTGKEGKSSQGCPIAKWVVRRSSSEEKLLCLVRERAGHTCEAAVIVILILVWEGI |
| TET2-CD- | PLSLADKLYSELTETLRKYGTLTNRRCALNEERTCACQGLDPETCGASESFGCSWS |
| T1372S | MYYNGCKFARSKIPRKFKLLGDDPKEEEKLESHLQNLSTLMAPTYKKLAPDAYNNQ |
| | IEYEHRAPECRLGLKEGRPFSGVSACLDFCAHAHRDLHNMQNGSTLVCTLTREDNR |
| | EFGGKPEDEQLHVLPLYKVSDVDEFGSVEAQEEKKRSGAIQVLSSERRKVRMLAEP |
| | VKTCRQRKLEAKKAAAEKLSSLENSSNKNEKEKSAPSRTKQTENASQAKQLAELLR |
| | LSGPVMQQSQQPQPLQKQPPQPQQQQRPQQQQPHHPQTESVNSYSASGSTNPYMRR |
| | PNPVSPYPNSSHTSDIYGSTSPMNFYSTSSQAAGSYLNSSNPMNPYPGLLNQNTQY |
| | PSYQCNGNLSVDNCSPYLGSYSPQSQPMDLYRYPSQDPLSKLSLPPIHTLYQPRFG |
| | NSQSFTSKYLGYGNQNMQGDGFSSCTIRPNVHHVGKLPPYPTHEMDGHEMGATSRL |
| | PPNLSNPNMDYKNGEHHSPSHIIHNYSAAPGMFNSSLHALHLQNKENDMLSHTANG |
| | LSKMLPALNHDRTACVQGGLHKLSDANGQEKQPLALVQGVASGAEDNDEVWSDSEQ |
| | SFLDPDIGGVAVAPTHGSILIECAKRELHATTPLKNPNRNHPTRISLVFYQHKSMN |
| | EPKHGLALWEAKMAEKAREKEEECEKYGPDYVPQKSHGKKVKREPAEPHETSEPTY |
| | LRFIKSLAERTMSVTTDSTVTTSPYAFTRVTGPYNRYI |
| SEQ ID NO: | FSGVTACLD |
| 13 | |
| SEQ ID NO: | FSGVSACLD |
| 14 | |
| TET2 T1372S | |

### III. Kits

Also provided are kits comprising the compositions as described herein. The kits can be useful in performing the methods as described herein. In some embodiments, a kit comprises reagents for capturing a plurality of target regions comprising sequence-variable target regions from the first subsample, thereby providing captured regions and for performing multiplex amplification of segments comprising recombined CDR3 sequences from the second subsample using a plurality of first primers that bind V regions and a plurality of second primers that bind J regions. In some embodiments, a kit comprises reagents for capturing a plurality of target regions comprising sequence-variable target regions from the first subsample, thereby providing captured regions and for performing multiplex amplification of a second plurality of target regions that may comprise a structural variation from the second subsample using a plurality of first primers and a plurality of second primers that anneal to the second plurality of target regions, thereby providing structural variation-enriched DNA. In some embodiments, the kit comprises a reagent for subjecting the sample or treated sample to a procedure that affects a first nucleobase differently from a second nucleobase, wherein the first nucleobase is a modified or unmodified nucleobase, the second nucleobase is a modified or unmodified nucleobase different from the first nucleobase, and the first nucleobase and the second nucleobase have the same base pairing specificity, wherein the procedure alters the base pairing specificity of the first or second nucleobase (e.g., any of the reagents described elsewhere herein for converting a nucleobase such as cytosine or methylated cytosine to a different nucleobase). In some embodiments, the kit comprises one or more conversion reagents. The conversion reagents may comprise reagents for any combination of steps described herein, including but not limited to in the numbered embodiments above and in any one of the workflows shown in the figures. The kit may comprise the first and second reagents and additional elements as discussed below and/or elsewhere herein.

In some embodiments, the kit further comprises a DNA polymerase. In some embodiments, the DNA polymerase of the kit has 5′-3′ exonuclease activity and/or is a strand displacing DNA polymerase. In some embodiments, the DNA polymerase of the kit does not have 5′-3′ exonuclease activity and/or is not a strand displacing DNA polymerase. In some embodiments, the DNA polymerase of the kit is T4 DNA polymerase, T7 DNA polymerase or Klenow fragment. In some embodiments, the DNA polymerase is a uracil-tolerant polymerase (e.g., Q5U® High-Fidelity DNA Polymerase).

In some embodiments, the kit comprises adapters. In some embodiments, the kit comprises PCR primers, wherein the PCR primers anneal to a target region or to an adapter. In some embodiments, the kit comprises additional elements elsewhere herein. In some embodiments, the kit comprises instructions for performing a method described herein.

In some embodiments, the kit further comprises an agent that recognizes methyl cytosine in DNA. In some such embodiments, the agent is an antibody or a methyl binding protein or methyl binding domain. In some embodiments, the kit comprises target-specific probes that specifically bind to sequence-variable target region sets. In some such embodiments, the target-specific probes comprise a capture moiety.

In some embodiments, the kit comprises one or more conversion reagents. The conversion reagents may comprise reagents for any combination of steps described herein, including but not limited to the numbered embodiments above. In some embodiments, the kit further comprises a deaminase. In some embodiments, the deaminase is a methyl-sensitive deaminase (e.g., MsddA or an MsddA-like deaminase) or methyl-insensitive deaminase (e.g., A3A).

In some embodiments, a kit comprises a first reagent for end repair to generate end-repaired DNA, wherein the first reagent comprises at least one type of dNTP that comprises a modified base. In some embodiments, the kit further comprises a second reagent for ligating adapters to the end-repaired DNA to generate adapted DNA, wherein the second reagent also seals nicks present in the end-repaired DNA. In some embodiments, the kit further comprises a third reagent for sequencing that is capable of identifying the base modification in the at least one type of dNTP. The kit may comprise the first, second, and/or third reagents and additional elements as discussed below and/or elsewhere herein. In some embodiments, a kit comprises instructions for performing a method described herein.

In some embodiments, the kit further comprises a plurality of oligonucleotide probes and/or primers for sequencing. In some embodiments, the first reagent of the kit comprises at least one type of dNTP that comprises a modified base selected from a dNTP comprising 5-carboxylcytosine (5-caC), a dNTP comprising 4-methylcytosine (4mC), a dNTP comprising 5-methylcytosine (5mC), a dNTP comprising 5-hydroxymethyl-cytosine (5hmC), a dNTP comprising N6-methyladenosine (6 mA), a dNTP comprising bromodeoxyuridine (BrdU), a dNTP comprising 8-oxoguanine (8oxoG), dUTP, a dNTP comprising fluorodeoxyuridine (FldU), a dNTP comprising iododeoxyuridine (IdU), and/or a dNTP comprising ethynyldeoxyuridine (EdU). In some embodiments, the kit further comprises a reagent for performing an A-tailing reaction. In some embodiments, the reagent for performing the A-tailing reaction comprises a DNA polymerase that does not possess 5′-3′ exonuclease activity and/or is not a strand displacing DNA polymerase, optionally the reagent for performing the A-tailing reaction is HemoKlen Taq. In some embodiments, the reagent for performing the A-tailing reaction comprises a Taq DNA polymerase, Tfl DNA Polymerase, Bst DNA Polymerase, Large Fragment or Tth DNA polymerase. In some embodiments, the reagent for performing the A-tailing reaction comprises a DNA polymerase that does not possess 3′-5′ exonuclease activity, optionally wherein the reagent for performing the A-tailing reaction is Klenow Fragment lacking 3′-5′ exonuclease activity. In some embodiments, the reagent for performing the A-tailing reaction comprises a DNA polymerase that has 5′-3′ exonuclease activity and/or is a strand displacing DNA polymerase.

Kits may further comprise a plurality of oligonucleotide probes that selectively hybridize to least 5, 6, 7, 8, 9, 10, 20, 30, 40 or all genes selected from the group consisting of ALK, APC, BRAF, CDKN2A, EGFR, ERBB2, FBXW7, KRAS, MYC, NOTCHI, NRAS, PIK3CA, PTEN, RBI, TP53, MET, AR, ABL1, AKT1, ATM, CDH1, CSFIR, CTNNBI, ERBB4, EZH2, FGFR1, FGFR2, FGFR3, FLT3, GNA11, GNAQ, GNAS, HNF1A, HRAS, IDH1, IDH2, JAK2, JAK3, KDR, KIT, MLH1, MPL, NPM1, PDGFRA, PROC, PTPN11, RET, SMAD4, SMARCB1, SMO, SRC, STK11, VHL, TERT, CCND1, CDK4, CDKN2B, RAF1, BRCA1, CCND2, CDK6, NF1, TP53, ARID 1 A, BRCA2, CCNE1, ESR1, RIT1, GATA3, MAP2K1, RHEB, ROS1, ARAF, MAP2K2, NFE2L2, RHOA, and NTRKI. The number genes to which the oligonucleotide probes can selectively hybridize can vary. For example, the number of genes can comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, or 54. The kit can include a container that includes the plurality of oligonucleotide probes and instructions for performing any of the methods described herein.

The oligonucleotide probes can selectively hybridize to exon regions of the genes, e.g., of the at least 5 genes. In some cases, the oligonucleotide probes can selectively hybridize to at least 30 exons of the genes, e.g., of the at least 5 genes. In some cases, the multiple probes can selectively hybridize to each of the at least 30 exons. The probes that hybridize to each exon can have sequences that overlap with at least 1 other probe. In some embodiments, the oligoprobes can selectively hybridize to non-coding regions of genes disclosed herein, for example, intronic regions of the genes. The oligoprobes can also selectively hybridize to regions of genes comprising both exonic and intronic regions of the genes disclosed herein.

Any number of exons can be targeted by the oligonucleotide probes. For example, at least 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 400, 500, 600, 700, 800, 900, 1,000, or more, exons can be targeted.

The kit can comprise at least 4, 5, 6, 7, or 8 different library adapters having distinct molecular barcodes and identical sample barcodes. The library adapters may not be sequencing adapters. For example, the library adapters do not include flow cell sequences or sequences that permit the formation of hairpin loops for sequencing. The different variations and combinations of molecular barcodes and sample barcodes are described throughout, and are applicable to the kit. Further, in some cases, the adapters are not sequencing adapters. Additionally, the adapters provided with the kit can also comprise sequencing adapters. A sequencing adapter can comprise a sequence hybridizing to one or more sequencing primers. A sequencing adapter can further comprise a sequence hybridizing to a solid support, e.g., a flow cell sequence. For example, a sequencing adapter can be a flow cell adapter. The sequencing adapters can be attached to one or both ends of a polynucleotide fragment. In some cases, the kit can comprise at least 8 different library adapters having distinct molecular barcodes and identical sample barcodes. The library adapters may not be sequencing adapters. The kit can further include a sequencing adapter having a first sequence that selectively hybridizes to the library adapters and a second sequence that selectively hybridizes to a flow cell sequence. In another example, a sequencing adapter can be hairpin shaped. For example, the hairpin shaped adapter can comprise a complementary double stranded portion and a loop portion, where the double stranded portion can be attached (e.g., ligated) to a double-stranded polynucleotide. Hairpin shaped sequencing adapters can be attached to both ends of a polynucleotide fragment to generate a circular molecule, which can be sequenced multiple times. A sequencing adapter can be up to 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more bases from end to end. The sequencing adapter can comprise 20-30, 20-40, 30-50, 30-60, 40-60, 40-70, 50-60, 50-70, bases from end to end. In a particular example, the sequencing adapter can comprise 20-30 bases from end to end. In another example, the sequencing adapter can comprise 50-60 bases from end to end. A sequencing adapter can comprise one or more barcodes. For example, a sequencing adapter can comprise a sample barcode. The sample barcode can comprise a pre-determined sequence. The sample barcodes can be used to identify the source of the polynucleotides. The sample barcode can be at least 1, 2, 3, 4, 5, 6, 7, 8, 9, 10, 11, 12, 13, 14, 15, 16, 17, 18, 19, 20, 21, 22, 23, 24, 25, or more (or any length as described throughout) nucleic acid bases, e.g., at least 8 bases. The barcode can be contiguous or non-contiguous sequences, as described above.

The library adapters can be blunt ended and Y-shaped and can be less than or equal to 40 nucleic acid bases in length. Other variations of the library adapters can be found throughout and are applicable to the kit.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the disclosure described herein may be employed in practicing the invention. It is therefore contemplated that the disclosure shall also cover any such alternatives, modifications, variations or equivalents. It is intended that the following claims define the scope of the invention and that methods and structures within the scope of these claims and their equivalents be covered thereby.

While the foregoing disclosure has been described in some detail by way of illustration and example for purposes of clarity and understanding, it will be clear to one of ordinary skill in the art from a reading of this disclosure that various changes in form and detail can be made without departing from the true scope of the disclosure and may be practiced within the scope of the appended claims. For example, all the methods, systems, computer readable media, and/or component features, steps, elements, or other aspects thereof can be used in various combinations.

All patents, patent applications, websites, other publications or documents, accession numbers and the like cited herein are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number, if applicable. Likewise, if different versions of a publication, website or the like are published at different times, the version most recently published at the effective filing date of the application is meant, unless otherwise indicated.

### EXAMPLES

Exemplary workflows for analyzing the modified nucleoside profile of nucleic acid in a sample and library preparation are provided herein. In some embodiments, some or all features of the partitioning and library preparation workflows may be used in combination.

Exemplary workflows are illustrated in FIGS. 1A and 1B.

The workflow of FIG. 1A comprises beginning with DNA isolated from a sample and ligated to adapters for sequencing library preparation and then amplified to provide an amplified DNA library. The amplified DNA library in turn is partitioned into at least a first subsample comprising sequence-variable target regions, a second subsample comprising recombined CDR3 sequences, and an optional third subsample retained as a backup. Sequence-variable target regions are captured and amplified and recombined CDR3 sequences are amplified through multiplex PCR. The captured regions and the CDR3-enriched DNA are then pooled and sequenced together. Alternatively, as shown in FIG. 1B, the captured regions and the CDR3-enriched DNA can be sequenced separately.

What is claimed is:

1. A method of analyzing DNA in an amplified, adapted library, the method comprising:
a) partitioning the amplified, adapted library into at least a first subsample and a second subsample;
b) capturing a plurality of target regions comprising sequence-variable target regions from the first subsample, thereby providing captured regions;
c) performing multiplex amplification of segments comprising recombined CDR3 sequences from the second subsample using a plurality of first primers that bind V regions and a plurality of second primers that bind J regions, thereby providing CDR3-enriched DNA; and
d) sequencing the captured regions and the CDR3-enriched DNA.

2. The method of claim 1, wherein the plurality of first primers comprises from 10 to 150, from 15 to 125, from 20 to 100, from 30 to 90, from 40 to 80, or from 50 to 70 primers that bind V regions.

3. The method of any one of the preceding claims, wherein the plurality of second primers comprises from 5 to 30, from 10 to 25, or from 10 to 20 primers that bind J regions.

4. The method of any one of the preceding claims, wherein the captured regions and the CDR3-enriched DNA are pooled and sequenced together.

5. The method of any one of claims 1 to 4, wherein the captured regions and the CDR3-enriched DNA are sequenced separately.

6. The method of any one of the preceding claims, comprising detecting a presence or absence of a DNA molecule that comprises a structural variation from the first subsample.

7. A method of analyzing DNA in an amplified, adapted library, the method comprising:
a) partitioning the amplified, adapted library into at least a first subsample and a second subsample;
b) capturing a first plurality of target regions comprising sequence-variable target regions from the first subsample, thereby providing captured regions;
c) performing multiplex amplification of a second plurality of target regions that may comprise a structural variation from the second subsample using a plurality of first primers and a plurality of second primers that anneal to the second plurality of target regions, thereby providing structural variation-enriched DNA; and
d) sequencing the captured regions and the structural variation-enriched DNA.

8. The method of claim 6, wherein the captured regions and the structural variation-enriched DNA are pooled and sequenced together.

9. The method of claim 6, wherein the captured regions and the structural variation-enriched DNA are sequenced separately.

10. The method of any one of claims 7 to 9, wherein at least a portion of the plurality of first primers and the plurality of second primers do not exponentially amplify a target region that does not comprise a structural variation.

11. The method of any of claims 6 to 10, wherein the structural variation comprises a rearrangement, an insertion, or a deletion.

12. The method of the immediately preceding claim, wherein the rearrangement comprises translocations, gene fusions, duplications, copy-number variants, or inversions.

13. The method of any one of the preceding claims, wherein a third subsample of the amplified, adapted library is retained as a backup.

14. The method of any one of the preceding claims, wherein the method comprises preparing the amplified, adapted library by ligating adaptors to DNA, thereby producing adapted DNA, followed by amplifying the adapted DNA.

15. The method of claim 14, wherein the adapted DNA comprises molecular barcodes.

16. The method of any one of the preceding claims, wherein the amplified, adapted library is prepared from cfDNA.

17. The method of claim 16, wherein the method comprises preparing the amplified, adapted library by ligating adaptors to cfDNA, thereby producing adapted cfDNA, followed by amplifying the adapted cfDNA.

18. The method of any one of the preceding claims, wherein the captured regions are amplified prior to sequencing.

19. The method of any one of the preceding claims, wherein the amplified, adapted library is prepared from DNA from a subject having or suspected of having a cancer.

20. The method of any one of the preceding claims, further comprising determining a likelihood that the subject has a cancer.

21. The method of any one of claims 19 to 20, wherein the cancer is a lymphocytic cancer.

22. The method of the immediately preceding claim, wherein the lymphocytic cancer is a leukemia, a lymphoma, or a myeloma.

23. The method of any one of claims 19 to 20, wherein the cancer is a lymphoma.

24. The method of claim 23, wherein the lymphoma is B-cell lymphoma, non-Hodgkin lymphoma, diffuse large B-cell lymphoma, Mantle cell lymphoma, T cell lymphoma, non-Hodgkin lymphoma, precursor T-lymphoblastic lymphoma/leukemia, or peripheral T cell lymphoma.

25. The method of any one of the preceding claims, wherein the CDR3 is a part of a T cell receptor (TCR), TCR beta chain, B cell receptor, immunoglobulin, B cell receptor heavy chain, or immunoglobulin heavy chain.

26. A method of analyzing DNA in an amplified, adapted library, the method comprising:
a) preparing the amplified, adapted library by ligating adaptors to cfDNA from a subject having or suspected of having a cancer, thereby producing adapted cfDNA, followed by amplifying the adapted cfDNA;
b) partitioning the amplified, adapted library into at least first and second subsamples;
c) capturing a plurality of target regions comprising sequence-variable target regions from the first subsample, thereby providing captured regions;
d) performing multiplex amplification of segments comprising recombined CDR3 sequences from the second subsample using a plurality of first primers that bind V regions and a plurality of second primers that bind J regions, thereby providing CDR3-enriched DNA; and
e) sequencing the captured regions and the CDR3-enriched DNA.

27. The method of claim 26, wherein the captured regions and the CDR3-enriched DNA are pooled and sequenced together.

28. The method of claim 26, wherein the captured regions and the CDR3-enriched DNA are sequenced separately.

29. The method of any one of the preceding claims, further comprising capturing epigenetic target regions from the DNA of the adapted, amplified DNA library and amplifying and sequencing the epigenetic target regions.

30. The method of the immediately preceding claim, wherein the epigenetic target regions comprise hypermethylation variable target regions, hypomethylation variable target regions, methylation control target regions, or fragmentation variable target regions.

31. The method of any one of the preceding claims, further comprising quantifying a somatic mutation load using a plurality of captured regions comprising the sequence-variable target regions.

32. The method of any one of the preceding claims, wherein each of the plurality of the first and second primers comprises at least 20 linked nucleosides.

33. The method of any one of the preceding claims, wherein each of the plurality of the first and second primers consists of 20 to 60 linked nucleosides.

34. The method of any of the preceding claims, wherein the plurality of the first and second primers are resistant to 5′ exonucleolysis.

35. The method of any of the preceding claims, comprising differentially tagging and pooling the first subsample and second subsample.

36. The method of claim 34, wherein the pool comprises less than or equal to about 45%, 40%, 35%, 30%, 25%, 20%, 15%, 10%, or 5% of the DNA of the second subsample.

37. The method of the immediately preceding claim, wherein the pool comprises about 70-90%, about 75-85%, or about 80% of the DNA of the second subsample.

38. The method of claim 34, wherein the pool comprises substantially all of the DNA of the first subsample.

39. The method of any one of the preceding claims, wherein the detecting comprises generating a plurality of sequencing reads; and the method further comprises mapping the plurality of sequence reads to one or more reference sequences to generate mapped sequence reads, and processing the mapped sequence reads to determine the likelihood that the subject has cancer.

40. The method of the immediately preceding claim, further comprising detecting a presence or absence of DNA originating or derived from a tumor cell using the mapped sequence reads.

41. The method of the immediately preceding claim, further comprising determining a cancer recurrence score that is indicative of the presence or absence of the DNA originating or derived from the tumor cell for the test subject, optionally further comprising determining a cancer recurrence status based on the cancer recurrence score, wherein the cancer recurrence status of the test subject is determined to be at risk for cancer recurrence when a cancer recurrence score is determined to be at or above a predetermined threshold or the cancer recurrence status of the test subject is determined to be at lower risk for cancer recurrence when the cancer recurrence score is below the predetermined threshold.

42. The method of the immediately preceding claim, further comprising comparing the cancer recurrence score of the test subject with a predetermined cancer recurrence threshold, wherein the test subject is classified as a candidate for a subsequent cancer treatment when the cancer recurrence score is above the cancer recurrence threshold or not a candidate for a subsequent cancer treatment when the cancer recurrence score is below the cancer recurrence threshold.
